(19) United States
(12) Patent Application Publication
(10) Pub. No.: US 2024/0315994 A1
(43) Pub. Date: Sep. 26, 2024

(54) SMALL MOLECULE MODULATORS OF FERROPTOSIS

(71) Applicant: ARPEGGIO BIOSCIENCES, INC., Boulder, CO (US)

(72) Inventors: Timothy READ, Boulder, CO (US); Zoe WINIGRAD, Seattle, DC (US); Ardeshir GOLIAEI, Boston, MA (US); Joseph AZOFEIFA, Boulder, CO (US); John K. DICKSON, Boulder, CO (US); Jason R. HARRIS, Boulder, CO (US); Eric MARTIN, Boulder, CO (US)

(21) Appl. No.: 18/441,446

(22) Filed: Feb. 14, 2024

Related U.S. Application Data
(63) Continuation of application No. PCT/US2022/040818, filed on Aug. 18, 2022.
(60) Provisional application No. 63/234,829, filed on Aug. 19, 2021.
(60) Provisional application No. 63/286,022, filed on Dec. 4, 2021.
(60) Provisional application No. 63/295,007, filed on Dec. 30, 2021.
(60) Provisional application No. 63/313,000, filed on Feb. 23, 2022.

(51) Int. Cl. A61K 31/167; A61K 31/16; A61K 31/192; A61K 31/277; A61K 31/341; A61K 31/351; A61K 31/404; A61K 31/415; A61K 31/416; A61K 31/4192; A61K 31/425; A61K 31/426; A61K 31/428; A61K 31/433; A61K 31/435; A61K 31/4355; A61K 31/437; A61K 31/4375; A61K 31/44; A61K 31/4439; A61K 31/47; A61K 31/472; A61K 31/498; A61K 31/4985; A61K 31/505; A61K 31/513; A61K 31/517; A61K 31/519; A61K 31/538; A61K 31/5415; C07C 233/06; C07D 217/18; C07D 217/20; C07D 239/545; C07D 265/36; C07D 279/16; C07D 471/04; C07D 487/04

(52) U.S. Cl. CPC A61K 31/167; A61K 31/16; A61K 31/192; A61K 31/277; A61K 31/341; A61K 31/351; A61K 31/404; A61K 31/415; A61K 31/416; A61K 31/4192; A61K 31/425; A61K 31/426; A61K 31/428; A61K 31/433; A61K 31/435; A61K 31/4355; A61K 31/437; A61K 31/4375; A61K 31/44; A61K 31/4439; A61K 31/47; A61K 31/472; A61K 31/498; A61K 31/4985; A61K 31/505; A61K 31/513; A61K 31/517; A61K 31/519; A61K 31/538; A61K 31/5415; C07C 233/06; C07D 217/18; C07D 217/20; C07D 239/545; C07D 265/36; C07D 279/16; C07D 471/04; C07D 487/04

(57) ABSTRACT

Provided herein are inhibitors of GPX4, pharmaceutical compositions comprising the inhibitory compounds, and methods for using the GPX4 inhibitory compounds for the modulation of ferroptosis and treatment of disease.

### CROSS REFERENCE

This application is a continuation application of International Application No. PCT/US2022/040818, filed Aug. 18, 2022, which claims the benefit of U.S. Provisional Application No. 63/313,000, filed Feb. 23, 2022; U.S. Provisional Application No. 63/295,007, filed Dec. 30, 2021; U.S. Provisional Application No. 63/286,022, filed Dec. 4, 2021; and U.S. Provisional Application No. 63/234,829, filed Aug. 19, 2021; each of which is incorporated by reference in its entirety.

### FIELD OF THE INVENTION

The invention relates to compounds, compositions, and methods for inducing ferroptosis in a cell.

### BACKGROUND OF THE INVENTION

Efforts to inhibit GPX4 have focused on the use of electrophiles capable of forming covalent bonds with a selenocysteine residue in the GPX4 active site. Despite its clear biological role in protecting cancer cells from ferroptosis, to date, no targeted GPX4 inhibitors have been profiled in the clinic. There is a need in the art for novel modulators of GPX4.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

FIG. 1 is a plot showing the relative viability of HT-1080 fibrosarcoma cells exposed to each compound in the inhibitor screening panel.

FIG. 2A, FIG. 2B show the relative viability of HT-1080 fibrosarcoma cells exposed to inhibitor compounds in the presence and absence of 1 μM Liproxstatin-1.

FIG. 3 is a panel of plots showing the relative viability of HT-1080 fibrosarcoma cells exposed to inhibitor compounds in the presence and absence of 1.5 μM ferrostatin-1.

FIG. 4A is a panel of plots showing fluorescence images of Biodipy-C11 staining of cells treated with the five ferroptosis-inducing compounds identified in FIG. 3.

FIG. 4B is a panel of plots showing fluorescence images of Biodipy-C11 staining of cells treated with compound 8216 and the controls RSL3 and sodium arsenite.

FIG. 5A is a plot showing the mass spectrum for positive control ML162 binding to recombinant GPX4 in vitro.

FIG. 5B shows the mass spectrum for the negative control, 0568, binding to recombinant GPX4 in vitro.

FIG. 5C shows the mass spectrum for the negative control, 2604, binding to recombinant GPX4 in vitro.

FIG. 6A is a plot showing the mass spectrums for the ferroptosis-inducing compound, 1962, binding to recombinant GPX4 in vitro.

FIG. 6B is a plot showing the mass spectrums for the ferroptosis-inducing compound, 1816, binding to recombinant GPX4 in vitro.

FIG. 6C is a plot showing the mass spectrums for the ferroptosis-inducing compound, 3362, binding to recombinant GPX4 in vitro.

FIG. 6D is a plot showing the mass spectrums for the ferroptosis-inducing compound, 0973, binding to recombinant GPX4 in vitro.

FIG. 6E is a plot showing the mass spectrums for the ferroptosis-inducing compound, 8216, binding to recombinant GPX4 in vitro.

FIG. 7 is a plot showing relative GPX4 activity in vitro for the five ferroptosis-inducing compounds 1816, 8216, 3362, 1962, and 0973.

FIG. 8 is a plot showing the relative viability of HK-2 renal epithelial cells exposed to the ferroptosis-inducing compounds 8216 or 1962 over a range of concentrations.

FIG. 9 is a panel of plots showing relative viability of HK-2 kidney epithelial cells exposed to the inhibitor compounds 8216 or 1962 over a range of concentrations in the presence and absence of ferrostatin-1.

FIG. 10A is a plot showing transcriptional activity in IMR90 lung fibroblast cells exposed to RSL3.

FIG. 10B is a plot showing transcriptional activity in IMR90 lung fibroblast cells exposed to ML162.

FIG. 10C is a plot showing transcriptional activity in IMR90 lung fibroblast cells exposed to erastin.

FIG. 11 is a plot showing fold HMOX1 induction in HT-1080 fibrosarcoma cells exposed to compounds RSL3, 8216, 1962, 1816, or 3362.

FIG. 12 is a plot showing relative viability of HT-1080 fibrosarcoma cells exposed to different 8216 derivative compounds.

FIG. 13 is a plot showing relative viability of HT-1080 fibrosarcoma cells exposed to compounds with similar structures to compounds 6666 or 1816.

FIG. 14A, FIG. 14B and FIG. 14C illustrate a panel of plots 1400 showing relative viability of HT-1080 fibrosarcoma cells exposed to 15 inhibitor compounds with similar structures to 8216, 6666, or 1816 tested at 8 concentrations from 50 μM to 5 nM at half log dilutions in the presence of (red line) and absence of (black line) 1.5 UM ferrostatin-1. Ferrostatin-1 is a lipophilic antioxidant which inhibits the process of ferroptosis and rescues cells from undergoing this form of cell death. HT-1080 fibrosarcoma cells were plated on side-blackened, clear bottom plates 24 hours prior to treatment, with or without the addition of 2× ferrostatin-1. Each compound was diluted in DMSO and media and then added to the cells for a total of 24 hours, at which point cell viability was read out via Cell Titer Glo. Several compounds show a clear shift in EC50 with the addition of ferrostatin-1 (red line shifted to the right) which is evidence to suggest that those compounds induce cell death via ferroptosis.

FIG. 15 is a plot showing fold HMOX1 induction in HT-1080 fibrosarcoma cells exposed to compounds 8147, 6047, 3793, Rsl3, 6666 or 8216.

FIG. 16 is a panel of plots showing percent activity of GPX4 in response to exposure to compounds 8216, 6666, 8147, or 3793.

FIG. 17 shows a panel of representative images from fluorescent confocal microscopy of HT-1080 fibrosarcoma cells treated with compounds 8147, 8216, 3973, 6047, or 6666 in the presence or absence of 1.5 μM ferrostatin-1.

FIG. 18 is a panel of plots showing the intact protein mass spectrometry analyses of compounds 8216, 6666, and 8147.

### DETAILED DESCRIPTION OF THE INVENTION

### Terminology

### Compound Terminology

“Isomer” means molecules with identical molecular formulas, that is the same number of atoms of each element, but distinct arrangements of atoms in space.

“Covalent inhibitor” means compounds that by design are intended to form a covalent bond with a specific molecular target.

### Composition Terminology

“Pharmaceutically acceptable” means on balance, safe for use in humans or animals, without undue side effects.

“Pharmaceutically acceptable salt” means a salt of the compounds of the present invention which is pharmaceutically acceptable and which possess the desired pharmacological activity. Such salts include, for example, acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

### Cell and Cell Death Terminology

“Ferroptosis” means regulated cell death that is iron-dependent. Ferroptosis is characterized by the iron-dependent accumulation of lethal lipid reactive oxygen species.

“GPX4” means the glutathione peroxidase 4, a glutathione metabolism enzyme.

“In vitro” means an artificial environment created outside a living multicellular organisms (e.g., a test tube or culture plate) used in experimental research to study a disease or process. As used herein, “in vitro” includes processes performed in intact cells growing in culture.

“In vivo” means that which takes place inside an organism and more specifically to a process performed in or on the living tissue of a whole, living multicellular organism (animal), such as a mammal, as opposed to a partial or dead one.

“Ex vivo” refers to a process performed in an artificial environment outside the organism on living cells or tissue which are removed from an organism and subsequently returned to an organism.

“Mesenchymal tumor” or “mesenchymal cancer” refers to tumors that arise from mesenchymal tissue or tumors that have undergone epithelial to mesenchymal transition. The epithelial-mesenchymal transition (EMT) is a process by which epithelial cells lose their cell polarity and cell-cell adhesion and gain migratory and invasive properties to become mesenchymal stem cells. EMT has also been shown to occur in the initiation of metastasis in cancer progression.

“Mesenchymal” refers to cells that develop into connective tissue, blood vessels, and lymphatic tissue.

### Small Molecule Inducers of Ferroptosis

The invention provides small molecule inducers of ferroptosis. In various embodiments, the invention provides compounds that target the active site of the GPX4 enzyme, wherein binding of the compound to the active site of GPX4 effectively inhibits the activity of the enzyme.

In one embodiment, the invention provides a composition comprising a compound of the invention or a pharmaceutically acceptable salt thereof and a pharmaceutically acceptable carrier, adjuvant, or vehicle.

In one embodiment, the invention provides a method for inducing ferroptosis in a cell, the method comprising contacting the cell with an effective amount of a compound of the invention or a pharmaceutically acceptable salt thereof.

In one embodiment, the invention provides a method for decreasing GPX4 activity in a cell, the method comprising contacting the cell with an effective amount of a compound of the invention or a pharmaceutically acceptable salt thereof.

The compounds of the invention are useful for inducing ferroptosis in a cell. In one embodiment, the compounds of the invention may be used in cancer therapy to induce ferroptosis in a cancer cell, such as a mesenchymal cancer cell.

### Compounds

In various embodiments, the invention provides compounds that target the active site of the GPX4 enzyme, wherein binding of the compound to the active site of GPX4 effectively inhibits the activity of the enzyme.

In one embodiment, compounds of the invention, or pharmaceutically acceptable salts thereof, have the structure indicated by Formula 1:

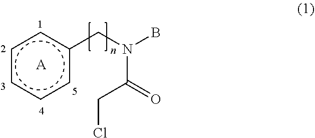

where n is 0 or 1; and
where B is selected from:

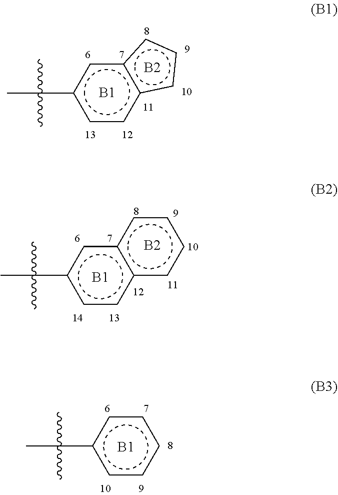

where each ring individually may be aliphatic or aromatic;
where each ring individually may substituted or unsubstituted;
where when n is 0 and B is B₁:
   there are no ring substituents or ring heteroatoms; or there are ring heteroatoms comprising [A:1,5 and/or (B1:6 or B1:13 or B1:6,13) and/or (B2:8,9 or B2:10 or B2:11,12) is a N]; and/or B2:8 is a S; and/or (B2:8 or B2:10 or B2:13 is an O); and/or
   if substituted, substituents on rings A and/or B₁ and/or B₂ are as follows:
      one or more F, Br, Cl, CN, Me, OMe at any position on the ring;
where when n is 0 and B is B₂:
   there are no ring substituents or ring heteroatoms; or there are ring heteroatoms comprising (B1:6 or B1:6,13 or B1:6,14) and/or B2:8 is an N; and/or
   if substituted, substituents on rings A and/or B₁ and/or B₂ are as follows:
      one or more F, Br, Cl, CN, Me at any position on the ring;
where when n is 1 and B is B₁:
   there are no ring substituents or ring heteroatoms; or
   there are ring heteroatoms comprising [(B1:6 or B1:13 or B1:6,13) and/or (B2:7,9 or B2:7,10 or B2:7,9,10 or B2:7,8,10 or B2:7,8,9,10 or B2:8 or B2:8,9 or B2:8,9,10 or B2:8,11 or B2:8,10,11 or B2:8,9,10,11 or B2:9,11 or B2:10 or B2:10,11) is an N]; and/or (B2:8 or B2:10 or B2:13 is an O); and/or B2:8 is an S; and/or
   if substituted, substituents on rings A and/or B₁ and/or B₂ are as follows:
      one or more F, Br, Cl, CN, Me at any position on the ring;
where when n is 1 and B is B₂:
   there are no ring substituents or ring heteroatoms; or
   there are ring heteroatoms comprising [(B1:6 or B1:13 or B1:6,13 or B1:6,14) and/or (B2:8 or B2:9,11 or B2:11) is an N]; and/or
   if substituted, substituents on rings A and/or B₁ and/or B₂ are as follows:
      one or more F, Br, Cl, CN, Me at any position on the ring;
where when n is 1 and B is B₃:
   there are no ring substituents or ring heteroatoms; or
   there are ring heteroatoms comprising (B1:6; or B1:6,8 or B1:6,10 or B1:7 or B1:8 is a N); and/or B1:8 is an O; and/or
   if substituted, substituents on rings A and/or B1 include:
      one or more F, Br, Cl, CN, Me, OMe, trihalomethane, OH at any position on the ring.

In one embodiment, compounds of the invention, or pharmaceutically acceptable salts thereof, have the structure of a compound provided in Table 1.

TABLE 1

| Working | | | MS Data |
|---|---|---|---|
| Example | Structure | Name | (M + H) |
| A1 | 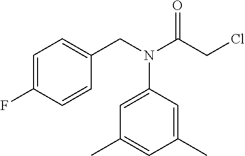 [SMILES: Cc1cc(C)cc(N(Cc2ccc(F)cc2)C(=O)CCl)c1] | 2-chloro-N-(3,5-dimethylphenyl)-N- [(4-fluorophenyl)methyl]acetamide | 306.1 |
| A2 | 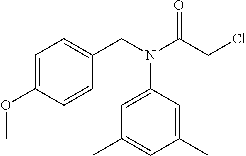 [SMILES: COc1ccc(CN(C(=O)CCl)c2cc(C)cc(C)c2)cc1] | 2-chloro-N-(3,5-dimethylphenyl)-N- [(4-methoxyphenyl)methyl]acetamide | 318.1 |
| A3 | 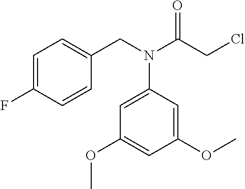 [SMILES: COc1cc(OC)cc(N(Cc2ccc(F)cc2)C(=O)CCl)c1] | 2-chloro-N-(3,5-dimethoxyphenyl)-N- [(4-fluorophenyl)methyl]acetamide | 338.0 |
| A4 | 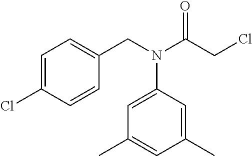 [SMILES: Cc1cc(C)cc(N(Cc2ccc(Cl)cc2)C(=O)CCl)c1] | 2-chloro-N-[(4-chlorophenyl)methyl]- N-(3,5-dimethylphenyl)acetamide | 332.0 |
| A5 | 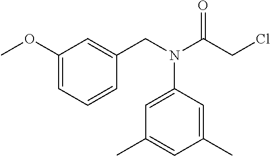 [SMILES: COc1cccc(CN(C(=O)CCl)c2cc(C)cc(C)c2)c1] | 2-chloro-N-(3,5-dimethylphenyl)-N- [(3-methoxyphenyl)methyl]acetamide | 318.0 |
| A6 | 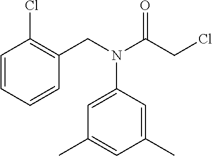 [SMILES: Cc1cc(C)cc(N(Cc2ccccc2Cl)C(=O)CCl)c1] | 2-chloro-N-[(2-chlorophenyl)methyl]- N-(3,5-dimethylphenyl)acetamide | 322.0 |
| A7 | 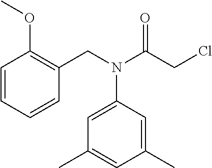 [SMILES: COc1ccccc1CN(C(=O)CCl)c1cc(C)cc(C)c1] | 2-chloro-N-(3,5-dimethylphenyl)-N- [(2-methoxyphenyl)methyl]acetamide | 318.1 |
| A8 | 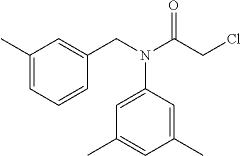 [SMILES: Cc1cccc(CN(C(=O)CCl)c2cc(C)cc(C)c2)c1] | 2-chloro-N-(3,5-dimethylphenyl)-N- (m-tolylmethyl)acetamide | 302.1 |
| A9 | 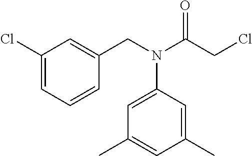 [SMILES: Cc1cc(C)cc(N(Cc2cccc(Cl)c2)C(=O)CCl)c1] | 2-chloro-N-[(3-chlorophenyl)methyl]- N-(3,5-dimethylphenyl)acetamide | 332.0 |
| A10 | 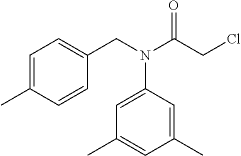 [SMILES: Cc1ccc(CN(C(=O)CCl)c2cc(C)cc(C)c2)cc1] | 2-chloro-N-(3,5-dimethylphenyl)-N-(p- tolylmethyl)acetamide | 302.1 |
| A11 | 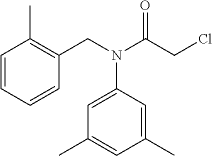 [SMILES: Cc1cc(C)cc(N(Cc2ccccc2C)C(=O)CCl)c1] | 2-chloro-N-(3,5-dimethylphenyl)-N-(o- tolylmethyl)acetamide | 302.1 |
| A12 | 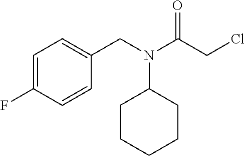 [SMILES: O=C(CCl)N(Cc1ccc(F)cc1)C1CCCCC1] | 2-chloro-N-cyclohexyl-N-[(4- fluorophenyl)methyl]acetamide | 284.1 |
| A13 | 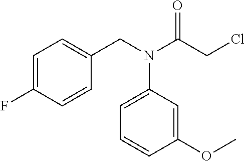 [SMILES: COc1cccc(N(Cc2ccc(F)cc2)C(=O)CCl)c1] | 2-chloro-N-[(4-fluorophenyl)methyl]- N-(3-methoxyphenyl)acetamide | 308.0 |
| A14 | 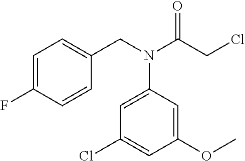 [SMILES: COc1cc(Cl)cc(N(Cc2ccc(F)cc2)C(=O)CCl)c1] | 2-chloro-N-(3-chloro-5-methoxy- phenyl)-N-[(4- fluorophenyl)methyl]acetamide | 342.0 |
| A15 | 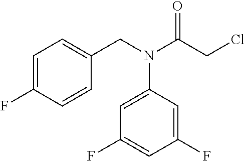 [SMILES: O=C(CCl)N(Cc1ccc(F)cc1)c1cc(F)cc(F)c1] | 2-chloro-N-(3,5-difluorophenyl)-N-[(4- fluorophenyl)methyl]acetamide | 314.0 |
| A16 | 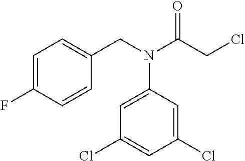 [SMILES: O=C(CCl)N(Cc1ccc(F)cc1)c1cc(Cl)cc(Cl)c1] | 2-chloro-N-(3,5-dichlorophenyl)-N-[(4- fluorophenyl)methyl]acetamide | 345.9 |
| A17 | 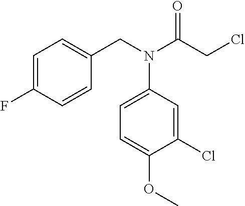 [SMILES: COc1ccc(N(Cc2ccc(F)cc2)C(=O)CCl)cc1Cl] | 2-chloro-N-(3-chloro-4-methoxy- phenyl)-N-[(4- fluorophenyl)methyl]acetamide | 342.0 |
| A18 | 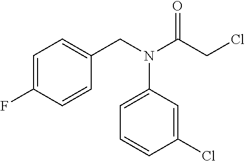 [SMILES: O=C(CCl)N(Cc1ccc(F)cc1)c1cccc(Cl)c1] | 2-chloro-N-(3-chlorophenyl)-N-[(4- fluorophenyl)methyl]acetamide | 312.0 |
| A19 | 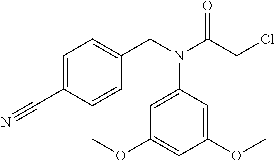 [SMILES: COc1cc(OC)cc(N(Cc2ccc(C#N)cc2)C(=O)CCl)c1] | 2-chloro-N-[(4-cyanophenyl)methyl]- N-(3,5-dimethoxyphenyl)acetamide | 345.1 |
| A20 | 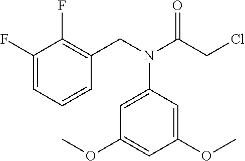 [SMILES: COc1cc(OC)cc(N(Cc2cccc(F)c2F)C(=O)CCl)c1] | 2-chloro-N-[(2,3- difluorophenyl)methyl]-N-(3,5- dimethoxyphenyl)acetamide | 356.0 |
| A21 | 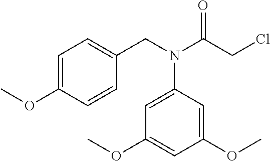 | 2-chloro-N-(3,5-dimethoxyphenyl)-N- [(4-methoxyphenyl)methyl]acetamide | 350.1 |
| A22 | 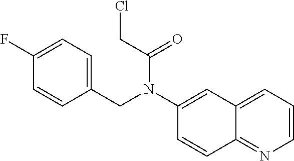 [SMILES: O=C(CCl)N(Cc1ccc(F)cc1)c1ccc2ncccc2c1] | 2-chloro-N-[(4-fluorophenyl)methyl]- N-(6-quinolyl)acetamide | 329.1 |
| A23 | 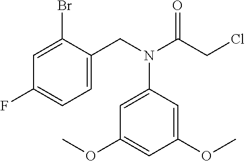 [SMILES: COc1cc(OC)cc(N(Cc2ccc(F)cc2Br)C(=O)CCl)c1] | N-[(2-bromo-4-fluoro-phenyl)methyl]- 2-chloro-N-(3,5- dimethoxyphenyl)acetamide | 416.0 |
| A24 | 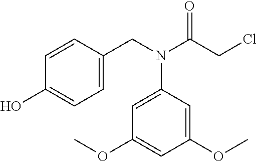 [SMILES: COc1cc(OC)cc(N(Cc2ccc(O)cc2)C(=O)CCl)c1] | 2-chloro-N-(3,5-dimethoxyphenyl)-N- [(4-hydroxyphenyl)methyl]acetamide | 336.1 |
| A25 | 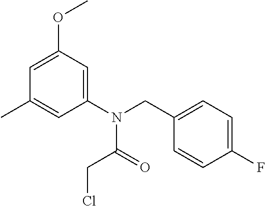 [SMILES: COc1cc(C)cc(N(Cc2ccc(F)cc2)C(=O)CCl)c1] | 2-chloro-N-[(4-fluorophenyl)methyl]- N-(3-methoxy-5-methyl- phenyl)acetamide | 322.1 |
| A26 | 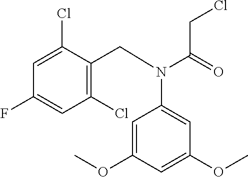 [SMILES: COc1cc(OC)cc(N(Cc2c(Cl)cc(F)cc2Cl)C(=O)CCl)c1] | 2-chloro-N-[(2,6-dichloro-4-fluoro- phenyl)methyl]-N-(3,5- dimethoxyphenyl)acetamide | 406.0 |
| A27 | 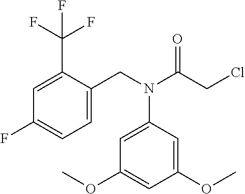 [SMILES: COc1cc(OC)cc(N(Cc2ccc(F)cc2C(F)(F)F)C(=O)CCl)c1] | 2-chloro-N-(3,5-dimethoxyphenyl)-N- [[4-fluoro-2-(trifluoro- methyl)phenyl]methyl]acetamide | 406.0 |
| A28 | 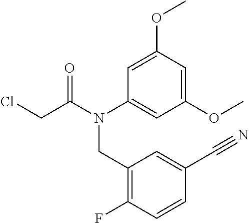 [SMILES: COc1cc(OC)cc(N(Cc2cc(C#N)ccc2F)C(=O)CCl)c1] | 2-chloro-N-[(5-cyano-2-fluoro- phenyl)methyl]-N-(3,5- dimethoxyphenyl)acetamide | 363.1 |
| A29 | 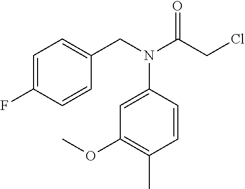 [SMILES: COc1cc(N(Cc2ccc(F)cc2)C(=O)CCl)ccc1C] | 2-chloro-N-[(4-fluorophenyl)methyl]- N-(3-methoxy-4-methyl- phenyl)acetamide | 322.1 |
| A30 | 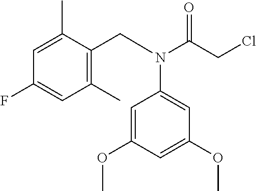 [SMILES: COc1cc(OC)cc(N(Cc2c(C)cc(F)cc2C)C(=O)CCl)c1] | 2-chloro-N-(3,5-dimethoxyphenyl)-N- [(4-fluoro-2,6-dimethyl- phenyl)methyl]acetamide | 366.1 |
| A31 | 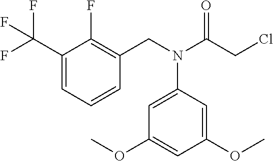 [SMILES: COc1cc(OC)cc(N(Cc2cccc(C(F)(F)F)c2F)C(=O)CCl)c1] | 2-chloro-N-(3,5-dimethoxyphenyl)-N- [[2-fluoro-3-(trifluoro- methyl)phenyl]methyl]acetamide | 406.1 |
| A32 | 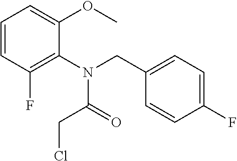 [SMILES: COc1cccc(F)c1N(Cc1ccc(F)cc1)C(=O)CCl] | 2-chloro-N-(2-fluoro-6-methoxy- phenyl)-N-[(4- fluorophenyl)methyl]acetamide | 326.1 |
| A33 | 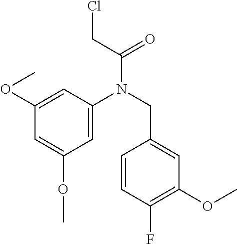 [SMILES: COc1cc(OC)cc(N(Cc2ccc(F)c(OC)c2)C(=O)CCl)c1] | 2-chloro-N-(3,5-dimethoxyphenyl)-N- [(4-fluoro-3-methoxy- phenyl)methyl]acetamide | 368.1 |
| A34 | 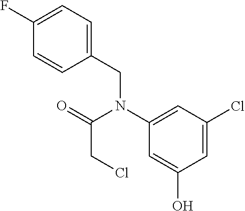 [SMILES: O=C(CCl)N(Cc1ccc(F)cc1)c1cc(O)cc(Cl)c1] | 2-chloro-N-(3-chloro-5-hydroxy- phenyl)-N-[(4- fluorophenyl)methyl]acetamide | 328.1 |
| A35 | 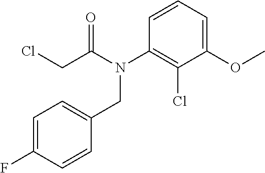 [SMILES: COc1cccc(N(Cc2ccc(F)cc2)C(=O)CCl)c1Cl] | 2-chloro-N-(2-chloro-3-methoxy- phenyl)-N-[(4- fluorophenyl)methyl]acetamide | 342.0 |
| A36 | 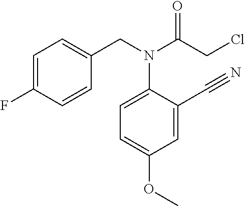 [SMILES: COc1ccc(N(Cc2ccc(F)cc2)C(=O)CCl)c(C#N)c1] | 2-chloro-N-(2-cyano-4-methoxy- phenyl)-N-[(4- fluorophenyl)methyl]acetamide | 333.1 |
| A37 | 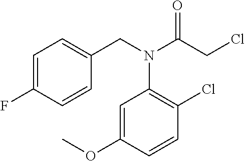 [SMILES: COc1ccc(Cl)c(N(Cc2ccc(F)cc2)C(=O)CCl)c1] | 2-chloro-N-(2-chloro-5-methoxy- phenyl)-N-[(4- fluorophenyl)methyl]acetamide | 342.0 |
| A38 | 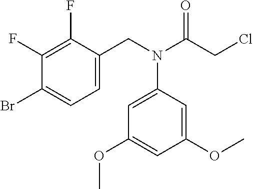 [SMILES: COc1cc(OC)cc(N(Cc2ccc(Br)c(F)c2F)C(=O)CCl)c1] | N-[(4-bromo-2,3-difluoro- phenyl)methyl]-2-chloro-N-(3,5- dimethoxyphenyl)acetamide | 434.0 |
| A39 | 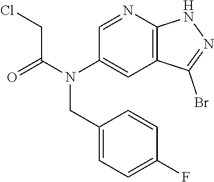 [SMILES: O=C(CCl)N(Cc1ccc(F)cc1)c1cnc2[nH]nc(Br)c2c1] | N-(3-bromo-1H-pyrazolo[3,4- b]pyridin-5-yl)-2-chloro-N-[(4- fluorophenyl)methyl]acetamide | 397.0 |
| A40 | 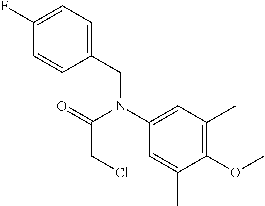 [SMILES: COc1c(C)cc(N(Cc2ccc(F)cc2)C(=O)CCl)cc1C] | 2-chloro-N-[(4-fluorophenyl)methyl]- N-(4-methoxy-3,5-dimethyl- phenyl)acetamide | 336.2 |
| A41 | 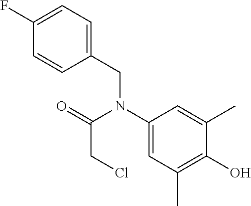 [SMILES: Cc1cc(N(Cc2ccc(F)cc2)C(=O)CCl)cc(C)c1O] | 2-chloro-N-[(4-fluorophenyl)methyl]- N-(4-hydroxy-3,5-dimethyl- phenyl)acetamide | 322.1 |
| A42 | 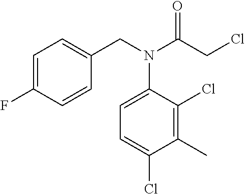 [SMILES: Cc1c(Cl)ccc(N(Cc2ccc(F)cc2)C(=O)CCl)c1Cl] | 2-chloro-N-(2,4-dichloro-3-methyl- phenyl)-N-[(4- fluorophenyl)methyl]acetamide | 360.0 |
| A43 | 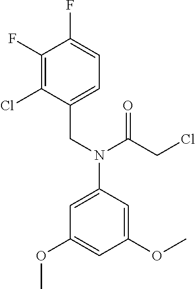 [SMILES: COc1cc(OC)cc(N(Cc2ccc(F)c(F)c2Cl)C(=O)CCl)c1] | 2-chloro-N-[(2-chloro-3,4-difluoro- phenyl)methyl]-N-(3,5- dimethoxyphenyl)acetamide | 390.1 |
| A44 | 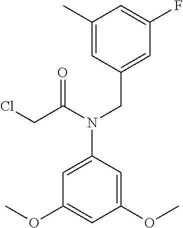 [SMILES: COc1cc(OC)cc(N(Cc2cc(C)cc(F)c2)C(=O)CCl)c1] | 2-chloro-N-(3,5-dimethoxyphenyl)-N- [(3-fluoro-5-methyl- phenyl) methyl]acetamide | 352.1 |
| A45 | 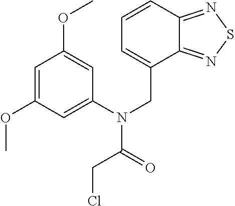 [SMILES: COc1cc(OC)cc(N(Cc2cccc3nsnc23)C(=O)CCl)c1] | N-(2,1,3-benzothiadiazol-4-ylmethyl)- 2-chloro-N-(3,5- dimethoxyphenyl)acetamide | 378.1 |
| A46 | 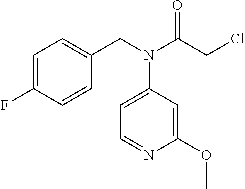 [SMILES: COc1cc(N(Cc2ccc(F)cc2)C(=O)CCl)ccn1] | 2-chloro-N-[(4-fluorophenyl)methyl]- N-(2-methoxy-4-pyridyl)acetamide | 309.1 |
| A47 | 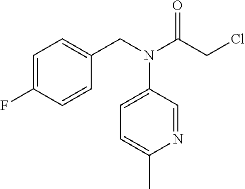 [SMILES: Cc1ccc(N(Cc2ccc(F)cc2)C(=O)CCl)cn1] | 2-chloro-N-[(4-fluorophenyl)methyl]- N-(6-methyl-3-pyridyl)acetamide | 293.1 |

In one embodiment, compounds of the invention, or pharmaceutically acceptable salts thereof, have the structure indicated by Formula 2:

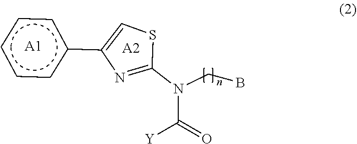

where n is 0 or 1; and
where B is selected from:

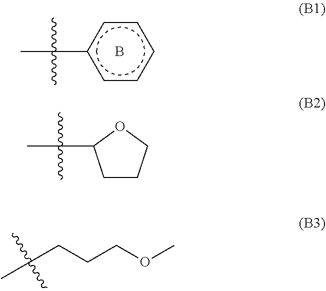

and where Y is selected from:

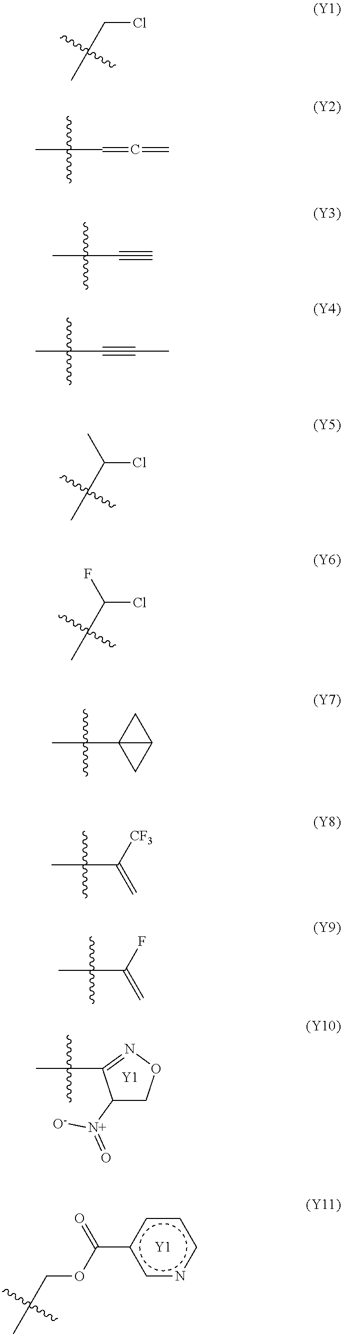

where each ring individually may be aliphatic or aromatic;
where each ring each ring individually may substituted or unsubstituted;
where when n is 0 or 1; and
where when B is B₁ or B₂; and
where when Y is any one of Y₁, Y₂, Y₃, Y₄, Y₅, Y₆, Y₇, Y₈, Y₉, Y₁₀, Y₁₁:
   there are no additional ring substituents or ring heteroatoms; or
   if substituted, substituents on A and/or B rings and/or ring Y1 are as follows:
      one or more F, Br, Cl, CN, OMe, Me at any position on the ring;
where when n is 0 and Y is Y₁:
   there are no additional ring substituents or ring heteroatoms; or
   B is a 2-propenyl substituent; or B is a hexyl substituent; or B is a hydrogen; and/or
   if substituted, substituents on ring A₁ are as follows:
      one or more F, Br, Cl, CN, Me, OMe at any position on the ring;
where when n is 0, B is B₁ and Y is Y₁:
   there are no additional ring substituents or ring heteroatoms; or
   there are one or more isopropyl substituents on any position on the B ring; and/or
   if substituted, substituents on rings A₁ and/or B₁ are as follows:
      one or more F, Br, Cl, CN, Me, OMe at any position on the ring;
where when n is 0, B is B₃ and Y is Y₁:
   there are no additional ring substituents or ring heteroatoms; or
   if substituted, substituents on ring A₁ are as follows:
      one or more F, Br, Cl, CN, Me, OMe at any position on the ring.

In one embodiment, compounds of the invention, or pharmaceutically acceptable salts thereof, have the structure of a compound provided in Table 2.

TABLE 2

| Working | | | MS Data |
|---|---|---|---|
| Example | Structure | Name | (M + H) |
| B1 | 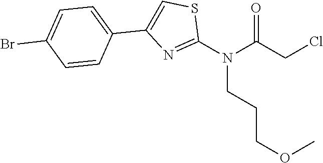 [SMILES: COCCCN(C(=O)CCl)c1nc(-c2ccc(Br)cc2)cs1] | N-[4-(4- bromophenyl)thiazol-2-yl]- 2-chloro-N-(3- methoxypropyl)acetamide | 403.1 |
| B2 | 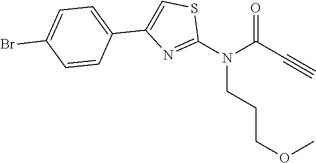 [SMILES: C#CC(=O)N(CCCOC)c1nc(-c2ccc(Br)cc2)cs1] | N-[4-(4- bromophenyl)thiazol-2-yl]- N-(3-methoxypropyl)prop-2- ynamide | 379.2 |
| B3 | 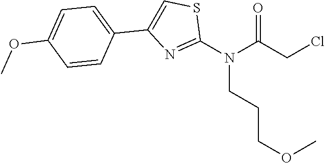 [SMILES: COCCCN(C(=O)CCl)c1nc(-c2ccc(OC)cc2)cs1] | 2-chloro-N-[4-(4- methoxyphenyl)thiazol-2- yl]-N-(3- methoxypropyl)acetamide | 355.1 |
| B4 | 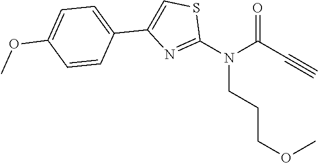 | N-[4-(4- methoxyphenyl)thiazol-2- yl]-N-(3- methoxypropyl)prop-2- ynamide | 331.2 |
| B5 | 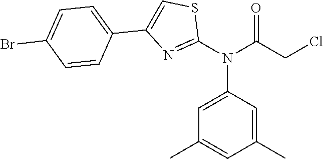 [SMILES: Cc1cc(C)cc(N(C(=O)CCl)c2nc(-c3ccc(Br)cc3)cs2)c1] | N-[4-(4- bromophenyl)thiazol-2-yl]- 2-chloro-N-(3,5- dimethylphenyl)acetamide | 435.0 |
| B6 | 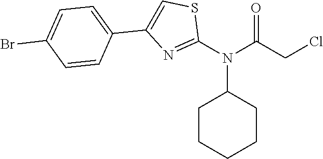 [SMILES: O=C(CCl)N(c1nc(-c2ccc(Br)cc2)cs1)C1CCCCC1] | N-[4-(4- bromophenyl)thiazol-2-yl]- 2-chloro-N-cyclohexyl- acetamide | 413.0 |
| B7 | 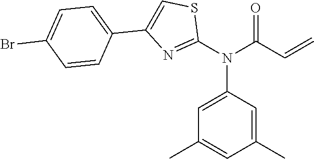 [SMILES: C=CC(=O)N(c1cc(C)cc(C)c1)c1nc(-c2ccc(Br)cc2)cs1] | N-[4-(4- bromophenyl)thiazol-2-yl]- N-(3,5- dimethylphenyl)prop-2- enamide | 413.0 |
| B8 | 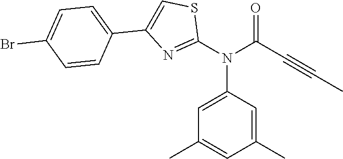 [SMILES: CC#CC(=O)N(c1cc(C)cc(C)c1)c1nc(-c2ccc(Br)cc2)cs1] | N-[4-(4- bromophenyl)thiazol-2-yl]- N-(3,5-dimethylphenyl)but- 2-ynamide | 425.2 |
| B9 | 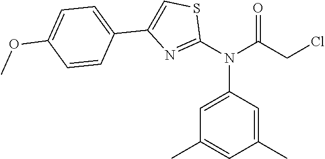 [SMILES: COc1ccc(-c2csc(N(C(=O)CCl)c3cc(C)cc(C)c3)n2)cc1] | 2-chloro-N-(3,5- dimethylphenyl)-N-[4-(4- methoxyphenyl)thiazol-2- yl]acetamide | 387.2 |
| B10 | 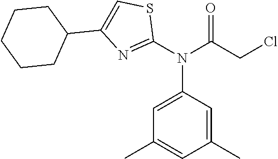 [SMILES: Cc1cc(C)cc(N(C(=O)CCl)c2nc(C3CCCCC3)cs2)c1] | 2-chloro-N-(4- cyclohexylthiazol-2-yl)-N- (3,5- dimethylphenyl)acetamide | 363.2 |
| B11 | 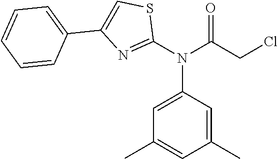 [SMILES: Cc1cc(C)cc(N(C(=O)CCl)c2nc(-c3ccccc3)cs2)c1] | 2-chloro-N-(3,5- dimethylphenyl)-N-(4- phenylthiazol-2- yl)acetamide | 357.1 |
| B12 | 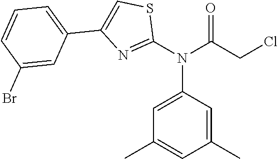 [SMILES: Cc1cc(C)cc(N(C(=O)CCl)c2nc(-c3cccc(Br)c3)cs2)c1] | N-[4-(3- bromophenyl)thiazol-2-yl]- 2-chloro-N-(3,5- dimethylphenyl)acetamide | 435.1 |
| B13 | 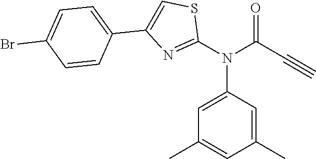 [SMILES: C#CC(=O)N(c1cc(C)cc(C)c1)c1nc(-c2ccc(Br)cc2)cs1] | N-[4-(4- bromophenyl)thiazol-2-yl]- N-(3,5- dimethylphenyl)prop-2- ynamide | 411.0 |
| B14 | 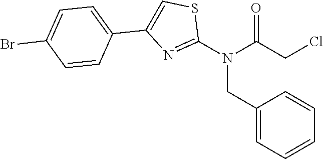 [SMILES: O=C(CCl)N(Cc1ccccc1)c1nc(-c2ccc(Br)cc2)cs1] | N-benzyl-N-[4-(4- bromophenyl)thiazol-2-yl]- 2-chloro-acetamide | 421.1 |
| B15 | 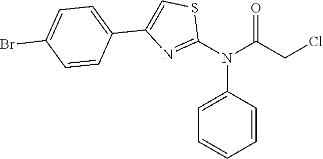 [SMILES: O=C(CCl)N(c1ccccc1)c1nc(-c2ccc(Br)cc2)cs1] | N-[4-(4- bromophenyl)thiazol-2-yl]- 2-chloro-N-phenyl- acetamide | 407.1 |
| B16 | 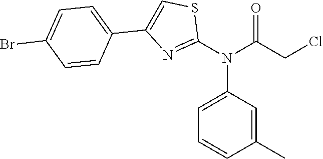 [SMILES: Cc1cccc(N(C(=O)CCl)c2nc(-c3ccc(Br)cc3)cs2)c1] | N-[4-(4- bromophenyl)thiazol-2-yl]- 2-chloro-N-(m- tolyl)acetamide | 421.1 |
| B17 | 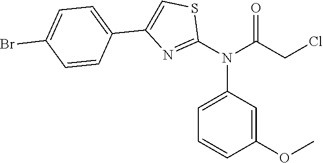 [SMILES: COc1cccc(N(C(=O)CCl)c2nc(-c3ccc(Br)cc3)cs2)c1] | N-[4-(4- bromophenyl)thiazol-2-yl]- 2-chloro-N-(3- methoxyphenyl)acetamide | 437.1 |
| B18 | 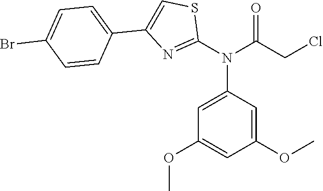 [SMILES: COc1cc(OC)cc(N(C(=O)CCl)c2nc(-c3ccc(Br)cc3)cs2)c1] | N-[4-(4- bromophenyl)thiazol-2-yl]- 2-chloro-N-(3,5- dimethoxyphenyl)acetamide | 467.1 |
| B19 | 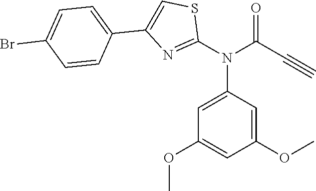 [SMILES: C#CC(=O)N(c1cc(OC)cc(OC)c1)c1nc(-c2ccc(Br)cc2)cs1] | N-[4-(4- bromophenyl)thiazol-2-yl]- N-(3,5- dimethoxyphenyl)prop-2- ynamide | 443.0 |
| B20 | 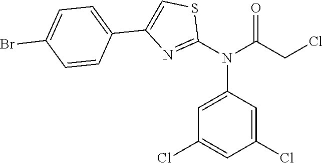 [SMILES: O=C(CCl)N(c1cc(Cl)cc(Cl)c1)c1nc(-c2ccc(Br)cc2)cs1] | N-[4-(4- bromophenyl)thiazol-2-yl]- 2-chloro-N-(3,5- dichlorophenyl)acetamide | 476.7 |
| B21 | 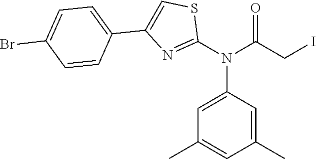 [SMILES: Cc1cc(C)cc(N(C(=O)CI)c2nc(-c3ccc(Br)cc3)cs2)c1] | N-[4-(4- bromophenyl)thiazol-2-yl]- N-(3,5-dimethylphenyl)-2- iodo-acetamide | 527.0 |
| B22 | 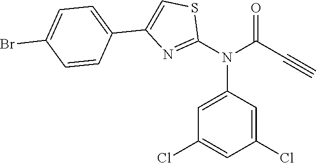 [SMILES: C#CC(=O)N(c1cc(Cl)cc(Cl)c1)c1nc(-c2ccc(Br)cc2)cs1] | N-[4-(4- bromophenyl)thiazol-2-yl]- N-(3,5-dichlorophenyl)prop- 2-ynamide | 452.9 |
| B23 | 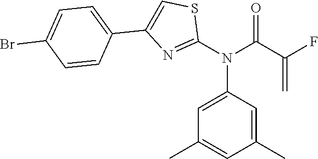 [SMILES: C=C(F)C(=O)N(c1cc(C)cc(C)c1)c1nc(-c2ccc(Br)cc2)cs1] | N-[4-(4- bromophenyl)thiazol-2-yl]- N-(3,5-dimethylphenyl)-2- fluoro-prop-2-enamide | 431.0 |
| B24 | 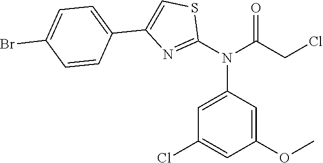 [SMILES: COc1cc(Cl)cc(N(C(=O)CCl)c2nc(-c3ccc(Br)cc3)cs2)c1] | N-[4-(4- bromophenyl)thiazol-2-yl]- 2-chloro-N-(3-chloro-5- methoxy-phenyl)acetamide | 472.9 |
| B25 | 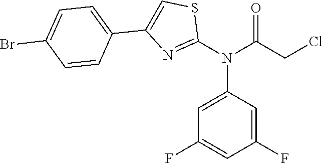 [SMILES: O=C(CCl)N(c1cc(F)cc(F)c1)c1nc(-c2ccc(Br)cc2)cs1] | N-[4-(4- bromophenyl)thiazol-2-yl]- 2-chloro-N-(3,5- difluorophenyl)acetamide | 444.9 |
| B26 | 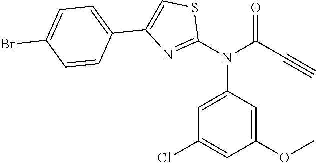 [SMILES: C#CC(=O)N(c1cc(Cl)cc(OC)c1)c1nc(-c2ccc(Br)cc2)cs1] | N-[4-(4- bromophenyl)thiazol-2-yl]- N-(3-chloro-5-methoxy- phenyl)prop-2-ynamide | 449.0 |
| B27 | 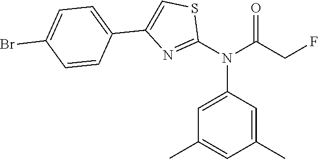 [SMILES: Cc1cc(C)cc(N(C(=O)CF)c2nc(-c3ccc(Br)cc3)cs2)c1] | N-[4-(4- bromophenyl)thiazol-2-yl]- N-(3,5-dimethylphenyl)-2- fluoro-acetamide | 419.0 |
| B28 | 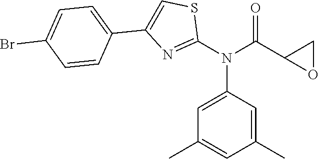 [SMILES: Cc1cc(C)cc(N(C(=O)C2CO2)c2nc(-c3ccc(Br)cc3)cs2)c1] | N-[4-(4- bromophenyl)thiazol-2-yl]- N-(3,5- dimethylphenyl)oxirane-2- carboxamide | 429.0 |

In one embodiment, compounds of the invention have the structure indicated by Formula 3:

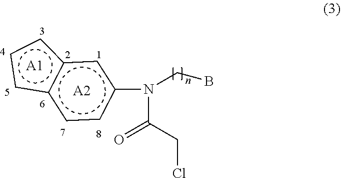

where n is 0 or 1; and
where B is selected from:

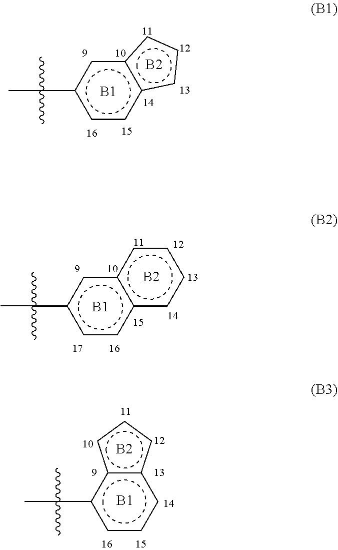

where each ring individually may be aliphatic or aromatic;
where each ring each ring individually may substituted or unsubstituted;
where when n is 0 and B is B₁:
   there are no ring substituents or ring heteroatoms; or
   there are ring heteroatoms comprising [(B1:9 or B1:16 or B1:9,16) and/or (B2:11,12 or B2:13) is an N]; and/or (B2:11 or B2:13 is an O); and/or B2:11 is an S;
   if substituted, substituents on rings A₁ and/or A₂ and/or B₁ and/or B₂ are as follows:
      one or more F, Br, Cl, CN, Me at any position on the ring;
where when n is 1 and B is B₁:
   there are no ring substituents or ring heteroatoms; or
   there are ring heteroatoms comprising [(A1:2,4 or A1:2,5 or A1:2,4,5 or A1:2,3,5 or A1:2,3,4,5 or A1:3,4 or A1:3,4,5 or A1:3,6 or A1:3,4,6 or A1:3,5,6 or A1:3,4,5,6 or A1:4,5 or A1:4,6 or A1:5 or A1:5,6) and/or (A2:1 or A2:1,8 or A2:8) is an N]; and/or A1:3 is an S; and/or (A1:3 or A1:5 is an O); and/or
   if substituted, substituents on rings A₁ and/or A₂ and/or B₁ and/or B₂ are as follows:
      one or more F, Br, Cl, CN, Me at any position on the ring;
where when n is 0 and B is B₂:
   there are no ring substituents or ring heteroatoms; or
   there are ring heteroatoms comprising [(B1:9 or B1:17 or B1:9,16 or B1:9,17) and/or B2:11 is an N]; and/or
   if substituted, substituents on rings A₁ and/or A₂ and/or B₁ and/or B₂ are as follows:
      one or more F, Br, Cl, CN, Me at any position on the ring;
where when n is 1 and B is B₂:
   there are no ring substituents or ring heteroatoms; or
   there are ring heteroatoms comprising [(A1:3 or A1:4 or A1:5 or A1:3,4 or A1:3,5 or A1:4,5 or A1:3,4,5) and/or (A2:2 or A2:6 or A2:8 or A2:6,8) and/or (B1:9 or B1:17 or B1:9,16 or B1:9,17) and/or (B2:11 or B2:12 or B2:14 or B2:12,14) is an N]; and/or A1:3 is an S; and/or
   if substituted, substituents on rings A₁ and/or A₂ and/or B₁ and/or B₂ are as follows:
      one or more F, Br, Cl, CN, Me at any position on the ring;
where when n is 1 and B is B₃:
   there are no ring substituents or ring heteroatoms; or
   there are ring heteroatoms comprising A1:3,4,6 and/or A2:8 is an N; and/or
   if substituted, substituents on rings A₁ and/or A₂ and/or B₁ and/or B₂ are as follows:
      one or more F, Br, Cl, CN, Me at any position on the ring.

In one embodiment, compounds of the invention have the structure indicated by Formula 4:

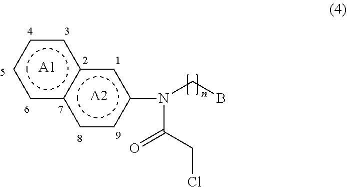

where n is 0 or 1; and
where B is selected from:

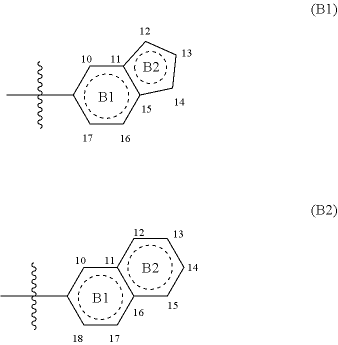

where each ring individually may be aliphatic or aromatic;
where each ring individually may substituted or unsubstituted;
where when n is 1 and B is B₁:
   there are no ring substituents or ring heteroatoms; or
   there are ring heteroatoms comprising [(A1:3 or A1:4 or A1:6) and/or (A2:1 or A2:1,8 or A2:1,9) is an N]; and/or
   if substituted, substituents on rings A1 and/or A2 and/or B1 and/or B2 are as follows:
      one or more F, Br, Cl, CN, Me at any position on the ring;
where when n is 1 and B is B₂:
   there are no ring substituents or ring heteroatoms; or
   there are ring heteroatoms comprising [(A1:4 or A1:6 or A1:4,6) and/or A2:1,8 and/or (B2:11 or B2:12 or B2:14 or B2:12,14) is an N]; and/or (A1:3 is an S); and/or
   if substituted, substituents on rings A1 and/or A2 and/or B1 and/or B2 are as follows:
      one or more F, Br, Cl, CN, Me at any position on the ring.

In one embodiment, compounds of the invention have the structure indicated by Formula 5:

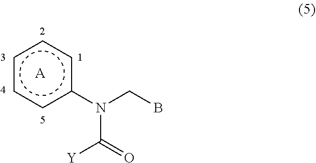

where B is selected from:

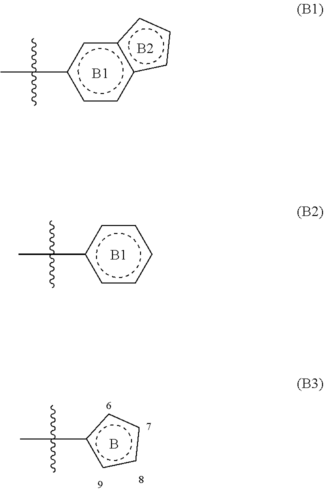

where each ring individually may be aliphatic or aromatic;
where each ring individually may substituted or unsubstituted;
and where Y is selected from:

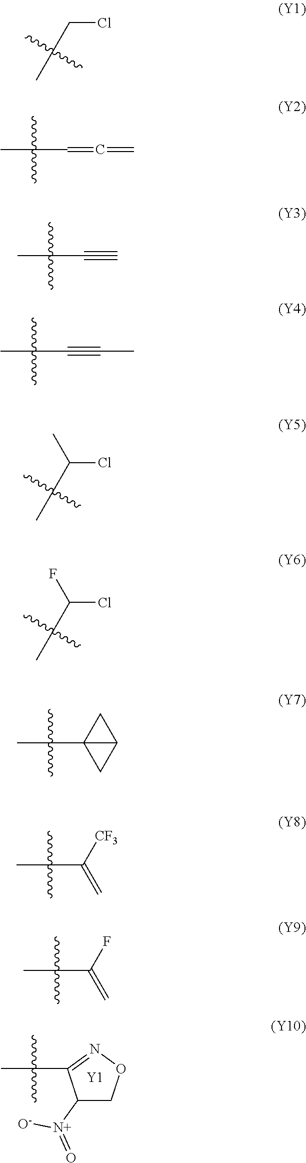

where when B is B₂; and
where when Y is any of Y₁, Y₂, Y₃, Y₄, Y₅, Y₆, Y₇, Y₈, Y₉, Y₁₀:
   there are no additional ring substituents or ring heteroatoms; or
   if substituted, substituents on rings A and/or B and/or Y1 are as follows:
      one or more F, Br, Cl, CN, Me, OMe at any position on the ring;
where when B is B₁; and
where when Y is Y₁:
   there are no additional ring substituents or ring heteroatoms; or
   there are ring heteroatoms comprising A:1,5 is a N; and/or
   if substituted, substituents on rings A and/or B are as follows:
      one or more F, Br, Cl, CN, Me, OMe at any position on the ring;
where when B is B₃; and
where when Y is Y₁:
   there are no additional ring substituents or ring heteroatoms; or
   there are ring heteroatoms comprising (B:6,7; and/or B:9 is a N); and/or B:7 is an O; and/or B:6 is an S; and/or
   if substituted, substituents on rings A and/or B are as follows:
      one or more F, Br, Cl, CN, Me, OMe at any position on the ring.

The compounds of the invention include the compounds of Formulas 1-5, and active derivatives and salts thereof.

In some embodiments, a compounds of the invention is a compound shown in Tables 1 and 2.

In some embodiments, a compounds of the invention is a compound shown in Table 3. The compounds of the invention include the compounds 1-643 and active derivatives and salts thereof.

Different versions of the compounds of the invention may be synthesized. For example, the base structure of Compound 90 (see Table 3) may be modified at one or more certain positions to include different “linker lengths” that may include different numbers of carbons at this position to result in Formula 91 of Table 3, where each n independently is 0, 1 or 2 and R is a 5, 6, 7 or 8 membered optionally substituted aromatic or non-aromatic carbon ring.

### Synthesis of Compounds 1-643

The Compounds 1-643 are either commercially available or may be synthesized using standard synthetic techniques known to those of ordinary skill in the art.

### Compositions

The invention provides a composition, the composition comprising a pharmaceutically acceptable carrier, adjuvant, or vehicle, and one or more compounds having any one of the structures of Compounds 1-643 or active derivatives or a pharmaceutically acceptable salt thereof.

A composition of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a composition of the present invention may be administered in conjunction with other treatments. A composition of the present invention may be encapsulated or otherwise protected against gastric or other secretions, if desired.

The compositions of the invention are pharmaceutically acceptable and may comprise one or more active ingredients in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art see, e.g., Remington, The Science and Practice of Pharmacy (21st Edition, Lippincott Williams and Wilkins, Philadelphia, Pa).

Pharmaceutically acceptable carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars {e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The compositions of the invention may, optionally, contain additional materials commonly used in such compositions. These ingredients and materials are well known in the art. Examples of pharmaceutically acceptable materials include:
fillers or extenders, such as starches, lactose, sucrose, glucose, mannitol, and silicic acid;
binders, such as carboxymethylcellulose, alginates, gelatin, polyvinyl pyrrolidone, hydroxypropylmethyl cellulose, sucrose, and acacia;
humectants, such as glycerol;
disintegrating agents, such as agar-agar, calcium carbonate, potato, or tapioca starch, alginic acid, certain silicates, sodium starch glycolate, cross-linked sodium carboxymethyl cellulose and sodium carbonate;
solution retarding agents, such as paraffin;
absorption accelerators, such as quaternary ammonium compounds;
wetting agents, such as cetyl alcohol and glycerol monostearate;
absorbents, such as kaolin and bentonite clay;
lubricants, such as talc, calcium stearate, magnesium stearate, solid polyethylene glycols, and sodium lauryl sulfate;
suspending agents, such as ethoxylated isostearyl alcohols, polyoxyethylene sorbitol and sorbitan esters, microcrystalline cellulose, aluminum metahydroxide, bentonite, agar-agar and tragacanth;
buffering agents;
excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder;
inert diluents, such as water or other solvents;
preservatives;
surface-active agents;
dispersing agents;
control-release or absorption-delaying agents, such as hydroxypropyl methyl cellulose, other polymer matrices, biodegradable polymers, liposomes, microspheres, aluminum monosterate, gelatin, and waxes;
opacifying agents;
adjuvants;
wetting agents;
emulsifying and suspending agents;
solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan;
propellants, such as chlorofluorohydrocarbons and volatile unsubstituted hydrocarbons, such as butane and propane;
antioxidants;
agents which render the formulation isotonic with the blood of the intended recipient, such as sugars and sodium chloride;
thickening agents;
coating materials, such as lecithin; and
sweetening, flavoring, coloring, perfuming and preservative agents.

Ingredients and materials suitable for a selected dosage form and intended route of administration are well known in the art, and acceptable ingredients and materials for a chosen dosage form and method of administration may be determined using ordinary skill in the art.

Compositions suitable for oral administration may be in the form of capsules, cachets, pills, tablets, powders, granules, a solution, or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragées, powders, granules, and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions which are suitable for vaginal administration also include pessaries, tampons, creams, gels, pastes, foams, or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents, and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers.

Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

### Inducing Ferroptosis in Cells

The invention provides a method of inducing ferroptosis in a cell, the method comprising contacting the cell with an effective amount of one or more compounds according to the present invention.

In one aspect of this embodiment, the cell may be mammalian, preferably human. In other aspects of this embodiment, the cell may be from a laboratory animal. In addition to humans, categories of mammals within the scope of the present invention include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include rats, mice, rabbits, guinea pigs, etc.

In one aspect of this embodiment, the method is carried out in vitro. In other aspects of this embodiment, the method is carried out in vivo or ex vivo.

In one embodiment, the cell is a cancer cell, such as a mesenchymal cancer cell. Mesenchymal tumors (i.e., either sarcomas or tumors that have undergone epithelial to mesenchymal transition) are typically characterized by a relatively high content of polyunsaturated fatty acids and iron. Because of the relatively high polyunsaturated fatty acid and iron content, cellular lipids are subjected to relatively high levels of oxidation to produce lipid peroxides, which in the absence of GPX4 can be toxic to the cell.

### Decreasing GPX4 Activity in Cells

The invention provides a method for decreasing GPX4 activity in a cell, the method comprising contacting the cell with an effective amount of one or more compounds of the present invention.

In one aspect of this embodiment, the cell may be mammalian, preferably human. In other aspects of this embodiment, the cell may be from a laboratory animal. In addition to humans, categories of mammals within the scope of the present invention include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include rats, mice, rabbits, guinea pigs, etc.

In one aspect of this embodiment, the method is carried out in vitro. In other aspects of this embodiment, the method is carried out in vivo or ex vivo.

### Cancer Therapy

The compounds of the invention may be used in cancer therapy to induce ferroptosis in a cancer cell.

In one embodiment, the invention provides a method for treating a cancer in a subject in need thereof, the method comprising, administering to the subject a pharmaceutically effective amount of a pharmaceutical composition including one or more compounds, or pharmaceutically acceptable salts thereof, of the present invention.

In one aspect, the invention provides a method for treating a mesenchymal cancer, e.g., a sarcoma, in a subject in need thereof, the method comprising, administering to the subject a pharmaceutically effective amount of a pharmaceutical composition including one or more compounds, or pharmaceutically acceptable salts thereof, of the present invention.

In one aspect, the method reduces the growth rate of a tumor, reduces the size of a tumor, eliminates a tumor, or delays progression of a cancer stage.

### EXAMPLES

The following examples are included for illustrative purposes only and are not intended to limit the scope of the inventive concepts herein.

### Identification of Ferroptosis-Inducing Compounds

To identify potential covalent inhibitors of GPX4, we screened a panel of 294 diverse small molecules from the Enamine covalent inhibitor library (Catalog No. CSL-10480-0-Z-10, Enamine, Monmouth, NJ). Each compound contains either a chloroacetamide or acrylamide moiety theoretically capable of forming a covalent bond with a selenocysteine. The small molecule compounds were selected for a diversity of backbone structures. The screening protocol used the cell viability assay Cell Titer Glo® (available from Promega, Madison, WI). Cell Titer Glo® is a luminescent cell viability that determines the number of viable cells in culture by quantifying ATP, which indicates the presence of metabolically active cells. Briefly, HT-1080 fibrosarcoma cells were plated 24 hours prior to treatment with 10 UM of each compound in the screening panel. Cell Titer Glo® was used to read out cell viability after 48 hours of compound exposure. Data was collected in duplicate.

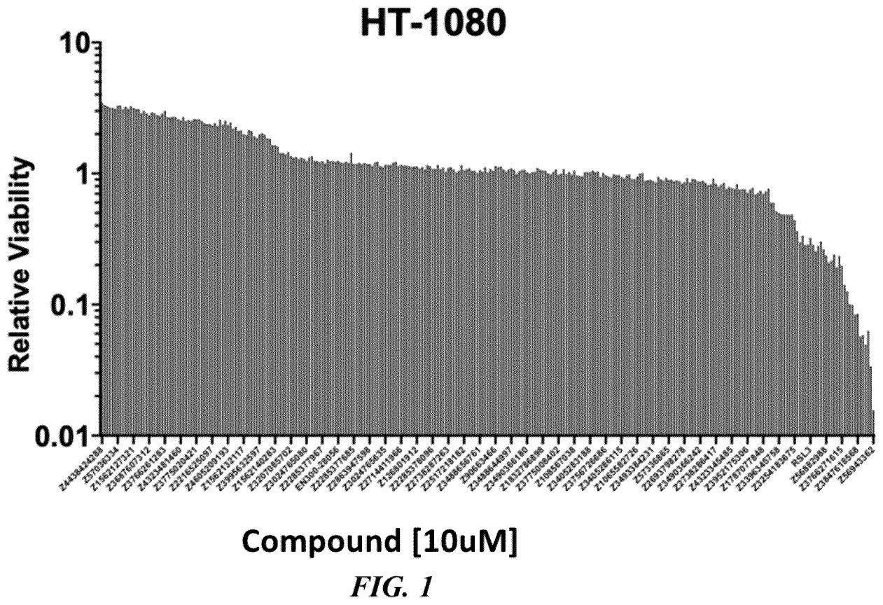

FIG. 1 is a plot 100 showing the relative viability of HT-1080 fibrosarcoma cells exposed to each compound in the inhibitor screening panel. We identified 36 out of the 294 (12%) small molecule compounds tested that resulted in greater than 50% reduction in cell viability at this dose. All but one of 36 small molecules identified in the screen contains a chloroacetamide moiety, which suggests that chloroacetamides are generally more toxic to HT-1080 fibrosarcoma cells than acrylamides.

To determine whether the reduction in cell viability by the compounds identified in FIG. 1 is driven by induction of ferroptosis, we performed dose response curves for each compound in the presence or absence of ferrostatin-1 or liproxstatin-1, which prevent ferroptosis by functioning as radical trapping antioxidants at cellular membranes. Briefly, HT-1080 fibrosarcoma cells were plated 24 hours prior to treatment with a range of concentrations of each inhibitor compound ranging from 100 μM to 100 nM in the presence or absence of 1 μM liproxstatin-1 or 1.5 UM ferrostatin-1. Cell Titer Glo® was used to read out cell viability after 48 hours compound exposure. Data was collected in triplicate.

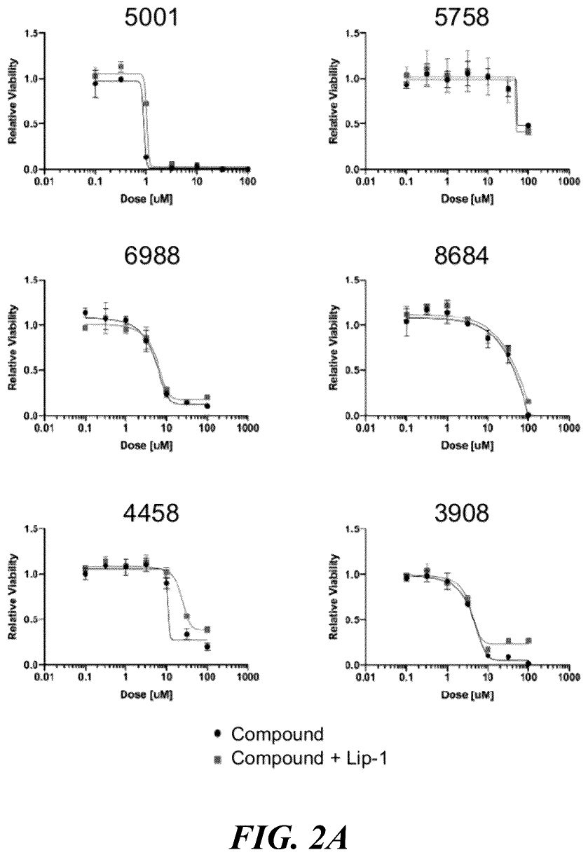

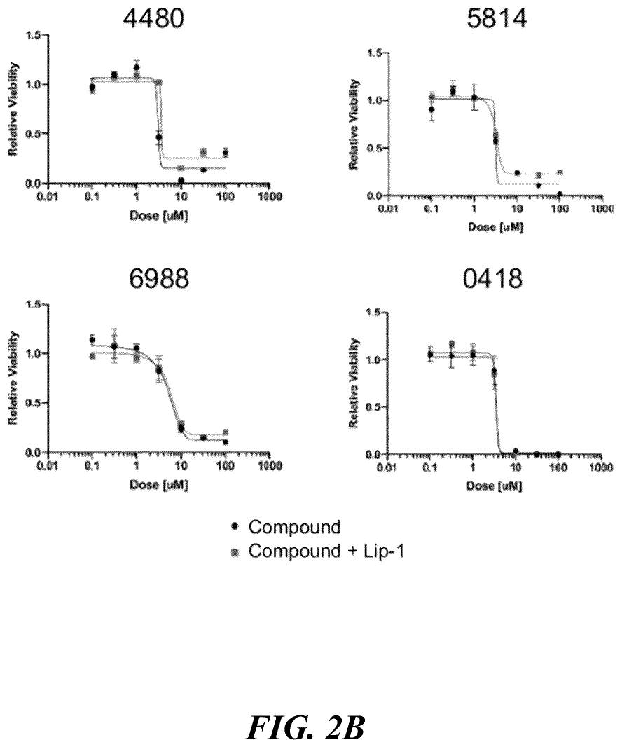

FIG. 2A and FIG. 2 B are plots 200 showing the relative viability of HT-1080 fibrosarcoma cells exposed to inhibitor compounds in the presence and absence of 1 μM Liproxstatin-1. In this example, viability data are shown for HT-1080 cells exposed to the compounds 5001, 5758, 4480, 5814, 6988, 8684, 0418, 4458, and 3908 in the presence (red line) or absence (black line) of Liproxstatin-1. The data show that the majority of compounds tested displayed cell killing both in the presence and absence of 1 μM liproxstatin-1, which suggests a non-ferroptotic mode of action.

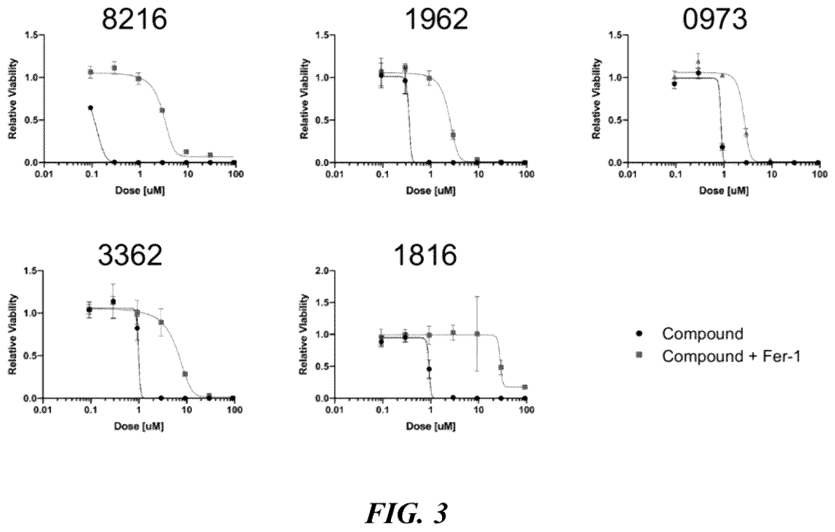

FIG. 3 is a panel of plots 300 showing the relative viability of HT-1080 fibrosarcoma cells exposed to inhibitor compounds in the presence (red line) and absence (black line) of 1.5 UM ferrostatin-1. In this example, viability data are shown for HT-1080 cells exposed to the compounds 8216, 1962, 0973, 3362, and 1816. The five compounds (8216, 1962, 0973, 3362, and 1816) resulted in cell death that was rescued by the addition of the antioxidant ferrostatin-1 to the media, as evidenced by the shift of the red line to the right. These results suggest that the cells are undergoing lipid peroxidation induced by these five inhibitor compounds (i.e., the five inhibitor compounds induce ferroptosis). The chemical structures of the five inhibitor compounds (8216, 1962, 0973, 3362, and 1816) are shown in Table 4.

To validate that the five ferroptosis-inducing compounds identified in FIG. 3 do cause lipid peroxidation, we used Bodipy-C11 staining in 786-O Renal Clear Cell Carcinoma cells to visualize using microscopy the presence (or absence) of oxidized lipids. Biodipy-C11 is a dye that traffics to lipid membranes and harbors a fluorophore that emits at different wavelengths depending on its oxidation status. Briefly, 786-O Renal Clear Cell Carcinoma cells were plated 24 hours prior to treatment with each of the five ferroptosis-inducing compounds (8216, 1816, 3362, 1962, and 0973) for one or two hours. DMSO was used as a control treatment. Following the treatment period, Hoechst stain (10 μM; a nuclear counterstain) and Bodipy-C11 (200 μM) were added to the cells for 30 minutes and then the cells were imaged using the Opera Phenix high content screening microscope.

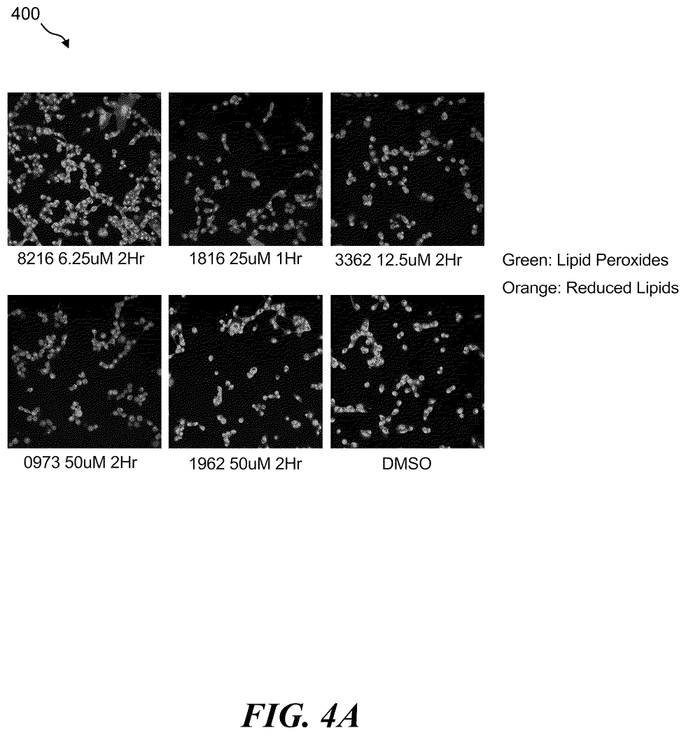

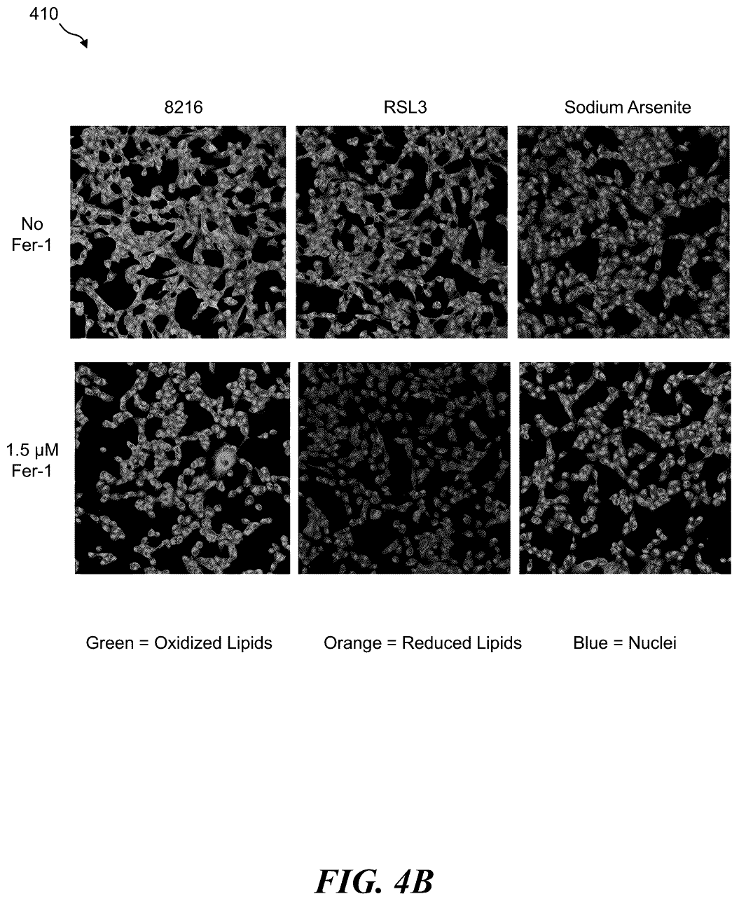

FIG. 4A is a panel of plots 400 showing fluorescence images of Biodipy-C11 staining of cells treated with the five ferroptosis-inducing compounds identified in FIG. 3. In this example, images are shown for cells exposed to compound 8216 (6.25 μM) for 2 hours, compound 1816 (25 μM) for 1 hour, compound 3362 (12.5 μM) for 2 hours, compound 0973 (50 μM) for 2 hours, and compound 1962 (50 μM) for 2 hours. Cells treated with DMSO were used as a non-lethal control. Green: oxidized lipids; orange: reduced lipids; blue: nuclei (Hoechst nuclear counterstain). In the DMSO treated control cell cultures, all cells appear as orange, which indicates that all lipids are in the reduced form. In cell cultures treated with the ferroptosis-inducing compounds, some degree of lipid peroxidation was observed for all five compounds (as evidenced by the green color), but to varying degrees and at different dosages and time points. The data demonstrates that cellular lipids are becoming oxidized in response to exposure to these five compounds, which suggests the compounds induce lipid peroxidation and hence ferroptosis.

FIG. 4B is a panel of plots 410 showing fluorescence images of Biodipy-C11 staining of cells treated with compound 8216 and the controls RSL3 and sodium arsenite. Briefly, 786-O Renal Clear Cell Carcinoma cells were plated 24 hours prior to treatment with compound 8216 and the controls RSL3 and sodium arsenite for 1 hour with and without 1.5 μM of ferrostatin-1. Bodipy C-11 undergoes a shift in emission when lipids become oxidized, non-oxidized lipids are represented in orange and oxidized lipids are green; Hoescht was included as nuclear stain to represent cell bodies. A hallmark of ferroptosis is the oxidation of lipids, therefore with the shift in emission color from orange to green in these cells with 8216 treatment is suggestive of active ferroptosis.

### Mechanism of Ferroptosis-Inducing Chloroacetamide Compounds

Mechanistically, ferroptosis can be induced by covalent inhibitors of GPX4 or by depletion of glutathione (GSH). Since GPX4 reduces lipid hydroperoxides using GSH as a co-substrate, both mechanisms ultimately result in loss of GPX4 activity, followed by elevated levels of reactive oxygen species which induces lipid peroxidation and subsequent cell death. In addition, changes in the levels of polyunsaturated fatty acids and/or iron in a cell in response to a potential covalent inhibitor of GPX4 activity may also induce or contribute to the ferroptotic phenotype.

To determine whether the five ferroptosis-inducing compounds identified in FIG. 3 and FIG. 4 could function through direct binding and inhibition of GPX4, we tested binding of each compound to recombinant GPX4 protein in vitro by intact protein mass spectrometry. Intact mass analysis is the assessment of a protein's total molecular weight by mass spectrometry without prior digestion or fragmentation of the molecule of interest. Covalent binding of a potential inhibitor to GPX4 can be detected by a shift in the mass of the intact protein. For the intact protein analysis, we used ML162, a well-characterized GPX4 inhibitor (available from Sigma Aldrich), as a positive control. As negative controls, we included two chloroacetamide-containing compounds (0568 and 2604) that did not kill HT-1080 fibrosarcoma cells in the initial 10 μM screen described with reference to FIG. 1. Briefly, compounds were incubated with recombinant GPX4 protein in vitro for 2 hours before quenching with trifluoroacetic acid (TFA) and analysis by mass spectroscopy.

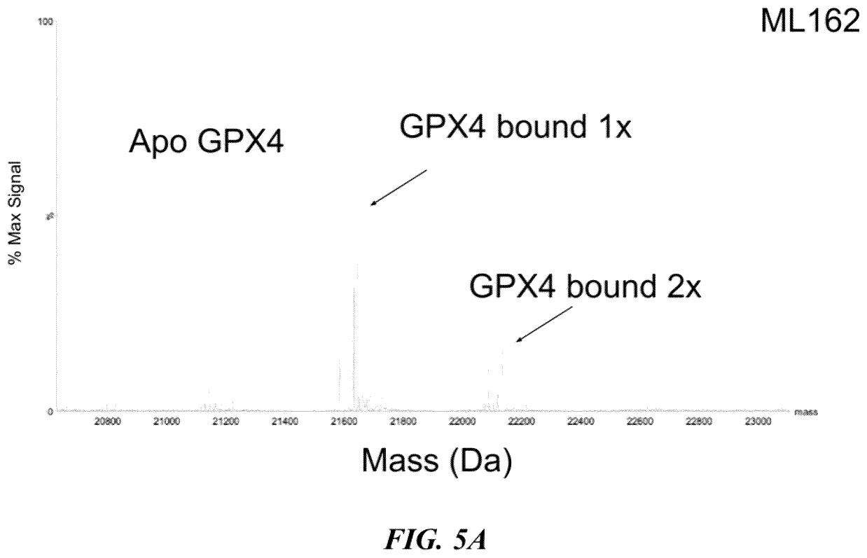

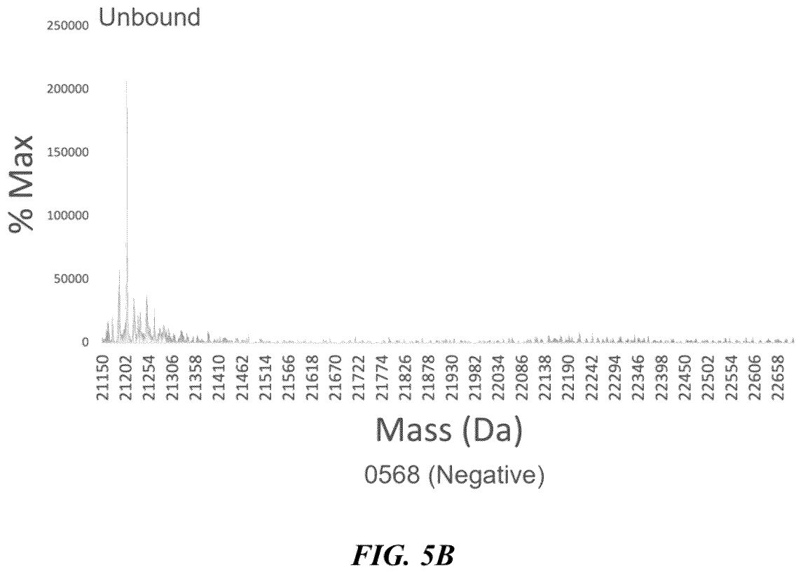

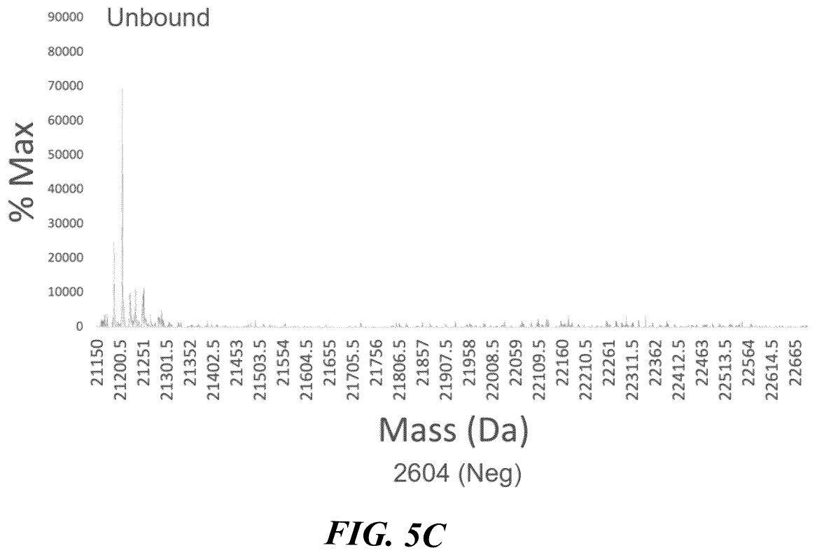

FIG. 5A is a plot 500 showing the mass spectrum for positive control ML162 binding to recombinant GPX4 in vitro. FIG. 5A and FIG. 5B are plots 510 showing the mass spectrums for the negative controls 0528 and 2604, respectively, binding to recombinant GPX4 in vitro. Recombinant GPX4 has a mass of 21206 DA. Referring now to FIG. 5A, the data show that all of the GPX4 protein is bound by ML162 as evidenced by the shift in peak size from 21206 Da to 21648.1992 Da, which corresponds to the mass of 1 molecule of ML162 (441.95 Da) binding to GPX4. A secondary site in GPX4 was also bound by ML162 as evidenced by a second peak at 22136.4004, which corresponds to the mass of 2 molecules of ML162 binding to GPX4. Referring now to FIG. 5B, neither negative control compound (0568 or 2604) bound to GPX4 in vitro. These results confirm that intact protein mass spectrometry can be used to detect binding of chloroacetamide-containing small molecules to GPX4, and that GPX4 likely harbors multiple potential binding sites for such compounds in vitro.

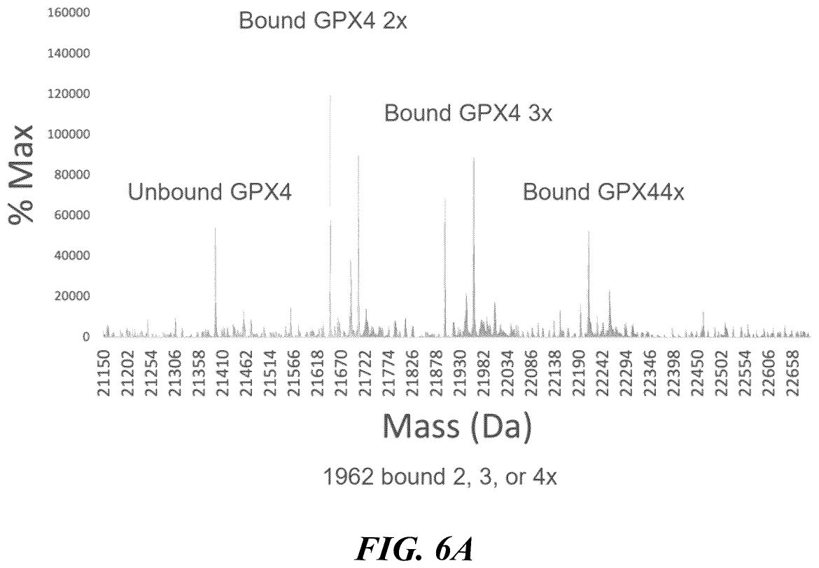

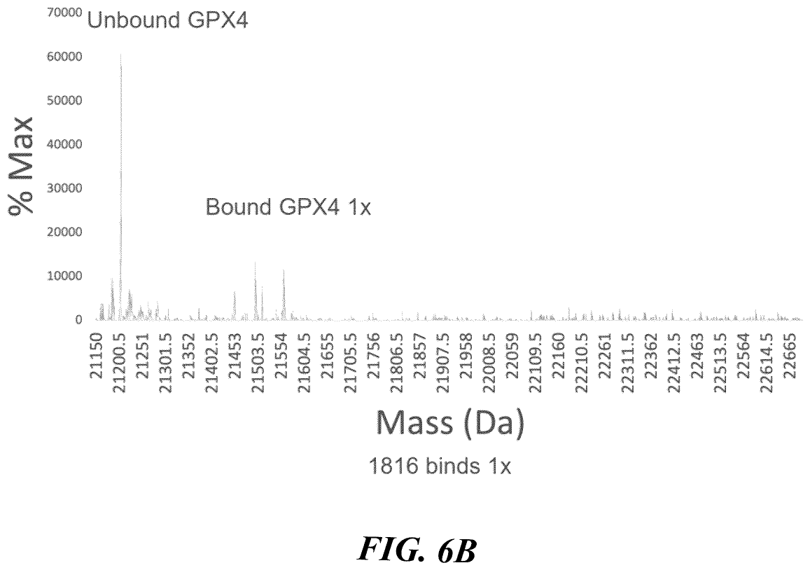

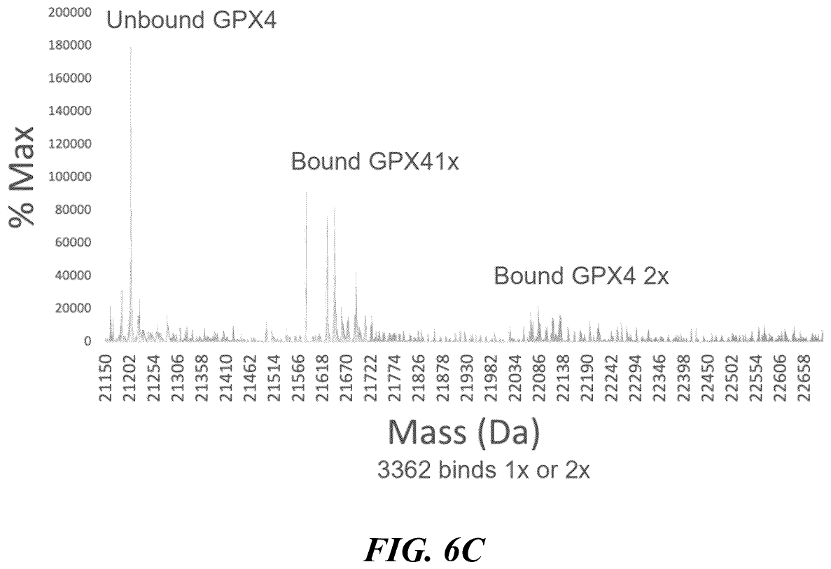

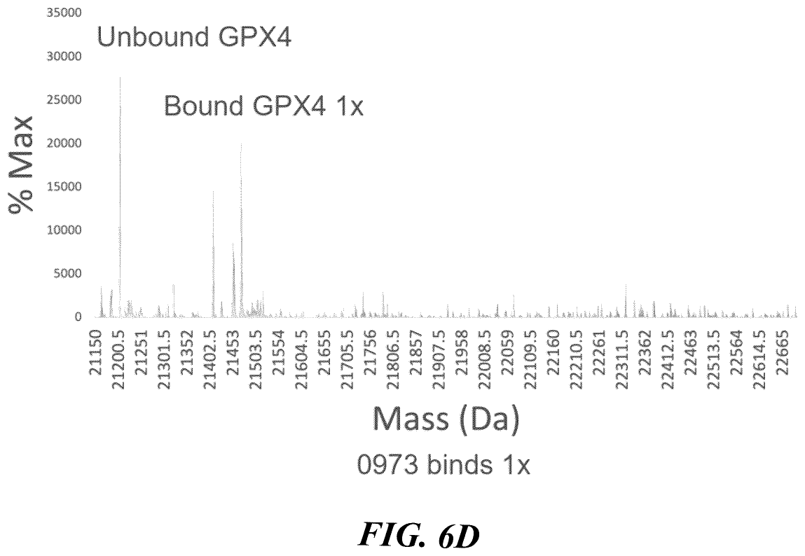

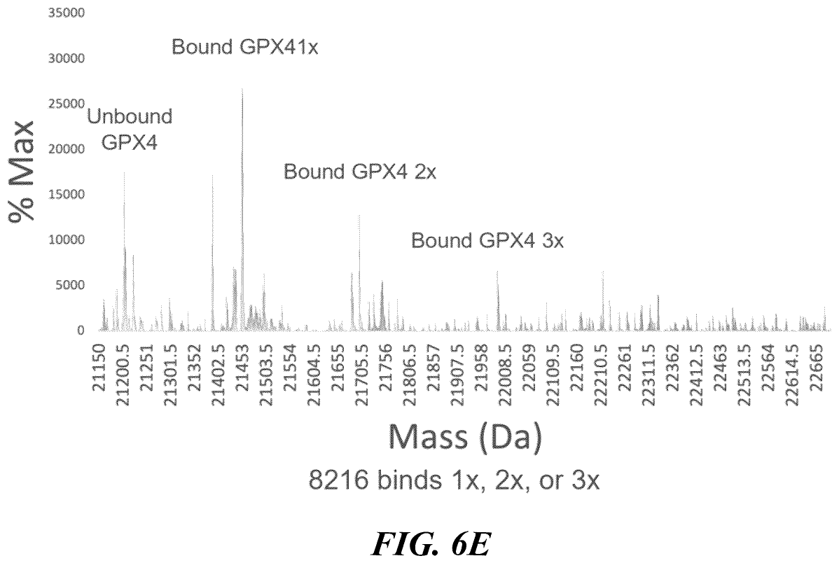

We tested each of the five ferroptosis-inducing compounds identified in FIG. 3 and FIG. 4 for the ability to bind GPX4 in vitro. FIG. 6A-6E are plots 600 showing the mass spectrums for the five ferroptosis-inducing compounds 1816, 8216, 3362, 1962, and 0973 binding to recombinant GPX4 in vitro. Peaks corresponding to unbound GPX4 are outlined in blue boxes (Unbound GPX4) and peaks corresponding to bound GPX4 are outlined in red boxes (Bound GPX4). The data show that all fie compounds were capable of binding GPX4, but with varying degrees of specificity. For example, compounds 0973 and 1816 display a single mass shift, implying that they bind GPX4 at a single site, while compounds 8216 and 3362 bind either 1, 2, or 3 times, which may suggest that they are less selective binders than compounds 0973 or 1816 in vitro. Compound 1962 appears to be the least selective binder, however, as no singly-bound protein was detected. Instead, compound 1962 can bind GPX4 either 2, 3, or 4 times, suggesting poor specificity for the GPX4 active site for this molecule.

### Inhibition of GPX4 Enzymatic Activity

To determine whether the five ferroptosis-inducing compounds (1816, 8216, 3362, 1962, and 0973) inhibit GPX4 enzymatic activity in vitro, we used a commercially available GPX4 inhibitor screening kit (Cayman Chemical cat #701880). This assay measures a compound's ability to prevent cumene hydroperoxide reduction by recombinant GPX4 protein. Briefly, the five ferroptosis-inducing compounds (25 μM) were incubated with recombinant GPX4 protein for one hour before being run in the Cayman Chemicals GPX4 inhibitor screening assay according to manufacturer's recommendations. The assay read out is a change in absorbance between NADPH and NADP. ML162 (25 μM) and RSL3 (25 μM) were used as positive control compounds. RSL3 (available from Apex Bio) is a GPX4 inhibitor that has been shown to require an adapter protein, 14-3-3, for efficient inhibitory activity in vitro.

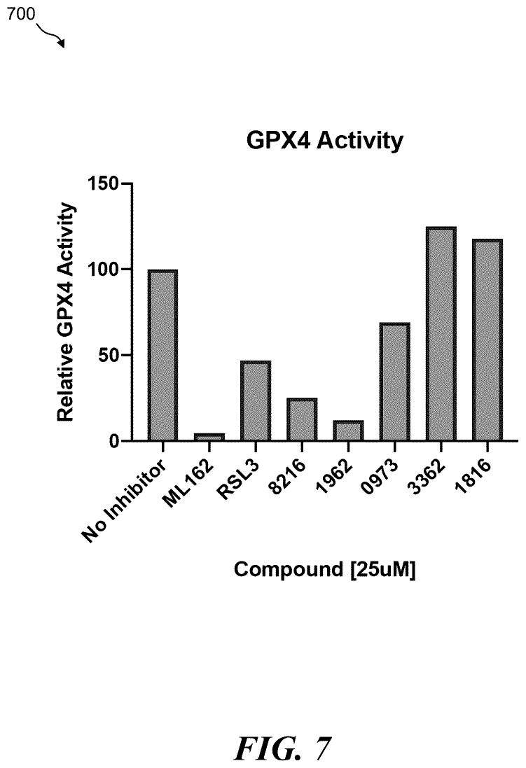

FIG. 7 is a plot 700 showing relative GPX4 activity in vitro for the five ferroptosis-inducing compounds 1816, 3362, 0973, 1962, 8216 compared with the ML162 and RSL3 positive control compounds. As expected, ML162 resulted in a reduction in GPX4 enzymatic activity of about 94%, while RSL3 (without the 14-3-3 adapter protein) showed a reduction of about 55% in GPX4 enzymatic activity at the concentration tested. Two of the candidate ferroptosis-inducers, 8216 and 1962, exhibit potent GPX4 inhibitory capacity, while 0973, 3362, and 1816 do not.

Referring now to FIG. 6 and FIG. 7, the binding and GPX4 enzymatic activity results suggest that compounds 8216 and 1962 may be direct GPX4 inhibitors, with EC50s (half maximal effective concentration) of about 100 nM and 440 nM, respectively. Interestingly, compounds 0973, 3362, and 1816 do not inhibit GPX4 enzymatic activity in vitro, suggesting that they may induce ferroptosis via alternative mechanisms, or that they require the cellular context for GPX4 inhibition. Understanding the targets of these non-GPX4-targeting compounds may present new opportunities for induction of ferroptosis in cells.

### Selectivity for Cancer Cells

To test whether the ferroptosis-inducing compounds 8216 or 1962 display selectivity for cancer cells we performed a dose response study in HK-2 kidney epithelial cells. HK-2 kidney epithelial cells are a non-cancerous cell line that is commonly used as a “healthy” control in ferroptosis experiments. Briefly, HK-2 kidney epithelial cells were plated 24 hours prior to treatment with concentrations of the ferroptosis-inducing compounds 8216 or 1962 ranging from 10 UM to 10 nM. The ferroptosis-inducing compound RSL3 was used as a positive control. As an additional control, we included a non-specific inducer of apoptosis, staurosporine. Cell Titer Glo® was used to read out cell viability after 48 hours of compound exposure. Data was collected in triplicate.

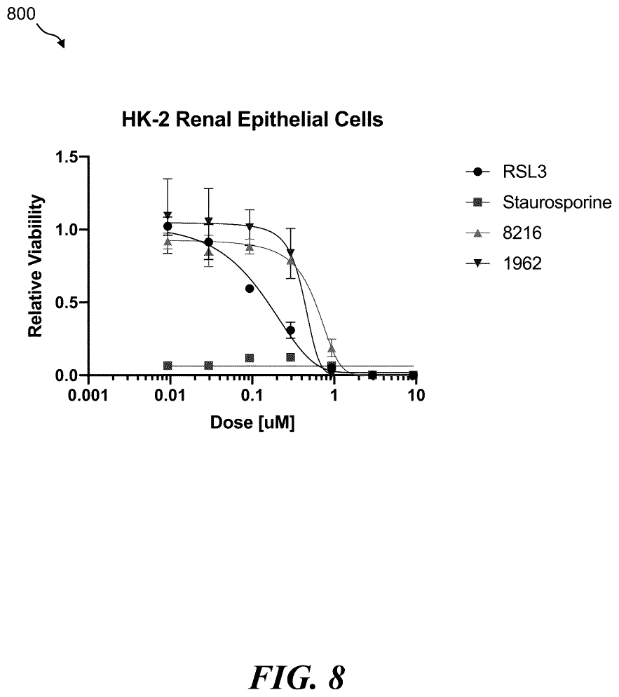

FIG. 8 is a plot 800 showing the relative viability of HK-2 renal epithelial cells exposed to the ferroptosis-inducing compounds 8216 or 1962 over a range of concentrations. The data show staurosporine killed HK-2 renal epithelial cells at all doses tested. The data also show that RSL3, which is used as a model compound for other inhibitors of GPX4 activity, also displays toxicity in HK-2 renal epithelial cells. In contrast, both 8216 and 1962 compounds were less toxic to the HK-2 renal epithelial cells than RSL3 or staurosporine.

To determine whether the reduction in viability of HK-2 renal epithelial cells exposed to compounds 8216 or 1962 is driven by induction of ferroptosis, we performed dose response curves for each compound in the presence or absence of ferrostatin-1. We also compared the EC50s for each compound between healthy cells (HK-2 renal epithelial cells) and cancer cells (HT-1080 fibrosarcoma cells) to determine a therapeutic window for healthy vs diseased cells. Briefly, HK-2 renal epithelial cells were plated 24 hours prior to treatment with concentrations of each inhibitor compound ranging from 10 UM to 10 nM in the presence or absence of 1.5 μM ferrostatin-1. The ferroptosis-inducing compound RSL3 was used as a positive control. Cell Titer Glo® was used to read out cell viability after 48 hours of compound exposure. Data was collected in triplicate.

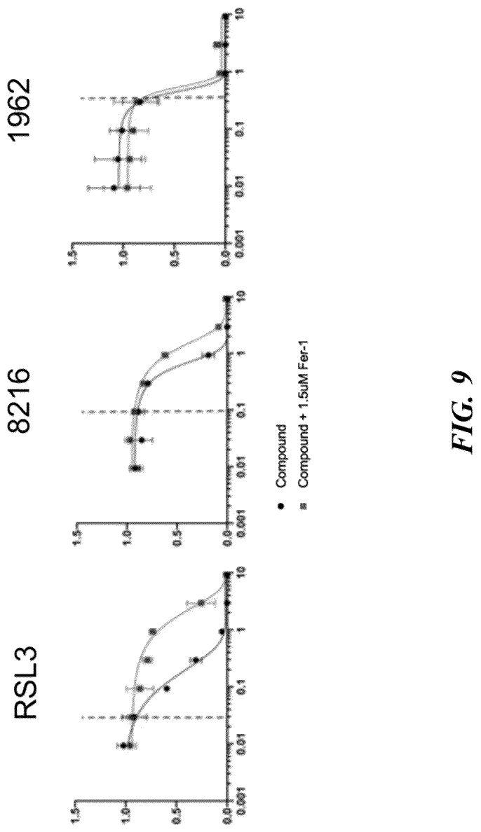

FIG. 9 is a panel of plots 900 showing relative viability of HK-2 kidney epithelial cells exposed to the inhibitor compounds 8216 or 1962 over a range of concentrations in the presence and absence of ferrostatin-1. The black line is the relative HK-2 kidney epithelial cell viability in the absence of ferrostatin-1 and the red line is the relative HK-2 kidney epithelial cell viability in the presence of ferrostatin-1. HT-1080 fibrosarcoma cell EC50s are denoted by the vertical red dotted line for each compound. Taking the difference between the dotted red line for HT-1080 fibrosarcoma cells and the HK-2 kidney epithelial cells (black line) provides an estimate of a therapeutic index. The data show that the EC50 of the 8216 and 1962 compounds is significantly shifted in HK-2 renal epithelial cells relative to HT-1080 fibrosarcoma cells, suggesting that these compounds are selective for cancer cells. Furthermore, the therapeutic window for the 8216 compound appears to be comparable to that of RSL3, given the differences in EC50 between healthy (HK-2 kidney epithelial cells) and cancer cells (HT-1080 fibrosarcoma cells) for both compounds.

We next examined whether the toxicity observed in HK-2 kidney epithelial cells is due to on- or off-target effects of each compound by calculating the degree of rescue by ferrostatin-1 in these cells. Referring still to FIG. 9, RSL3 toxicity in HK-2 kidney epithelial cells was effectively rescued by addition of ferrostatin-1, suggesting that the toxicity imparted to HK-2 cells by RSL3 is likely due to partial induction of ferroptosis. Toxicity of compound 8216 is also modestly rescued by ferrostatin-1, whereas 1962 is not protected, suggesting that the toxicity imparted by these compounds is at least partly due to off-target effects.

### Transcriptional Signature of GPX4 Inhibition

Experimental results described above suggest that five of our compounds induce ferroptosis, though likely through alternative mechanisms. This is expected as ferroptosis can be induced as a result of several mechanisms besides direct inhibition of GPX4. For example, inhibition of system Xc leads to the depletion of glutathione and subsequent inactivation of GPX4. Ferroptosis can also be induced by increasing the amount of polyunsaturated fatty acids or labile iron within a cell. Methods to distinguish these different modes (direct vs indirect) of ferroptosis-induction could be helpful in the development of ferroptosis-inducing small molecule compounds.

Expression of heme oxygenase 1 (HMOX1) mRNA may be used as a molecular response biomarker for induction of ferroptosis by direct inhibition of GPX4. To distinguish the transcriptional responses elicited by direct versus indirect inhibitors of GPX4, we profiled two direct GPX4 inhibitors, RSL3 and ML162, as well as one system Xc inhibitor, erastin, by Precision Run On followed by sequencing (PRO-seq) in IMR90 lung fibroblast cells. Briefly, IMR90 lung fibroblasts cells (a ferroptosis sensitive cell line) were treated with GPX inhibitors (RSL3 1 μM, ML162 1 μM) or system Xc inhibitor (erastin 10 UM) for one hour and samples were subjected to PRO-seq.

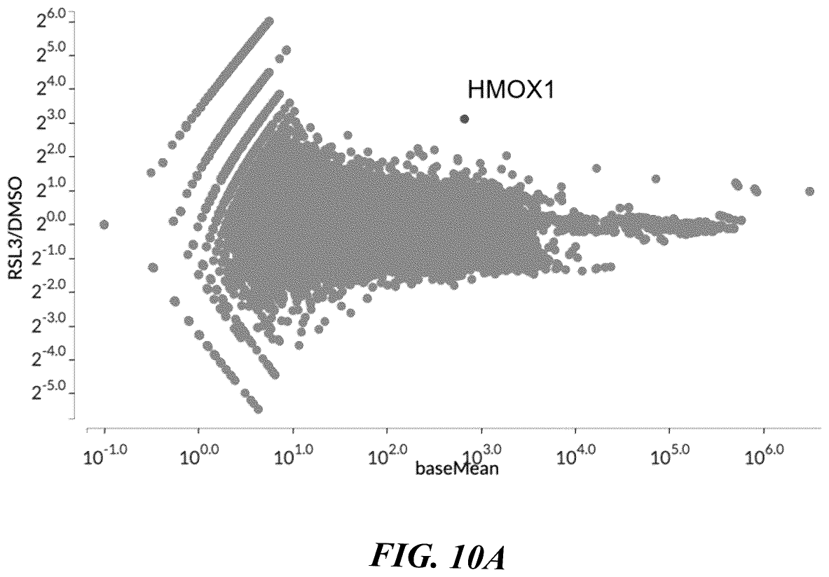

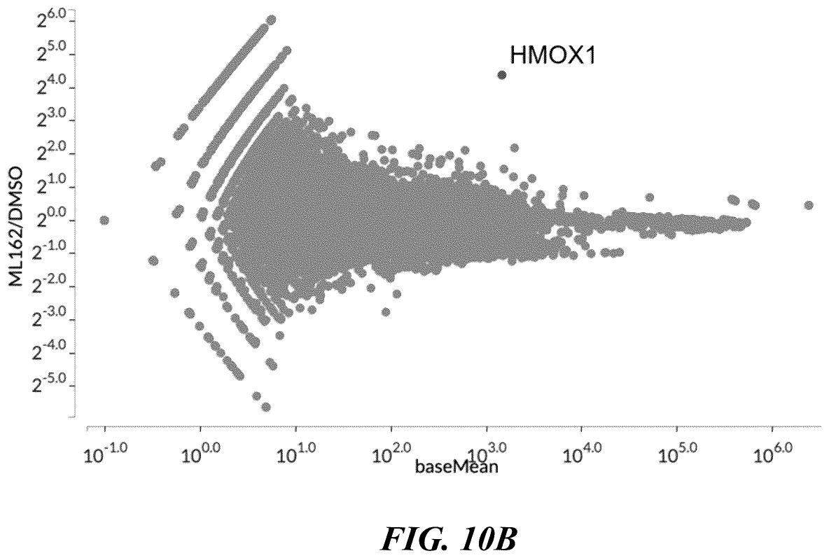

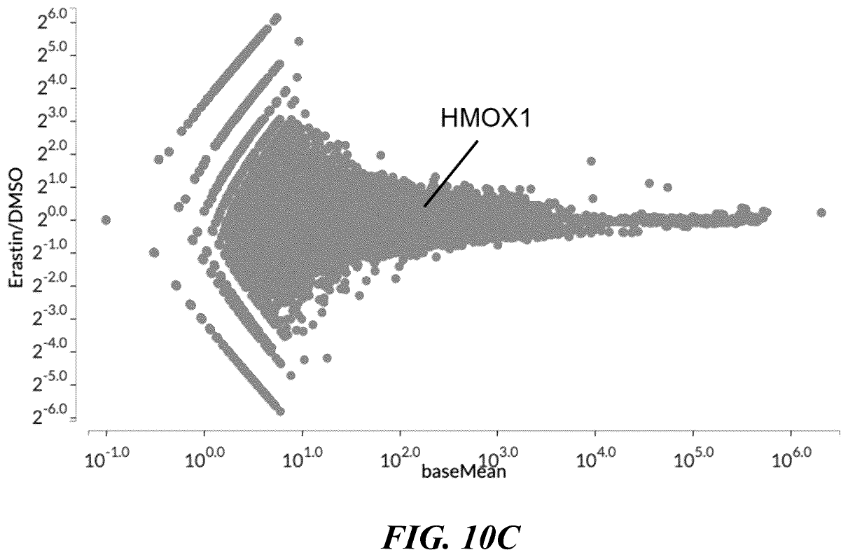

FIG. 10A, FIG. 10B, and FIG. 10C are plots 1000 showing transcriptional activity in IMR90 lung fibroblast cells exposed to RSL3, ML162, or erastin. Blue dots represent genes; HMOX1 is highlighted and shown in red in each graph. The Y-axis is log fold change; the X-axis is Base Mean. The data show that exposure of IMR90 lung fibroblast cells to the direct GPX4 inhibitors RSL3, or ML162 resulted in robust transcriptional activation of the HMOX1 gene at one hour. By contrast, no HMOX1 induction was observed in the erastin treated samples at the same time point, suggesting that rapid HMOX1 induction may be a biomarker of ferroptosis induced by direct GPX4 inhibition rather than through system Xc inhibition.

We tested several of our ferroptosis-inducing compounds for their ability to rapidly induce HMOX1 by RT-qPCR. Briefly, HT-1080 fibrosarcoma cells were treated with either 10 UM or 5 UM of compound RSL3, 8216, 1962, 1816, or 3362 for four hours. RNA was isolated and reverse transcribed and HMOX1 expression quantified by qPCR. Each sample was normalized to an ACTB housekeeping gene control and fold change was calculated relative to a DMSO control.

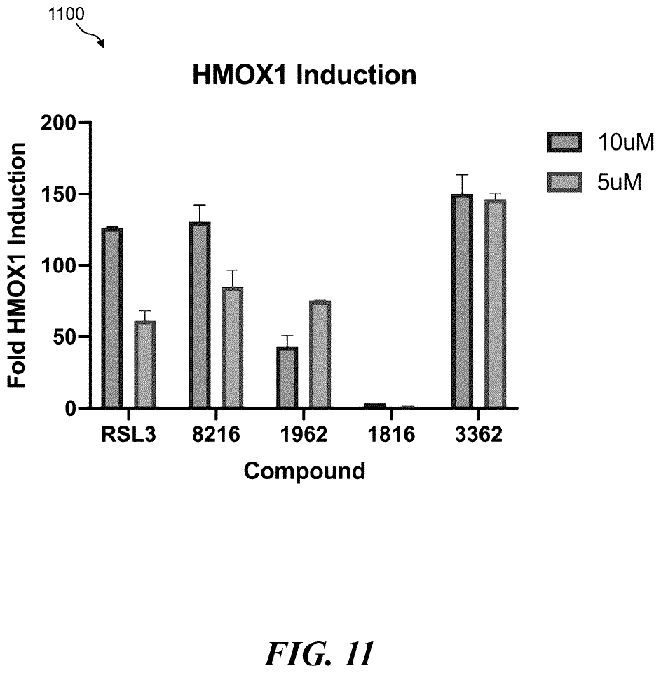

FIG. 11 is a plot 1100 showing fold HMOX1 induction in HT-1080 fibrosarcoma cells exposed to compounds RSL3, 8216, 1962, 1816, or 3362. The data show that, as expected, RSL3 induces HMOX1 dose-dependently at 10 μM (purple bars) and 5 μM (blue bars). Compound 8216 results in a remarkably similar dose-dependent HMOX1 induction, suggesting that RSL3 and 8216 may function to induce ferroptosis through similar mechanisms, namely GPX4 inhibition. Compound 1962, which binds and inhibits GPX4 in vitro, did not lead to dose responsive induction of HMOX1, suggesting a potentially non-linear relationship between target inhibition and induction of ferroptosis, potentially through interactions with off-target proteins. Compound 1816 did not induce HMOX1, consistent with the model that it does not directly inhibit GPX4, but likely induces ferroptosis through an alternate mechanism. Compound 3362 also results in induction of HMOX1, but our binding results suggest that 3362 does not bind GPX4 in vitro. Whether 3362-mediated HMOX1 activation is due to direct inhibition of GPX4 or by an alternative mechanism will be investigated in future experiments.

### Evaluating Derivatives of Compounds 8216, 6666, and 1816

Experimental results described hereinabove suggest that compound 8216 is a potent and selective inhibitor of GPX4. To determine whether similar molecules could offer other starting points for a medicinal chemistry campaign, we screened 95 additional compounds that contain a similar base structure to that of 8216. Briefly, HT-1080 fibrosarcoma cells were treated with 10 UM of a compound for 48 hours and cell viability was measured by Cell Titer Glo®. Data was collected in duplicate. A list of the compounds tested, and a summary of the average relative cell viability data is shown in Table 5.

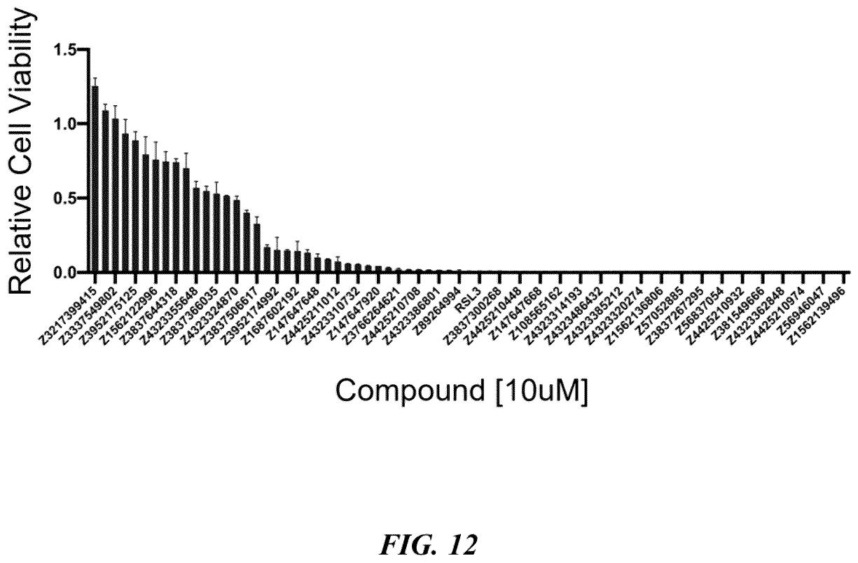

FIG. 12 is a plot 1200 showing relative viability of HT-1080 fibrosarcoma cells exposed to different 8216 derivative compounds. The data show that at 10 μM, 60 of the 74 compounds tested result in a greater than 50% reduction in HT-1080 fibrosarcoma cell viability. This result suggests that the 8216 is a good starting point for a medicinal chemistry campaign.

From the screen of 8216 derivative compounds, compound 6666 (see Table 5) was selected for further study.

An additional 34 compounds from the Enamine covalent inhibitor library with similar structures to compounds 6666 or 1816 were screened for inducing cell death. Briefly, HT-1080 fibrosarcoma cells were plated 24 hours prior to treatment on 96-well black-sided clear bottom plates. Each compound in the screening panel was tested at a 10 UM concentration for 24 hours prior to addition of Cell Titer Glo to evaluate relative cell viability.

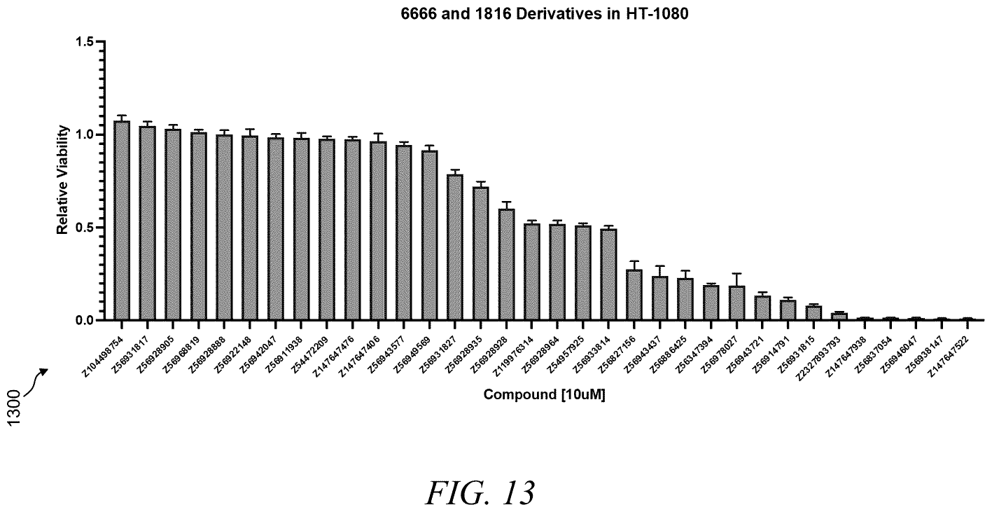

FIG. 13 is a plot 1300 showing relative viability of HT-1080 fibrosarcoma cells exposed to compounds with similar structures to compounds 6666 or 1816. The data show that 41% of the compounds tested in this screen demonstrated a relative viability >50%, with a majority of the compounds representing structures more similar to compound 6666. The compounds identified in this figure will be further evaluated in subsequent figures.

Compounds from the Enamine covalent inhibitor library with similar structures to compounds 8216, 6666 or 1816 were further screened for inducing cell death via ferroptosis. Briefly, HT-1080 fibrosarcoma cells were plated on black-sided clear bottom plates 24 hours prior to treatment, with or without the addition of 2× ferrostatin-1, which inhibits the process of ferroptosis and rescues cells from undergoing this form of cell death. Compounds were diluted in DMSO and media then added to the cells for a total of 24 hours at which point cell viability was read out via Cell Titer Glo. Compounds were tested at 7 concentrations ranging from 50 μM to 5 nM at half log dilutions.

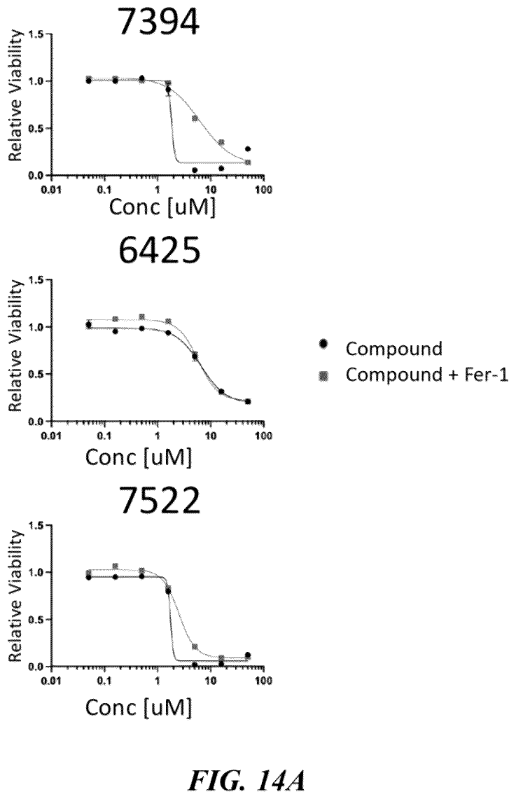

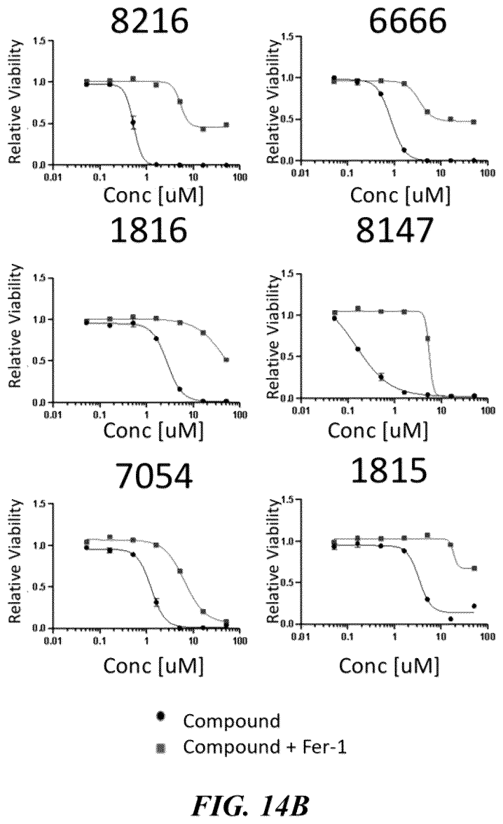

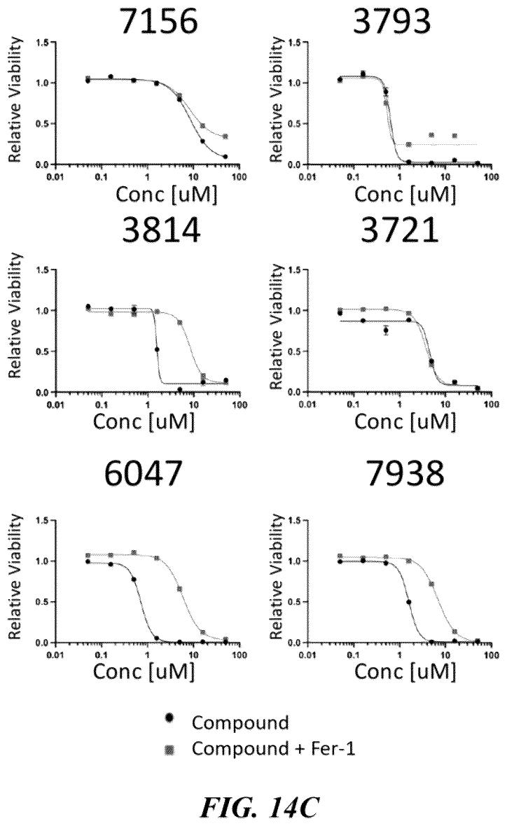

FIG. 14 is a panel of plots 1400 showing relative viability of HT-1080 fibrosarcoma cells exposed to 15 inhibitor compounds with similar structures to 8216, 6666, or 1816 tested over a range of concentrations from 50 μM to 5 nM at half log dilutions in the presence of (red line) and absence of (black line) 1.5 μM ferrostatin-1. Ferrostatin-1 is a lipophilic antioxidant which inhibits the process of ferroptosis and rescues cells from undergoing this form of cell death. The black line is the relative HT-1080 fibrosarcoma cell viability in the absence of ferrostatin-1 and the red line is the relative HT-1080 fibrosarcoma cell viability in the presence of 1.5 μM ferrostatin-1. HT-1080 fibrosarcoma cells were plated on side-blackened, clear bottom plates 24 hours prior to treatment, with or without the addition of 2× ferrostatin-1. Each compound was diluted in DMSO and media and then added to the cells for a total of 24 hours, at which point cell viability was read out via Cell Titer Glo. The data show that several compounds cause a clear shift in EC50 with the addition of ferrostatin-1 (red line shifted to the right) which is evidence to suggest that those compounds induce cell death via ferroptosis.

We tested several of the ferroptosis-inducing compounds for their ability to rapidly induce expression of HMOX1. Briefly, HT-1080 fibrosarcoma cells were plated in 6-well dishes 24 hours prior to treatment with 5 μM of 8147, 6047, 3793, Rsl3, 6666 or 8216 with and without the addition of ferrostatin-1 to the media. Cells were harvested from the plate after 4 hours of compound treatment by adding Trizol reagent directly to the plate to lyse the cells and preserve the RNA. RNA was isolated using the Direct-zol 96-well RNA kit (Zymo cat #R2054). Subsequently purity and quantity of RNA in the samples were measured using a Nanodrop (Thermo Fisher cat #ND-ONE-W). RNA samples were normalized to 2 μg and reverse transcribed to cDNA using the Multiscribe High-Capacity Reverse Transcription kit (Thermo Fisher cat #4368814) following manufacturer's instructions. Quantitative real time PCR was carried out in triplicate using SYBR Select Master Mix (Thermo Fisher cat #4472903) following manufacturer's instructions with primers designed against HMOX1 and ActB from IDT. HMOX1 expression levels were normalized to ActB for each sample then fold change was determined against the vehicle control, DMSO.

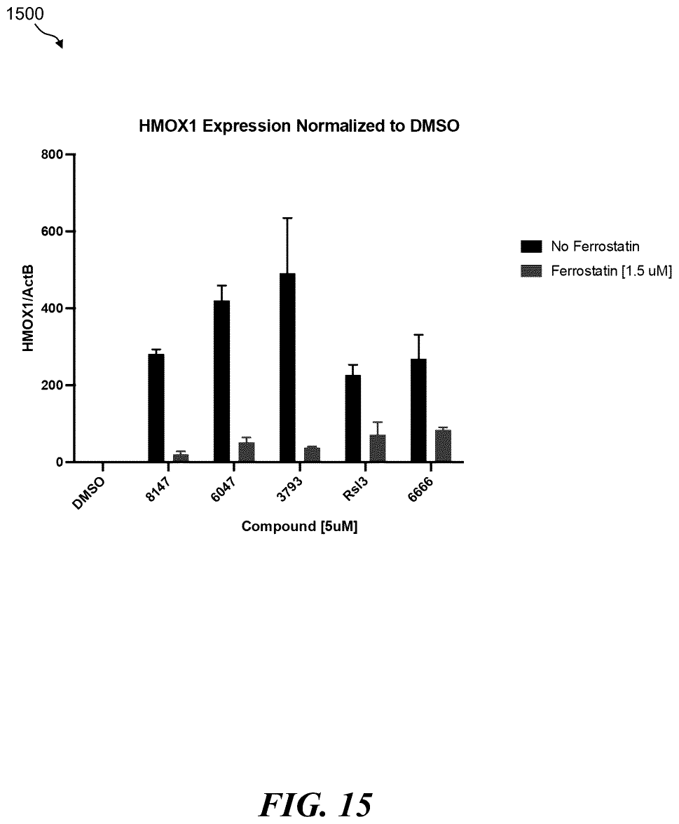

FIG. 15 is a plot 1500 showing fold HMOX1 induction in HT-1080 fibrosarcoma cells exposed to compounds 8147, 6047, 3793, Rsl3, 6666 or 8216. The data show that all of the compounds tested demonstrated an upregulation of HMOX1 which was rescued by the addition of ferrostatin-1, to varying degrees, suggesting that the compounds induce ferroptosis.

An enzymatic assay that measures the reduction of glutathione by GPX4 was used to test the ability of compounds 8216, 6666, 8147, and 3793 to inhibit the activity of GPX4. The enzymatic assay was performed using a commercially available kit (Cayman cat #701880). The compounds were tested at 8 concentrations in duplicate with the compound being diluted following manufacturer's instructions. The enzymatic reaction was allowed to continue for 1 hour prior to reading out the kinetic shift in absorbance that occurs as result of GPX4 reducing glutathione.

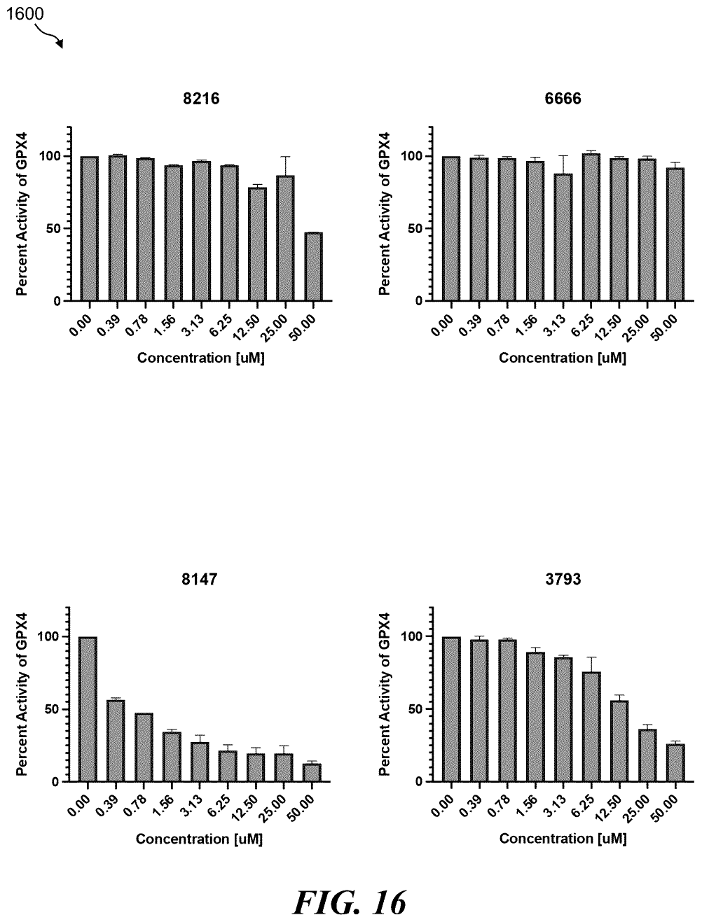

FIG. 16 is a panel of plots 1600 showing percent activity of GPX4 in response to exposure to compounds 8216, 6666, 8147, or 3793. The data show that compounds 8147 and 3793 display a concentration-dependent inhibition of GPX4 activity, suggesting that these compounds could induce ferroptosis by directly inhibiting GPX4 in vivo.

Compounds 6666, 6047, 3973, 8216, and 8147 were also evaluated for inducing lipid peroxidation in HT-1080 fibrosarcoma cells in the presence or absence of 1.5 UM ferrostatin-1. Briefly, HT-1080 fibrosarcoma cells were treated with 10 UM of compound with or without 1.5 μM ferrostatin-1 and incubated at 37° C. for 1 hour. Bodipy C-11 and Hoescht stain were then added to the media, the cells were allowed to incubate for an additional 30 minutes and then were imaged on the Opera Phenix confocal screening system.

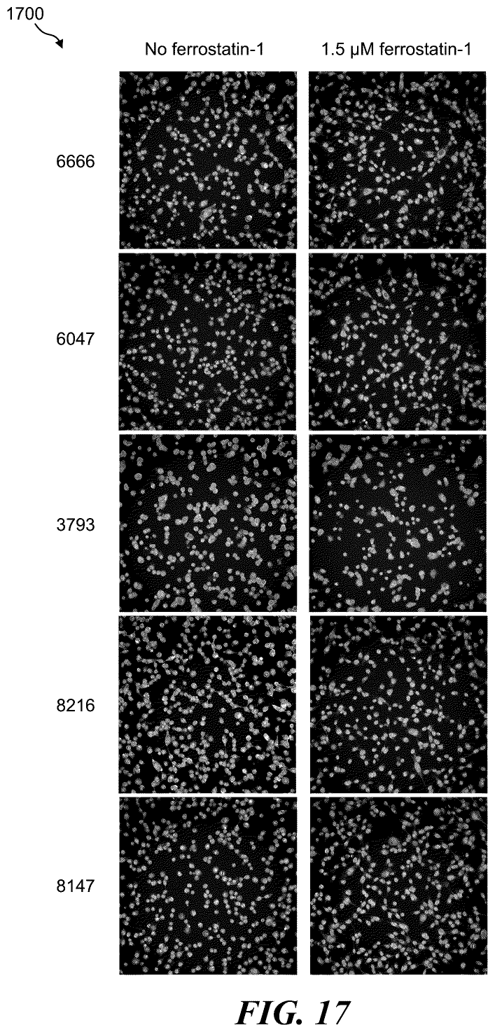

FIG. 17 shows a panel of representative images 1700 from fluorescent confocal microscopy of HT-1080 fibrosarcoma cells treated with compounds 6666, 6047, 3973, 8216, or 8147 in the presence or absence of 1.5 UM ferrostatin-1. The data show that all compounds tested display a shift in emission reflective of lipid peroxidation, with ferrostatin-1 rescuing the cells from undergoing lipid peroxidation (indicated by orange i.e., no shift in emission). Blue is the nuclear stain Hoescht to show cell bodies.

To determine whether the ferroptosis-inducing compounds 6666 and 8147 could function through direct binding and inhibition of GPX4, we tested binding of each compound to recombinant GPX4 protein in vitro by intact protein mass spectrometry. Compound 8216 was used as a positive control (see FIG. 6). Briefly, 15 μL of GPX4 protein (Cayman Chemical cat #SO960203]) was incubated with 100 UM of each compound (4.5 L) and diluted with 25.5 μl of PBS. The incubation was performed at room temperature for 2 hours and then the reaction was quenched with 3 μL of trifluoroacetic acid. Samples were subjected to UPLC coupled to Synapt G2 mass spectrometer to evaluate if binding of the test compound to GPX4 occurs. Binding is evident by a mass shift equivalent to the mass size of the compound minus a chlorine atom plus the mass size of the GPX4 protein, as described by the manufacturer.

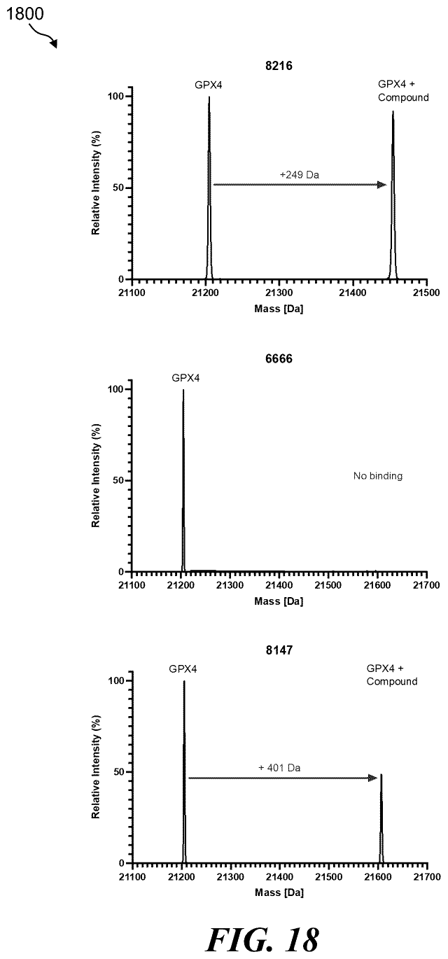

FIG. 18 is a panel of plots 1800 showing the intact protein mass spectrometry analyses of compounds 8216, 6666, and 8147. The data show that compounds 8216 and 8147 demonstrate a mass shift. However, compound 6666 did not show a mass shift which suggests that compound 6666 does not bind to GPX4 under these conditions, while compounds 8216 and 8147 do.

### Computer-Aided Design of Compound 8216 Derivatives

As an orthogonal approach, we used the Autogrow4 algorithm to predict molecules that could bind to the GPX4 active site. Autogrow4 is a genetic algorithm that takes in a protein structure and through a series of generations (max of 30 generations) builds molecules using commonly used small molecule fragments. Throughout each generation small fragments that bind with high predicted affinity to the target will have a chance to move forward to the next level in the algorithm and interact with other small fragments to build a larger compound. At the end of each generation filters are applied to molecules so that toxic and mutant compounds are eliminated and don't proceed to the next level. In addition, Lipinski's rule was applied in order to keep the size of the molecules below 500 Da.

We ran the algorithm independently 15 times on the active site of GPX4 and recovered several compounds predicted to interact with the site with estimated binding energies ranging from −8 to −6 kcal/mol. In one of the runs, 68 compounds contained the 8216 structure as part of the molecule. We applied a clustering analysis to these compounds to divide them into similar groups. Once the clustering was done, the centroid of the clusters was selected (n=9) and among those, 4 compounds were synthesizable. These independently-derived molecules are currently being synthesized. The chemical structures of the nine compounds identified are shown in Table 4.

### SYNTHESIS EXAMPLES

### Synthesis reaction scheme of N-[4-(4-bromophenyl)thiazol-2-yl]-2-chloro-N-(3,5-dimethylphenyl)acetamide (Compound 90)

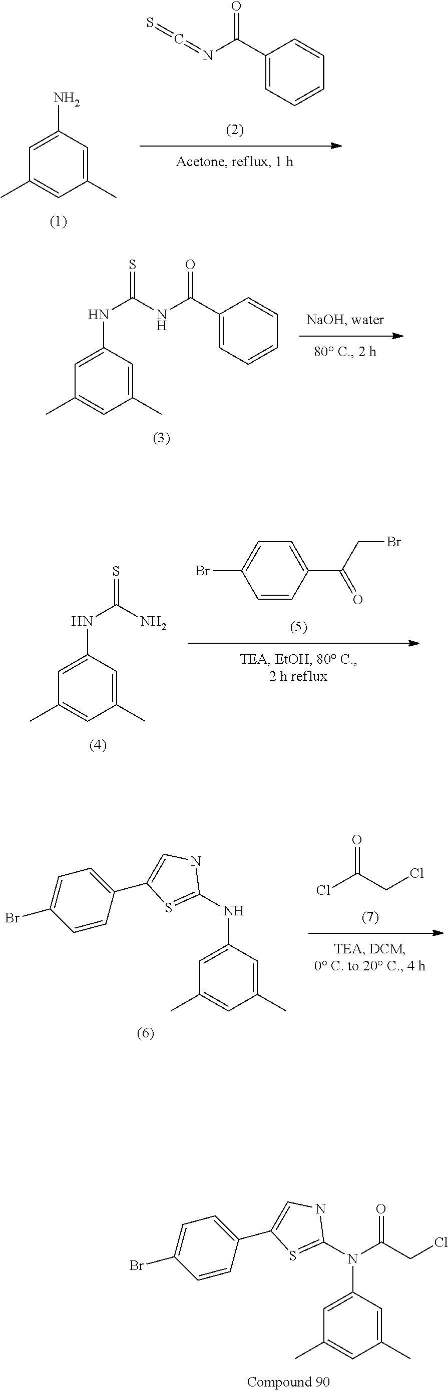

### Detailed Synthesis of N-[4-(4-bromophenyl)thiazol-2-yl]-2-chloro-N-(3,5-dimethylphenyl)acetamide (Compound 90)

### Step (a): Synthesis of N-[(3,5-dimethylphenyl)carbamothioyl]benzamide

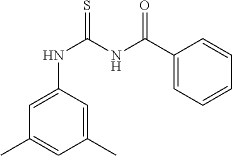 [SMILES: Cc1cc(C)cc(NC(=S)NC(=O)c2ccccc2)c1]

22.2 g (136 mmol) of 3,5-dimethylaniline (1) was added in one portion to a solution of benzoyl isothiocyanate (2) at 20° C., prepared by dissolving 15 g (123 mmol) in 60 mL acetone. The mixture was heated to 60° C. and stirred for 1 h. LC-MS showed 3,5-dimethylaniline (1) was consumed completely and one main peak with desired MS (285.1, (M+H)⁺) was detected. The reaction mixture was then cooled to room temperature and poured into crushed ice, whereupon a white precipitate formed. The precipitate was collected by filtration, washed with 100 ml water and dried over air to give 28 g of crude white solid intermediate product N-[(3,5-dimethylphenyl)carbamothioyl]benzamide (3).

MS (ESI): calculated 285.1 [(M+H)⁺]; measured 285.1 [(M+H)⁺].

### Step (b): Synthesis of 3,5-dimethylphenyl)thiourea

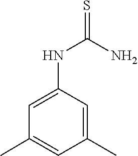

28 g of N-[(3,5-dimethylphenyl)carbamothioyl]benzamide (3) was added in portions to a solution of NaOH in water, prepared by dissolving 5 g NaOH (140 mmol) in 200 ml of water. The mixture was heated at 80° C. and stirred for 2 hr. LC-MS showed N-[(3,5-dimethylphenyl)carbamothioyl]benzamide (3) was consumed completely and one main peak with desired MS (181.1, [M+H]⁺) was detected. The mixture was then cooled to room temperature and added dropwise to 200 ml of a 1N aqueous hydrochloric acid solution. The resulting mixture was diluted with 100 ml of water and extracted with EtOAc (300 mL×3). The combined organic layers were washed with saturated brine solution (300 mL×3), dried over Na₂SO₄, filtered and concentrated under reduced pressure to give a residue. The residue was triturated in 50 mL of methyl tert-butyl ether to yield 15 g (83.2 mmol) of the white solid intermediate product (3,5-dimethylphenyl)thiourea (4).

MS (ESI): calculated 181.1 [(M+H)⁺]; measured 181.1 [(M+H)⁺].

¹H NMR (400 MHZ, DMSO-d₆) δ 9.55 (br s, 1H), 7.94-7.08 (m, 2H), 6.96 (br s, 2H), 6.76 (br s, 1H), 2.24 (s, 6H).

### Step (c): Synthesis of 4-(4-bromophenyl)-N-(3,5-dimethylphenyl)thiazol-2-amine

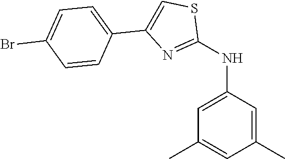 [SMILES: Cc1cc(C)cc(Nc2nc(-c3ccc(Br)cc3)cs2)c1]

15.4 g (55.5 mmol) of 2-bromo-1-(4-bromophenyl)ethanone (5) and 27.5 mL (20.0 g, 197 mmol) triethylamine was added to a 20° C. solution of (3,5-dimethylphenyl)thiourea (4), prepared by dissolving 10 g (55.5 mmol) in 100 mL ethanol. The mixture was heated to 80° C. and stirred for 2 h. LC-MS showed (3,5-dimethylphenyl)thiourea (4) was consumed completely and one main peak with desired MS (358.9 [M (⁷⁹Br)+H]⁺, 360.9 [M (⁸¹Br)+H]⁺) was detected. The reaction mixture was then cooled to room temperature, quenched with 100 ml water at 20° C. and extracted with 3 aliquots of 100 mL EtOAc. The combined organic layers were washed with 3 aliquots of 100 mL saturated brine, dried over Na₂SO₄, filtered and concentrated under reduced pressure to give a residue. The residue was purified by silica gel flash chromatography (300 g SepaFlash® silica column, ethyl acetate/petroleum, gradient 0%-30% ethyl acetate/petroleum ether @ 200 mL/min) to yield 19 g (52.9 mmol) of the yellow solid intermediate product 4-(4-bromophenyl)-N-(3,5-dimethylphenyl)thiazol-2-amine (6).

MS (ESI): calculated 359.0 [M (⁷⁹Br)+H]⁺, 361.0 [M (⁸¹Br)+H]⁺; measured 358.9 [M (⁷⁹Br)+H]⁺, 360.9 [M (⁸¹Br)+H]⁺.

¹H NMR (400 MHZ, DMSO-d₆) δ 10.14 (s, 1H), 7.86 (d, J=8.4 Hz, 2H), 7.62 (d, J=8.4 Hz, 2H), 7.38 (s, 1H), 7.30 (s, 1H), 7.31 (s, 1H), 6.62 (s, 1H), 2.27 (s, 6H).

### Step (d): Synthesis of N-[4-(4-bromophenyl)thiazol-2-yl]-2-chloro-N-(3,5-dimethylphenyl)acetamide

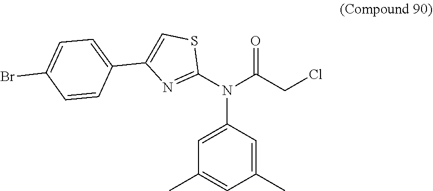 [SMILES: Cc1cc(C)cc(N(C(=O)CCl)c2nc(-c3ccc(Br)cc3)cs2)c1]

84.5 mg (0.835 mmol) triethylamine was added to a solution of 4-(4-bromophenyl)-N-(3,5-dimethylphenyl)thiazol-2-amine (6), which was prepared by dissolving 100 mg (0.278 mmol) in 2 mL dichloromethane. The mixture was cooled to 0° C. and 62.9 mg 2-chloroacetyl chloride (0.557 mmol) (7) was added dropwise. The mixture was then heated to 20° C. and stirred for 4 h. LC-MS showed 4-(4-bromophenyl)-N-(3,5-dimethylphenyl)thiazol-2-amine (6) was consumed completely and desired MS (435.0 [M (⁷⁹Br)+H]⁺, 437.0 [M (⁸¹Br)+H]⁺) was detected. The reaction was concentrated under reduced pressure to give a residue. The residue was purified by preparative HPLC (column: Waters Xbridge, 5 μm, 150×25 mm; mobile phase A: water containing 0.1% trifluoroacetic acid, mobile phase B: acetonitrile; gradient elution of 60%-90% B:A over 9 min) and lyophilized to give 21 mg (0.048 mmol) of the white solid final product N-[4-(4-bromophenyl) thiazol-2-yl]-2-chloro-N-(3,5-dimethylphenyl)acetamide.

MS (ESI): calculated 435.0 [M (⁷⁹Br)+H]⁺, 437.0 [M (⁸¹Br)+H]⁺; measured 435.0 [M (⁷⁹Br)+H]⁺, 437.0 [M (⁸¹Br)+H]⁺.

¹H NMR (400 MHZ, DMSO-d₆) δ 7.85 (s, 1H), 7.61-7.56 (m, 2H), 7.56-7.52 (m, 2H), 7.20 (s, 1H), 7.17 (s, 2H), 4.29 (s, 2H), 2.35 (s, 6H).

### Synthesis Reaction Scheme of Compound 637

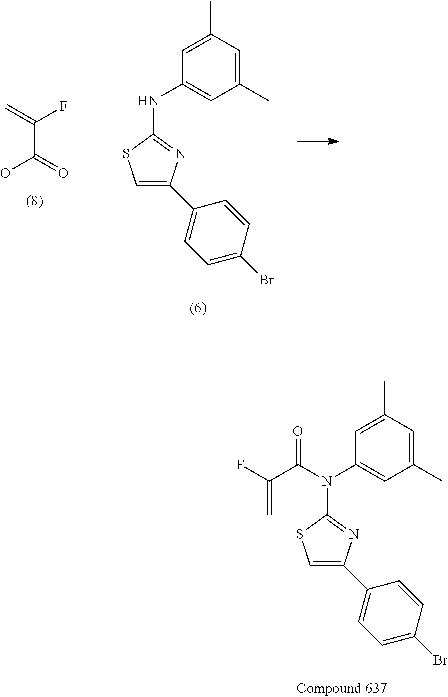

### Detailed Synthesis of Compound 637

0.06 g (0.67 mmol, 1.2 eq) of compound (8) was dissolved in DCM (10 mL) under an argon atmosphere. The solution was cooled to 0° C. and the following were added sequentially 0.2 g (0.56 mmol, 1 eq) of compound (6) and 0.172 g (0.83 mmol, 1.5 eq) DCC. The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain 0.061 g (0.141 mmol, 25.5% yield) of compound 637.

### Synthesis Reaction Scheme of Compound 635

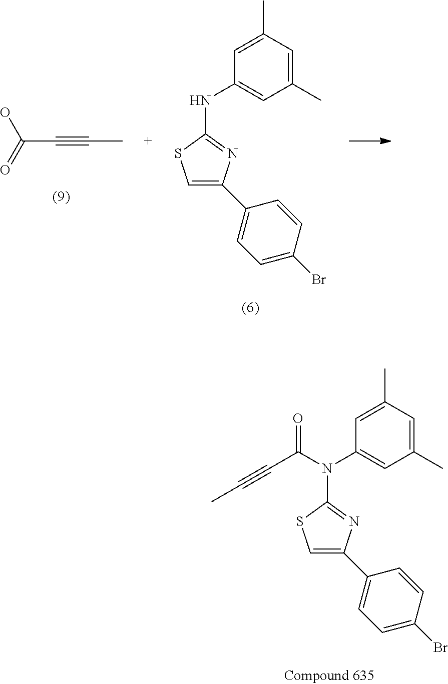

### Detailed Synthesis of Compound 635

0.056 g (0.67 mmol, 1.2 eq) compound (9) was dissolved in DCM (10 mL) under an argon atmosphere. The solution was cooled to 0° C. and the following were added sequentially 0.2 g (0.56 mmol, 1 eq) compound (6) and DCC (0.172 g, 0.83 mmol, 1.5 eq). The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain compound 635 (0.012 g, 0.0282 mmol, 5% yield).

### Synthesis Reaction Scheme of Compound 636

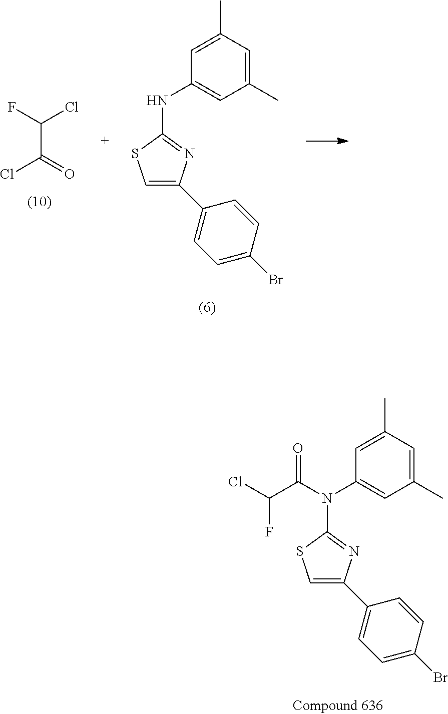 [SMILES: O=C(Cl)C(F)Cl ; Cc1cc(C)cc(Nc2nc(-c3ccc(Br)cc3)cs2)c1 ; Cc1cc(C)cc(N(C(=O)C(F)Cl)c2nc(-c3ccc(Br)cc3)cs2)c1]

### Detailed Synthesis of Compound 636

0.2 g (0.56 mmol, 1 eq) of compound (6) was dissolved in DCM (10 mL) under an argon atmosphere. The solution was cooled to 0° C. and the following were added sequentially 0.36 g (2.78 mmol, 5 eq) DIPEA and 0.095 g (0.72 mmol, 1.3 eq) compound (10) dropwise. The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain compound 636 (0.094 g, 0.2 mmol, 37.2% yield).

### Synthesis Reaction Scheme of Compound 634

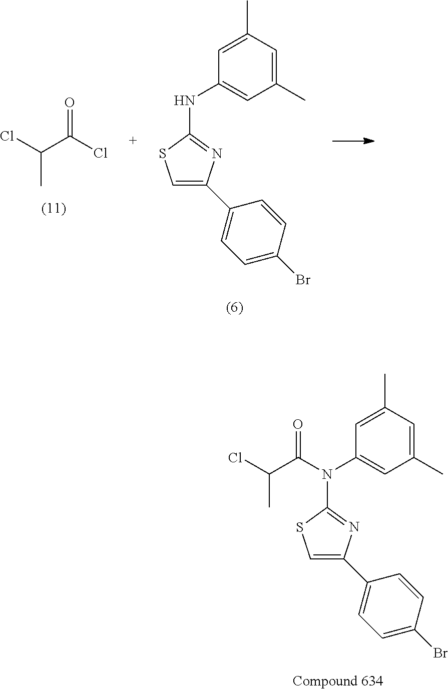

### Detailed Synthesis of Compound 634

0.2 g (0.56 mmol, 1 eq) compound (6) was dissolved in DCM (10 mL) under an argon atmosphere. The solution was cooled to 0° C. and the following were added sequentially 0.36 g (2.78 mmol, 5 eq) DIPEA and 0.092 g (0.72 mmol, 1.3 eq) compound (11) dropwise. The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain compound 634 (0.099 g, 0.22 mmol, 39.5% yield).

### Synthesis Reaction Scheme of Compound 641

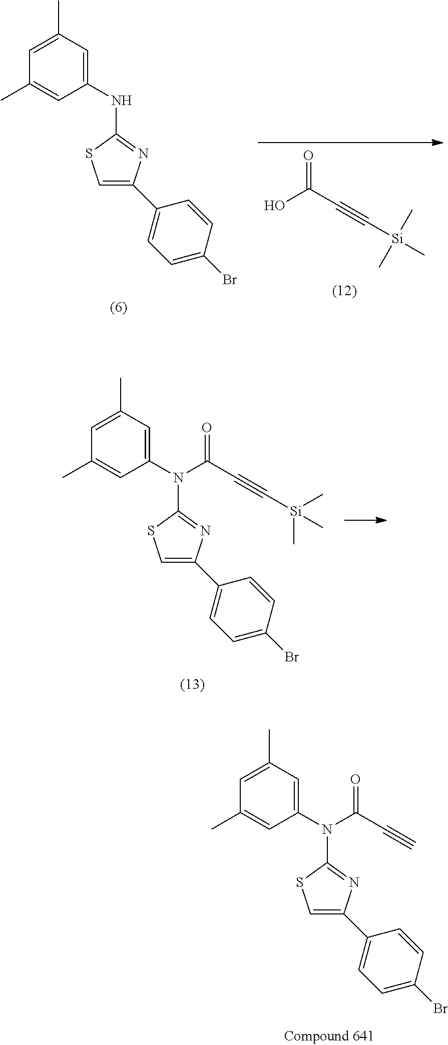

### Detailed Synthesis of Compound 641

0.20 g (0.56 mmol, 1 eq) compound (6) was dissolved in DCM (10 mL) under an argon atmosphere. To the solution were added sequentially compound (12) 3-(trimethylsilyl)propiolic acid (0.095 g, 0.670 mmol, 1.2 eq) and DCC (0.172 g, 0.830 mmol, 1.5 eq) in a basic solution. The reaction mixture was stirred overnight at room temperature. The resulting mixture was diluted with 20 mL DCM. The DCU byproduct was filtered through a pad of celite and washed with minimal amount of DCM. The combined DCM extracts were washed with water (3×20 mL), dried over anhydrous Na₂SO₄, and evaporated under reduced pressure to obtain desired intermediate compound (13) (0.20 g, 0.41 mmol, 74.3% yield), which was used in the next step without further purification.

0.20 g (0.41 mmol, 1 eq) compound (13) was dissolved in THF (10 mL) under an argon atmosphere. The solution was cooled to 0° C. and TBAF (0.46 mL, 1 M in THF, 1.1 eq) was added. The reaction mixture was warmed to room temperature and stirred overnight. The resulting mixture was diluted with DCM (30 mL), washed with water (3×20 mL). The organic layer was dried over anhydrous Na₂SO₄ and evaporated under reduced pressure. The residue was purified by HPLC to obtain compound 634 (0.0103 g, 0.025 mmol, 6% yield).

### Synthesis Reaction Scheme of Compound 639

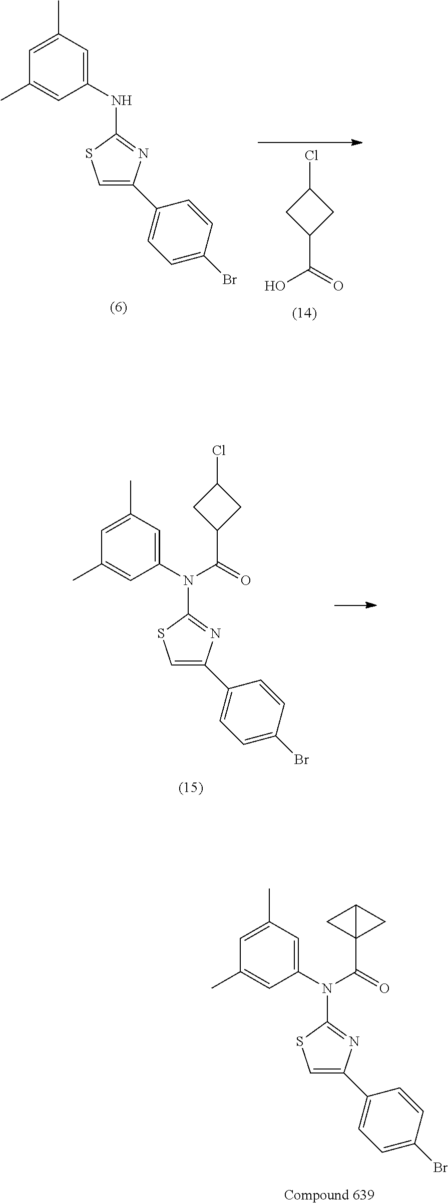 [SMILES: Cc1cc(C)cc(Nc2nc(-c3ccc(Br)cc3)cs2)c1 ; O=C(O)C1CC(Cl)C1 ; Cc1cc(C)cc(N(C(=O)C2CC(Cl)C2)c2nc(-c3ccc(Br)cc3)cs2)c1 ; Cc1cc(C)cc(N(C(=O)C23CC2C3)c2nc(-c3ccc(Br)cc3)cs2)c1]

### Detailed Synthesis of Compound 639

0.50 g (1.39 mmol, 1 eq) compound (6) was dissolved in DCM (20 mL) under an argon atmosphere. To the solution were added sequentially compound (14) 3-chlorocyclobutane-1-carboxylic acid (0.225 g, 1.670 mmol, 1.2 eq) and DCC (0.431 g, 2.09 mmol, 1.5 eq) in a basic solution. The reaction mixture was stirred overnight at room temperature. The resulting mixture was diluted with DCM (30 mL). The DCU byproduct was filtered through a pad of celite and washed with minimal amount of DCM. The combined DCM extracts was washed with water (3×30 mL), dried over anhydrous Na₂SO₄, and evaporated under reduced pressure to obtain desired intermediate compound (15) (0.40 g, 0.84 mmol, 60.4% yield), which was used in the next step without further purification.

0.4 g (0.84 mmol, 1 eq) compound (15) was dissolved in THF (15 mL) under an argon atmosphere. The solution was cooled to −40° C. and the following were added dropwise LiHMDS (0.92 mL, 1.1 M in THF, 1.1 eq) and DMAP. The reaction mixture was slowly warmed to 0° C. and stirred for 30 min. The resulting mixture was quenched with a saturated solution of NH₄Cl and extracted with EtOAc (3×30 mL). The combined organic layer was dried over anhydrous Na₂SO₄ and evaporated under reduced pressure. The residue was purified by HPLC to obtain compound 639 (0.0092 g, 0.02 mmol, 2.44% yield).

### Synthesis Reaction Scheme of Compound 640

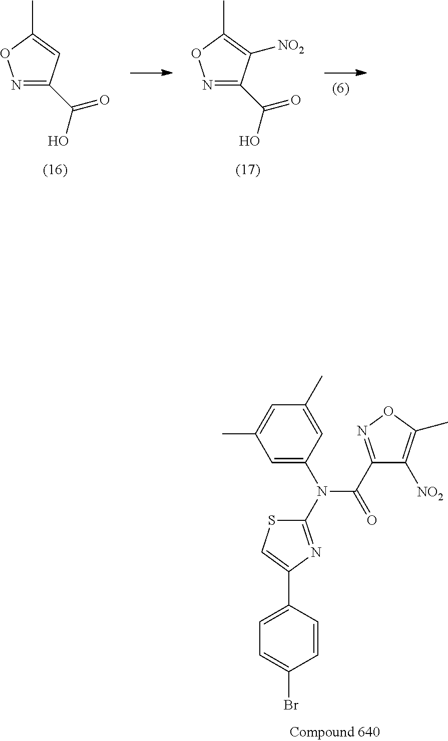

### Detailed Synthesis of Compound 640

1 g (7.87 mmol, 1 eq) compound (16) was dissolved in 20 mL concentrated H₂SO₄ under an argon atmosphere. 1.19 g (11.8 mmol, 1.5 eq) KNO₃ was added to the solution. The reaction mixture was stirred overnight at 50° C. The resulting mixture was poured in ice water and extracted with DCM (3×50 mL). The combined organic layer was dried over anhydrous Na₂SO₄ and evaporated under reduced pressure to obtain compound (17) (1.1 g, 6.3 mmol, 81.2% yield).

0.20 g (0.56 mmol, 1 eq) compound (6) was dissolved in DCM (10 mL) under an argon atmosphere. To the solution were added sequentially 0.115 g (0.670 mmol, 1.2 eq) compound (17) and 0.172 g (0.830 mmol, 1.5 eq) DCC in a basic solution. The reaction mixture was stirred overnight at room temperature. The resulting mixture was diluted with DCM (20 mL). The DCU byproduct was filtered through a pad of celite and washed with minimal amount of DCM. The combined DCM extracts was washed with water (3×20 mL), dried over anhydrous Na₂SO₄, and evaporated under reduced pressure. The residue was purified by HPLC to obtain compound 640 (0.0473 g, 0.092 mmol, 16.5% yield).

Compounds B1-B28 were prepared by an analogous synthetic route to compounds 90, 634, 635, 636, 637, 639, 640, and 641 as illustrated above.

### Synthesis Reaction Scheme of Compound 593

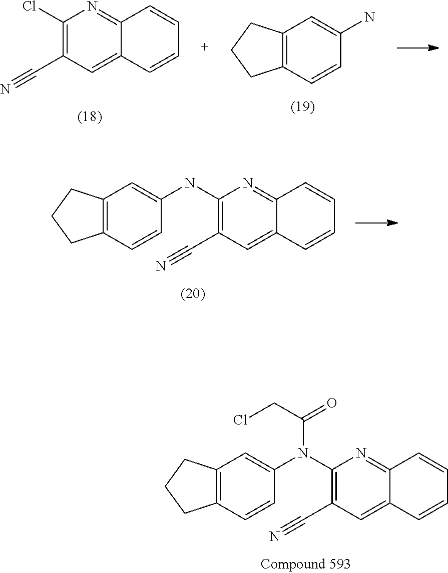 [SMILES: N#Cc1cc2ccccc2nc1Cl ; Nc1ccc2c(c1)CCC2 ; N#Cc1cc2ccccc2nc1Nc1ccc2c(c1)CCC2 ; N#Cc1cc2ccccc2nc1N(C(=O)CCl)c1ccc2c(c1)CCC2]

### Detailed Synthesis of Compound 593

1 g, 5.3 mmol, 1 eq) compound (18) was dissolved in 50 mL dioxane under an argon atmosphere. To the solution were added sequentially compound (19) (0.706 g, 5.3 mmol, 1 eq), tris(dibenzylideneacetone)dipalladium(0) (0.485 g, 0.53, mol, 0.1 eq), [5-(diphenylphosphanyl)-9,9-dimethyl-9H-xanthen-4-yl]diphenylphosphane (0.46 g, 0.80 mmol, 0.15 eq), and Cs₂CO₃ (3.4 g, 10.4 mol, 3 eq). The reaction mixture was stirred overnight at 100° C. The resulting mixture was diluted with EtOAc (200 mL), washed with water and brine, dried over anhydrous Na₂SO₄, and evaporated under reduced pressure. The residue was purified by column chromatography to obtain compound (20) (0.8 g, 2.8 mmol, 52.8% yield).

0.1 g (0.35 mmol, 1 eq) compound (20) was dissolved in DCM (10 mL) under an argon atmosphere. The solution was cooled to 0° C. and the following were added sequentially and dropwise: DIPEA (0.045 g, 0.35 mmol, 1 eq) and 2-chloroacetyl chloride (0.04 g, 0.35 mmol, 1 eq). The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain compound 593 (0.008 g, 0.0221 mmol, 6.3% yield).

### Synthesis Reaction Scheme of Compound 587

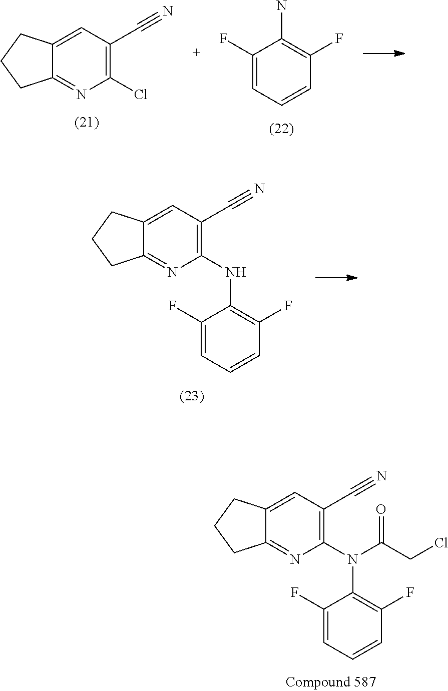 [SMILES: N#Cc1cc2c(nc1Cl)CCC2 ; Nc1c(F)cccc1F ; N#Cc1cc2c(nc1Nc1c(F)cccc1F)CCC2 ; N#Cc1cc2c(nc1N(C(=O)CCl)c1c(F)cccc1F)CCC2]

### Detailed Synthesis of Compound 587

1 g (5.6 mmol, 1 eq) compound (21) was dissolved in dioxane (50 mL) under an argon atmosphere. To the solution were added sequentially compound (22) (0.723 g, 5.6 mmol, 1 eq), tris(dibenzylideneacetone)dipalladium(0) (0.513 g, 0.56, mol, 0.1 eq), [5-(diphenylphosphanyl)-9,9-dimethyl-9H-xanthen-4-yl]diphenylphosphane (0.486 g, 0.84 mmol, 0.15 eq), and Cs₂CO₃ (4 g, 12.2 mol, 3 eq). The reaction mixture was stirred overnight at 100° C. The resulting mixture was diluted with EtOAc (200 mL), washed with water and brine, dried over anhydrous Na₂SO₄, and evaporated under reduced pressure. The residue was purified by column chromatography to obtain compound (23) (0.12 g, 0.442 mmol, 7.9% yield).

0.12 g (0.44 mmol, 1 eq) Compound (23) was dissolved in DCM (10 mL) under an argon atmosphere. The solution was cooled to 0° C. and the following were added sequentially DIPEA (0.057 g, 0.44 mmol, 1 eq) and 2-chloroacetyl chloride (0.05 g, 0.44 mmol, 1 eq) dropwise. The reaction mixture was warmed to room temperature and stirred overnight. The resulting mixture was diluted with DCM, washed with water and brine, dried over anhydrous Na₂SO₄, and evaporated under reduced pressure. The residue was purified by HPLC to obtain compound 587 (0.0114 g, 0.032 mmol, 7.4% yield).

Compounds A1, A2, and A4-A47 were prepared by an analogous synthetic route to compound A3 as illustrated below.

### Example A3: 2-Chloro-N-(3,5-dimethoxyphenyl)-N-(4-fluorobenzyl)acetamide

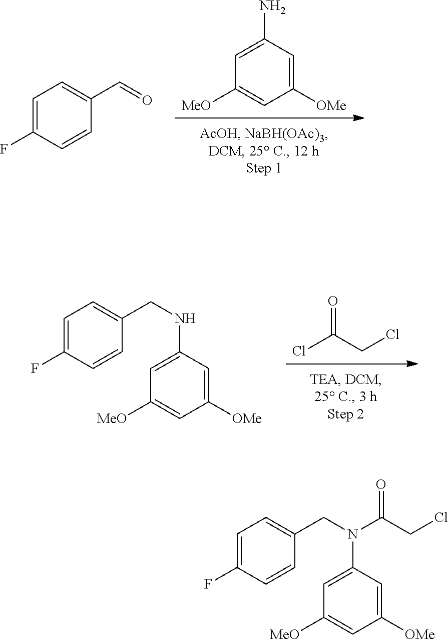 [SMILES: O=Cc1ccc(F)cc1 ; COc1cc(N)cc(OC)c1 ; COc1cc(NCc2ccc(F)cc2)cc(OC)c1 ; O=C(Cl)CCl ; COc1cc(OC)cc(N(Cc2ccc(F)cc2)C(=O)CCl)c1]

### Step 1: N-(4-Fluorobenzyl)-3,5-dimethoxyaniline

To a solution of 4-fluorobenzaldehyde (2 g, 16.1 mmol, 1.6 mL) in DCM (30 mL) was added 3,5-dimethoxyaniline (2.2 g, 14.5 mmol), acetic acid (968 mg, 16.1 mmol, 0.922 mL) and NaBH(OAc)₃ (4.6 g, 21.9 mmol). The mixture was stirred at 25° C. for 12 h. The reaction mixture was partitioned between aqueous 1M NaOH (20 mL) and DCM (20 mL). The aqueous phase was extracted with DCM (20 mL×3), dried over Na₂SO₄, filtered, and concentrated under reduced pressure to give a residue which was purified by flash silica gel chromatography (20 g silica) eluting with a gradient of 0-10% EtOAc/petroleum ether at a flow rate of 120 mL/min. N-(4-Fluorobenzyl)-3,5-dimethoxyaniline (3.1 g, 75% yield) was obtained as a yellow oil. M+H⁺=262.2 (LCMS); ¹H NMR (400 MHZ, DMSO-d₆) δ 7.36 (dd, J=5.8, 8.1 Hz, 2H), 7.13 (t, J=8.8 Hz, 2H), 6.26 (br t, J=5.9 Hz, 1H), 5.78-5.68 (m, 3H), 4.20 (br d, J=5.9 Hz, 2H), 3.61 (s, 6H).

### Step 2: 2-Chloro-N-(3,5-dimethoxyphenyl)-N-(4-fluorobenzyl)acetamide

To a solution of N-(4-fluorobenzyl)-3,5-dimethoxyaniline (200 mg, 0.765 mmol) in DCM (4 mL) was added triethylamine (155 mg, 1.5 mmol, 0.213 mL) and 2-chloroacetyl chloride (173 mg, 1.5 mmol, 0.122 mL). The mixture was stirred at 25° C. for 3 h. The reaction mixture was concentrated under reduced pressure to give a residue. The residue was partitioned between water (10 mL) and EtOAc (10 mL). The aqueous phase was extracted with EtOAc (10 mL×3), dried over Na₂SO₄, filtered, and concentrated under reduced pressure to give a residue which was purified by preparative TLC (silica) eluting with 20% EtOAc/petroleum ether. The title compound (131 mg, 49% yield) was obtained as a white solid. M+H⁺=338.0 (LCMS); 1H NMR (400 MHZ, CD₃CN) δ=7.27-7.21 (m, 2H), 7.06-7.00 (m, 2H), 6.46 (t, J=2.3 Hz, 1H), 6.30 (d, J=2.3 Hz, 2H), 4.85 (s, 2H), 4.02 (s, 2H), 3.70 (s, 6H).

TABLE 3
Additional Compounds of the invention

| | |
|---|---|
| 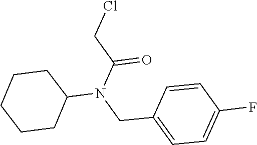 [SMILES: O=C(CCl)N(Cc1ccc(F)cc1)C1CCCCC1] | (Compound 1) |
| 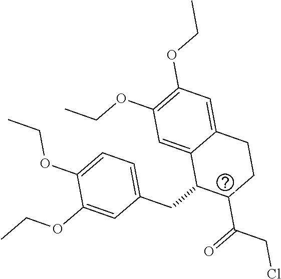 [SMILES: CCOc1ccc(C[C@@H]2c3cc(OCC)c(OCC)cc3CCC2C(=O)CCl)cc1OCC] | (Compound 2) |
| 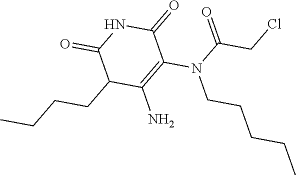 [SMILES: CCCCCN(C(=O)CCl)C1=C(N)C(CCCC)C(=O)NC1=O] | (Compound 3) |
| 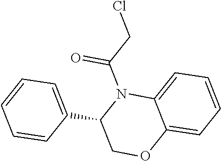 [SMILES: O=C(CCl)N1c2ccccc2OC[C@@H]1c1ccccc1] | (Compound 4) |
| 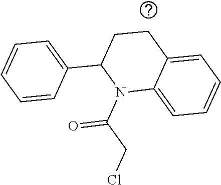 [SMILES: O=C(CCl)N1c2ccccc2CCC1c1ccccc1] | (Compound 5) |
| 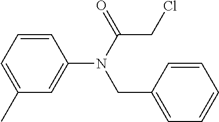 [SMILES: Cc1cccc(N(Cc2ccccc2)C(=O)CCl)c1] | (Compound 6) |
| 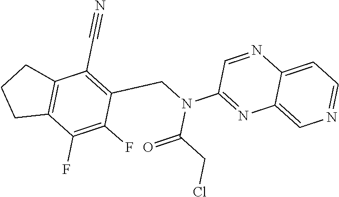 [SMILES: N#Cc1c(CN(C(=O)CCl)c2cnc3ccncc3n2)c(F)c(F)c2c1CCC2] | (Compound 7) |
| 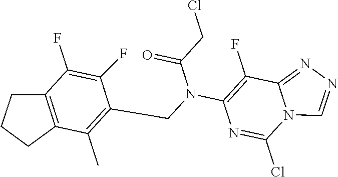 [SMILES: Cc1c(CN(C(=O)CCl)c2nc(Cl)n3cnnc3c2F)c(F)c(F)c2c1CCC2] | (Compound 8) |
| 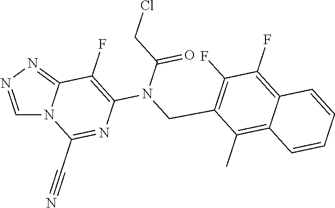 [SMILES: Cc1c(CN(C(=O)CCl)c2nc(C#N)n3cnnc3c2F)c(F)c(F)c2ccccc12] | (Compound 9) |
| 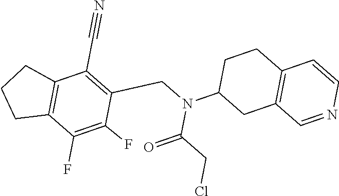 [SMILES: N#Cc1c(CN(C(=O)CCl)C2CCc3ccncc3C2)c(F)c(F)c2c1CCC2] | (Compound 10) |
| 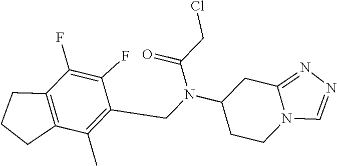 [SMILES: Cc1c(CN(C(=O)CCl)C2CCn3cnnc3C2)c(F)c(F)c2c1CCC2] | (Compound 11) |
| 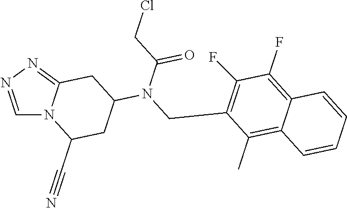 [SMILES: Cc1c(CN(C(=O)CCl)C2Cc3nncn3C(C#N)C2)c(F)c(F)c2ccccc12] | (Compound 12) |
| 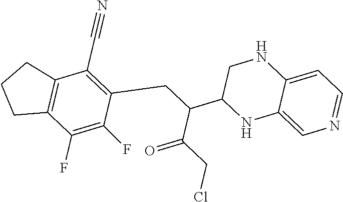 [SMILES: N#Cc1c(CC(C(=O)CCl)C2CNc3ccncc3N2)c(F)c(F)c2c1CCC2] | (Compound 13) |
| 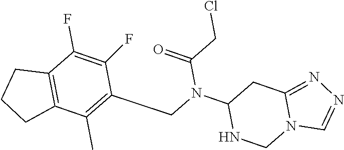 [SMILES: Cc1c(CN(C(=O)CCl)C2Cc3nncn3CN2)c(F)c(F)c2c1CCC2] | (Compound 14) |
| 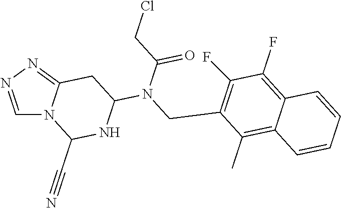 [SMILES: Cc1c(CN(C(=O)CCl)C2Cc3nncn3C(C#N)N2)c(F)c(F)c2ccccc12] | (Compound 15) |
| 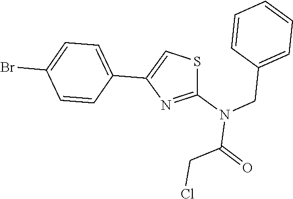 [SMILES: O=C(CCl)N(Cc1ccccc1)c1nc(-c2ccc(Br)cc2)cs1] | (Compound 16) |
| 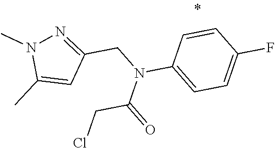 [SMILES: Cc1cc(CN(C(=O)CCl)c2ccc(F)cc2)nn1C] | (Compound 17) |
| 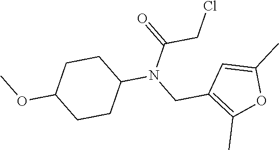 | (Compound 18) |
| 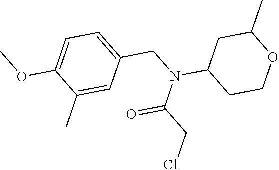 [SMILES: COc1ccc(CN(C(=O)CCl)C2CCOC(C)C2)cc1C] | (Compound 19) |
| 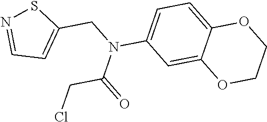 [SMILES: O=C(CCl)N(Cc1ccns1)c1ccc2c(c1)OCCO2] | (Compound 20) |
| 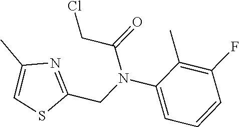 [SMILES: Cc1csc(CN(C(=O)CCl)c2cccc(F)c2C)n1] | (Compound 21) |
| 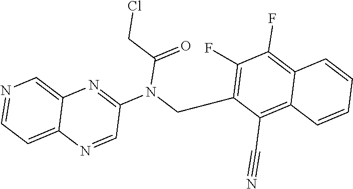 [SMILES: N#Cc1c(CN(C(=O)CCl)c2cnc3ccncc3n2)c(F)c(F)c2ccccc12] | (Compound 22) |
| 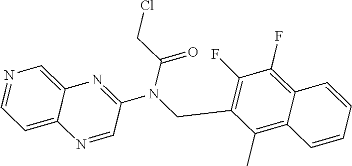 [SMILES: Cc1c(CN(C(=O)CCl)c2cnc3ccncc3n2)c(F)c(F)c2ccccc12] | (Compound 23) |
| 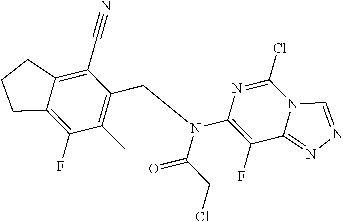 [SMILES: Cc1c(F)c2c(c(C#N)c1CN(C(=O)CCl)c1nc(Cl)n3cnnc3c1F)CCC2] | (Compound 24) |
| 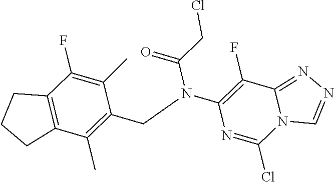 [SMILES: Cc1c(F)c2c(c(C)c1CN(C(=O)CCl)c1nc(Cl)n3cnnc3c1F)CCC2] | (Compound 25) |
| 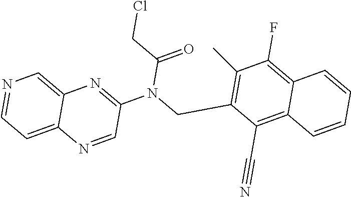 [SMILES: Cc1c(CN(C(=O)CCl)c2cnc3ccncc3n2)c(C#N)c2ccccc2c1F] | (Compound 27) |
| 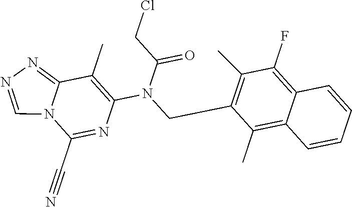 [SMILES: Cc1c(CN(C(=O)CCl)c2nc(C#N)n3cnnc3c2C)c(C)c2ccccc2c1F] | (Compound 28) |
| 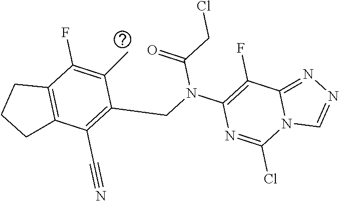 [SMILES: Cc1c(F)c2c(c(C#N)c1CN(C(=O)CCl)c1nc(Cl)n3cnnc3c1F)CCC2] | (Compound 29) |
| 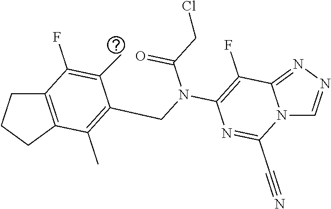 [SMILES: Cc1c(F)c2c(c(C)c1CN(C(=O)CCl)c1nc(C#N)n3cnnc3c1F)CCC2] | (Compound 30) |
| 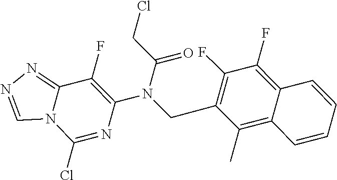 [SMILES: Cc1c(CN(C(=O)CCl)c2nc(Cl)n3cnnc3c2F)c(F)c(F)c2ccccc12] | (Compound 33) |
| 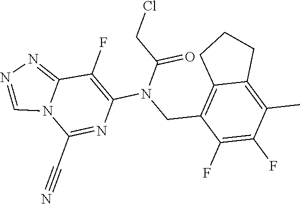 [SMILES: Cc1c(F)c(F)c(CN(C(=O)CCl)c2nc(C#N)n3cnnc3c2F)c2c1CCC2] | (Compound 43) |
| 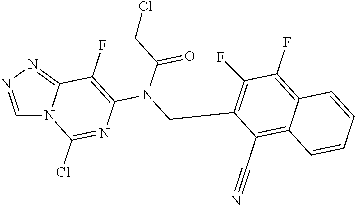 [SMILES: N#Cc1c(CN(C(=O)CCl)c2nc(Cl)n3cnnc3c2F)c(F)c(F)c2ccccc12] | (Compound 44) |
| 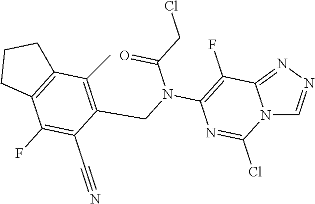 [SMILES: Cc1c(CN(C(=O)CCl)c2nc(Cl)n3cnnc3c2F)c(C#N)c(F)c2c1CCC2] | (Compound 47) |
| 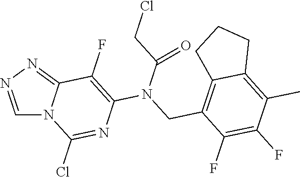 [SMILES: Cc1c(F)c(F)c(CN(C(=O)CCl)c2nc(Cl)n3cnnc3c2F)c2c1CCC2] | (Compound 51) |
| 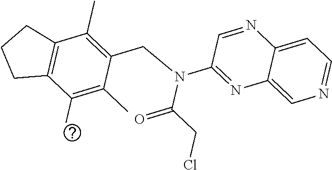 [SMILES: Cc1c(C)c2c(c(C)c1CN(C(=O)CCl)c1cnc3ccncc3n1)CCC2] | (Compound 68) |
| 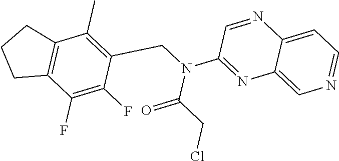 [SMILES: Cc1c(CN(C(=O)CCl)c2cnc3ccncc3n2)c(F)c(F)c2c1CCC2] | (Compound 70) |
| 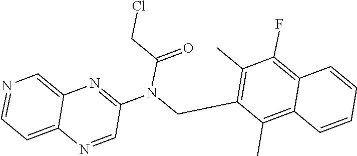 [SMILES: Cc1c(CN(C(=O)CCl)c2cnc3ccncc3n2)c(C)c2ccccc2c1F] | (Compound 73) |
| 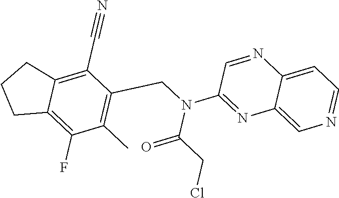 [SMILES: Cc1c(F)c2c(c(C#N)c1CN(C(=O)CCl)c1cnc3ccncc3n1)CCC2] | (Compound 75) |
| 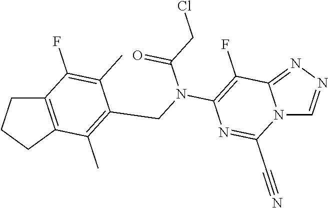 [SMILES: Cc1c(F)c2c(c(C)c1CN(C(=O)CCl)c1nc(C#N)n3cnnc3c1F)CCC2] | (Compound 79) |
| 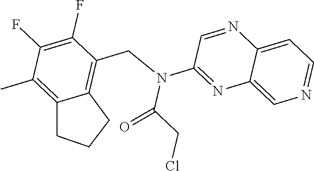 [SMILES: Cc1c(F)c(F)c(CN(C(=O)CCl)c2cnc3ccncc3n2)c2c1CCC2] | (Compound 84) |
| 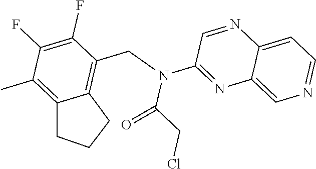 [SMILES: Cc1c(F)c(F)c(CN(C(=O)CCl)c2cnc3ccncc3n2)c2c1CCC2] | (Compound 85) |
| 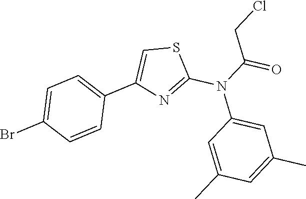 [SMILES: Cc1cc(C)cc(N(C(=O)CCl)c2nc(-c3ccc(Br)cc3)cs2)c1] | (Compound 90) |
| 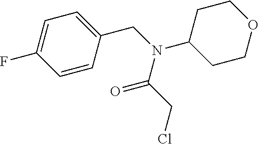 [SMILES: O=C(CCl)N(Cc1ccc(F)cc1)C1CCOCC1] | (Compound 92) |
| 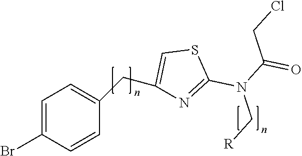 | (Formula 91) |
| 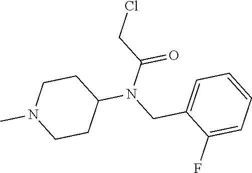 [SMILES: CN1CCC(N(Cc2ccccc2F)C(=O)CCl)CC1] | (Compound 94) |
| 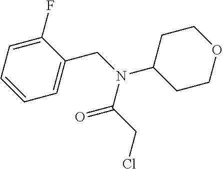 [SMILES: O=C(CCl)N(Cc1ccccc1F)C1CCOCC1] | (Compound 93) |
| 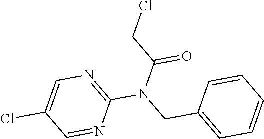 [SMILES: O=C(CCl)N(Cc1ccccc1)c1ncc(Cl)cn1] | (Compound 96) |
| 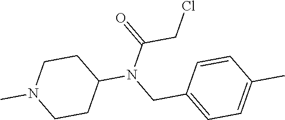 | (Compound 95) |
| 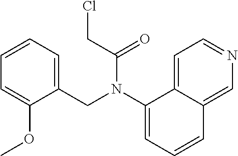 [SMILES: COc1ccccc1CN(C(=O)CCl)c1cccc2cnccc12] | (Compound 98) |
| 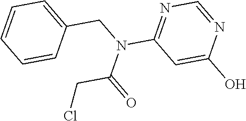 [SMILES: O=C(CCl)N(Cc1ccccc1)c1cc(O)ncn1] | (Compound 97) |
| 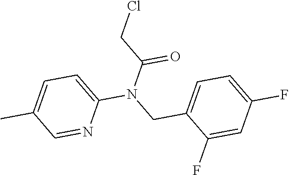 [SMILES: Cc1ccc(N(Cc2ccc(F)cc2F)C(=O)CCl)nc1] | (Compound 100) |
| 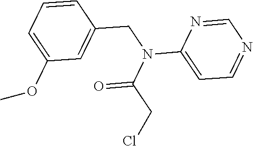 [SMILES: COc1cccc(CN(C(=O)CCl)c2ccncn2)c1] | (Compound 99) |
| 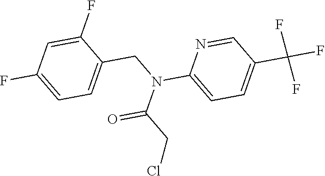 [SMILES: O=C(CCl)N(Cc1ccc(F)cc1F)c1ccc(C(F)(F)F)cn1] | (Compound 102) |
| 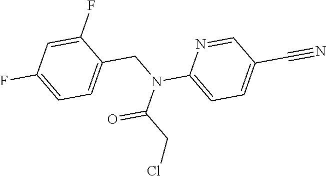 [SMILES: N#Cc1ccc(N(Cc2ccc(F)cc2F)C(=O)CCl)nc1] | (Compound 101) |
| 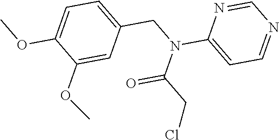 [SMILES: COc1ccc(CN(C(=O)CCl)c2ccncn2)cc1OC] | (Compound 104) |
| 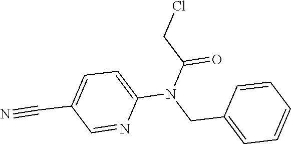 [SMILES: N#Cc1ccc(N(Cc2ccccc2)C(=O)CCl)nc1] | (Compound 103) |
| 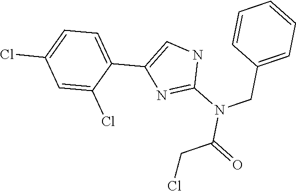 [SMILES: O=C(CCl)N(Cc1ccccc1)c1nc(-c2ccc(Cl)cc2Cl)c[nH]1] | (Compound 106) |
| 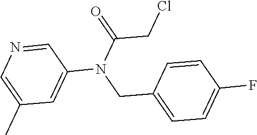 [SMILES: Cc1cncc(N(Cc2ccc(F)cc2)C(=O)CCl)c1] | (Compound 105) |
| 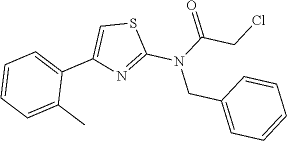 [SMILES: Cc1ccccc1-c1csc(N(Cc2ccccc2)C(=O)CCl)n1] | (Compound 108) |
| 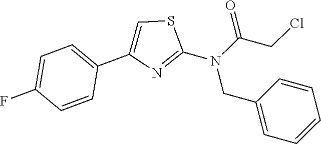 [SMILES: O=C(CCl)N(Cc1ccccc1)c1nc(-c2ccc(F)cc2)cs1] | (Compound 107) |
| 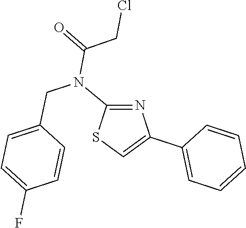 [SMILES: O=C(CCl)N(Cc1ccc(F)cc1)c1nc(-c2ccccc2)cs1] | (Compound 110) |
| 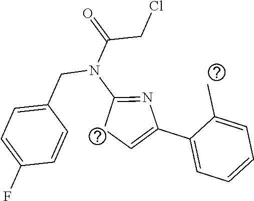 [SMILES: Cc1ccccc1C1=CCC(N(Cc2ccc(F)cc2)C(=O)CCl)=N1] | (Compound 109) |
| 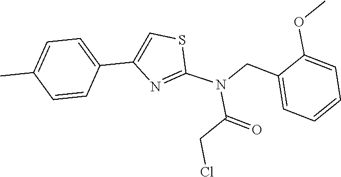 [SMILES: COc1ccccc1CN(C(=O)CCl)c1nc(-c2ccc(C)cc2)cs1] | (Compound 112) |
| 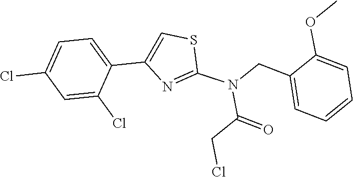 [SMILES: COc1ccccc1CN(C(=O)CCl)c1nc(-c2ccc(Cl)cc2Cl)cs1] | (Compound 111) |
| 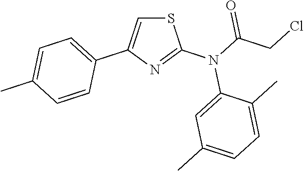 [SMILES: Cc1ccc(-c2csc(N(C(=O)CCl)c3cc(C)ccc3C)n2)cc1] | (Compound 114) |
| 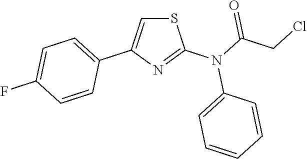 [SMILES: O=C(CCl)N(c1ccccc1)c1nc(-c2ccc(F)cc2)cs1] | (Compound 113) |
| 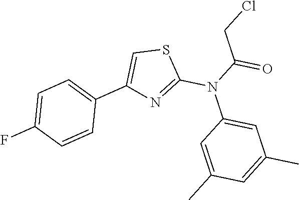 [SMILES: Cc1cc(C)cc(N(C(=O)CCl)c2nc(-c3ccc(F)cc3)cs2)c1] | (Compound 116) |
| 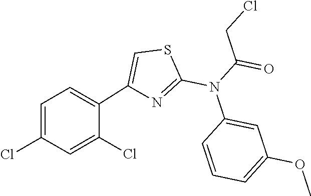 [SMILES: COc1cccc(N(C(=O)CCl)c2nc(-c3ccc(Cl)cc3Cl)cs2)c1] | (Compound 115) |
| 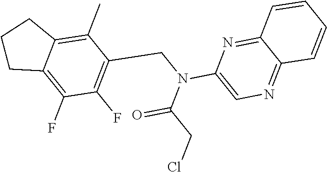 [SMILES: Cc1c(CN(C(=O)CCl)c2cnc3ccccc3n2)c(F)c(F)c2c1CCC2] | (Compound 118) |
| 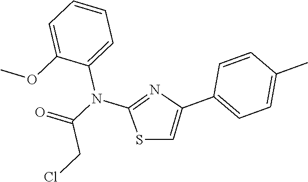 | (Compound 117) |
| 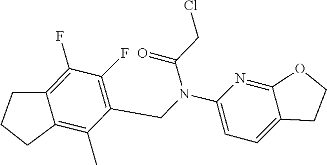 [SMILES: Cc1c(CN(C(=O)CCl)c2ccc3c(n2)OCC3)c(F)c(F)c2c1CCC2] | (Compound 120) |
| 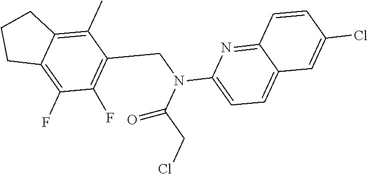 [SMILES: Cc1c(CN(C(=O)CCl)c2ccc3cc(Cl)ccc3n2)c(F)c(F)c2c1CCC2] | (Compound 119) |
| 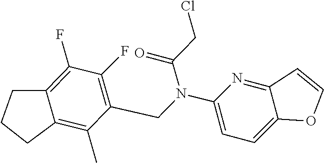 [SMILES: Cc1c(CN(C(=O)CCl)c2ccc3occc3n2)c(F)c(F)c2c1CCC2] | (Compound 122) |
| 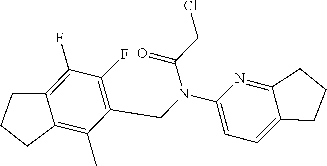 [SMILES: Cc1c(CN(C(=O)CCl)c2ccc3c(n2)CCC3)c(F)c(F)c2c1CCC2] | (Compound 121) |
| 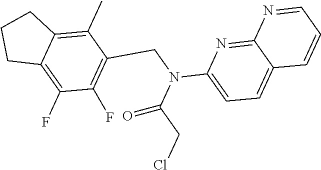 [SMILES: Cc1c(CN(C(=O)CCl)c2ccc3cccnc3n2)c(F)c(F)c2c1CCC2] | (Compound 124) |
| 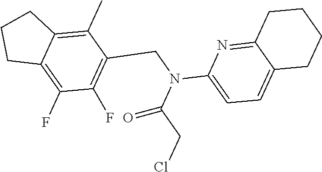 [SMILES: Cc1c(CN(C(=O)CCl)c2ccc3c(n2)CCCC3)c(F)c(F)c2c1CCC2] | (Compound 123) |
| 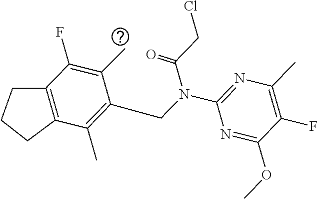 [SMILES: COc1nc(N(Cc2c(C)c(F)c3c(c2C)CCC3)C(=O)CCl)nc(C)c1F] | (Compound 126) |
| 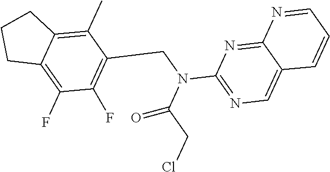 [SMILES: Cc1c(CN(C(=O)CCl)c2ncc3cccnc3n2)c(F)c(F)c2c1CCC2] | (Compound 125) |
| 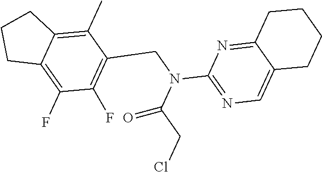 [SMILES: Cc1c(CN(C(=O)CCl)c2ncc3c(n2)CCCC3)c(F)c(F)c2c1CCC2] | (Compound 128) |
| 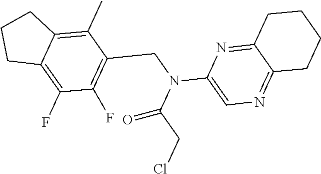 [SMILES: Cc1c(CN(C(=O)CCl)c2cnc3c(n2)CCCC3)c(F)c(F)c2c1CCC2] | (Compound 127) |
| 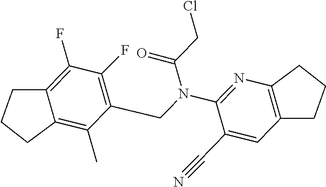 [SMILES: Cc1c(CN(C(=O)CCl)c2nc3c(cc2C#N)CCC3)c(F)c(F)c2c1CCC2] | (Compound 130) |
| 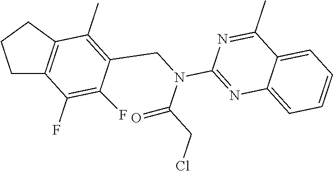 [SMILES: Cc1c(CN(C(=O)CCl)c2nc(C)c3ccccc3n2)c(F)c(F)c2c1CCC2] | (Compound 129) |
| 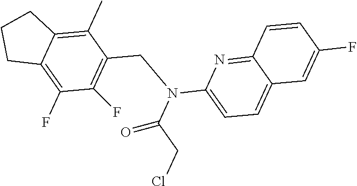 [SMILES: Cc1c(CN(C(=O)CCl)c2ccc3cc(F)ccc3n2)c(F)c(F)c2c1CCC2] | (Compound 132) |
| 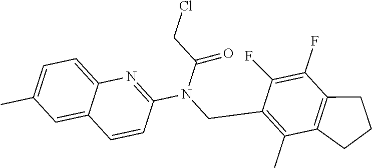 [SMILES: Cc1ccc2nc(N(Cc3c(C)c4c(c(F)c3F)CCC4)C(=O)CCl)ccc2c1] | (Compound 131) |
| 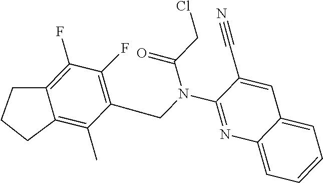 [SMILES: Cc1c(CN(C(=O)CCl)c2nc3ccccc3cc2C#N)c(F)c(F)c2c1CCC2] | (Compound 134) |
| 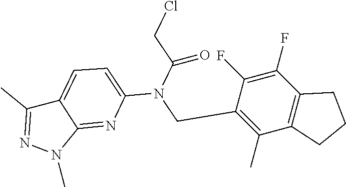 [SMILES: Cc1c(CN(C(=O)CCl)c2ccc3c(C)nn(C)c3n2)c(F)c(F)c2c1CCC2] | (Compound 133) |
| 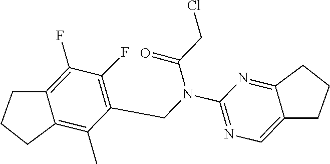 [SMILES: Cc1c(CN(C(=O)CCl)c2ncc3c(n2)CCC3)c(F)c(F)c2c1CCC2] | (Compound 136) |
| 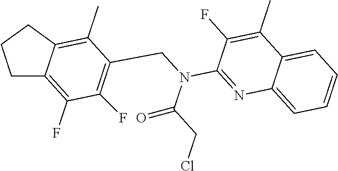 [SMILES: Cc1c(CN(C(=O)CCl)c2nc3ccccc3c(C)c2F)c(F)c(F)c2c1CCC2] | (Compound 135) |
| 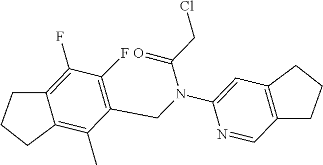 [SMILES: Cc1c(CN(C(=O)CCl)c2cc3c(cn2)CCC3)c(F)c(F)c2c1CCC2] | (Compound 138) |
| 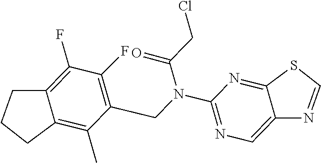 [SMILES: Cc1c(CN(C(=O)CCl)c2ncc3ncsc3n2)c(F)c(F)c2c1CCC2] | (Compound 137) |
| 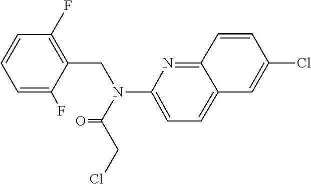 [SMILES: O=C(CCl)N(Cc1c(F)cccc1F)c1ccc2cc(Cl)ccc2n1] | (Compound 140) |
| 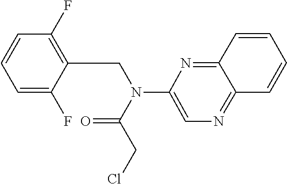 [SMILES: O=C(CCl)N(Cc1c(F)cccc1F)c1cnc2ccccc2n1] | (Compound 139) |
| 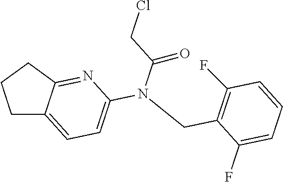 [SMILES: O=C(CCl)N(Cc1c(F)cccc1F)c1ccc2c(n1)CCC2] | (Compound 142) |
| 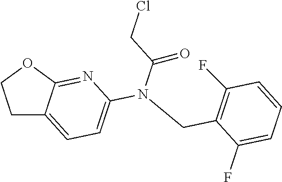 [SMILES: O=C(CCl)N(Cc1c(F)cccc1F)c1ccc2c(n1)OCC2] | (Compound 141) |
| 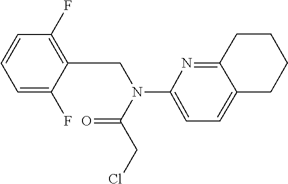 [SMILES: O=C(CCl)N(Cc1c(F)cccc1F)c1ccc2c(n1)CCCC2] | (Compound 144) |
| 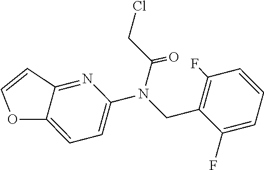 [SMILES: O=C(CCl)N(Cc1c(F)cccc1F)c1ccc2occc2n1] | (Compound 143) |
| 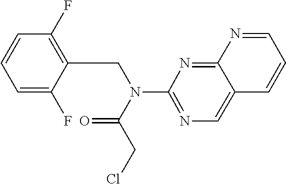 [SMILES: O=C(CCl)N(Cc1c(F)cccc1F)c1ncc2cccnc2n1] | (Compound 146) |
| 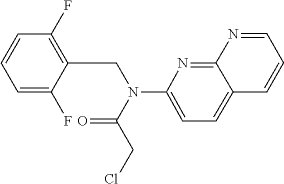 [SMILES: O=C(CCl)N(Cc1c(F)cccc1F)c1ccc2cccnc2n1] | (Compound 145) |
| 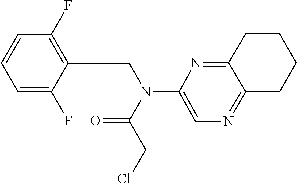 [SMILES: O=C(CCl)N(Cc1c(F)cccc1F)c1cnc2c(n1)CCCC2] | (Compound 148) |
| 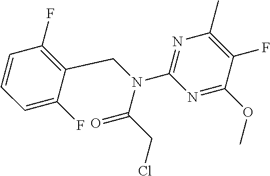 [SMILES: COc1nc(N(Cc2c(F)cccc2F)C(=O)CCl)nc(C)c1F] | (Compound 147) |
| 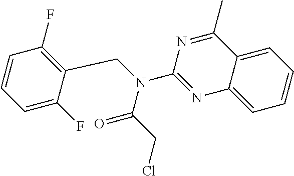 [SMILES: Cc1nc(N(Cc2c(F)cccc2F)C(=O)CCl)nc2ccccc12] | (Compound 150) |
| 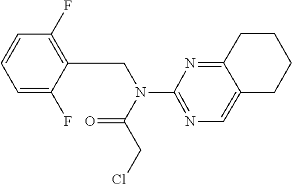 [SMILES: O=C(CCl)N(Cc1c(F)cccc1F)c1ncc2c(n1)CCCC2] | (Compound 149) |
| 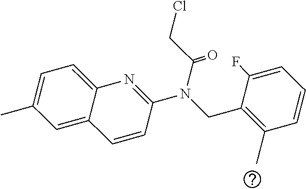 [SMILES: Cc1ccc2nc(N(Cc3c(C)cccc3F)C(=O)CCl)ccc2c1] | (Compound 152) |
| 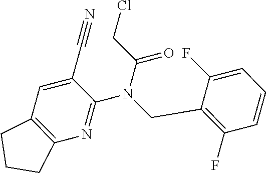 [SMILES: N#Cc1cc2c(nc1N(Cc1c(F)cccc1F)C(=O)CCl)CCC2] | (Compound 151) |
| 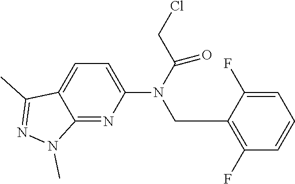 [SMILES: Cc1nn(C)c2nc(N(Cc3c(F)cccc3F)C(=O)CCl)ccc12] | (Compound 154) |
| 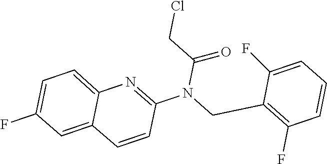 [SMILES: O=C(CCl)N(Cc1c(F)cccc1F)c1ccc2cc(F)ccc2n1] | (Compound 153) |
| 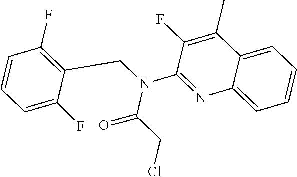 [SMILES: Cc1c(F)c(N(Cc2c(F)cccc2F)C(=O)CCl)nc2ccccc12] | (Compound 156) |
| 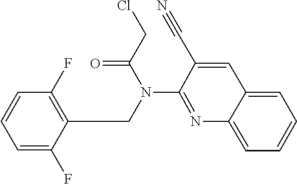 [SMILES: N#Cc1cc2ccccc2nc1N(Cc1c(F)cccc1F)C(=O)CCl] | (Compound 155) |
| 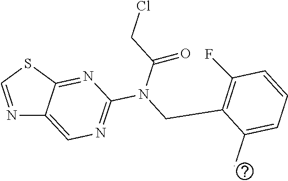 [SMILES: Cc1cccc(F)c1CN(C(=O)CCl)c1ncc2ncsc2n1] | (Compound 158) |
| 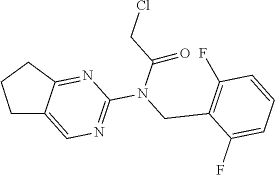 [SMILES: O=C(CCl)N(Cc1c(F)cccc1F)c1ncc2c(n1)CCC2] | (Compound 157) |
| 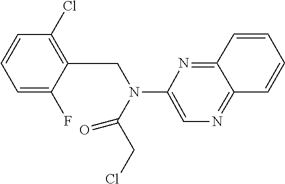 [SMILES: O=C(CCl)N(Cc1c(F)cccc1Cl)c1cnc2ccccc2n1] | (Compound 160) |
| 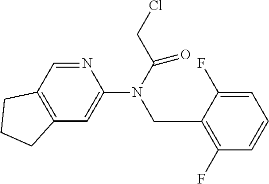 [SMILES: O=C(CCl)N(Cc1c(F)cccc1F)c1cc2c(cn1)CCC2] | (Compound 159) |
| 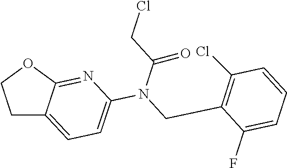 [SMILES: O=C(CCl)N(Cc1c(F)cccc1Cl)c1ccc2c(n1)OCC2] | (Compound 162) |
| 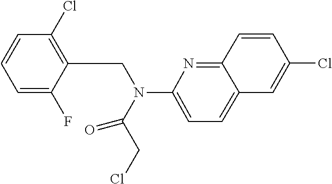 [SMILES: O=C(CCl)N(Cc1c(F)cccc1Cl)c1ccc2cc(Cl)ccc2n1] | (Compound 161) |
| 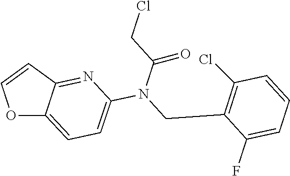 [SMILES: O=C(CCl)N(Cc1c(F)cccc1Cl)c1ccc2occc2n1] | (Compound 164) |
| 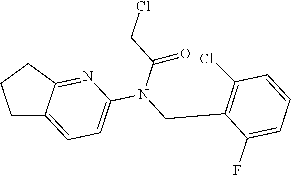 [SMILES: O=C(CCl)N(Cc1c(F)cccc1Cl)c1ccc2c(n1)CCC2] | (Compound 163) |
| 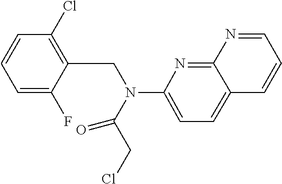 [SMILES: O=C(CCl)N(Cc1c(F)cccc1Cl)c1ccc2cccnc2n1] | (Compound 166) |
| 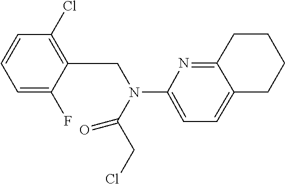 [SMILES: O=C(CCl)N(Cc1c(F)cccc1Cl)c1ccc2c(n1)CCCC2] | (Compound 165) |
| 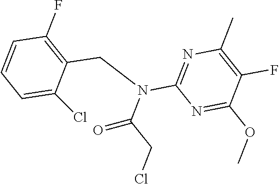 [SMILES: COc1nc(N(Cc2c(F)cccc2Cl)C(=O)CCl)nc(C)c1F] | (Compound 168) |
| 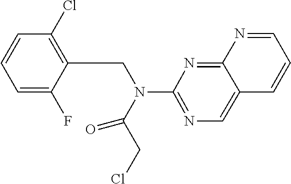 [SMILES: O=C(CCl)N(Cc1c(F)cccc1Cl)c1ncc2cccnc2n1] | (Compound 167) |
| 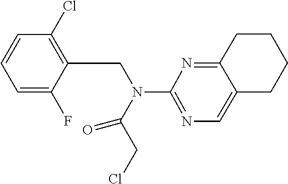 [SMILES: O=C(CCl)N(Cc1c(F)cccc1Cl)c1ncc2c(n1)CCCC2] | (Compound 170) |
| 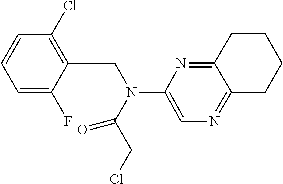 [SMILES: O=C(CCl)N(Cc1c(F)cccc1Cl)c1cnc2c(n1)CCCC2] | (Compound 169) |
| 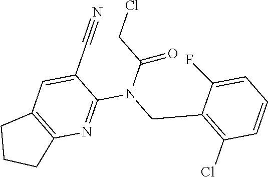 [SMILES: N#Cc1cc2c(nc1N(Cc1c(F)cccc1Cl)C(=O)CCl)CCC2] | (Compound 172) |
| 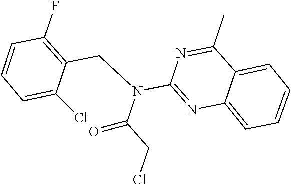 [SMILES: Cc1nc(N(Cc2c(F)cccc2Cl)C(=O)CCl)nc2ccccc12] | (Compound 171) |
| 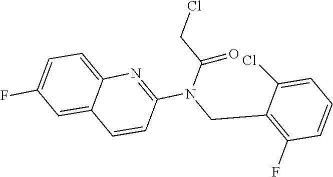 [SMILES: O=C(CCl)N(Cc1c(F)cccc1Cl)c1ccc2cc(F)ccc2n1] | (Compound 174) |
| 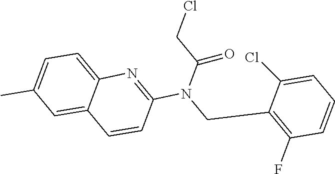 [SMILES: Cc1ccc2nc(N(Cc3c(F)cccc3Cl)C(=O)CCl)ccc2c1] | (Compound 173) |
| 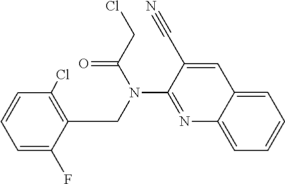 [SMILES: N#Cc1cc2ccccc2nc1N(Cc1c(F)cccc1Cl)C(=O)CCl] | (Compound 176) |
| 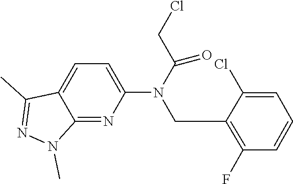 [SMILES: Cc1nn(C)c2nc(N(Cc3c(F)cccc3Cl)C(=O)CCl)ccc12] | (Compound 175) |
| 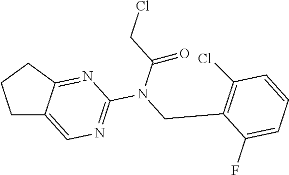 [SMILES: O=C(CCl)N(Cc1c(F)cccc1Cl)c1ncc2c(n1)CCC2] | (Compound 178) |
| 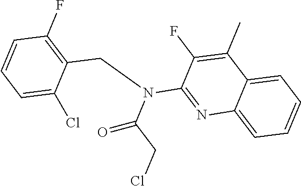 [SMILES: Cc1c(F)c(N(Cc2c(F)cccc2Cl)C(=O)CCl)nc2ccccc12] | (Compound 177) |
| 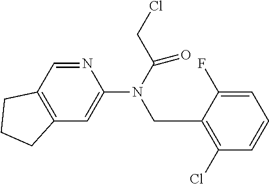 [SMILES: O=C(CCl)N(Cc1c(F)cccc1Cl)c1cc2c(cn1)CCC2] | (Compound 180) |
| 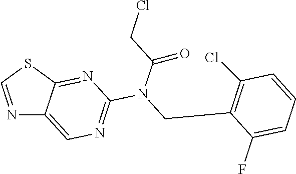 [SMILES: O=C(CCl)N(Cc1c(F)cccc1Cl)c1ncc2ncsc2n1] | (Compound 179) |
| 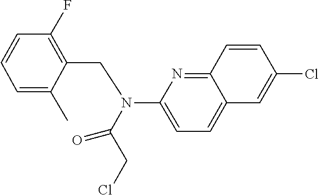 [SMILES: Cc1cccc(F)c1CN(C(=O)CCl)c1ccc2cc(Cl)ccc2n1] | (Compound 182) |
| 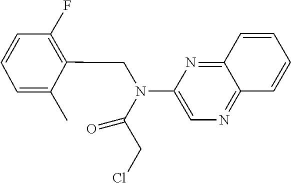 [SMILES: Cc1cccc(F)c1CN(C(=O)CCl)c1cnc2ccccc2n1] | (Compound 181) |
| 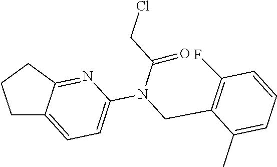 [SMILES: Cc1cccc(F)c1CN(C(=O)CCl)c1ccc2c(n1)CCC2] | (Compound 184) |
| 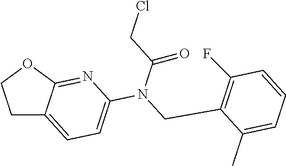 [SMILES: Cc1cccc(F)c1CN(C(=O)CCl)c1ccc2c(n1)OCC2] | (Compound 183) |
| 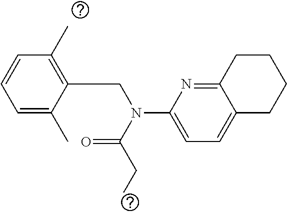 [SMILES: CCC(=O)N(Cc1c(C)cccc1C)c1ccc2c(n1)CCCC2] | (Compound 186) |
| 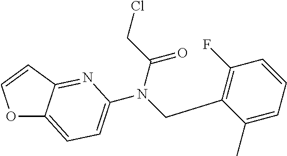 [SMILES: Cc1cccc(F)c1CN(C(=O)CCl)c1ccc2occc2n1] | (Compound 185) |
| 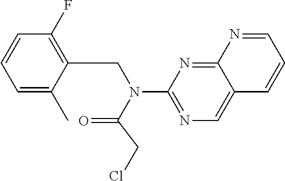 [SMILES: Cc1cccc(F)c1CN(C(=O)CCl)c1ncc2cccnc2n1] | (Compound 188) |
| 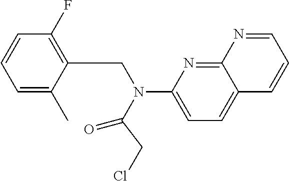 [SMILES: Cc1cccc(F)c1CN(C(=O)CCl)c1ccc2cccnc2n1] | (Compound 187) |
| 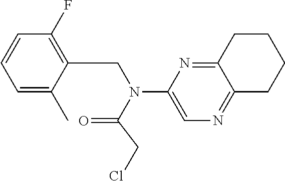 [SMILES: Cc1cccc(F)c1CN(C(=O)CCl)c1cnc2c(n1)CCCC2] | (Compound 190) |
| 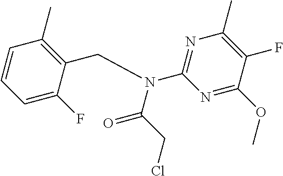 [SMILES: COc1nc(N(Cc2c(C)cccc2F)C(=O)CCl)nc(C)c1F] | (Compound 189) |
| 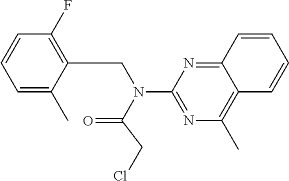 [SMILES: Cc1cccc(F)c1CN(C(=O)CCl)c1nc(C)c2ccccc2n1] | (Compound 192) |
| 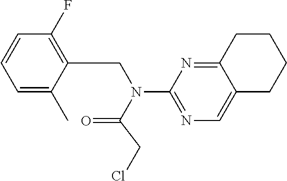 [SMILES: Cc1cccc(F)c1CN(C(=O)CCl)c1ncc2c(n1)CCCC2] | (Compound 191) |
| 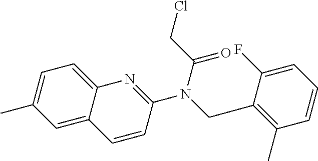 [SMILES: Cc1ccc2nc(N(Cc3c(C)cccc3F)C(=O)CCl)ccc2c1] | (Compound 194) |
| 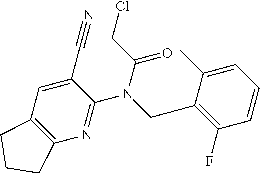 [SMILES: Cc1cccc(F)c1CN(C(=O)CCl)c1nc2c(cc1C#N)CCC2] | (Compound 193) |
| 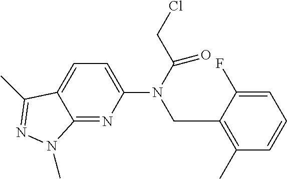 [SMILES: Cc1cccc(F)c1CN(C(=O)CCl)c1ccc2c(C)nn(C)c2n1] | (Compound 196) |
| 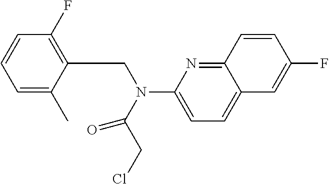 [SMILES: Cc1cccc(F)c1CN(C(=O)CCl)c1ccc2cc(F)ccc2n1] | (Compound 195) |
| 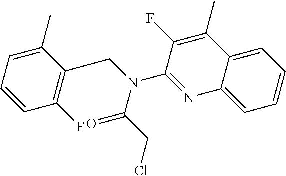 [SMILES: Cc1cccc(F)c1CN(C(=O)CCl)c1nc2ccccc2c(C)c1F] | (Compound 198) |
| 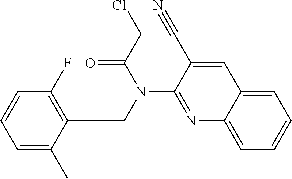 [SMILES: Cc1cccc(F)c1CN(C(=O)CCl)c1nc2ccccc2cc1C#N] | (Compound 197) |
| 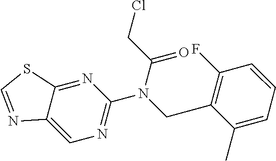 [SMILES: Cc1cccc(F)c1CN(C(=O)CCl)c1ncc2ncsc2n1] | (Compound 200) |
| 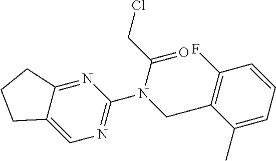 [SMILES: Cc1cccc(F)c1CN(C(=O)CCl)c1ncc2c(n1)CCC2] | (Compound 199) |
| 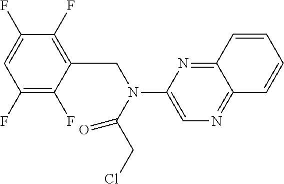 [SMILES: O=C(CCl)N(Cc1c(F)c(F)cc(F)c1F)c1cnc2ccccc2n1] | (Compound 202) |
| 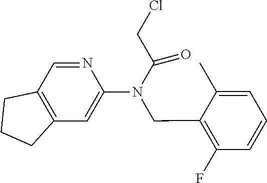 [SMILES: Cc1cccc(F)c1CN(C(=O)CCl)c1cc2c(cn1)CCC2] | (Compound 201) |
| 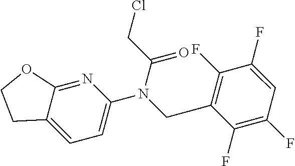 [SMILES: O=C(CCl)N(Cc1c(F)c(F)cc(F)c1F)c1ccc2c(n1)OCC2] | (Compound 204) |
| 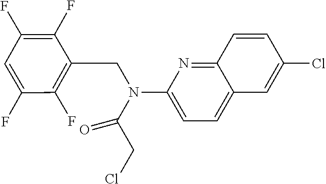 [SMILES: O=C(CCl)N(Cc1c(F)c(F)cc(F)c1F)c1ccc2cc(Cl)ccc2n1] | (Compound 203) |
| 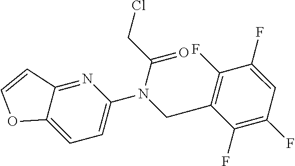 [SMILES: O=C(CCl)N(Cc1c(F)c(F)cc(F)c1F)c1ccc2occc2n1] | (Compound 206) |
| 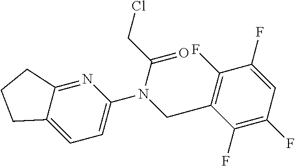 [SMILES: O=C(CCl)N(Cc1c(F)c(F)cc(F)c1F)c1ccc2c(n1)CCC2] | (Compound 205) |
| 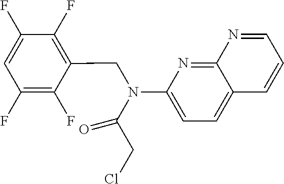 [SMILES: O=C(CCl)N(Cc1c(F)c(F)cc(F)c1F)c1ccc2cccnc2n1] | (Compound 208) |
| 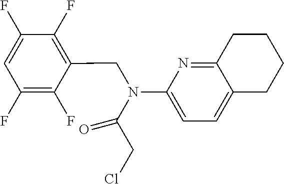 [SMILES: O=C(CCl)N(Cc1c(F)c(F)cc(F)c1F)c1ccc2c(n1)CCCC2] | (Compound 207) |
| 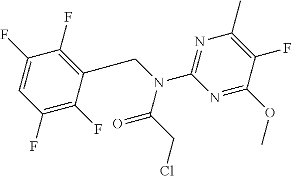 [SMILES: COc1nc(N(Cc2c(F)c(F)cc(F)c2F)C(=O)CCl)nc(C)c1F] | (Compound 210) |
| 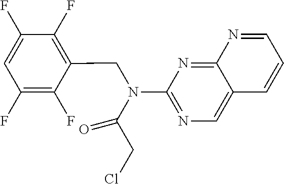 [SMILES: O=C(CCl)N(Cc1c(F)c(F)cc(F)c1F)c1ncc2cccnc2n1] | (Compound 209) |
| 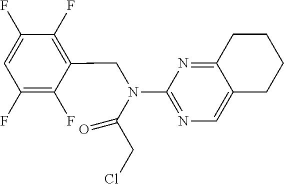 [SMILES: O=C(CCl)N(Cc1c(F)c(F)cc(F)c1F)c1ncc2c(n1)CCCC2] | (Compound 212) |
| 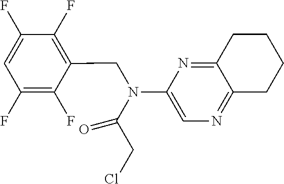 [SMILES: O=C(CCl)N(Cc1c(F)c(F)cc(F)c1F)c1cnc2c(n1)CCCC2] | (Compound 211) |
| 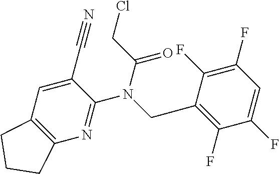 [SMILES: N#Cc1cc2c(nc1N(Cc1c(F)c(F)cc(F)c1F)C(=O)CCl)CCC2] | (Compound 214) |
| 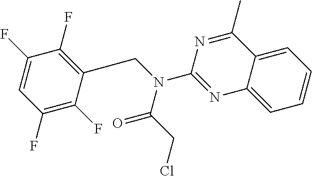 [SMILES: Cc1nc(N(Cc2c(F)c(F)cc(F)c2F)C(=O)CCl)nc2ccccc12] | (Compound 213) |
| 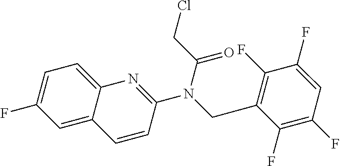 [SMILES: O=C(CCl)N(Cc1c(F)c(F)cc(F)c1F)c1ccc2cc(F)ccc2n1] | (Compound 216) |
| 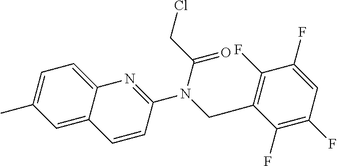 [SMILES: Cc1ccc2nc(N(Cc3c(F)c(F)cc(F)c3F)C(=O)CCl)ccc2c1] | (Compound 215) |
| 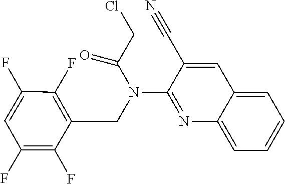 [SMILES: N#Cc1cc2ccccc2nc1N(Cc1c(F)c(F)cc(F)c1F)C(=O)CCl] | (Compound 218) |
| 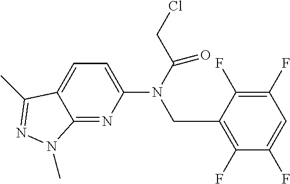 [SMILES: Cc1nn(C)c2nc(N(Cc3c(F)c(F)cc(F)c3F)C(=O)CCl)ccc12] | (Compound 217) |
| 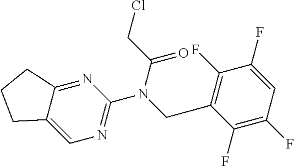 [SMILES: O=C(CCl)N(Cc1c(F)c(F)cc(F)c1F)c1ncc2c(n1)CCC2] | (Compound 220) |
| 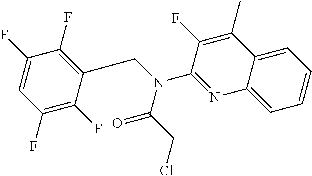 [SMILES: Cc1c(F)c(N(Cc2c(F)c(F)cc(F)c2F)C(=O)CCl)nc2ccccc12] | (Compound 219) |
| 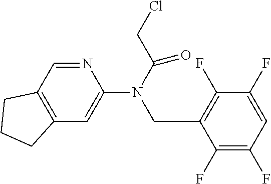 [SMILES: O=C(CCl)N(Cc1c(F)c(F)cc(F)c1F)c1cc2c(cn1)CCC2] | (Compound 222) |
| 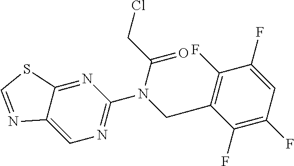 [SMILES: O=C(CCl)N(Cc1c(F)c(F)cc(F)c1F)c1ncc2ncsc2n1] | (Compound 221) |
| 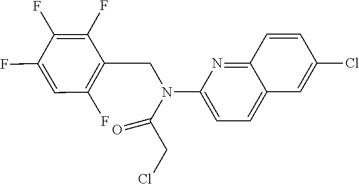 [SMILES: O=C(CCl)N(Cc1c(F)cc(F)c(F)c1F)c1ccc2cc(Cl)ccc2n1] | (Compound 224) |
| 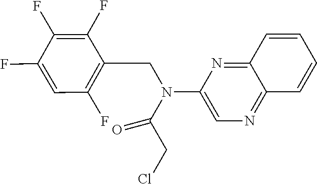 [SMILES: O=C(CCl)N(Cc1c(F)cc(F)c(F)c1F)c1cnc2ccccc2n1] | (Compound 223) |
| 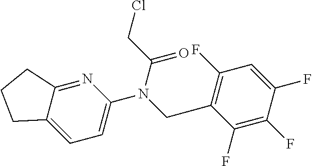 [SMILES: O=C(CCl)N(Cc1c(F)cc(F)c(F)c1F)c1ccc2c(n1)CCC2] | (Compound 226) |
| 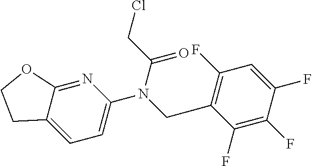 [SMILES: O=C(CCl)N(Cc1c(F)cc(F)c(F)c1F)c1ccc2c(n1)OCC2] | (Compound 225) |
| 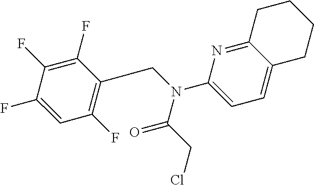 [SMILES: O=C(CCl)N(Cc1c(F)cc(F)c(F)c1F)c1ccc2c(n1)CCCC2] | (Compound 228) |
| 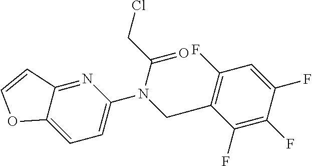 [SMILES: O=C(CCl)N(Cc1c(F)cc(F)c(F)c1F)c1ccc2occc2n1] | (Compound 227) |
| 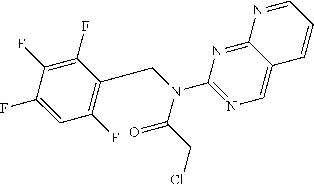 [SMILES: O=C(CCl)N(Cc1c(F)cc(F)c(F)c1F)c1ncc2cccnc2n1] | (Compound 230) |
| 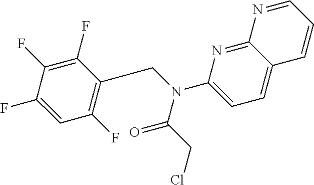 [SMILES: O=C(CCl)N(Cc1c(F)cc(F)c(F)c1F)c1ccc2cccnc2n1] | (Compound 229) |
| 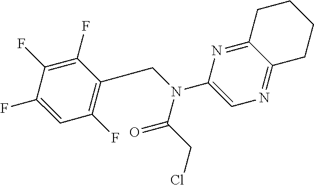 [SMILES: O=C(CCl)N(Cc1c(F)cc(F)c(F)c1F)c1cnc2c(n1)CCCC2] | (Compound 232) |
| 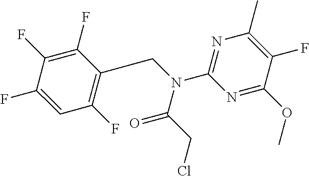 [SMILES: COc1nc(N(Cc2c(F)cc(F)c(F)c2F)C(=O)CCl)nc(C)c1F] | (Compound 231) |
| 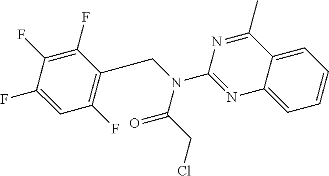 [SMILES: Cc1nc(N(Cc2c(F)cc(F)c(F)c2F)C(=O)CCl)nc2ccccc12] | (Compound 234) |
| 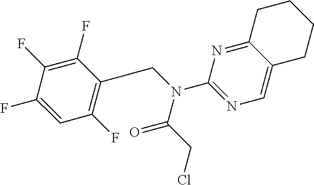 [SMILES: O=C(CCl)N(Cc1c(F)cc(F)c(F)c1F)c1ncc2c(n1)CCCC2] | (Compound 233) |
| 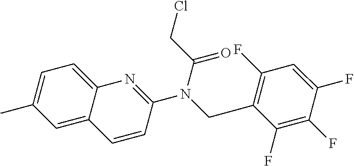 [SMILES: Cc1ccc2nc(N(Cc3c(F)cc(F)c(F)c3F)C(=O)CCl)ccc2c1] | (Compound 236) |
| 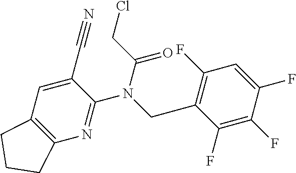 [SMILES: N#Cc1cc2c(nc1N(Cc1c(F)cc(F)c(F)c1F)C(=O)CCl)CCC2] | (Compound 235) |
| 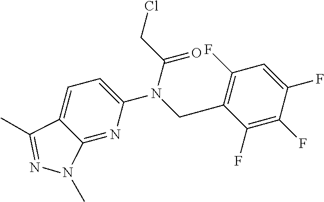 [SMILES: Cc1nn(C)c2nc(N(Cc3c(F)cc(F)c(F)c3F)C(=O)CCl)ccc12] | (Compound 238) |
| 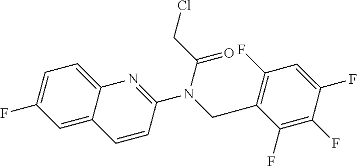 [SMILES: O=C(CCl)N(Cc1c(F)cc(F)c(F)c1F)c1ccc2cc(F)ccc2n1] | (Compound 237) |
| 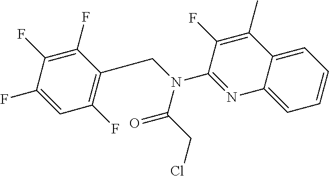 [SMILES: Cc1c(F)c(N(Cc2c(F)cc(F)c(F)c2F)C(=O)CCl)nc2ccccc12] | (Compound 240) |
| 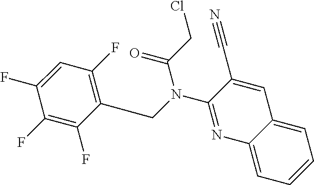 [SMILES: N#Cc1cc2ccccc2nc1N(Cc1c(F)cc(F)c(F)c1F)C(=O)CCl] | (Compound 239) |
| 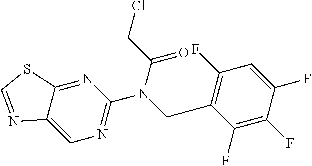 [SMILES: O=C(CCl)N(Cc1c(F)cc(F)c(F)c1F)c1ncc2ncsc2n1] | (Compound 242) |
| 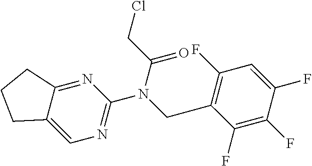 [SMILES: O=C(CCl)N(Cc1c(F)cc(F)c(F)c1F)c1ncc2c(n1)CCC2] | (Compound 241) |
| 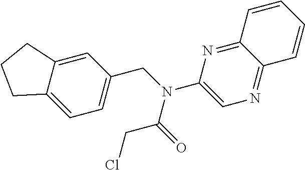 [SMILES: O=C(CCl)N(Cc1ccc2c(c1)CCC2)c1cnc2ccccc2n1] | (Compound 244) |
| 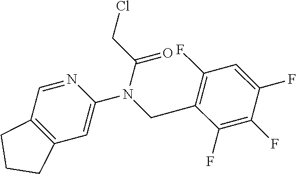 [SMILES: O=C(CCl)N(Cc1c(F)cc(F)c(F)c1F)c1cc2c(cn1)CCC2] | (Compound 243) |
| 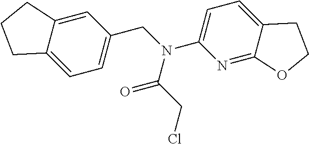 [SMILES: O=C(CCl)N(Cc1ccc2c(c1)CCC2)c1ccc2c(n1)OCC2] | (Compound 246) |
| 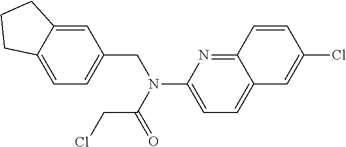 [SMILES: O=C(CCl)N(Cc1ccc2c(c1)CCC2)c1ccc2cc(Cl)ccc2n1] | (Compound 245) |
| 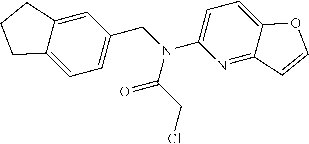 [SMILES: O=C(CCl)N(Cc1ccc2c(c1)CCC2)c1ccc2occc2n1] | (Compound 248) |
| 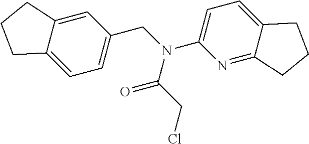 [SMILES: O=C(CCl)N(Cc1ccc2c(c1)CCC2)c1ccc2c(n1)CCC2] | (Compound 247) |
| 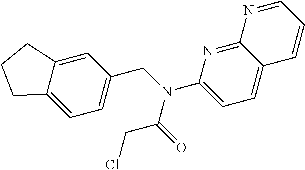 [SMILES: O=C(CCl)N(Cc1ccc2c(c1)CCC2)c1ccc2cccnc2n1] | (Compound 250) |
| 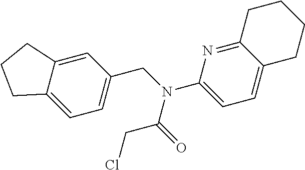 [SMILES: O=C(CCl)N(Cc1ccc2c(c1)CCC2)c1ccc2c(n1)CCCC2] | (Compound 249) |
| 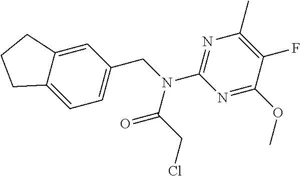 [SMILES: COc1nc(N(Cc2ccc3c(c2)CCC3)C(=O)CCl)nc(C)c1F] | (Compound 252) |
| 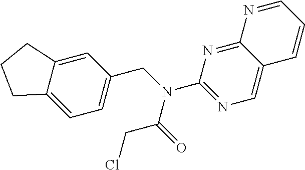 [SMILES: O=C(CCl)N(Cc1ccc2c(c1)CCC2)c1ncc2cccnc2n1] | (Compound 251) |
| 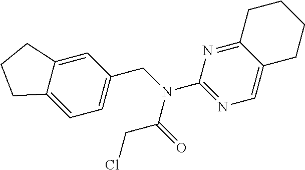 [SMILES: O=C(CCl)N(Cc1ccc2c(c1)CCC2)c1ncc2c(n1)CCCC2] | (Compound 254) |
| 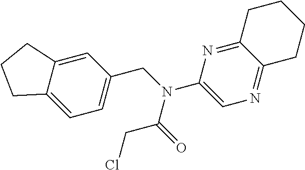 [SMILES: O=C(CCl)N(Cc1ccc2c(c1)CCC2)c1cnc2c(n1)CCCC2] | (Compound 253) |
| 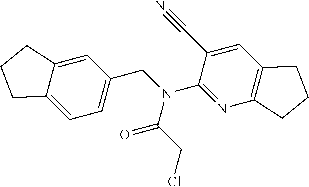 [SMILES: N#Cc1cc2c(nc1N(Cc1ccc3c(c1)CCC3)C(=O)CCl)CCC2] | (Compound 256) |
| 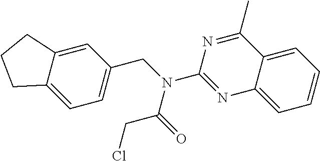 [SMILES: Cc1nc(N(Cc2ccc3c(c2)CCC3)C(=O)CCl)nc2ccccc12] | (Compound 255) |
| 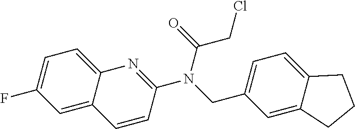 [SMILES: O=C(CCl)N(Cc1ccc2c(c1)CCC2)c1ccc2cc(F)ccc2n1] | (Compound 258) |
| 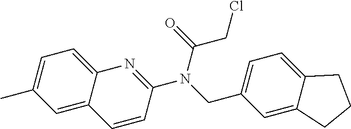 [SMILES: Cc1ccc2nc(N(Cc3ccc4c(c3)CCC4)C(=O)CCl)ccc2c1] | (Compound 257) |
| 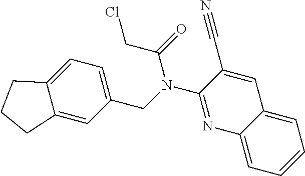 [SMILES: N#Cc1cc2ccccc2nc1N(Cc1ccc2c(c1)CCC2)C(=O)CCl] | (Compound 260) |
| 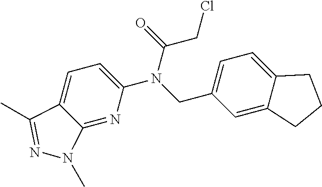 [SMILES: Cc1nn(C)c2nc(N(Cc3ccc4c(c3)CCC4)C(=O)CCl)ccc12] | (Compound 259) |
| 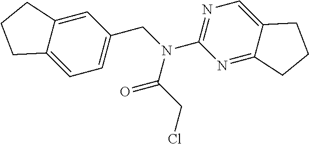 [SMILES: O=C(CCl)N(Cc1ccc2c(c1)CCC2)c1ncc2c(n1)CCC2] | (Compound 262) |
| 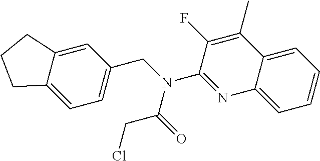 [SMILES: Cc1c(F)c(N(Cc2ccc3c(c2)CCC3)C(=O)CCl)nc2ccccc12] | (Compound 261) |
| 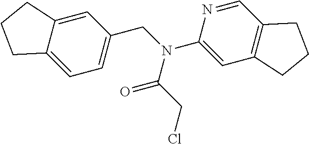 [SMILES: O=C(CCl)N(Cc1ccc2c(c1)CCC2)c1cc2c(cn1)CCC2] | (Compound 264) |
| 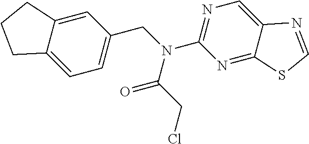 [SMILES: O=C(CCl)N(Cc1ccc2c(c1)CCC2)c1ncc2ncsc2n1] | (Compound 263) |
| 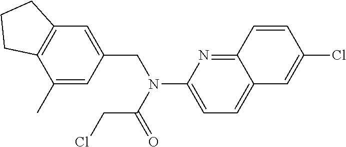 [SMILES: Cc1cc(CN(C(=O)CCl)c2ccc3cc(Cl)ccc3n2)cc2c1CCC2] | (Compound 266) |
| 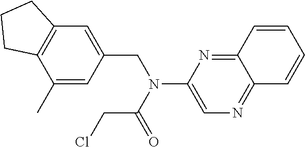 [SMILES: Cc1cc(CN(C(=O)CCl)c2cnc3ccccc3n2)cc2c1CCC2] | (Compound 265) |
| 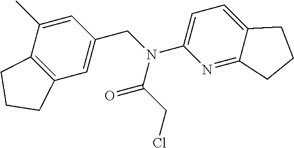 [SMILES: Cc1cc(CN(C(=O)CCl)c2ccc3c(n2)CCC3)cc2c1CCC2] | (Compound 268) |
| 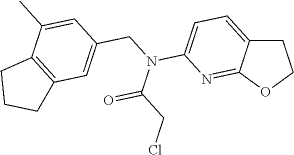 [SMILES: Cc1cc(CN(C(=O)CCl)c2ccc3c(n2)OCC3)cc2c1CCC2] | (Compound 267) |
| 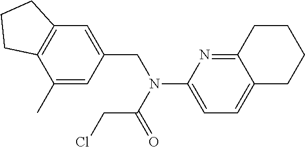 [SMILES: Cc1cc(CN(C(=O)CCl)c2ccc3c(n2)CCCC3)cc2c1CCC2] | (Compound 270) |
| 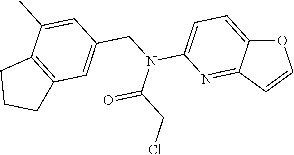 [SMILES: Cc1cc(CN(C(=O)CCl)c2ccc3occc3n2)cc2c1CCC2] | (Compound 269) |
| 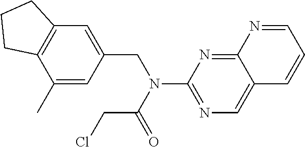 [SMILES: Cc1cc(CN(C(=O)CCl)c2ncc3cccnc3n2)cc2c1CCC2] | (Compound 272) |
| 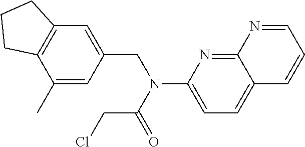 [SMILES: Cc1cc(CN(C(=O)CCl)c2ccc3cccnc3n2)cc2c1CCC2] | (Compound 271) |
| 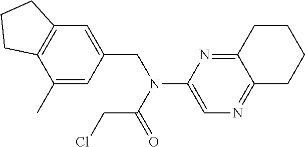 [SMILES: Cc1cc(CN(C(=O)CCl)c2cnc3c(n2)CCCC3)cc2c1CCC2] | (Compound 274) |
| 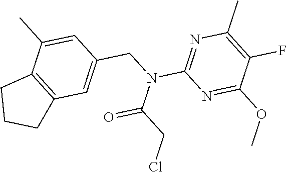 [SMILES: COc1nc(N(Cc2cc(C)c3c(c2)CCC3)C(=O)CCl)nc(C)c1F] | (Compound 273) |
| 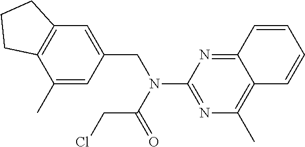 [SMILES: Cc1cc(CN(C(=O)CCl)c2nc(C)c3ccccc3n2)cc2c1CCC2] | (Compound 276) |
| 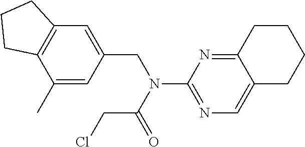 [SMILES: Cc1cc(CN(C(=O)CCl)c2ncc3c(n2)CCCC3)cc2c1CCC2] | (Compound 275) |
| 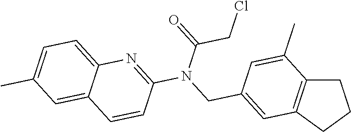 [SMILES: Cc1ccc2nc(N(Cc3cc(C)c4c(c3)CCC4)C(=O)CCl)ccc2c1] | (Compound 278) |
| 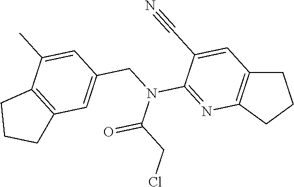 [SMILES: Cc1cc(CN(C(=O)CCl)c2nc3c(cc2C#N)CCC3)cc2c1CCC2] | (Compound 277) |
| 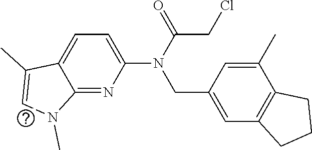 [SMILES: Cc1cc(CN(C(=O)CCl)c2ccc3c(C)cn(C)c3n2)cc2c1CCC2] | (Compound 280) |
| 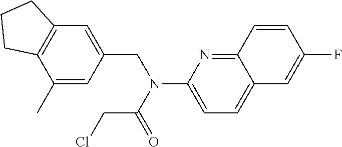 [SMILES: Cc1cc(CN(C(=O)CCl)c2ccc3cc(F)ccc3n2)cc2c1CCC2] | (Compound 279) |
| 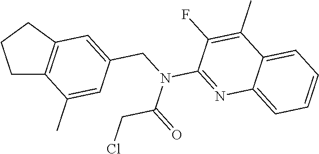 [SMILES: Cc1cc(CN(C(=O)CCl)c2nc3ccccc3c(C)c2F)cc2c1CCC2] | (Compound 282) |
| 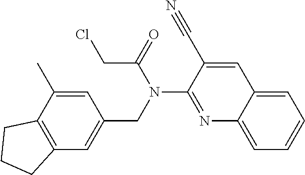 [SMILES: Cc1cc(CN(C(=O)CCl)c2nc3ccccc3cc2C#N)cc2c1CCC2] | (Compound 281) |
| 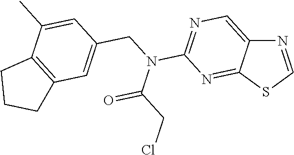 [SMILES: Cc1cc(CN(C(=O)CCl)c2ncc3ncsc3n2)cc2c1CCC2] | (Compound 284) |
| 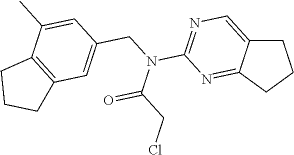 [SMILES: Cc1cc(CN(C(=O)CCl)c2ncc3c(n2)CCC3)cc2c1CCC2] | (Compound 283) |
| 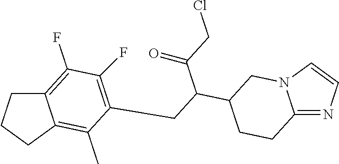 [SMILES: Cc1c(CC(C(=O)CCl)C2CCc3nccn3C2)c(F)c(F)c2c1CCC2] | (Compound 286) |
| 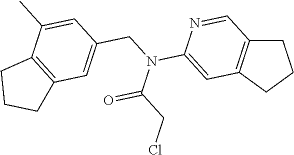 [SMILES: Cc1cc(CN(C(=O)CCl)c2cc3c(cn2)CCC3)cc2c1CCC2] | (Compound 285) |
| 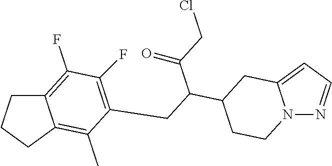 [SMILES: Cc1c(CC(C(=O)CCl)C2CCn3nccc3C2)c(F)c(F)c2c1CCC2] | (Compound 288) |
| 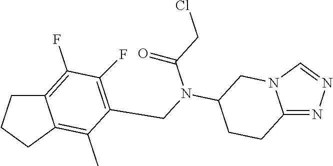 [SMILES: Cc1c(CN(C(=O)CCl)C2CCc3nncn3C2)c(F)c(F)c2c1CCC2] | (Compound 287) |
| 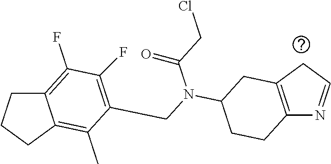 [SMILES: Cc1c(CN(C(=O)CCl)C2CCC3=C(CC=N3)C2)c(F)c(F)c2c1CCC2] | (Compound 290) |
| 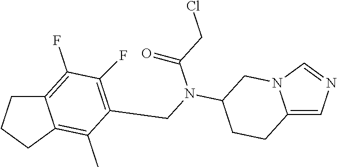 [SMILES: Cc1c(CN(C(=O)CCl)C2CCc3cncn3C2)c(F)c(F)c2c1CCC2] | (Compound 289) |
| 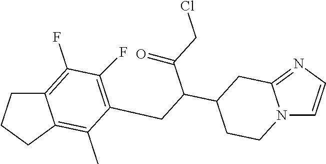 [SMILES: Cc1c(CC(C(=O)CCl)C2CCn3ccnc3C2)c(F)c(F)c2c1CCC2] | (Compound 292) |
| 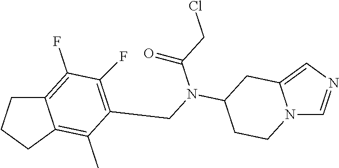 [SMILES: Cc1c(CN(C(=O)CCl)C2CCn3cncc3C2)c(F)c(F)c2c1CCC2] | (Compound 291) |
| 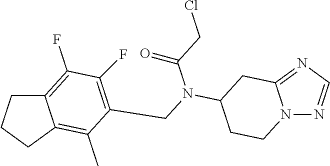 [SMILES: Cc1c(CN(C(=O)CCl)C2CCn3ncnc3C2)c(F)c(F)c2c1CCC2] | (Compound 294) |
| 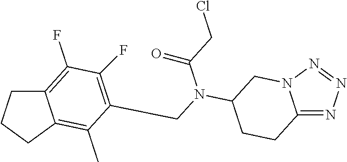 [SMILES: Cc1c(CN(C(=O)CCl)C2CCc3nnnn3C2)c(F)c(F)c2c1CCC2] | (Compound 293) |
| 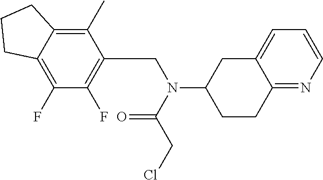 [SMILES: Cc1c(CN(C(=O)CCl)C2CCc3ncccc3C2)c(F)c(F)c2c1CCC2] | (Compound 296) |
| 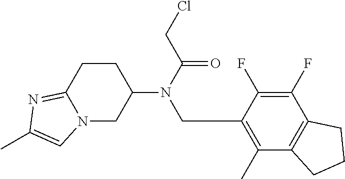 [SMILES: Cc1cn2c(n1)CCC(N(Cc1c(C)c3c(c(F)c1F)CCC3)C(=O)CCl)C2] | (Compound 295) |
| 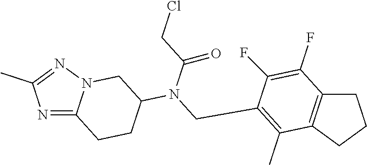 [SMILES: Cc1nc2n(n1)CC(N(Cc1c(C)c3c(c(F)c1F)CCC3)C(=O)CCl)CC2] | (Compound 298) |
| 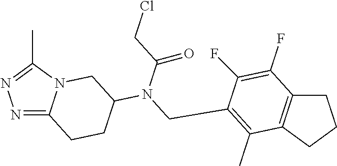 [SMILES: Cc1c(CN(C(=O)CCl)C2CCc3nnc(C)n3C2)c(F)c(F)c2c1CCC2] | (Compound 297) |
| 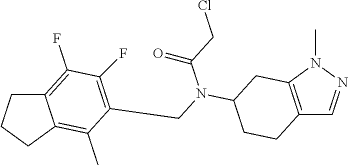 [SMILES: Cc1c(CN(C(=O)CCl)C2CCc3cnn(C)c3C2)c(F)c(F)c2c1CCC2] | (Compound 300) |
| 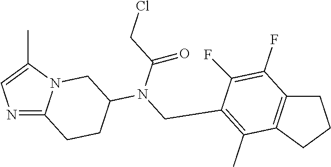 [SMILES: Cc1c(CN(C(=O)CCl)C2CCc3ncc(C)n3C2)c(F)c(F)c2c1CCC2] | (Compound 299) |
| 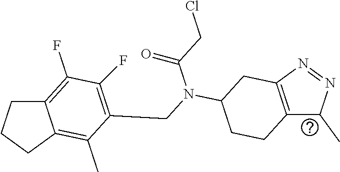 [SMILES: Cc1c(CN(C(=O)CCl)C2CCC3=C(C2)N=NC3C)c(F)c(F)c2c1CCC2] | (Compound 302) |
| 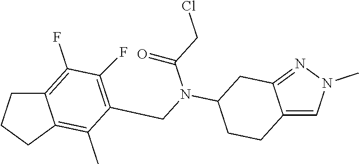 [SMILES: Cc1c(CN(C(=O)CCl)C2CCc3cn(C)nc3C2)c(F)c(F)c2c1CCC2] | (Compound 301) |
| 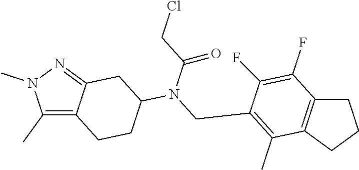 [SMILES: Cc1c(CN(C(=O)CCl)C2CCc3c(nn(C)c3C)C2)c(F)c(F)c2c1CCC2] | (Compound 304) |
| 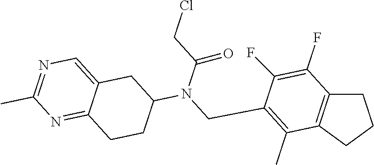 [SMILES: Cc1ncc2c(n1)CCC(N(Cc1c(C)c3c(c(F)c1F)CCC3)C(=O)CCl)C2] | (Compound 303) |
| 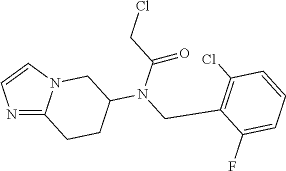 [SMILES: O=C(CCl)N(Cc1c(F)cccc1Cl)C1CCc2nccn2C1] | (Compound 306) |
| 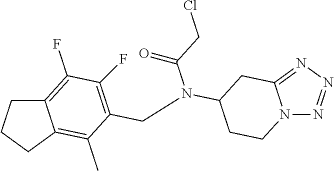 [SMILES: Cc1c(CN(C(=O)CCl)C2CCn3nnnc3C2)c(F)c(F)c2c1CCC2] | (Compound 305) |
| 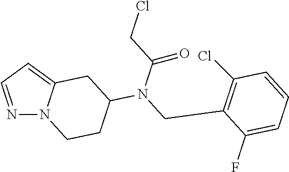 [SMILES: O=C(CCl)N(Cc1c(F)cccc1Cl)C1CCn2nccc2C1] | (Compound 308) |
| 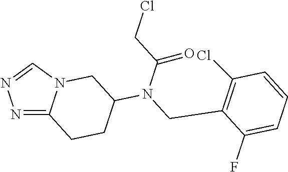 [SMILES: O=C(CCl)N(Cc1c(F)cccc1Cl)C1CCc2nncn2C1] | (Compound 307) |
| 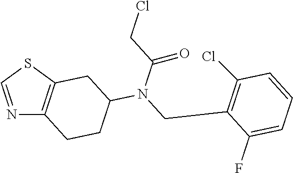 [SMILES: O=C(CCl)N(Cc1c(F)cccc1Cl)C1CCc2ncsc2C1] | (Compound 310) |
| 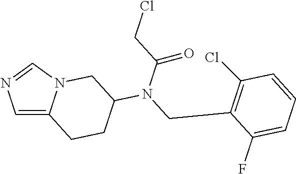 [SMILES: O=C(CCl)N(Cc1c(F)cccc1Cl)C1CCc2cncn2C1] | (Compound 309) |
| 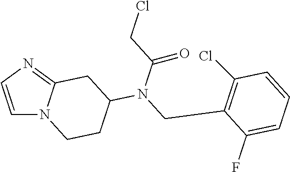 [SMILES: O=C(CCl)N(Cc1c(F)cccc1Cl)C1CCn2ccnc2C1] | (Compound 312) |
| 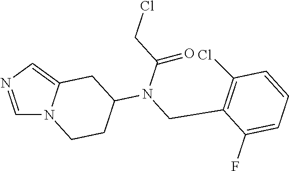 [SMILES: O=C(CCl)N(Cc1c(F)cccc1Cl)C1CCn2cncc2C1] | (Compound 311) |
| 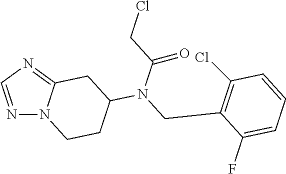 [SMILES: O=C(CCl)N(Cc1c(F)cccc1Cl)C1CCn2ncnc2C1] | (Compound 314) |
| 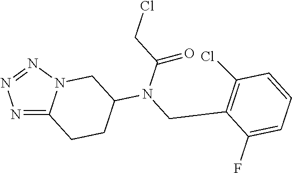 [SMILES: O=C(CCl)N(Cc1c(F)cccc1Cl)C1CCc2nnnn2C1] | (Compound 313) |
| 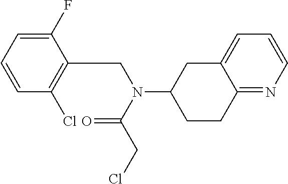 [SMILES: O=C(CCl)N(Cc1c(F)cccc1Cl)C1CCc2ncccc2C1] | (Compound 316) |
| 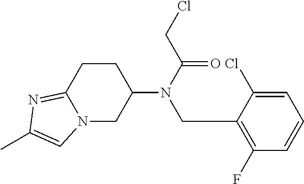 [SMILES: Cc1cn2c(n1)CCC(N(Cc1c(F)cccc1Cl)C(=O)CCl)C2] | (Compound 315) |
| 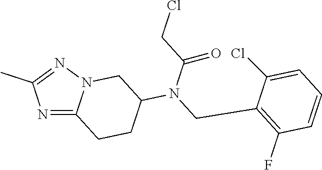 [SMILES: Cc1nc2n(n1)CC(N(Cc1c(F)cccc1Cl)C(=O)CCl)CC2] | (Compound 318) |
| 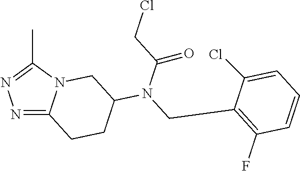 [SMILES: Cc1nnc2n1CC(N(Cc1c(F)cccc1Cl)C(=O)CCl)CC2] | (Compound 317) |
| 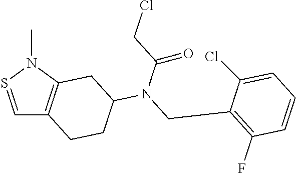 | (Compound 320) |
| 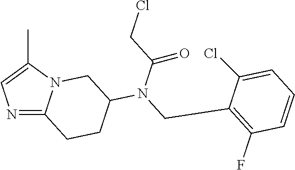 [SMILES: Cc1cnc2n1CC(N(Cc1c(F)cccc1Cl)C(=O)CCl)CC2] | (Compound 319) |
| 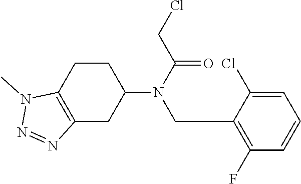 [SMILES: Cn1nnc2c1CCC(N(Cc1c(F)cccc1Cl)C(=O)CCl)C2] | (Compound 322) |
| 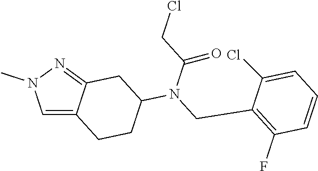 [SMILES: Cn1cc2c(n1)CC(N(Cc1c(F)cccc1Cl)C(=O)CCl)CC2] | (Compound 321) |
| 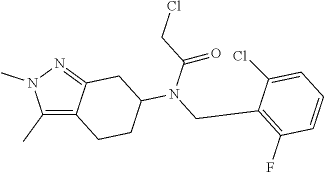 [SMILES: Cc1c2c(nn1C)CC(N(Cc1c(F)cccc1Cl)C(=O)CCl)CC2] | (Compound 324) |
| 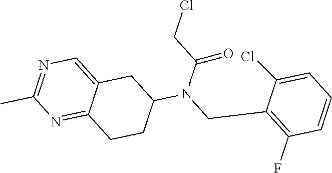 [SMILES: Cc1ncc2c(n1)CCC(N(Cc1c(F)cccc1Cl)C(=O)CCl)C2] | (Compound 323) |
| 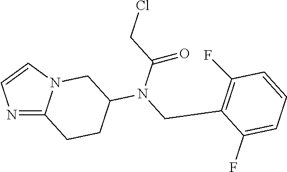 [SMILES: O=C(CCl)N(Cc1c(F)cccc1F)C1CCc2nccn2C1] | (Compound 326) |
| 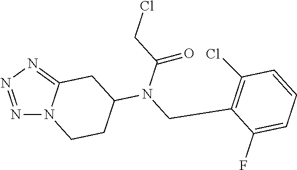 [SMILES: O=C(CCl)N(Cc1c(F)cccc1Cl)C1CCn2nnnc2C1] | (Compound 325) |
| 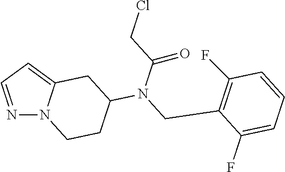 [SMILES: O=C(CCl)N(Cc1c(F)cccc1F)C1CCn2nccc2C1] | (Compound 328) |
| 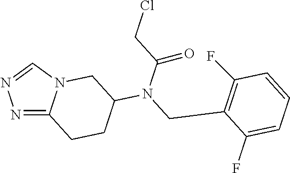 [SMILES: O=C(CCl)N(Cc1c(F)cccc1F)C1CCc2nncn2C1] | (Compound 327) |
| 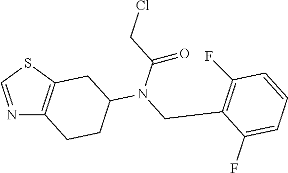 [SMILES: O=C(CCl)N(Cc1c(F)cccc1F)C1CCc2ncsc2C1] | (Compound 330) |
| 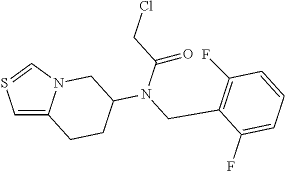 | (Compound 329) |
| 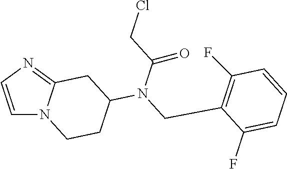 [SMILES: O=C(CCl)N(Cc1c(F)cccc1F)C1CCn2ccnc2C1] | (Compound 332) |
| 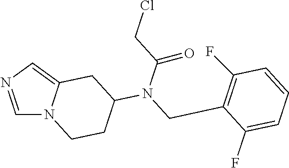 [SMILES: O=C(CCl)N(Cc1c(F)cccc1F)C1CCn2cncc2C1] | (Compound 331) |
| 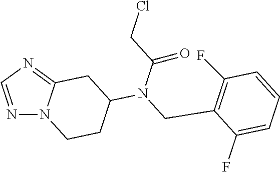 [SMILES: O=C(CCl)N(Cc1c(F)cccc1F)C1CCn2ncnc2C1] | (Compound 334) |
| 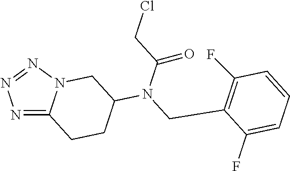 [SMILES: O=C(CCl)N(Cc1c(F)cccc1F)C1CCc2nnnn2C1] | (Compound 333) |
| 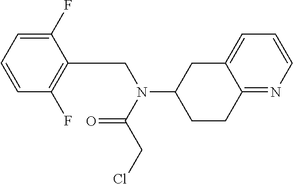 [SMILES: O=C(CCl)N(Cc1c(F)cccc1F)C1CCc2ncccc2C1] | (Compound 336) |
| 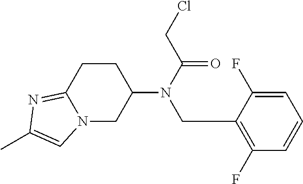 [SMILES: Cc1cn2c(n1)CCC(N(Cc1c(F)cccc1F)C(=O)CCl)C2] | (Compound 335) |
| 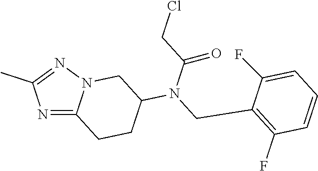 [SMILES: Cc1nc2n(n1)CC(N(Cc1c(F)cccc1F)C(=O)CCl)CC2] | (Compound 338) |
| 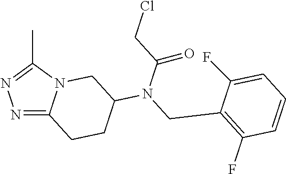 [SMILES: Cc1nnc2n1CC(N(Cc1c(F)cccc1F)C(=O)CCl)CC2] | (Compound 337) |
| 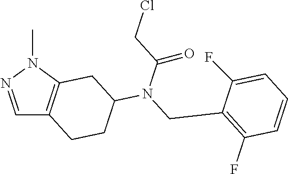 [SMILES: Cn1ncc2c1CC(N(Cc1c(F)cccc1F)C(=O)CCl)CC2] | (Compound 340) |
| 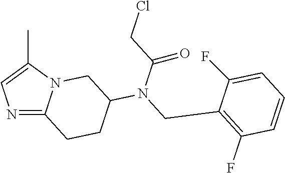 [SMILES: Cc1cnc2n1CC(N(Cc1c(F)cccc1F)C(=O)CCl)CC2] | (Compound 339) |
| 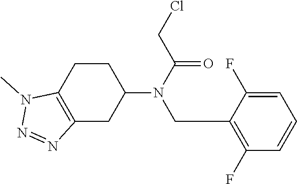 [SMILES: Cn1nnc2c1CCC(N(Cc1c(F)cccc1F)C(=O)CCl)C2] | (Compound 342) |
| 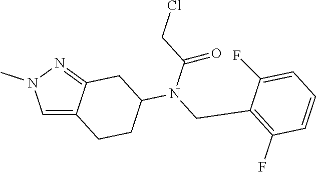 [SMILES: Cn1cc2c(n1)CC(N(Cc1c(F)cccc1F)C(=O)CCl)CC2] | (Compound 341) |
| 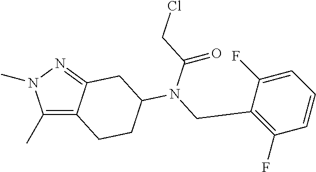 [SMILES: Cc1c2c(nn1C)CC(N(Cc1c(F)cccc1F)C(=O)CCl)CC2] | (Compound 344) |
| 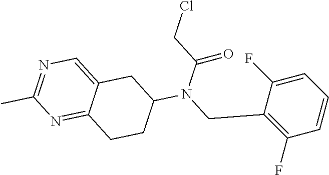 [SMILES: Cc1ncc2c(n1)CCC(N(Cc1c(F)cccc1F)C(=O)CCl)C2] | (Compound 343) |
| 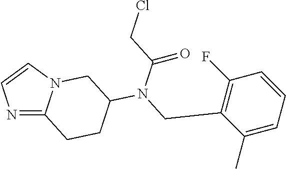 [SMILES: Cc1cccc(F)c1CN(C(=O)CCl)C1CCc2nccn2C1] | (Compound 346) |
| 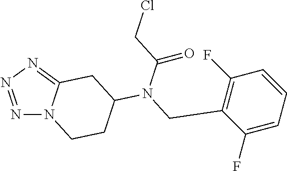 [SMILES: O=C(CCl)N(Cc1c(F)cccc1F)C1CCn2nnnc2C1] | (Compound 345) |
| 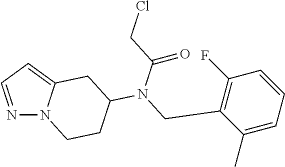 [SMILES: Cc1cccc(F)c1CN(C(=O)CCl)C1CCn2nccc2C1] | (Compound 348) |
| 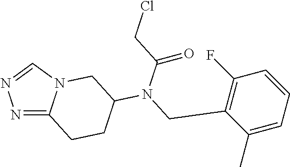 [SMILES: Cc1cccc(F)c1CN(C(=O)CCl)C1CCc2nncn2C1] | (Compound 347) |
| 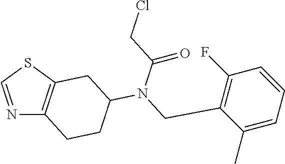 [SMILES: Cc1cccc(F)c1CN(C(=O)CCl)C1CCc2ncsc2C1] | (Compound 350) |
| 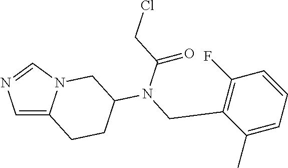 [SMILES: Cc1cccc(F)c1CN(C(=O)CCl)C1CCc2cncn2C1] | (Compound 349) |
| 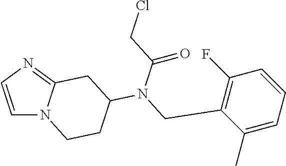 [SMILES: Cc1cccc(F)c1CN(C(=O)CCl)C1CCn2ccnc2C1] | (Compound 352) |
| 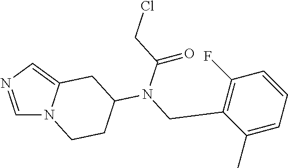 [SMILES: Cc1cccc(F)c1CN(C(=O)CCl)C1CCn2cncc2C1] | (Compound 351) |
| 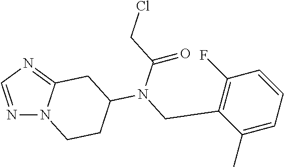 [SMILES: Cc1cccc(F)c1CN(C(=O)CCl)C1CCn2ncnc2C1] | (Compound 354) |
| 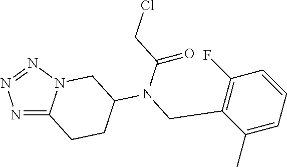 [SMILES: Cc1cccc(F)c1CN(C(=O)CCl)C1CCc2nnnn2C1] | (Compound 353) |
| 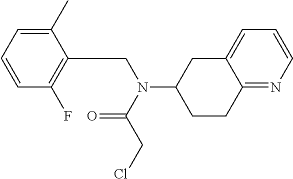 [SMILES: Cc1cccc(F)c1CN(C(=O)CCl)C1CCc2ncccc2C1] | (Compound 356) |
| 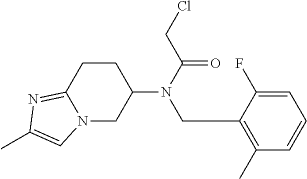 [SMILES: Cc1cn2c(n1)CCC(N(Cc1c(C)cccc1F)C(=O)CCl)C2] | (Compound 355) |
| 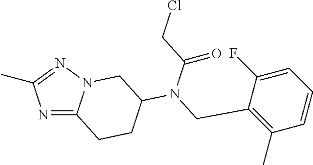 [SMILES: Cc1nc2n(n1)CC(N(Cc1c(C)cccc1F)C(=O)CCl)CC2] | (Compound 358) |
| 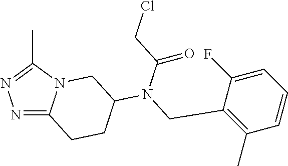 [SMILES: Cc1cccc(F)c1CN(C(=O)CCl)C1CCc2nnc(C)n2C1] | (Compound 357) |
| 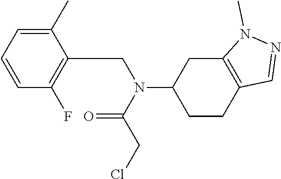 [SMILES: Cc1cccc(F)c1CN(C(=O)CCl)C1CCc2cnn(C)c2C1] | (Compound 360) |
| 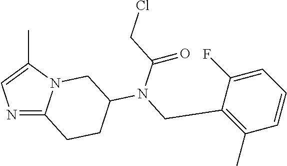 [SMILES: Cc1cccc(F)c1CN(C(=O)CCl)C1CCc2ncc(C)n2C1] | (Compound 359) |
| 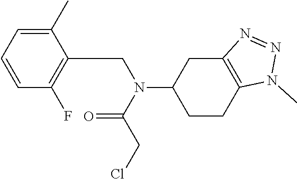 [SMILES: Cc1cccc(F)c1CN(C(=O)CCl)C1CCc2c(nnn2C)C1] | (Compound 362) |
| 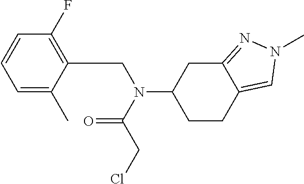 [SMILES: Cc1cccc(F)c1CN(C(=O)CCl)C1CCc2cn(C)nc2C1] | (Compound 361) |
| 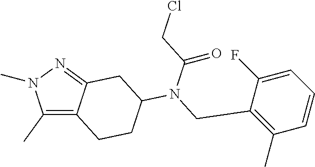 [SMILES: Cc1cccc(F)c1CN(C(=O)CCl)C1CCc2c(nn(C)c2C)C1] | (Compound 364) |
| 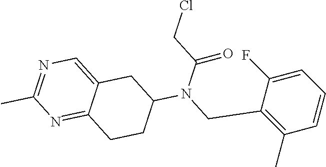 [SMILES: Cc1ncc2c(n1)CCC(N(Cc1c(C)cccc1F)C(=O)CCl)C2] | (Compound 363) |
| 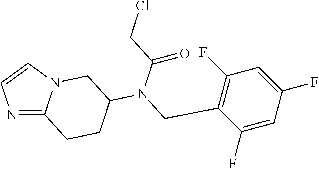 [SMILES: O=C(CCl)N(Cc1c(F)cc(F)cc1F)C1CCc2nccn2C1] | (Compound 366) |
| 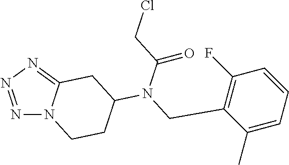 [SMILES: Cc1cccc(F)c1CN(C(=O)CCl)C1CCn2nnnc2C1] | (Compound 365) |
| 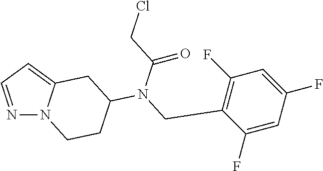 [SMILES: O=C(CCl)N(Cc1c(F)cc(F)cc1F)C1CCn2nccc2C1] | (Compound 368) |
| 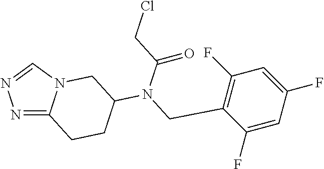 [SMILES: O=C(CCl)N(Cc1c(F)cc(F)cc1F)C1CCc2nncn2C1] | (Compound 367) |
| 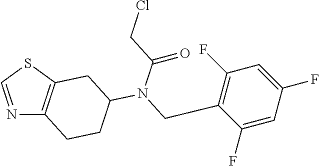 [SMILES: O=C(CCl)N(Cc1c(F)cc(F)cc1F)C1CCc2ncsc2C1] | (Compound 370) |
| 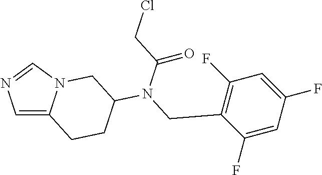 [SMILES: O=C(CCl)N(Cc1c(F)cc(F)cc1F)C1CCc2cncn2C1] | (Compound 369) |
| 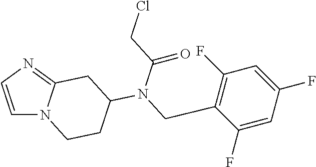 [SMILES: O=C(CCl)N(Cc1c(F)cc(F)cc1F)C1CCn2ccnc2C1] | (Compound 372) |
| 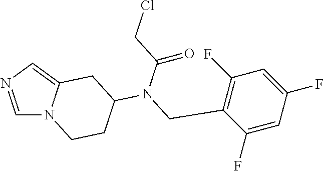 [SMILES: O=C(CCl)N(Cc1c(F)cc(F)cc1F)C1CCn2cncc2C1] | (Compound 371) |
| 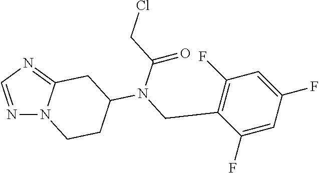 [SMILES: O=C(CCl)N(Cc1c(F)cc(F)cc1F)C1CCn2ncnc2C1] | (Compound 374) |
| 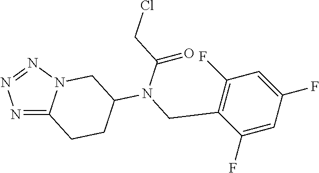 [SMILES: O=C(CCl)N(Cc1c(F)cc(F)cc1F)C1CCc2nnnn2C1] | (Compound 373) |
| 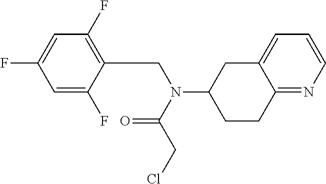 [SMILES: O=C(CCl)N(Cc1c(F)cc(F)cc1F)C1CCc2ncccc2C1] | (Compound 376) |
| 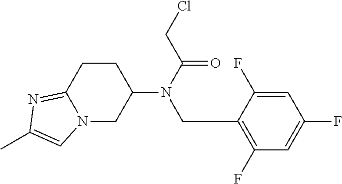 [SMILES: Cc1cn2c(n1)CCC(N(Cc1c(F)cc(F)cc1F)C(=O)CCl)C2] | (Compound 375) |
| 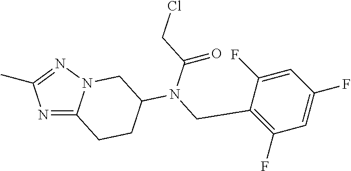 [SMILES: Cc1nc2n(n1)CC(N(Cc1c(F)cc(F)cc1F)C(=O)CCl)CC2] | (Compound 378) |
| 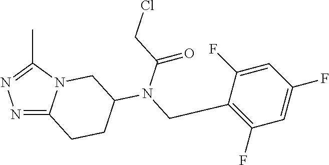 [SMILES: Cc1nnc2n1CC(N(Cc1c(F)cc(F)cc1F)C(=O)CCl)CC2] | (Compound 377) |
| 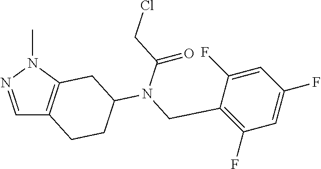 [SMILES: Cn1ncc2c1CC(N(Cc1c(F)cc(F)cc1F)C(=O)CCl)CC2] | (Compound 380) |
| 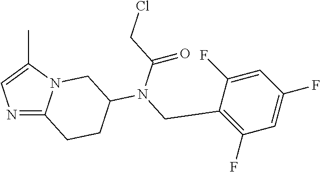 [SMILES: Cc1cnc2n1CC(N(Cc1c(F)cc(F)cc1F)C(=O)CCl)CC2] | (Compound 379) |
| 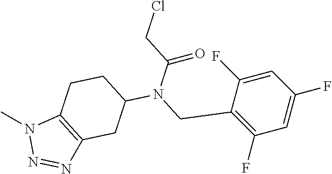 [SMILES: Cn1nnc2c1CCC(N(Cc1c(F)cc(F)cc1F)C(=O)CCl)C2] | (Compound 382) |
| 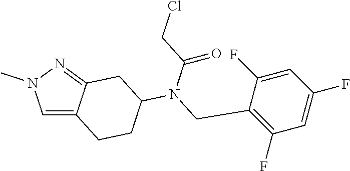 [SMILES: Cn1cc2c(n1)CC(N(Cc1c(F)cc(F)cc1F)C(=O)CCl)CC2] | (Compound 381) |
| 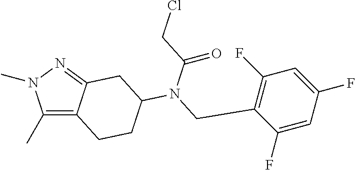 [SMILES: Cc1c2c(nn1C)CC(N(Cc1c(F)cc(F)cc1F)C(=O)CCl)CC2] | (Compound 384) |
| 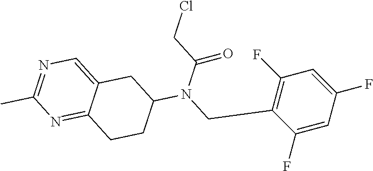 [SMILES: Cc1ncc2c(n1)CCC(N(Cc1c(F)cc(F)cc1F)C(=O)CCl)C2] | (Compound 383) |
| 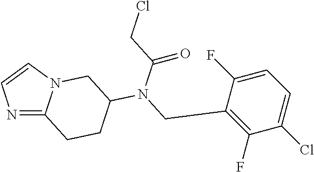 [SMILES: O=C(CCl)N(Cc1c(F)ccc(Cl)c1F)C1CCc2nccn2C1] | (Compound 386) |
| 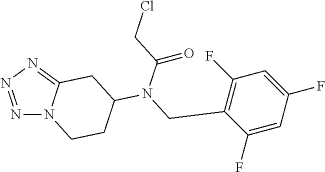 [SMILES: O=C(CCl)N(Cc1c(F)cc(F)cc1F)C1CCn2nnnc2C1] | (Compound 385) |
| 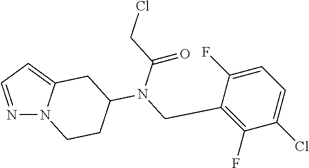 [SMILES: O=C(CCl)N(Cc1c(F)ccc(Cl)c1F)C1CCn2nccc2C1] | (Compound 388) |
| 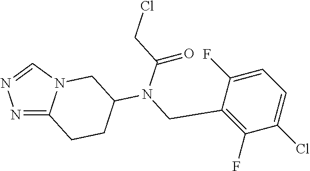 [SMILES: O=C(CCl)N(Cc1c(F)ccc(Cl)c1F)C1CCc2nncn2C1] | (Compound 387) |
| 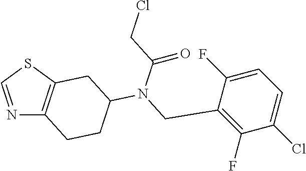 [SMILES: O=C(CCl)N(Cc1c(F)ccc(Cl)c1F)C1CCc2ncsc2C1] | (Compound 390) |
| 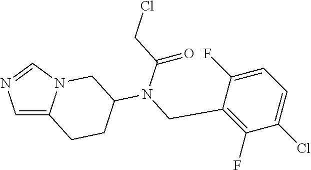 [SMILES: O=C(CCl)N(Cc1c(F)ccc(Cl)c1F)C1CCc2cncn2C1] | (Compound 389) |
| 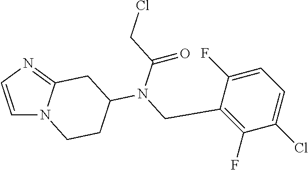 [SMILES: O=C(CCl)N(Cc1c(F)ccc(Cl)c1F)C1CCn2ccnc2C1] | (Compound 392) |
| 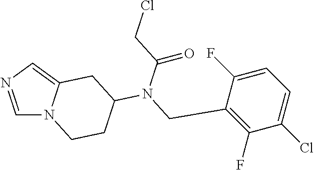 [SMILES: O=C(CCl)N(Cc1c(F)ccc(Cl)c1F)C1CCn2cncc2C1] | (Compound 391) |
| 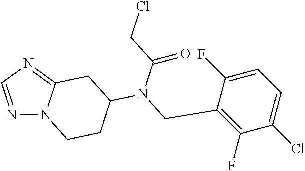 [SMILES: O=C(CCl)N(Cc1c(F)ccc(Cl)c1F)C1CCn2ncnc2C1] | (Compound 394) |
| 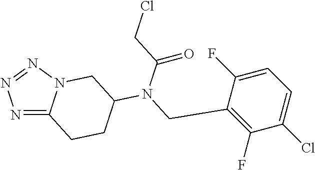 [SMILES: O=C(CCl)N(Cc1c(F)ccc(Cl)c1F)C1CCc2nnnn2C1] | (Compound 393) |
| 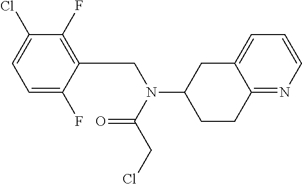 [SMILES: O=C(CCl)N(Cc1c(F)ccc(Cl)c1F)C1CCc2ncccc2C1] | (Compound 396) |
| 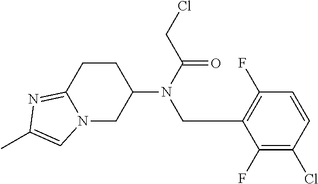 [SMILES: Cc1cn2c(n1)CCC(N(Cc1c(F)ccc(Cl)c1F)C(=O)CCl)C2] | (Compound 395) |
| 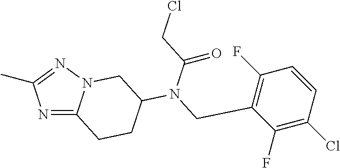 [SMILES: Cc1nc2n(n1)CC(N(Cc1c(F)ccc(Cl)c1F)C(=O)CCl)CC2] | (Compound 398) |
| 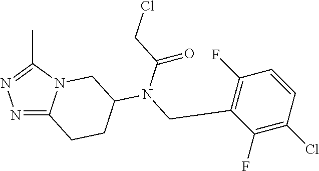 [SMILES: Cc1nnc2n1CC(N(Cc1c(F)ccc(Cl)c1F)C(=O)CCl)CC2] | (Compound 397) |
| 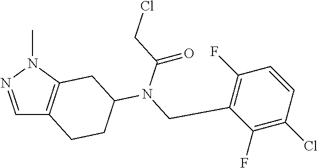 [SMILES: Cn1ncc2c1CC(N(Cc1c(F)ccc(Cl)c1F)C(=O)CCl)CC2] | (Compound 400) |
| 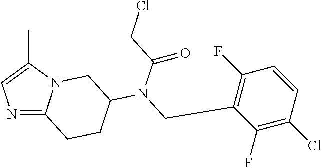 [SMILES: Cc1cnc2n1CC(N(Cc1c(F)ccc(Cl)c1F)C(=O)CCl)CC2] | (Compound 399) |
| 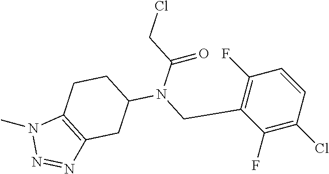 [SMILES: Cn1nnc2c1CCC(N(Cc1c(F)ccc(Cl)c1F)C(=O)CCl)C2] | (Compound 402) |
| 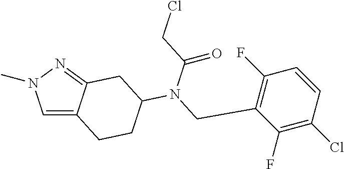 [SMILES: Cn1cc2c(n1)CC(N(Cc1c(F)ccc(Cl)c1F)C(=O)CCl)CC2] | (Compound 401) |
| 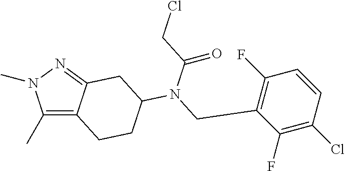 [SMILES: Cc1c2c(nn1C)CC(N(Cc1c(F)ccc(Cl)c1F)C(=O)CCl)CC2] | (Compound 404) |
| 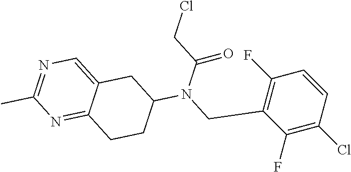 [SMILES: Cc1ncc2c(n1)CCC(N(Cc1c(F)ccc(Cl)c1F)C(=O)CCl)C2] | (Compound 403) |
| 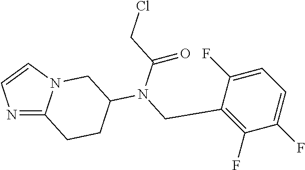 [SMILES: O=C(CCl)N(Cc1c(F)ccc(F)c1F)C1CCc2nccn2C1] | (Compound 406) |
| 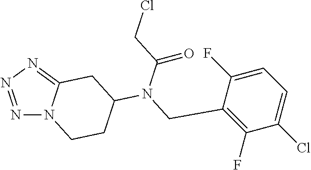 [SMILES: O=C(CCl)N(Cc1c(F)ccc(Cl)c1F)C1CCn2nnnc2C1] | (Compound 405) |
| 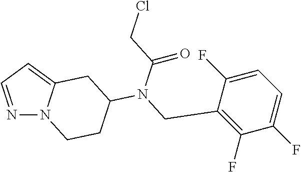 [SMILES: O=C(CCl)N(Cc1c(F)ccc(F)c1F)C1CCn2nccc2C1] | (Compound 408) |
| 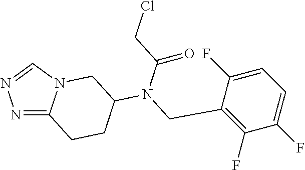 [SMILES: O=C(CCl)N(Cc1c(F)ccc(F)c1F)C1CCc2nncn2C1] | (Compound 407) |
| 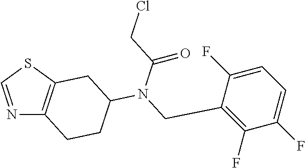 [SMILES: O=C(CCl)N(Cc1c(F)ccc(F)c1F)C1CCc2ncsc2C1] | (Compound 410) |
| 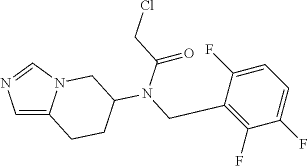 [SMILES: O=C(CCl)N(Cc1c(F)ccc(F)c1F)C1CCc2cncn2C1] | (Compound 409) |
| 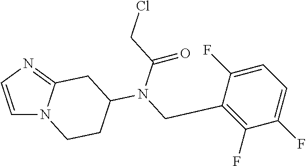 [SMILES: O=C(CCl)N(Cc1c(F)ccc(F)c1F)C1CCn2ccnc2C1] | (Compound 412) |
| 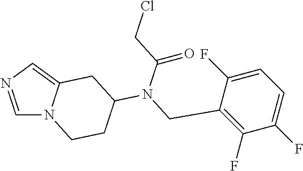 [SMILES: O=C(CCl)N(Cc1c(F)ccc(F)c1F)C1CCn2cncc2C1] | (Compound 411) |
| 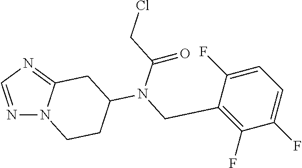 [SMILES: O=C(CCl)N(Cc1c(F)ccc(F)c1F)C1CCn2ncnc2C1] | (Compound 414) |
| 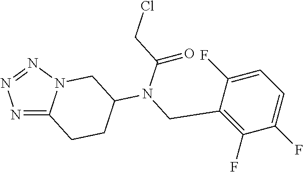 [SMILES: O=C(CCl)N(Cc1c(F)ccc(F)c1F)C1CCc2nnnn2C1] | (Compound 413) |
| 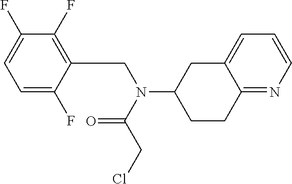 [SMILES: O=C(CCl)N(Cc1c(F)ccc(F)c1F)C1CCc2ncccc2C1] | (Compound 416) |
| 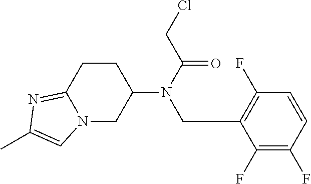 [SMILES: Cc1cn2c(n1)CCC(N(Cc1c(F)ccc(F)c1F)C(=O)CCl)C2] | (Compound 415) |
| 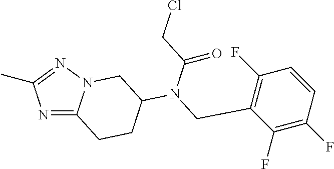 [SMILES: Cc1nc2n(n1)CC(N(Cc1c(F)ccc(F)c1F)C(=O)CCl)CC2] | (Compound 418) |
| 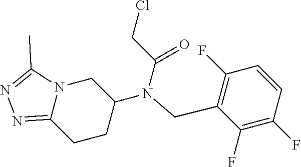 [SMILES: Cc1nnc2n1CC(N(Cc1c(F)ccc(F)c1F)C(=O)CCl)CC2] | (Compound 417) |
| 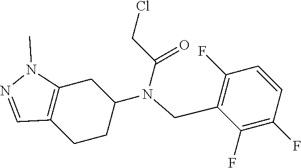 [SMILES: Cn1ncc2c1CC(N(Cc1c(F)ccc(F)c1F)C(=O)CCl)CC2] | (Compound 420) |
| 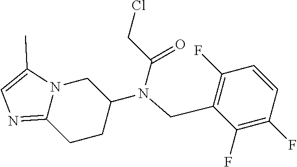 [SMILES: Cc1cnc2n1CC(N(Cc1c(F)ccc(F)c1F)C(=O)CCl)CC2] | (Compound 419) |
| 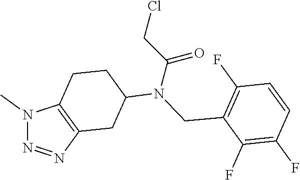 [SMILES: Cn1nnc2c1CCC(N(Cc1c(F)ccc(F)c1F)C(=O)CCl)C2] | (Compound 422) |
| 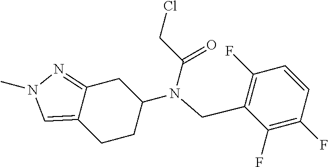 [SMILES: Cn1cc2c(n1)CC(N(Cc1c(F)ccc(F)c1F)C(=O)CCl)CC2] | (Compound 421) |
| 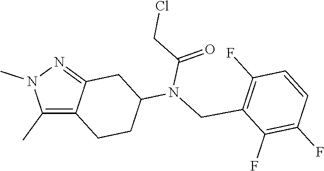 [SMILES: Cc1c2c(nn1C)CC(N(Cc1c(F)ccc(F)c1F)C(=O)CCl)CC2] | (Compound 424) |
| 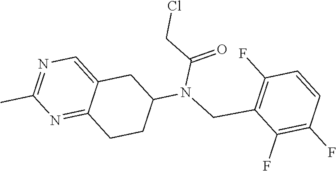 [SMILES: Cc1ncc2c(n1)CCC(N(Cc1c(F)ccc(F)c1F)C(=O)CCl)C2] | (Compound 423) |
| 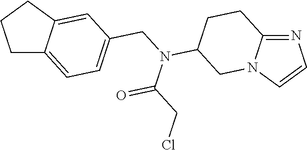 [SMILES: O=C(CCl)N(Cc1ccc2c(c1)CCC2)C1CCc2nccn2C1] | (Compound 426) |
| 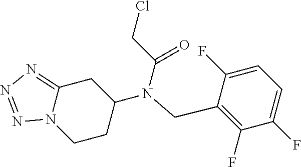 [SMILES: O=C(CCl)N(Cc1c(F)ccc(F)c1F)C1CCn2nnnc2C1] | (Compound 425) |
| 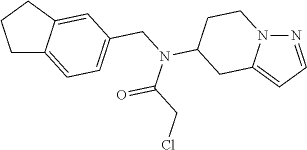 [SMILES: O=C(CCl)N(Cc1ccc2c(c1)CCC2)C1CCn2nccc2C1] | (Compound 428) |
| 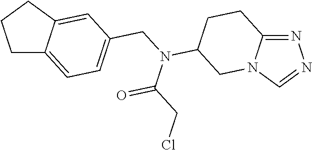 [SMILES: O=C(CCl)N(Cc1ccc2c(c1)CCC2)C1CCc2nncn2C1] | (Compound 427) |
| 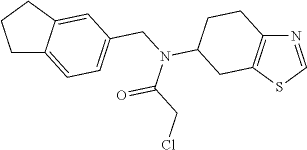 [SMILES: O=C(CCl)N(Cc1ccc2c(c1)CCC2)C1CCc2ncsc2C1] | (Compound 430) |
| 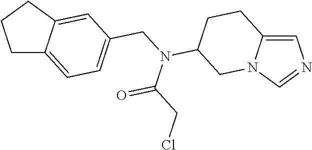 [SMILES: O=C(CCl)N(Cc1ccc2c(c1)CCC2)C1CCc2cncn2C1] | (Compound 429) |
| 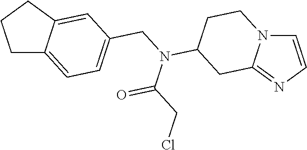 [SMILES: O=C(CCl)N(Cc1ccc2c(c1)CCC2)C1CCn2ccnc2C1] | (Compound 432) |
| 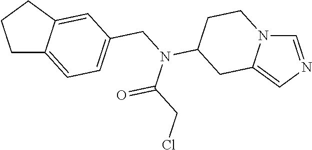 [SMILES: O=C(CCl)N(Cc1ccc2c(c1)CCC2)C1CCn2cncc2C1] | (Compound 431) |
| 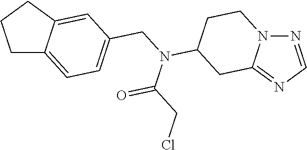 [SMILES: O=C(CCl)N(Cc1ccc2c(c1)CCC2)C1CCn2ncnc2C1] | (Compound 434) |
| 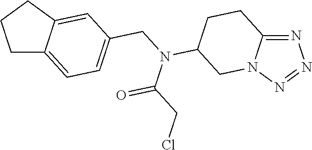 [SMILES: O=C(CCl)N(Cc1ccc2c(c1)CCC2)C1CCc2nnnn2C1] | (Compound 433) |
| 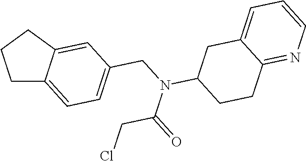 [SMILES: O=C(CCl)N(Cc1ccc2c(c1)CCC2)C1CCc2ncccc2C1] | (Compound 436) |
| 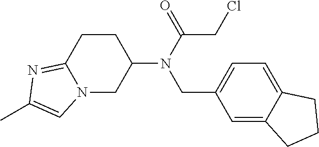 [SMILES: Cc1cn2c(n1)CCC(N(Cc1ccc3c(c1)CCC3)C(=O)CCl)C2] | (Compound 435) |
| 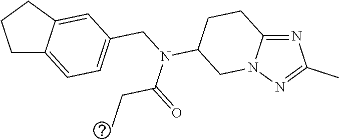 [SMILES: CCC(=O)N(Cc1ccc2c(c1)CCC2)C1CCc2nc(C)nn2C1] | (Compound 438) |
| 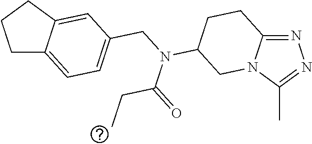 [SMILES: CCC(=O)N(Cc1ccc2c(c1)CCC2)C1CCc2nnc(C)n2C1] | (Compound 437) |
| 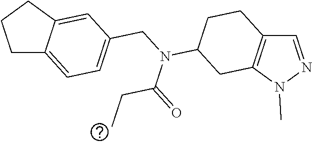 [SMILES: CCC(=O)N(Cc1ccc2c(c1)CCC2)C1CCc2cnn(C)c2C1] | (Compound 440) |
| 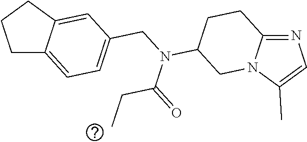 [SMILES: CCC(=O)N(Cc1ccc2c(c1)CCC2)C1CCc2ncc(C)n2C1] | (Compound 439) |
| 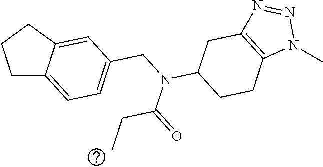 [SMILES: CCC(=O)N(Cc1ccc2c(c1)CCC2)C1CCc2c(nnn2C)C1] | (Compound 442) |
| 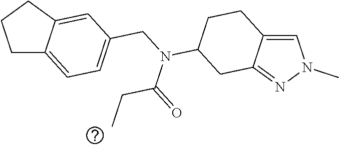 [SMILES: CCC(=O)N(Cc1ccc2c(c1)CCC2)C1CCc2cn(C)nc2C1] | (Compound 441) |
| 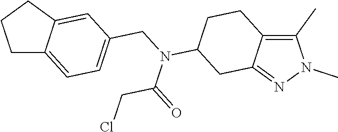 [SMILES: Cc1c2c(nn1C)CC(N(Cc1ccc3c(c1)CCC3)C(=O)CCl)CC2] | (Compound 444) |
| 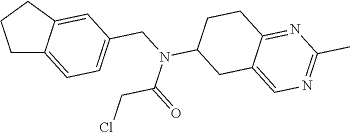 [SMILES: Cc1ncc2c(n1)CCC(N(Cc1ccc3c(c1)CCC3)C(=O)CCl)C2] | (Compound 443) |
| 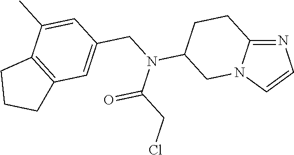 [SMILES: Cc1cc(CN(C(=O)CCl)C2CCc3nccn3C2)cc2c1CCC2] | (Compound 446) |
| 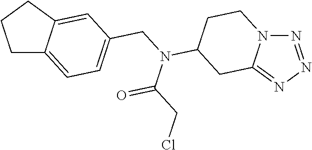 [SMILES: O=C(CCl)N(Cc1ccc2c(c1)CCC2)C1CCn2nnnc2C1] | (Compound 445) |
| 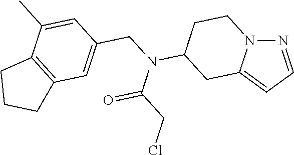 [SMILES: Cc1cc(CN(C(=O)CCl)C2CCn3nccc3C2)cc2c1CCC2] | (Compound 448) |
| 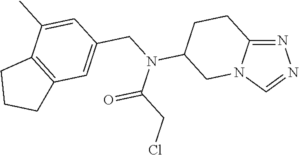 [SMILES: Cc1cc(CN(C(=O)CCl)C2CCc3nncn3C2)cc2c1CCC2] | (Compound 447) |
| 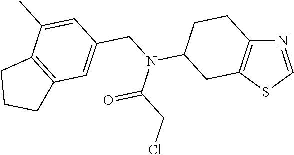 [SMILES: Cc1cc(CN(C(=O)CCl)C2CCc3ncsc3C2)cc2c1CCC2] | (Compound 450) |
| 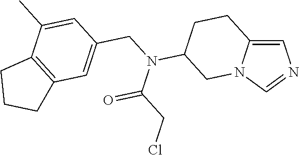 [SMILES: Cc1cc(CN(C(=O)CCl)C2CCc3cncn3C2)cc2c1CCC2] | (Compound 449) |
| 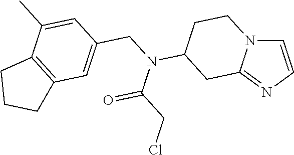 [SMILES: Cc1cc(CN(C(=O)CCl)C2CCn3ccnc3C2)cc2c1CCC2] | (Compound 452) |
| 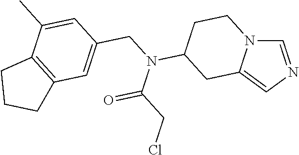 [SMILES: Cc1cc(CN(C(=O)CCl)C2CCn3cncc3C2)cc2c1CCC2] | (Compound 451) |
| 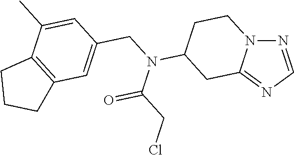 [SMILES: Cc1cc(CN(C(=O)CCl)C2CCn3ncnc3C2)cc2c1CCC2] | (Compound 454) |
| 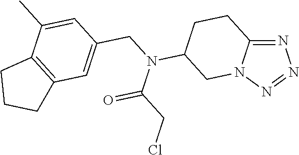 [SMILES: Cc1cc(CN(C(=O)CCl)C2CCc3nnnn3C2)cc2c1CCC2] | (Compound 453) |
| 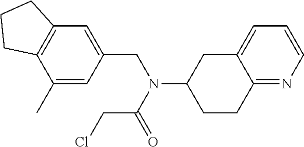 [SMILES: Cc1cc(CN(C(=O)CCl)C2CCc3ncccc3C2)cc2c1CCC2] | (Compound 456) |
| 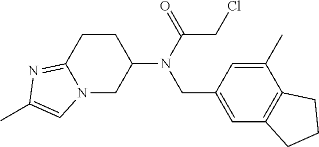 [SMILES: Cc1cn2c(n1)CCC(N(Cc1cc(C)c3c(c1)CCC3)C(=O)CCl)C2] | (Compound 455) |
| 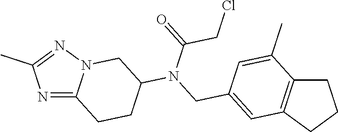 [SMILES: Cc1nc2n(n1)CC(N(Cc1cc(C)c3c(c1)CCC3)C(=O)CCl)CC2] | (Compound 458) |
| 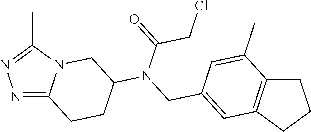 [SMILES: Cc1cc(CN(C(=O)CCl)C2CCc3nnc(C)n3C2)cc2c1CCC2] | (Compound 457) |
| 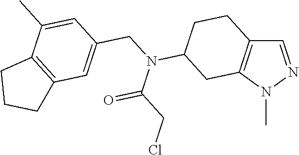 [SMILES: Cc1cc(CN(C(=O)CCl)C2CCc3cnn(C)c3C2)cc2c1CCC2] | (Compound 460) |
| 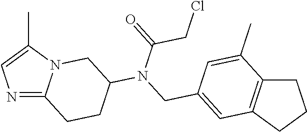 [SMILES: Cc1cc(CN(C(=O)CCl)C2CCc3ncc(C)n3C2)cc2c1CCC2] | (Compound 459) |
| 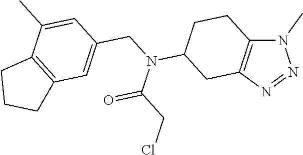 [SMILES: Cc1cc(CN(C(=O)CCl)C2CCc3c(nnn3C)C2)cc2c1CCC2] | (Compound 462) |
| 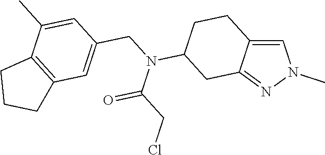 [SMILES: Cc1cc(CN(C(=O)CCl)C2CCc3cn(C)nc3C2)cc2c1CCC2] | (Compound 461) |
| 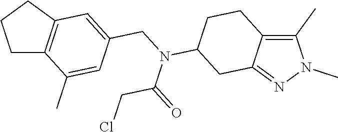 [SMILES: Cc1cc(CN(C(=O)CCl)C2CCc3c(nn(C)c3C)C2)cc2c1CCC2] | (Compound 464) |
| 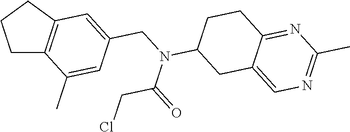 [SMILES: Cc1ncc2c(n1)CCC(N(Cc1cc(C)c3c(c1)CCC3)C(=O)CCl)C2] | (Compound 463) |
| 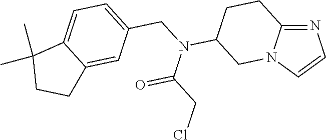 [SMILES: CC1(C)CCc2cc(CN(C(=O)CCl)C3CCc4nccn4C3)ccc21] | (Compound 466) |
| 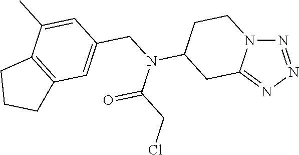 [SMILES: Cc1cc(CN(C(=O)CCl)C2CCn3nnnc3C2)cc2c1CCC2] | (Compound 465) |
| 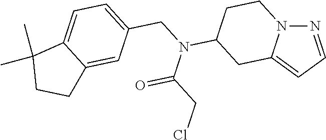 [SMILES: CC1(C)CCc2cc(CN(C(=O)CCl)C3CCn4nccc4C3)ccc21] | (Compound 468) |
| 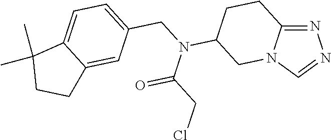 [SMILES: CC1(C)CCc2cc(CN(C(=O)CCl)C3CCc4nncn4C3)ccc21] | (Compound 467) |
| 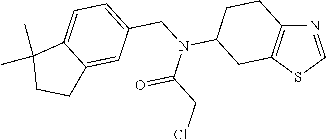 [SMILES: CC1(C)CCc2cc(CN(C(=O)CCl)C3CCc4ncsc4C3)ccc21] | (Compound 470) |
| 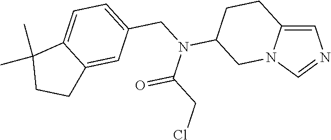 [SMILES: CC1(C)CCc2cc(CN(C(=O)CCl)C3CCc4cncn4C3)ccc21] | (Compound 469) |
| 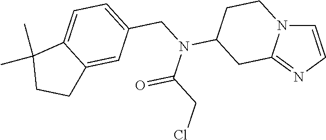 [SMILES: CC1(C)CCc2cc(CN(C(=O)CCl)C3CCn4ccnc4C3)ccc21] | (Compound 472) |
| 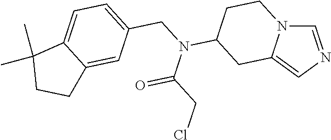 [SMILES: CC1(C)CCc2cc(CN(C(=O)CCl)C3CCn4cncc4C3)ccc21] | (Compound 471) |
| 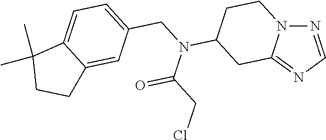 [SMILES: CC1(C)CCc2cc(CN(C(=O)CCl)C3CCn4ncnc4C3)ccc21] | (Compound 474) |
| 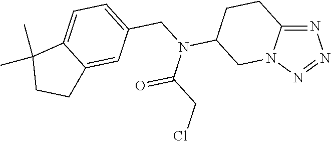 [SMILES: CC1(C)CCc2cc(CN(C(=O)CCl)C3CCc4nnnn4C3)ccc21] | (Compound 473) |
| 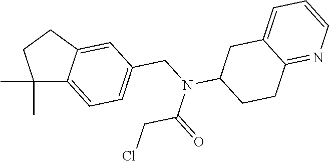 [SMILES: CC1(C)CCc2cc(CN(C(=O)CCl)C3CCc4ncccc4C3)ccc21] | (Compound 476) |
| 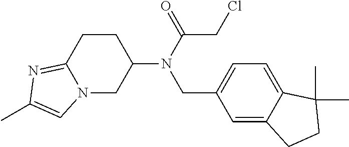 | (Compound 475) |
| 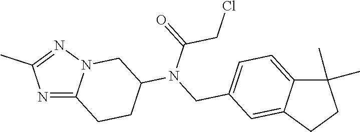 | (Compound 478) |
| 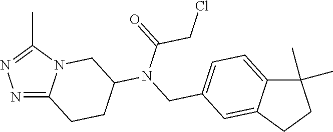 [SMILES: Cc1nnc2n1CC(N(Cc1ccc3c(c1)CCC3(C)C)C(=O)CCl)CC2] | (Compound 477) |
| 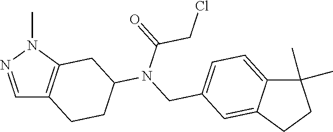 [SMILES: Cn1ncc2c1CC(N(Cc1ccc3c(c1)CCC3(C)C)C(=O)CCl)CC2] | (Compound 480) |
| 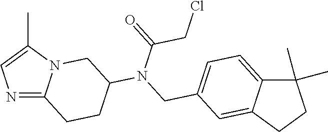 [SMILES: Cc1cnc2n1CC(N(Cc1ccc3c(c1)CCC3(C)C)C(=O)CCl)CC2] | (Compound 479) |
| 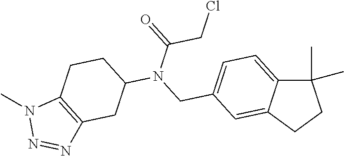 | (Compound 482) |
| 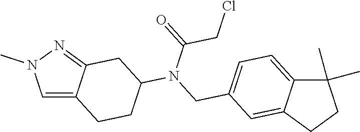 | (Compound 481) |
| 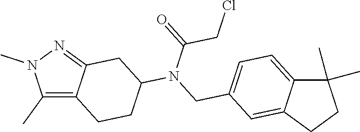 | (Compound 484) |
| 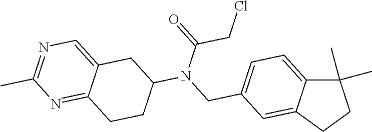 | (Compound 483) |
| 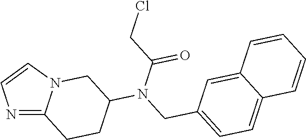 [SMILES: O=C(CCl)N(Cc1ccc2ccccc2c1)C1CCc2nccn2C1] | (Compound 486) |
| 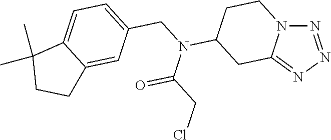 [SMILES: CC1(C)CCc2cc(CN(C(=O)CCl)C3CCn4nnnc4C3)ccc21] | (Compound 485) |
| 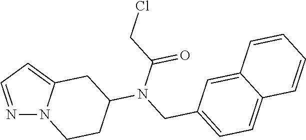 [SMILES: O=C(CCl)N(Cc1ccc2ccccc2c1)C1CCn2nccc2C1] | (Compound 488) |
| 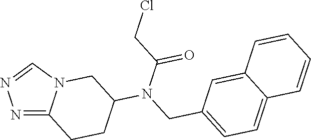 [SMILES: O=C(CCl)N(Cc1ccc2ccccc2c1)C1CCc2nncn2C1] | (Compound 487) |
| 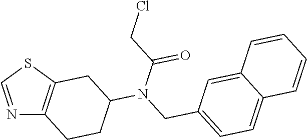 [SMILES: O=C(CCl)N(Cc1ccc2ccccc2c1)C1CCc2ncsc2C1] | (Compound 490) |
| 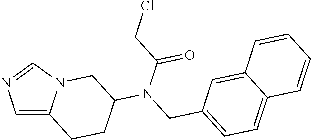 [SMILES: O=C(CCl)N(Cc1ccc2ccccc2c1)C1CCc2cncn2C1] | (Compound 489) |
| 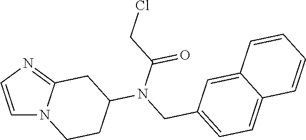 [SMILES: O=C(CCl)N(Cc1ccc2ccccc2c1)C1CCn2ccnc2C1] | (Compound 492) |
| 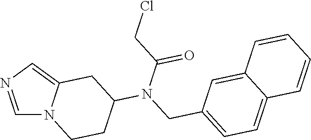 [SMILES: O=C(CCl)N(Cc1ccc2ccccc2c1)C1CCn2cncc2C1] | (Compound 491) |
| 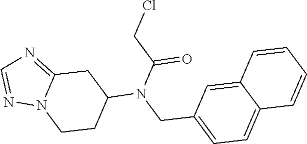 [SMILES: O=C(CCl)N(Cc1ccc2ccccc2c1)C1CCn2ncnc2C1] | (Compound 494) |
| 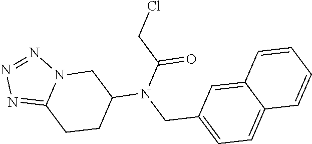 [SMILES: O=C(CCl)N(Cc1ccc2ccccc2c1)C1CCc2nnnn2C1] | (Compound 493) |
| 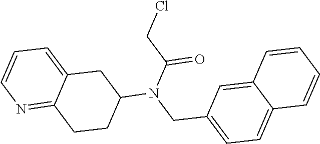 [SMILES: O=C(CCl)N(Cc1ccc2ccccc2c1)C1CCc2ncccc2C1] | (Compound 496) |
| 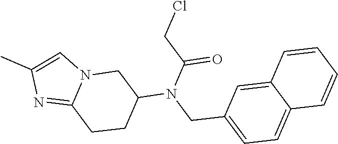 [SMILES: Cc1cn2c(n1)CCC(N(Cc1ccc3ccccc3c1)C(=O)CCl)C2] | (Compound 495) |
| 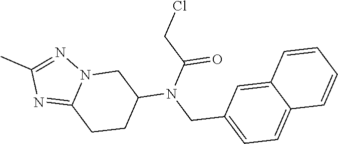 [SMILES: Cc1nc2n(n1)CC(N(Cc1ccc3ccccc3c1)C(=O)CCl)CC2] | (Compound 498) |
| 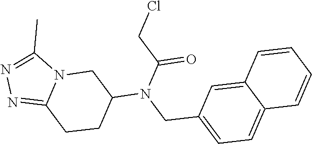 [SMILES: Cc1nnc2n1CC(N(Cc1ccc3ccccc3c1)C(=O)CCl)CC2] | (Compound 497) |
| 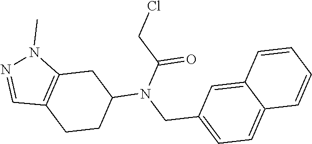 [SMILES: Cn1ncc2c1CC(N(Cc1ccc3ccccc3c1)C(=O)CCl)CC2] | (Compound 500) |
| 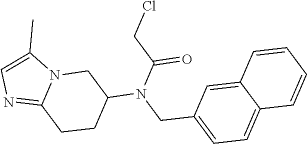 [SMILES: Cc1cnc2n1CC(N(Cc1ccc3ccccc3c1)C(=O)CCl)CC2] | (Compound 499) |
| 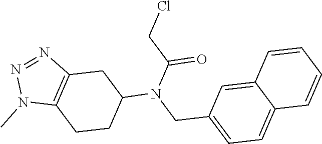 [SMILES: Cn1nnc2c1CCC(N(Cc1ccc3ccccc3c1)C(=O)CCl)C2] | (Compound 502) |
| 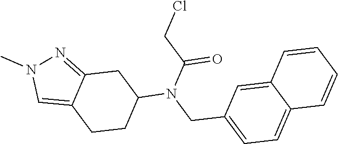 [SMILES: Cn1cc2c(n1)CC(N(Cc1ccc3ccccc3c1)C(=O)CCl)CC2] | (Compound 501) |
| 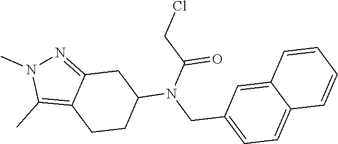 [SMILES: Cc1c2c(nn1C)CC(N(Cc1ccc3ccccc3c1)C(=O)CCl)CC2] | (Compound 504) |
| 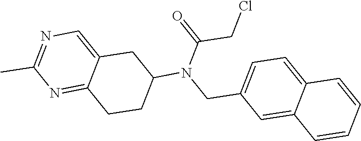 [SMILES: Cc1ncc2c(n1)CCC(N(Cc1ccc3ccccc3c1)C(=O)CCl)C2] | (Compound 503) |
| 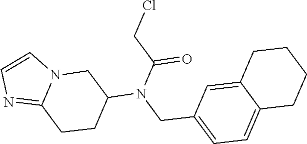 [SMILES: O=C(CCl)N(Cc1ccc2c(c1)CCCC2)C1CCc2nccn2C1] | (Compound 506) |
| 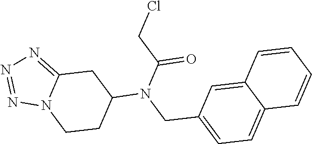 [SMILES: O=C(CCl)N(Cc1ccc2ccccc2c1)C1CCn2nnnc2C1] | (Compound 505) |
| 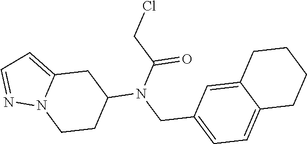 [SMILES: O=C(CCl)N(Cc1ccc2c(c1)CCCC2)C1CCn2nccc2C1] | (Compound 508) |
| 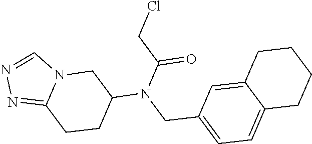 [SMILES: O=C(CCl)N(Cc1ccc2c(c1)CCCC2)C1CCc2nncn2C1] | (Compound 507) |
| 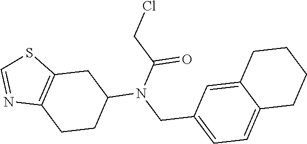 [SMILES: O=C(CCl)N(Cc1ccc2c(c1)CCCC2)C1CCc2ncsc2C1] | (Compound 510) |
| 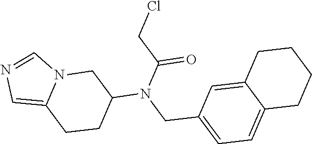 [SMILES: O=C(CCl)N(Cc1ccc2c(c1)CCCC2)C1CCc2cncn2C1] | (Compound 509) |
| 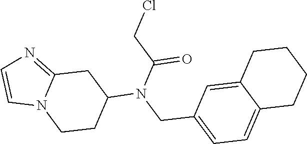 [SMILES: O=C(CCl)N(Cc1ccc2c(c1)CCCC2)C1CCn2ccnc2C1] | (Compound 512) |
| 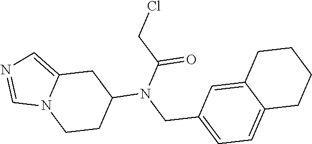 [SMILES: O=C(CCl)N(Cc1ccc2c(c1)CCCC2)C1CCn2cncc2C1] | (Compound 511) |
| 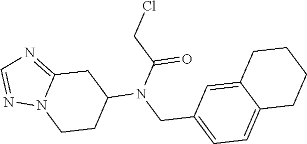 [SMILES: O=C(CCl)N(Cc1ccc2c(c1)CCCC2)C1CCn2ncnc2C1] | (Compound 514) |
| 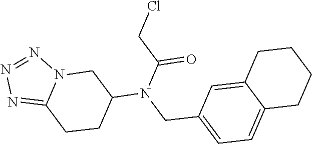 [SMILES: O=C(CCl)N(Cc1ccc2c(c1)CCCC2)C1CCc2nnnn2C1] | (Compound 513) |
| 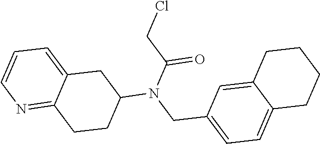 [SMILES: O=C(CCl)N(Cc1ccc2c(c1)CCCC2)C1CCc2ncccc2C1] | (Compound 516) |
| 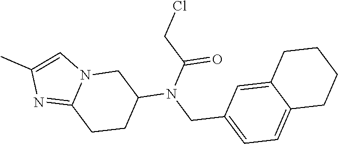 [SMILES: Cc1cn2c(n1)CCC(N(Cc1ccc3c(c1)CCCC3)C(=O)CCl)C2] | (Compound 515) |
| 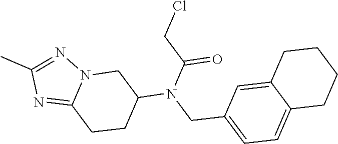 [SMILES: Cc1nc2n(n1)CC(N(Cc1ccc3c(c1)CCCC3)C(=O)CCl)CC2] | (Compound 518) |
| 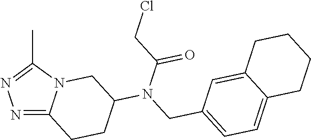 [SMILES: Cc1nnc2n1CC(N(Cc1ccc3c(c1)CCCC3)C(=O)CCl)CC2] | (Compound 517) |
| 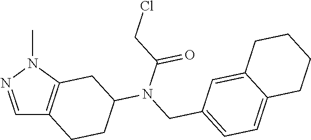 [SMILES: Cn1ncc2c1CC(N(Cc1ccc3c(c1)CCCC3)C(=O)CCl)CC2] | (Compound 520) |
| 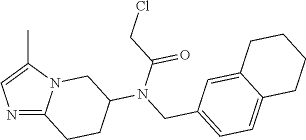 [SMILES: Cc1cnc2n1CC(N(Cc1ccc3c(c1)CCCC3)C(=O)CCl)CC2] | (Compound 519) |
| 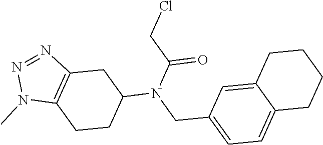 [SMILES: Cn1nnc2c1CCC(N(Cc1ccc3c(c1)CCCC3)C(=O)CCl)C2] | (Compound 522) |
| 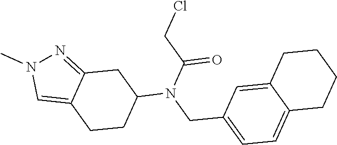 [SMILES: Cn1cc2c(n1)CC(N(Cc1ccc3c(c1)CCCC3)C(=O)CCl)CC2] | (Compound 521) |
| 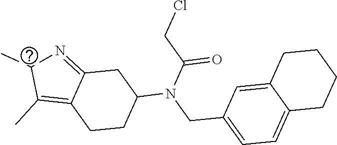 [SMILES: CC1=C2CCC(N(Cc3ccc4c(c3)CCCC4)C(=O)CCl)CC2=NC1C] | (Compound 524) |
| 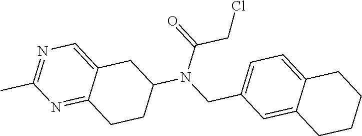 [SMILES: Cc1ncc2c(n1)CCC(N(Cc1ccc3c(c1)CCCC3)C(=O)CCl)C2] | (Compound 523) |
| 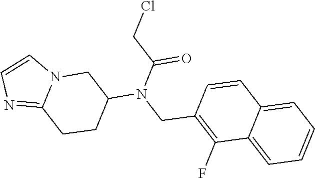 [SMILES: O=C(CCl)N(Cc1ccc2ccccc2c1F)C1CCc2nccn2C1] | (Compound 526) |
| 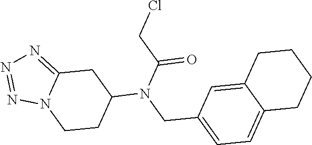 [SMILES: O=C(CCl)N(Cc1ccc2c(c1)CCCC2)C1CCn2nnnc2C1] | (Compound 525) |
| 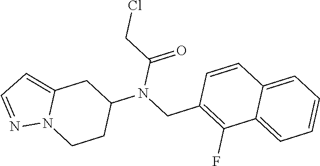 [SMILES: O=C(CCl)N(Cc1ccc2ccccc2c1F)C1CCn2nccc2C1] | (Compound 528) |
| 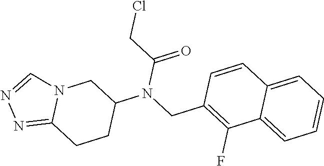 [SMILES: O=C(CCl)N(Cc1ccc2ccccc2c1F)C1CCc2nncn2C1] | (Compound 527) |
| 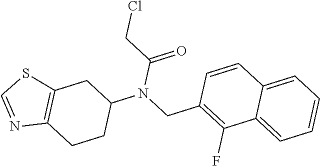 [SMILES: O=C(CCl)N(Cc1ccc2ccccc2c1F)C1CCc2ncsc2C1] | (Compound 530) |
| 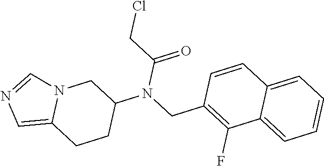 [SMILES: O=C(CCl)N(Cc1ccc2ccccc2c1F)C1CCc2cncn2C1] | (Compound 529) |
| 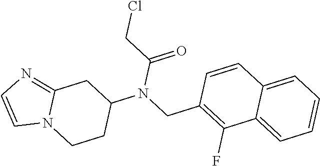 [SMILES: O=C(CCl)N(Cc1ccc2ccccc2c1F)C1CCn2ccnc2C1] | (Compound 532) |
| 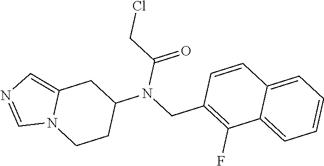 [SMILES: O=C(CCl)N(Cc1ccc2ccccc2c1F)C1CCn2cncc2C1] | (Compound 531) |
| 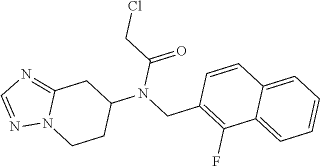 [SMILES: O=C(CCl)N(Cc1ccc2ccccc2c1F)C1CCn2ncnc2C1] | (Compound 534) |
| 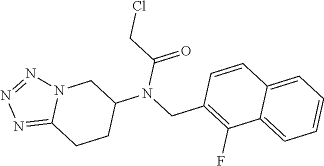 [SMILES: O=C(CCl)N(Cc1ccc2ccccc2c1F)C1CCc2nnnn2C1] | (Compound 533) |
| 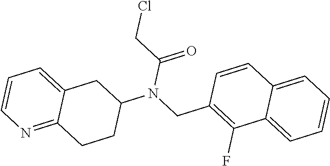 [SMILES: O=C(CCl)N(Cc1ccc2ccccc2c1F)C1CCc2ncccc2C1] | (Compound 536) |
| 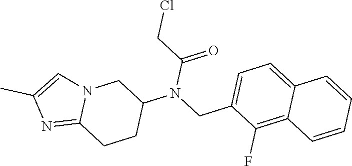 [SMILES: Cc1cn2c(n1)CCC(N(Cc1ccc3ccccc3c1F)C(=O)CCl)C2] | (Compound 535) |
| 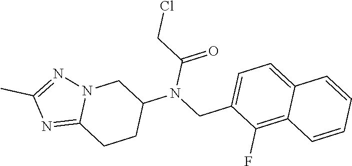 [SMILES: Cc1nc2n(n1)CC(N(Cc1ccc3ccccc3c1F)C(=O)CCl)CC2] | (Compound 538) |
| 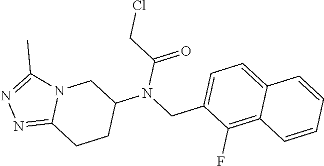 [SMILES: Cc1nnc2n1CC(N(Cc1ccc3ccccc3c1F)C(=O)CCl)CC2] | (Compound 537) |
| 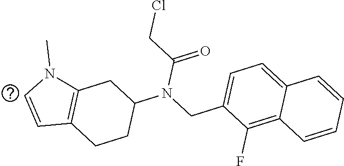 [SMILES: Cn1ccc2c1CC(N(Cc1ccc3ccccc3c1F)C(=O)CCl)CC2] | (Compound 540) |
| 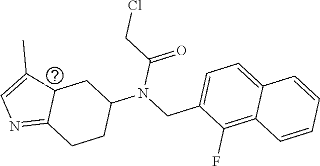 [SMILES: CC1=CN=C2CCC(N(Cc3ccc4ccccc4c3F)C(=O)CCl)CC12] | (Compound 539) |
| 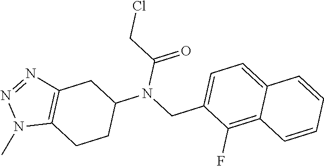 [SMILES: Cn1nnc2c1CCC(N(Cc1ccc3ccccc3c1F)C(=O)CCl)C2] | (Compound 542) |
| 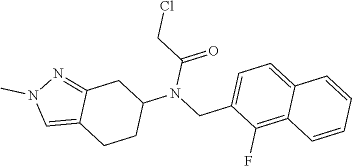 [SMILES: Cn1cc2c(n1)CC(N(Cc1ccc3ccccc3c1F)C(=O)CCl)CC2] | (Compound 541) |
| 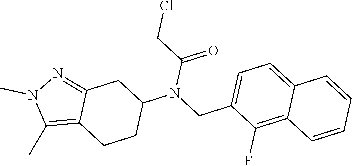 [SMILES: Cc1c2c(nn1C)CC(N(Cc1ccc3ccccc3c1F)C(=O)CCl)CC2] | (Compound 544) |
| 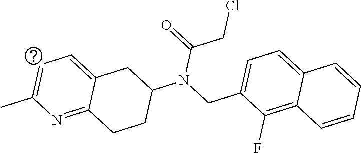 [SMILES: Cc1ccc2c(n1)CCC(N(Cc1ccc3ccccc3c1F)C(=O)CCl)C2] | (Compound 543) |
| 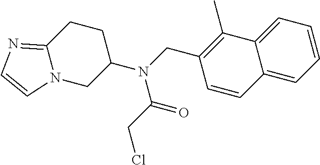 [SMILES: Cc1c(CN(C(=O)CCl)C2CCc3nccn3C2)ccc2ccccc12] | (Compound 546) |
| 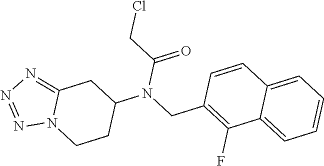 [SMILES: O=C(CCl)N(Cc1ccc2ccccc2c1F)C1CCn2nnnc2C1] | (Compound 545) |
| 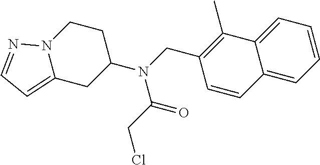 [SMILES: Cc1c(CN(C(=O)CCl)C2CCn3nccc3C2)ccc2ccccc12] | (Compound 548) |
| 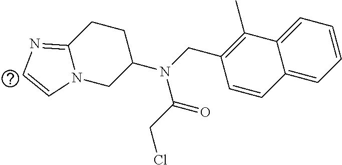 [SMILES: Cc1c(CN(C(=O)CCl)C2CCc3nccn3C2)ccc2ccccc12] | (Compound 547) |
| 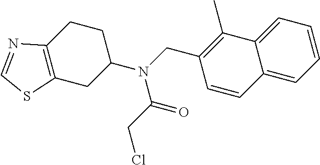 [SMILES: Cc1c(CN(C(=O)CCl)C2CCc3ncsc3C2)ccc2ccccc12] | (Compound 550) |
| 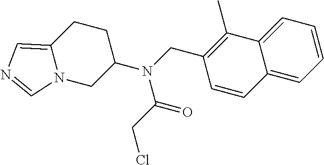 [SMILES: Cc1c(CN(C(=O)CCl)C2CCc3cncn3C2)ccc2ccccc12] | (Compound 549) |
| 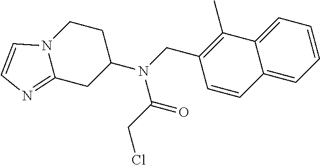 [SMILES: Cc1c(CN(C(=O)CCl)C2CCn3ccnc3C2)ccc2ccccc12] | (Compound 552) |
| 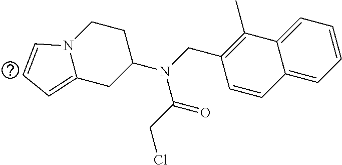 [SMILES: Cc1c(CN(C(=O)CCl)C2CCn3cccc3C2)ccc2ccccc12] | (Compound 551) |
| 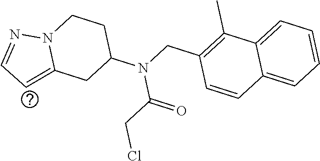 [SMILES: Cc1c(CN(C(=O)CCl)C2CCn3nccc3C2)ccc2ccccc12] | (Compound 554) |
| 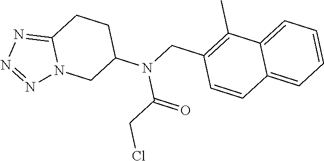 [SMILES: Cc1c(CN(C(=O)CCl)C2CCc3nnnn3C2)ccc2ccccc12] | (Compound 553) |
| 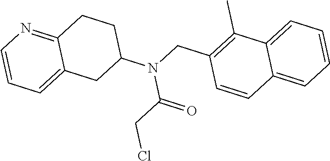 [SMILES: Cc1c(CN(C(=O)CCl)C2CCc3ncccc3C2)ccc2ccccc12] | (Forumla 556) |
| 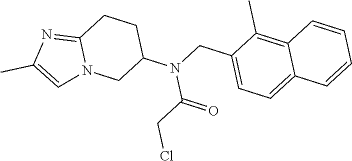 [SMILES: Cc1cn2c(n1)CCC(N(Cc1ccc3ccccc3c1C)C(=O)CCl)C2] | (Compound 555) |
| 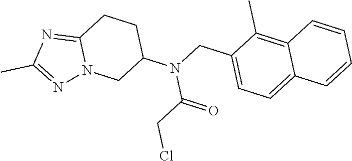 [SMILES: Cc1nc2n(n1)CC(N(Cc1ccc3ccccc3c1C)C(=O)CCl)CC2] | (Compound 558) |
| 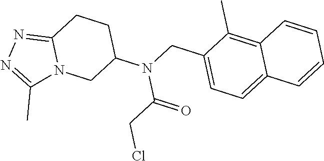 [SMILES: Cc1c(CN(C(=O)CCl)C2CCc3nnc(C)n3C2)ccc2ccccc12] | (Compound 557) |
| 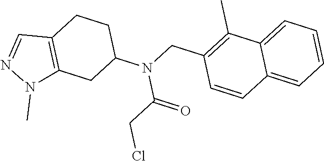 [SMILES: Cc1c(CN(C(=O)CCl)C2CCc3cnn(C)c3C2)ccc2ccccc12] | (Compound 560) |
| 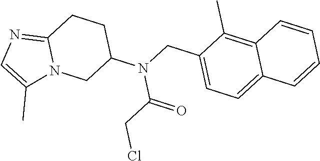 [SMILES: Cc1c(CN(C(=O)CCl)C2CCc3ncc(C)n3C2)ccc2ccccc12] | (Compound 559) |
| 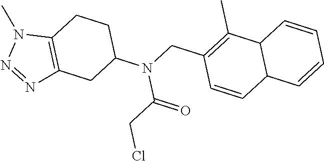 [SMILES: CC1=C(CN(C(=O)CCl)C2CCc3c(nnn3C)C2)C=CC2C=CC=CC12] | (Compound 562) |
| 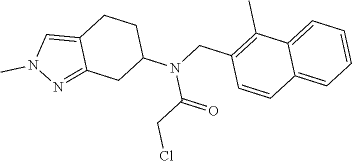 [SMILES: Cc1c(CN(C(=O)CCl)C2CCc3cn(C)nc3C2)ccc2ccccc12] | (Compound 561) |
| 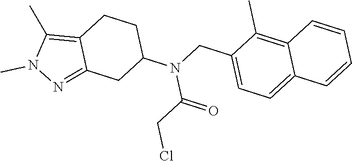 [SMILES: Cc1c(CN(C(=O)CCl)C2CCc3c(nn(C)c3C)C2)ccc2ccccc12] | (Compound 564) |
| 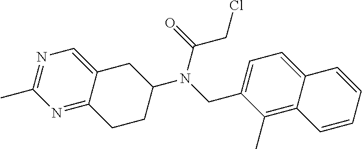 [SMILES: Cc1ncc2c(n1)CCC(N(Cc1ccc3ccccc3c1C)C(=O)CCl)C2] | (Compound 563) |
| 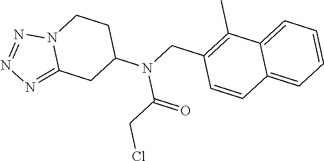 [SMILES: Cc1c(CN(C(=O)CCl)C2CCn3nnnc3C2)ccc2ccccc12] | (Compound 565) |
| 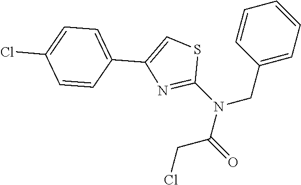 [SMILES: O=C(CCl)N(Cc1ccccc1)c1nc(-c2ccc(Cl)cc2)cs1] | (Compound 566) |
| 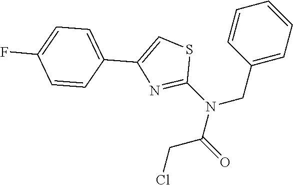 [SMILES: O=C(CCl)N(Cc1ccccc1)c1nc(-c2ccc(F)cc2)cs1] | (Compound 567) |
| 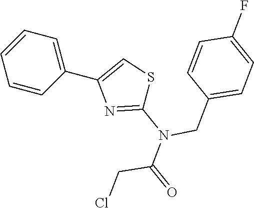 [SMILES: O=C(CCl)N(Cc1ccc(F)cc1)c1nc(-c2ccccc2)cs1] | (Compound 568) |
| 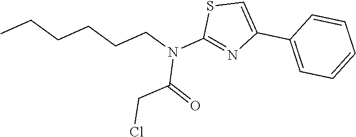 [SMILES: CCCCCCN(C(=O)CCl)c1nc(-c2ccccc2)cs1] | (Compound 569) |
| 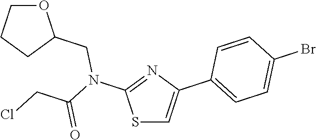 [SMILES: O=C(CCl)N(CC1CCCO1)c1nc(-c2ccc(Br)cc2)cs1] | (Compound 570) |
| 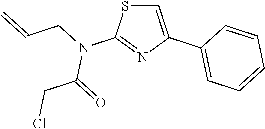 [SMILES: C=CCN(C(=O)CCl)c1nc(-c2ccccc2)cs1] | (Compound 571) |
| 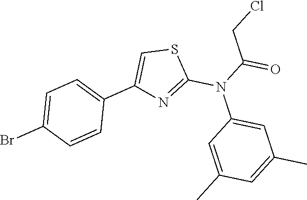 [SMILES: Cc1cc(C)cc(N(C(=O)CCl)c2nc(-c3ccc(Br)cc3)cs2)c1] | (Compound 572) |
| 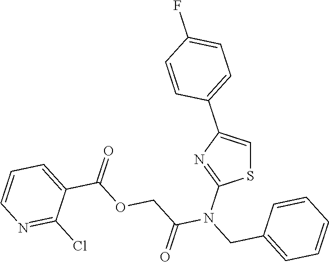 [SMILES: O=C(OCC(=O)N(Cc1ccccc1)c1nc(-c2ccc(F)cc2)cs1)c1cccnc1Cl] | (Compound 573) |
| 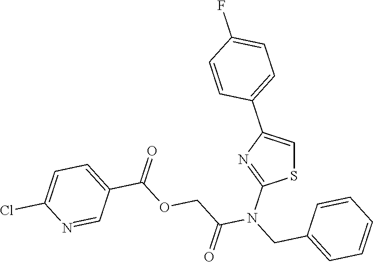 [SMILES: O=C(OCC(=O)N(Cc1ccccc1)c1nc(-c2ccc(F)cc2)cs1)c1ccc(Cl)nc1] | (Compound 574) |
| 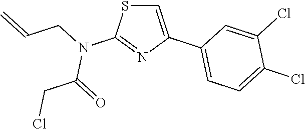 [SMILES: C=CCN(C(=O)CCl)c1nc(-c2ccc(Cl)c(Cl)c2)cs1] | (Compound 575) |
| 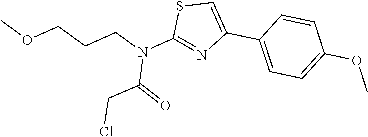 | (Compound 576) |
| 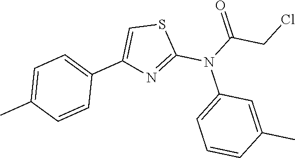 | (Compound 577) |
| 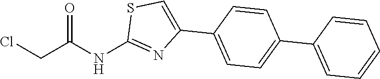 [SMILES: O=C(CCl)Nc1nc(-c2ccc(-c3ccccc3)cc2)cs1] | (Compound 578) |
| 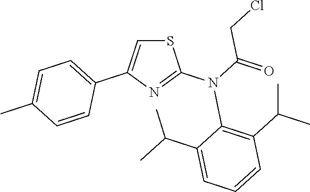 [SMILES: Cc1ccc(-c2csc(N(C(=O)CCl)c3c(C(C)C)cccc3C(C)C)n2)cc1] | (Compound 579) |
| 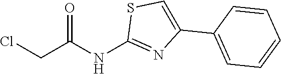 [SMILES: O=C(CCl)Nc1nc(-c2ccccc2)cs1] | (Compound 580) |
| 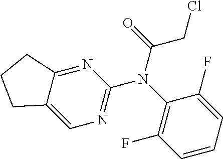 [SMILES: O=C(CCl)N(c1ncc2c(n1)CCC2)c1c(F)cccc1F] | (Compound 581) |
| 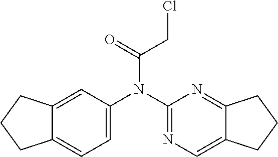 [SMILES: O=C(CCl)N(c1ccc2c(c1)CCC2)c1ncc2c(n1)CCC2] | (Compound 582) |
| 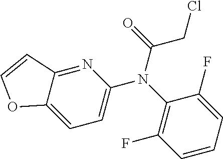 [SMILES: O=C(CCl)N(c1ccc2occc2n1)c1c(F)cccc1F] | (Compound 583) |
| 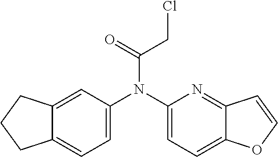 [SMILES: O=C(CCl)N(c1ccc2c(c1)CCC2)c1ccc2occc2n1] | (Compound 584) |
| 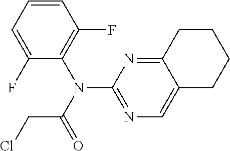 [SMILES: O=C(CCl)N(c1ncc2c(n1)CCCC2)c1c(F)cccc1F] | (Compound 585) |
| 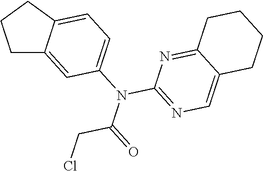 [SMILES: O=C(CCl)N(c1ccc2c(c1)CCC2)c1ncc2c(n1)CCCC2] | (Compound 586) |
| 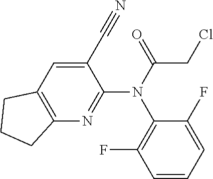 [SMILES: N#Cc1cc2c(nc1N(C(=O)CCl)c1c(F)cccc1F)CCC2] | (Compound 587) |
| 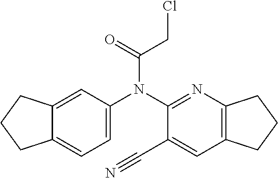 [SMILES: N#Cc1cc2c(nc1N(C(=O)CCl)c1ccc3c(c1)CCC3)CCC2] | (Compound 588) |
| 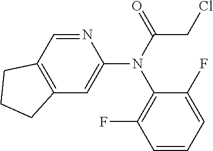 [SMILES: O=C(CCl)N(c1cc2c(cn1)CCC2)c1c(F)cccc1F] | (Compound 589) |
| 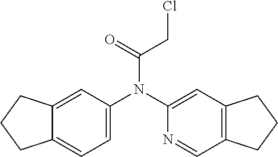 [SMILES: O=C(CCl)N(c1ccc2c(c1)CCC2)c1cc2c(cn1)CCC2] | (Compound 590) |
| 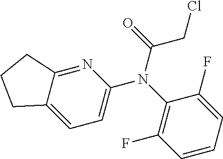 [SMILES: O=C(CCl)N(c1ccc2c(n1)CCC2)c1c(F)cccc1F] | (Compound 591) |
| 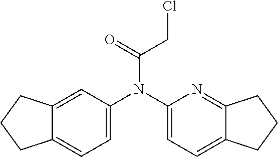 [SMILES: O=C(CCl)N(c1ccc2c(c1)CCC2)c1ccc2c(n1)CCC2] | (Compound 592) |
| 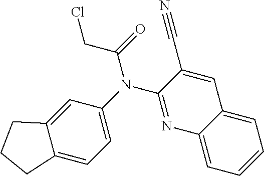 [SMILES: N#Cc1cc2ccccc2nc1N(C(=O)CCl)c1ccc2c(c1)CCC2] | (Compound 593) |
| 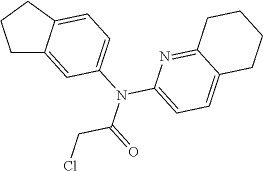 [SMILES: O=C(CCl)N(c1ccc2c(c1)CCC2)c1ccc2c(n1)CCCC2] | (Compound 594) |
| 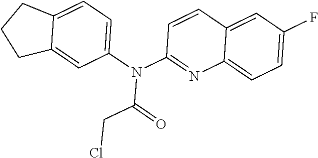 [SMILES: O=C(CCl)N(c1ccc2c(c1)CCC2)c1ccc2cc(F)ccc2n1] | (Compound 595) |
| 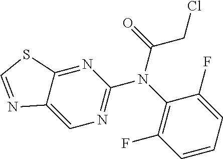 [SMILES: O=C(CCl)N(c1ncc2ncsc2n1)c1c(F)cccc1F] | (Compound 596) |
| 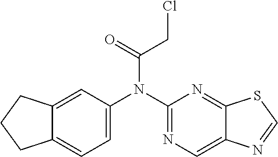 [SMILES: O=C(CCl)N(c1ccc2c(c1)CCC2)c1ncc2ncsc2n1] | (Compound 597) |
| 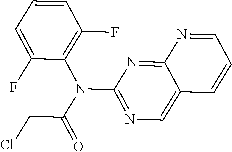 [SMILES: O=C(CCl)N(c1ncc2cccnc2n1)c1c(F)cccc1F] | (Compound 598) |
| 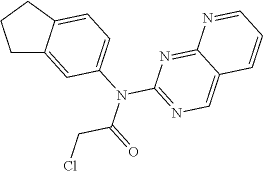 [SMILES: O=C(CCl)N(c1ccc2c(c1)CCC2)c1ncc2cccnc2n1] | (Compound 599) |
| 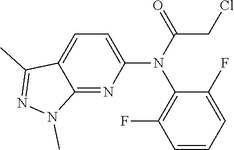 [SMILES: Cc1nn(C)c2nc(N(C(=O)CCl)c3c(F)cccc3F)ccc12] | (Compound 600) |
| 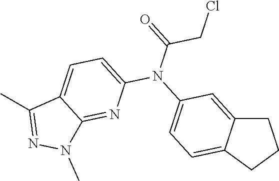 [SMILES: Cc1nn(C)c2nc(N(C(=O)CCl)c3ccc4c(c3)CCC4)ccc12] | (Compound 601) |
| 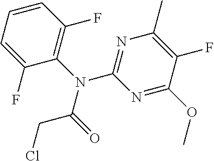 [SMILES: COc1nc(N(C(=O)CCl)c2c(F)cccc2F)nc(C)c1F] | (Compound 602) |
| 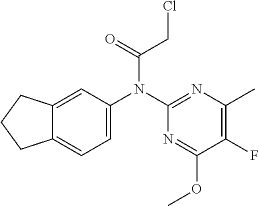 [SMILES: COc1nc(N(C(=O)CCl)c2ccc3c(c2)CCC3)nc(C)c1F] | (Compound 603) |
| 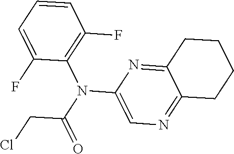 [SMILES: O=C(CCl)N(c1cnc2c(n1)CCCC2)c1c(F)cccc1F] | (Compound 604) |
| 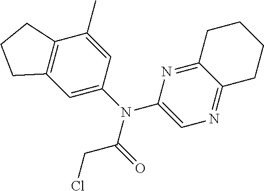 [SMILES: Cc1cc(N(C(=O)CCl)c2cnc3c(n2)CCCC3)cc2c1CCC2] | (Compound 605) |
| 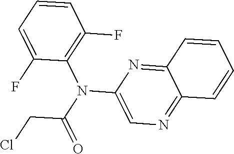 [SMILES: O=C(CCl)N(c1cnc2ccccc2n1)c1c(F)cccc1F] | (Compound 606) |
| 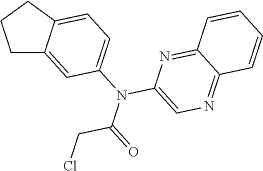 [SMILES: O=C(CCl)N(c1ccc2c(c1)CCC2)c1cnc2ccccc2n1] | (Compound 607) |
| 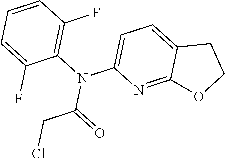 [SMILES: O=C(CCl)N(c1ccc2c(n1)OCC2)c1c(F)cccc1F] | (Compound 608) |
| 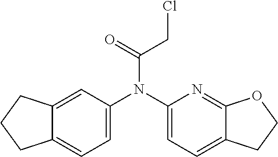 [SMILES: O=C(CCl)N(c1ccc2c(c1)CCC2)c1ccc2c(n1)OCC2] | (Compound 609) |
| 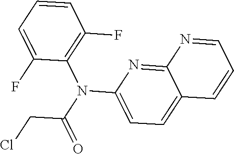 [SMILES: O=C(CCl)N(c1ccc2cccnc2n1)c1c(F)cccc1F] | (Compound 610) |
| 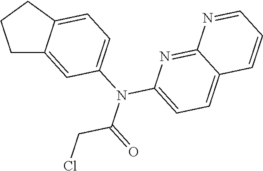 [SMILES: O=C(CCl)N(c1ccc2c(c1)CCC2)c1ccc2cccnc2n1] | (Compound 611) |
| 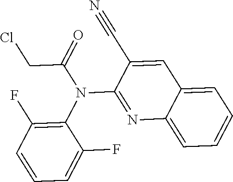 [SMILES: N#Cc1cc2ccccc2nc1N(C(=O)CCl)c1c(F)cccc1F] | (Compound 612) |
| 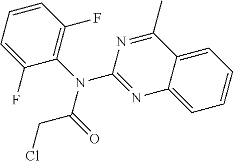 [SMILES: Cc1nc(N(C(=O)CCl)c2c(F)cccc2F)nc2ccccc12] | (Compound 613) |
| 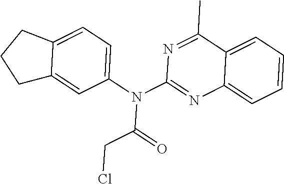 [SMILES: Cc1nc(N(C(=O)CCl)c2ccc3c(c2)CCC3)nc2ccccc12] | (Compound 614) |
| 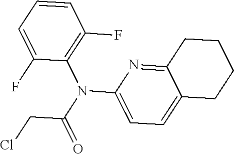 [SMILES: O=C(CCl)N(c1ccc2c(n1)CCCC2)c1c(F)cccc1F] | (Compound 615) |
| 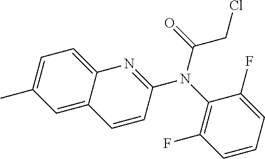 [SMILES: Cc1ccc2nc(N(C(=O)CCl)c3c(F)cccc3F)ccc2c1] | (Compound 616) |
| 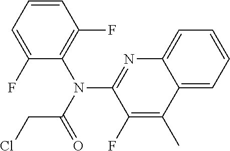 [SMILES: Cc1c(F)c(N(C(=O)CCl)c2c(F)cccc2F)nc2ccccc12] | (Compound 617) |
| 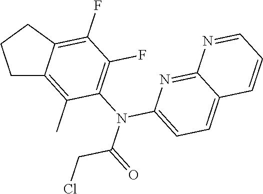 [SMILES: Cc1c2c(c(F)c(F)c1N(C(=O)CCl)c1ccc3cccnc3n1)CCC2] | (Compound 618) |
| 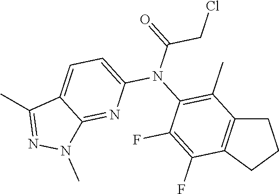 [SMILES: Cc1c2c(c(F)c(F)c1N(C(=O)CCl)c1ccc3c(C)nn(C)c3n1)CCC2] | (Compound 619) |
| 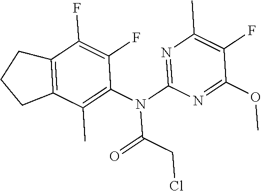 [SMILES: COc1nc(N(C(=O)CCl)c2c(C)c3c(c(F)c2F)CCC3)nc(C)c1F] | (Compound 620) |
| 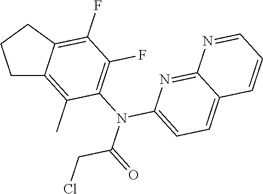 [SMILES: Cc1c2c(c(F)c(F)c1N(C(=O)CCl)c1ccc3cccnc3n1)CCC2] | (Compound 621) |
| 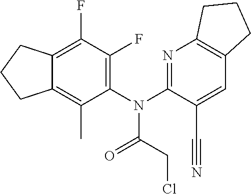 [SMILES: Cc1c2c(c(F)c(F)c1N(C(=O)CCl)c1nc3c(cc1C#N)CCC3)CCC2] | (Compound 622) |
| 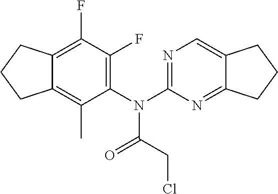 [SMILES: Cc1c2c(c(F)c(F)c1N(C(=O)CCl)c1ncc3c(n1)CCC3)CCC2] | (Compound 623) |
| 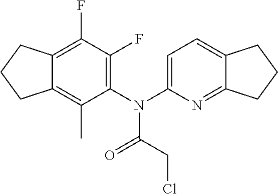 [SMILES: Cc1c2c(c(F)c(F)c1N(C(=O)CCl)c1ccc3c(n1)CCC3)CCC2] | (Compound 624) |
| 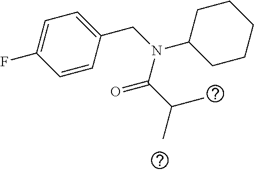 [SMILES: CC(C)C(=O)N(Cc1ccc(F)cc1)C1CCCCC1] | (Compound 625) |
| 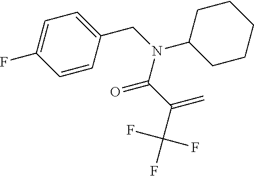 [SMILES: C=C(C(=O)N(Cc1ccc(F)cc1)C1CCCCC1)C(F)(F)F] | (Compound 626) |
| 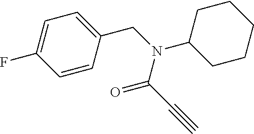 [SMILES: C#CC(=O)N(Cc1ccc(F)cc1)C1CCCCC1] | (Compound 627) |
| 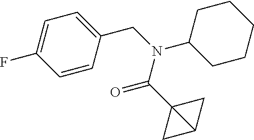 [SMILES: O=C(N(Cc1ccc(F)cc1)C1CCCCC1)C12CC1C2] | (Compound 628) |
| 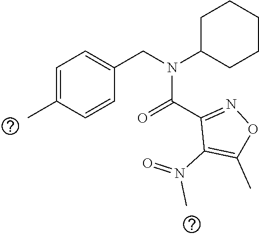 | (Compound 629) |
| 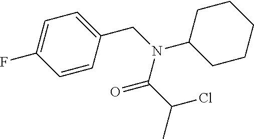 [SMILES: CC(Cl)C(=O)N(Cc1ccc(F)cc1)C1CCCCC1] | (Compound 630) |
| 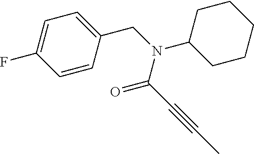 [SMILES: CC#CC(=O)N(Cc1ccc(F)cc1)C1CCCCC1] | (Compound 631) |
| 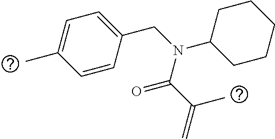 [SMILES: C=C(C)C(=O)N(Cc1ccc(C)cc1)C1CCCCC1] | (Compound 632) |
| 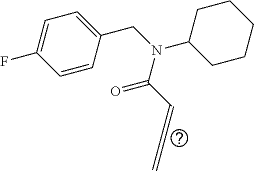 [SMILES: C=C=CC(=O)N(Cc1ccc(F)cc1)C1CCCCC1] | (Compound 633) |
| 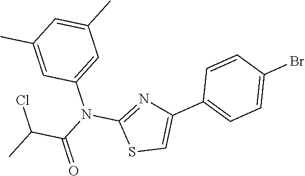 [SMILES: Cc1cc(C)cc(N(C(=O)C(C)Cl)c2nc(-c3ccc(Br)cc3)cs2)c1] | (Compound 634) |
| 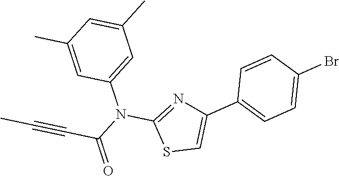 [SMILES: CC#CC(=O)N(c1cc(C)cc(C)c1)c1nc(-c2ccc(Br)cc2)cs1] | (Compound 635) |
| 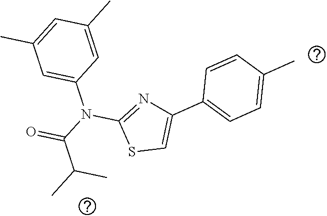 | (Compound 636) |
| 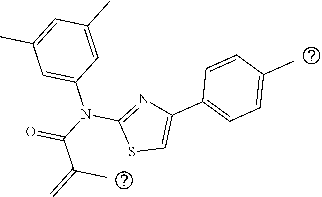 | (Compound 637) |
| 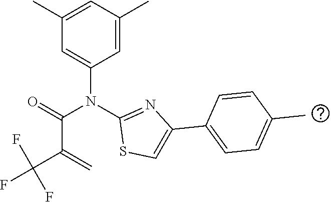 [SMILES: C=C(C(=O)N(c1cc(C)cc(C)c1)c1nc(-c2ccc(C)cc2)cs1)C(F)(F)F] | (Compound 638) |
| 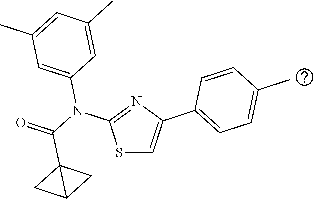 | (Compound 639) |
| 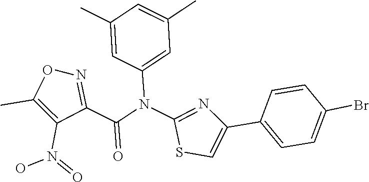 | (Compound 640) |
| 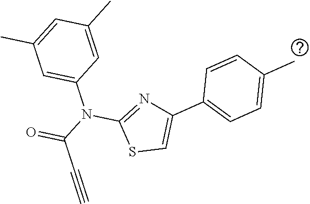 | (Compound 641) |
| 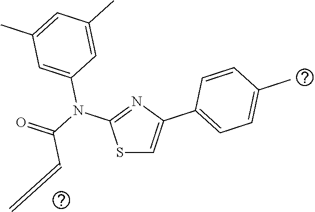 | (Compound 642) |

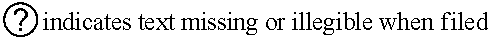

TABLE 4
Chemical structures of five ferroptosis-inducing compounds from
Enamine covalent inhibitors

| | |
|---|---|
| 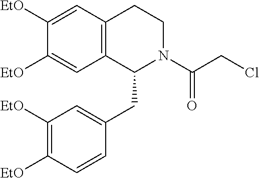 [SMILES: CCOc1ccc(C[C@@H]2c3cc(OCC)c(OCC)cc3CCN2C(=O)CCl)cc1OCC] | Z56943362 |
| 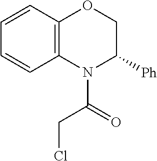 [SMILES: O=C(CCl)N1c2ccccc2OC[C@@H]1c1ccccc1] | Z85921962 |
| 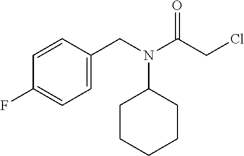 [SMILES: O=C(CCl)N(Cc1ccc(F)cc1)C1CCCCC1] | Z104378216 |
| 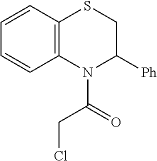 [SMILES: O=C(CCl)N1c2ccccc2SCC1c1ccccc1] | Z90660973 |
| 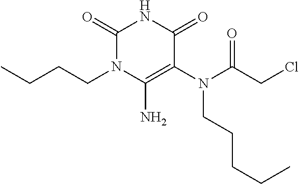 [SMILES: CCCCCN(C(=O)CCl)c1c(N)n(CCCC)c(=O)[nH]c1=O] | Z56931816 |

TABLE 5
Compound 8216 derivatives

| Compound | Average | Stdev |
|---|---|---|
| Z1562139496 | 0.000174 | 5.9399E−05 |
| Z199424090 | 0.00039001 | 0.00011031 |
| Z56946047 | 0.00062402 | 6.7884E−05 |
| Z147647938 | 0.00093603 | 0.00022062 |
| Z4425210974 | 0.00128404 | 0.00015274 |
| Z3837055210 | 0.00158404 | 0.00088249 |
| Z4323362848 | 0.00162605 | 0.00019517 |
| Z3629871044 | 0.00176405 | 0.00186681 |
| Z381549666 | 0.00183605 | 0.00030548 |
| Z56891378 | 0.00187205 | 0.00074673 |
| Z4425210932 | 0.00192605 | 8.4855E−06 |
| Z4425211258 | 0.00193805 | 0.00044973 |
| Z56837054 | 0.00199206 | 0.00098432 |
| Z4323349920 | 0.00202806 | 0.00071278 |
| Z3837267295 | 0.00211206 | 3.3942E−05 |
| Z3766260223 | 0.00223806 | 0.0008231 |
| Z57052885 | 0.00224406 | 0.00015274 |
| Z4425210755 | 0.00256207 | 0.00092492 |
| Z1562136806 | 0.00264007 | 0.00074673 |
| Z3766269910 | 0.00290408 | 0.00173105 |
| Z4323320274 | 0.00292208 | 0.00021214 |
| Z3766270794 | 0.0034741 | 0.00109463 |
| Z4323385212 | 0.00376811 | 0.00049216 |
| Z4425210756 | 0.00393011 | 0.0008231 |
| Z4323486432 | 0.00442812 | 0.00210441 |
| Z56837076 | 0.00469813 | 0.002639 |
| Z4323314193 | 0.00483014 | 0.00104372 |
| Z4425210609 | 0.00525615 | 0.00110312 |
| Z108565162 | 0.00539415 | 0.00067036 |
| Z3766271747 | 0.00561616 | 0.00218926 |
| Z147647668 | 0.00583816 | 0.00206198 |
| Z147647684 | 0.00657618 | 0.00113706 |
| Z4425210448 | 0.00756021 | 0.00032245 |
| Z3837152585 | 0.00768622 | 0.00092492 |
| Z3837300268 | 0.00828023 | 0.00280022 |
| Z56926666 | 0.00862224 | 0.00039882 |
| RSL3 | 0.00933026 | 0.00107766 |
| Z3837046637 | 0.00972027 | 0.00123889 |
| Z89264994 | 0.01228234 | 0.00423427 |
| Z3837197646 | 0.01316437 | 0.00268142 |
| Z4323386801 | 0.01527043 | 0.00060247 |
| Z4323373087 | 0.01670447 | 0.00079764 |
| Z4425210708 | 0.0178325 | 0.00302084 |
| Z4425210960 | 0.01924854 | 0.00156134 |
| Z3766264621 | 0.0215166 | 0.00610957 |
| Z3952175509 | 0.03046885 | 0.00453127 |
| Z147647920 | 0.04392123 | 5.0913E−05 |
| Z4323422705 | 0.04398123 | 0.00352998 |
| Z4323310732 | 0.05331749 | 0.00432761 |
| Z90122115 | 0.05781762 | 0.00098432 |
| Z4425211012 | 0.07480409 | 0.03001328 |
| Z4170009630 | 0.0891565 | 0.0032839 |
| Z147647648 | 0.10065882 | 0.02391219 |
| Z3837255186 | 0.13307773 | 0.01967792 |
| Z1687602192 | 0.14450805 | 0.06494816 |
| Z4425210882 | 0.1465421 | 0.00657628 |
| Z3952174992 | 0.15167825 | 0.0843206 |
| Z3837096793 | 0.17009876 | 0.01541819 |
| Z3837506617 | 0.32674515 | 0.04773953 |
| Z2596884436 | 0.40261127 | 0.01491754 |
| Z4323324870 | 0.48795766 | 0.02530382 |
| Z3837050450 | 0.51481441 | 0.0032245 |
| Z3837366035 | 0.53099687 | 0.07527504 |
| Z3766262414 | 0.54812735 | 0.03192252 |
| Z4323355648 | 0.56906193 | 0.04350526 |
| Z4323341765 | 0.70080162 | 0.10054491 |
| Z3837644318 | 0.74069074 | 0.0241413 |
| Z147652330 | 0.74589289 | 0.06537244 |
| Z1562122996 | 0.75753921 | 0.11933185 |
| Z3887631385 | 0.79383423 | 0.11932337 |
| Z3952175125 | 0.88756285 | 0.05898284 |
| Z4283095766 | 0.93423216 | 0.09513115 |
| Z3337549802 | 1.03385295 | 0.08728205 |
| Z2020497058 | 1.0891505 | 0.04266519 |
| Z3217399415 | 1.25468913 | 0.0540273 |

TABLE 6
Autogrow4 independently discovers nine 8216 derivatives
as potential GPX4 binders.

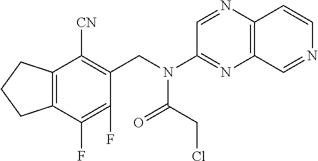 [SMILES: N#Cc1c(CN(C(=O)CCl)c2cnc3ccncc3n2)c(F)c(F)c2c1CCC2]

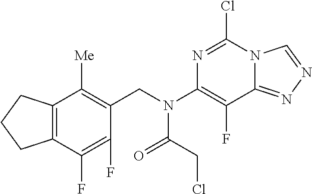 [SMILES: Cc1c(CN(C(=O)CCl)c2nc(Cl)n3cnnc3c2F)c(F)c(F)c2c1CCC2]

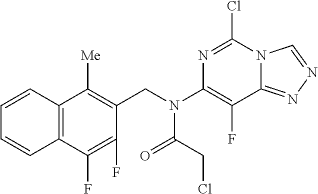 [SMILES: Cc1c(CN(C(=O)CCl)c2nc(Cl)n3cnnc3c2F)c(F)c(F)c2ccccc12]

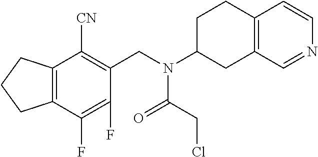 [SMILES: N#Cc1c(CN(C(=O)CCl)C2CCc3ccncc3C2)c(F)c(F)c2c1CCC2]

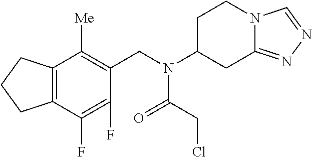 [SMILES: Cc1c(CN(C(=O)CCl)C2CCn3cnnc3C2)c(F)c(F)c2c1CCC2]

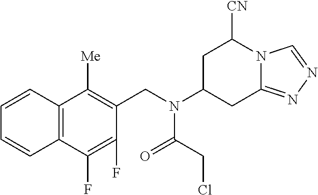 [SMILES: Cc1c(CN(C(=O)CCl)C2Cc3nncn3C(C#N)C2)c(F)c(F)c2ccccc12]

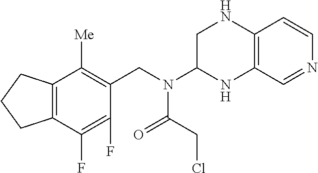 [SMILES: Cc1c(CN(C(=O)CCl)C2CNc3ccncc3N2)c(F)c(F)c2c1CCC2]

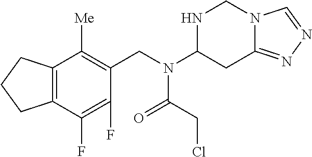 [SMILES: Cc1c(CN(C(=O)CCl)C2Cc3nncn3CN2)c(F)c(F)c2c1CCC2]

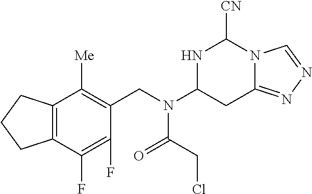 [SMILES: Cc1c(CN(C(=O)CCl)C2Cc3nncn3C(C#N)N2)c(F)c(F)c2c1CCC2]

Biological evaluation of working examples A1-A47 and B1-B28 was performed in HT1080 cells (ATCC, cat #CCL-121) according to the procedure provided below.

### 1. Cell Seeding

1.1 Cells were harvested from flask into cell culture medium and then the cell number count was determined.
1.2 The cells were suspended with 1.5 μM ferrostatin-1 and without ferrostatin-1.
1.3 Cells were plated at a density of 1,500 cells per well in a 384-well black sided clear bottom plate, in 25 μL, using electronic multichannel pipette.
1.4 Plates were covered with lid and spun for 1 min at 1000 rpm, then allowed to incubate overnight at 37° C. under 5% CO2.

### 2. Compound Treatment

2.1 Compounds were dissolved at 10 mM stock solution and 10 μl of diluted solution was transferred to a 384 LDV-plate (LABCYTE, LP-0200). A 3 fold, 11-point dilution was performed.
2.2 Cells were treated with compounds by plate reformat Echo software and spun for 1 min at 1000 rpm. Then incubated at 37° C. for 24 hrs in a CO2 incubator.

### 3. Readout

3.1 The plates were removed from the incubator and allowed to equilibrate to room temperature, at least 30 minutes.
3.2 CellTiter-Glo® 2.0 Reagent (Promega, G9242) was added (25 μL) to the assay plates and spun for 1 min at 1000 rpm. Then allowed to stand for about 20 minutes before reading luminescence signal on Envision.
3.3 The IC50 calculations were determined with a nonlinear fit, variable slope (four parameters) log(inhibitor) vs. response model using GraphPad PRISM. Results for compounds of the invention are provided in Tables 7 and 8.

TABLE 7

| Working | | IC₅₀ |
|---|---|---|
| Example | Name | (nM) |
| A1 | 2-chloro-N-(3,5-dimethylphenyl)-N-[(4- | C |
| | fluorophenyl)methyl]acetamide | |
| A2 | 2-chloro-N-(3,5-dimethylphenyl)-N-[(4- | B |
| | methoxyphenyl)methyl]acetamide | |
| A3 | 2-chloro-N-(3,5-dimethoxyphenyl)-N-[(4- | B |
| | fluorophenyl)methyl]acetamide | |
| A4 | 2-chloro-N-[(4-chlorophenyl)methyl]-N-(3,5- | C |
| | dimethylphenyl)acetamide | |
| A5 | 2-chloro-N-(3,5-dimethylphenyl)-N-[(3- | B |
| | methoxyphenyl)methyl]acetamide | |
| A6 | 2-chloro-N-[(2-chlorophenyl)methyl]-N-(3,5- | C |
| | dimethylphenyl)acetamide | |
| A7 | 2-chloro-N-(3,5-dimethylphenyl)-N-[(2- | B |
| | methoxyphenyl)methyl]acetamide | |
| A8 | 2-chloro-N-(3,5-dimethylphenyl)-N-(m- | B |
| | tolylmethyl)acetamide | |
| A9 | 2-chloro-N-[(3-chlorophenyl)methyl]-N-(3,5- | B |
| | dimethylphenyl)acetamide | |
| A10 | 2-chloro-N-(3,5-dimethylphenyl)-N-(p- | C |
| | tolylmethyl)acetamide | |
| A11 | 2-chloro-N-(3,5-dimethylphenyl)-N-(o- | C |
| | tolylmethyl)acetamide | |
| A12 | 2-chloro-N-cyclohexyl-N-[(4- | C |
| | fluorophenyl)methyl]acetamide | |
| A13 | 2-chloro-N-[(4-fluorophenyl)methyl]-N-(3- | C |
| | methoxyphenyl)acetamide | |
| A14 | 2-chloro-N-(3-chloro-5-methoxy-phenyl)-N-[(4- | C |
| | fluorophenyl)methyl]acetamide | |
| A15 | 2-chloro-N-(3,5-difluorophenyl)-N-[(4- | D |
| | fluorophenyl)methyl]acetamide | |
| A16 | 2-chloro-N-(3,5-dichlorophenyl)-N-[(4- | C |
| | fluorophenyl)methyl]acetamide | |
| A17 | 2-chloro-N-(3-chloro-4-methoxy-phenyl)-N-[(4- | C |
| | fluorophenyl)methyl]acetamide | |
| A18 | 2-chloro-N-(3-chlorophenyl)-N-[(4- | C |
| | fluorophenyl)methyl]acetamide | |
| A19 | 2-chloro-N-[(4-cyanophenyl)methyl]-N-(3,5- | B |
| | dimethoxyphenyl)acetamide | |
| A20 | 2-chloro-N-[(2,3-difluorophenyl)methyl]-N-(3,5- | B |
| | dimethoxyphenyl)acetamide | |
| A21 | 2-chloro-N-(3,5-dimethoxyphenyl)-N-[(4- | B |
| | methoxyphenyl)methyl]acetamide | |
| A22 | 2-chloro-N-[(4-fluorophenyl) methyl]-N-(6- | C |
| | quinolyl)acetamide | |
| A23 | N-[(2-bromo-4-fluoro-phenyl)methyl]-2-chloro- | B |
| | N-(3,5-dimethoxyphenyl)acetamide | |
| A24 | 2-chloro-N-(3,5-dimethoxyphenyl)-N-[(4- | C |
| | hydroxyphenyl)methyl]acetamide | |
| A25 | 2-chloro-N-[(4-fluorophenyl)methyl]-N-(3- | C |
| | methoxy-5-methyl-phenyl)acetamide | |
| A26 | 2-chloro-N-[(2,6-dichloro-4-fluoro-phenyl)methyl]- | B |
| | N-(3,5-dimethoxyphenyl)acetamide | |
| A27 | 2-chloro-N-(3,5-dimethoxyphenyl)-N-[[4-fluoro-2- | B |
| | (trifluoromethyl)phenyl]methyl]acetamide | |
| A28 | 2-chloro-N-[(5-cyano-2-fluoro-phenyl)methyl]-N- | B |
| | (3,5-dimethoxyphenyl)acetamide | |
| A29 | 2-chloro-N-[(4-fluorophenyl)methyl]-N-(3-methoxy- | B |
| | 4-methyl-phenyl)acetamide | |
| A30 | 2-chloro-N-(3,5-dimethoxyphenyl)-N-[(4-fluoro- | A |
| | 2,6-dimethyl-phenyl)methyl]acetamide | |
| A31 | 2-chloro-N-(3,5-dimethoxyphenyl)-N-[[2-fluoro- | B |
| | 3-(trifluoromethyl)phenyl]methyl]acetamide | |
| A32 | 2-chloro-N-(2-fluoro-6-methoxy-phenyl)-N-[(4- | D |
| | fluorophenyl)methyl]acetamide | |
| A33 | 2-chloro-N-(3,5-dimethoxyphenyl)-N-[(4-fluoro- | A |
| | 3-methoxy-phenyl)methyl]acetamide | |
| A34 | 2-chloro-N-(3-chloro-5-hydroxy-phenyl)-N-[(4- | C |
| | fluorophenyl)methyl]acetamide | |
| A35 | 2-chloro-N-(2-chloro-3-methoxy-phenyl)-N-[(4- | C |
| | fluorophenyl)methyl]acetamide | |
| A36 | 2-chloro-N-(2-cyano-4-methoxy-phenyl)-N-[(4- | D |
| | fluorophenyl)methyl]acetamide | |
| A37 | 2-chloro-N-(2-chloro-5-methoxy-phenyl)-N-[(4- | C |
| | fluorophenyl)methyl]acetamide | |
| A38 | N-[(4-bromo-2,3-difluoro-phenyl)methyl]-2- | A |
| | chloro-N-(3,5-dimethoxyphenyl)acetamide | |
| A39 | N-(3-bromo-1H-pyrazolo[3,4-b]pyridin-5-yl)- | D |
| | 2-chloro-N-[(4-fluorophenyl)methyl]acetamide | |
| A40 | 2-chloro-N-[(4-fluorophenyl)methyl]-N- | B |
| | (4-methoxy-3,5-dimethyl-phenyl)acetamide | |
| A41 | 2-chloro-N-[(4-fluorophenyl)methyl]-N- | E |
| | (4-hydroxy-3,5-dimethyl-phenyl)acetamide | |
| A42 | 2-chloro-N-(2,4-dichloro-3-methyl-phenyl)- | C |
| | N-[(4-fluorophenyl)methyl]acetamide | |
| A43 | 2-chloro-N-[(2-chloro-3,4-difluoro-phenyl)methyl]- | A |
| | N-(3,5-dimethoxyphenyl)acetamide | |
| A44 | 2-chloro-N-(3,5-dimethoxyphenyl)-N-[(3- | B |
| | fluoro-5-methyl-phenyl)methyl]acetamide | |
| A45 | N-(2,1,3-benzothiadiazol-4-ylmethyl)-2- | B |
| | chloro-N-(3,5-dimethoxyphenyl)acetamide | |
| A46 | 2-chloro-N-[(4-fluorophenyl)methyl]-N- | C |
| | (2-methoxy-4-pyridyl)acetamide | |
| A47 | 2-chloro-N-[(4-fluorophenyl)methyl]-N- | E |
| | (6-methyl-3-pyridyl)acetamide | |

Note:
Assay IC50 data are designated within the following ranges:
A: ≤10 nM
B: >10 nM to ≤100 nM
C: >100 nM to ≤1,000 Nm
D: >1 μM to ≤10 μM
E: >10 μM

TABLE 8

| Working | | IC₅₀ |
|---|---|---|
| Example | Name | (nM) |
| B1 | N-[4-(4-bromophenyl)thiazol-2-yl]-2-chloro-N-(3- | — |
| | methoxypropyl)acetamide | |
| B2 | N-[4-(4-bromophenyl)thiazol-2-yl]-N-(3- | D |
| | methoxypropyl)prop-2-ynamide | |
| B3 | 2-chloro-N-[4-(4-methoxyphenyl)thiazol-2-yl]- | D |
| | N-(3-methoxypropyl)acetamide | |
| B4 | N-[4-(4-methoxyphenyl)thiazol-2-yl]-N-(3- | — |
| | methoxypropyl)prop-2-ynamide | |
| B5 | N-[4-(4-bromophenyl)thiazol-2-yl]-2-chloro- | C |
| | N-(3,5-dimethylphenyl)acetamide | |
| B6 | N-[4-(4-bromophenyl)thiazol-2-yl]-2-chloro- | — |
| | N-cyclohexyl-acetamide | |
| B7 | N-[4-(4-bromophenyl)thiazol-2-yl]-N-(3,5- | E |
| | dimethylphenyl)prop-2-enamide | |
| B8 | N-[4-(4-bromophenyl)thiazol-2-yl]-N-(3,5- | E |
| | dimethylphenyl)but-2-ynamide | |
| B9 | 2-chloro-N-(3,5-dimethylphenyl)-N-[4-(4- | C |
| | methoxyphenyl)thiazol-2-yl]acetamide | |
| B10 | 2-chloro-N-(4-cyclohexylthiazol-2-yl)-N-(3,5- | C |
| | dimethylphenyl)acetamide | |
| B11 | 2-chloro-N-(3,5-dimethylphenyl)-N-(4- | C |
| | phenylthiazol-2-yl)acetamide | |
| B12 | N-[4-(3-bromophenyl)thiazol-2-yl]-2-chloro- | C |
| | N-(3,5-dimethylphenyl)acetamide | |
| B13 | N-[4-(4-bromophenyl)thiazol-2-yl]-N-(3,5- | B |
| | dimethylphenyl)prop-2-ynamide | |
| B14 | N-benzyl-N-[4-(4-bromophenyl)thiazol-2-yl]- | D |
| | 2-chloro-acetamide | |
| B15 | N-[4-(4-bromophenyl)thiazol-2-yl]-2-chloro- | E |
| | N-phenyl-acetamide | |
| B16 | N-[4-(4-bromophenyl)thiazol-2-yl]-2-chloro- | E |
| | N-(m-tolyl)acetamide | |
| B17 | N-[4-(4-bromophenyl)thiazol-2-yl]-2-chloro- | C |
| | N-(3-methoxyphenyl)acetamide | |
| B18 | N-[4-(4-bromophenyl)thiazol-2-yl]-2-chloro- | — |
| | N-(3,5-dimethoxyphenyl)acetamide | |
| B19 | N-[4-(4-bromophenyl)thiazol-2-yl]- | B |
| | N-(3,5-dimethoxyphenyl)prop-2-ynamide | |
| B20 | N-[4-(4-bromophenyl)thiazol-2-yl]-2-chloro- | D |
| | N-(3,5-dichlorophenyl)acetamide | |
| B21 | N-[4-(4-bromophenyl)thiazol-2-yl]-N-(3,5- | B |
| | dimethylphenyl)-2-iodo-acetamide | |
| B22 | N-[4-(4-bromophenyl)thiazol-2-yl]-N-(3,5- | B |
| | dichlorophenyl)prop-2-ynamide | |
| B23 | N-[4-(4-bromophenyl)thiazol-2-yl]-N-(3,5- | E |
| | dimethylphenyl)-2-fluoro-prop-2-enamide | |
| B24 | N-[4-(4-bromophenyl)thiazol-2-yl]-2-chloro- | C |
| | N-(3-chloro-5-methoxy-phenyl)acetamide | |
| B25 | N-[4-(4-bromophenyl)thiazol-2-yl]-2-chloro- | E |
| | N-(3,5-difluorophenyl)acetamide | |
| B26 | N-[4-(4-bromophenyl)thiazol-2-yl]-N-(3-chloro- | B |
| | 5-methoxy-phenyl)prop-2-ynamide | |
| B27 | N-[4-(4-bromophenyl)thiazol-2-yl]-N-(3,5- | E |
| | dimethylphenyl)-2-fluoro-acetamide | |
| B28 | N-[4-(4-bromophenyl)thiazol-2-yl]-N-(3,5- | E |
| | dimethylphenyl)oxirane-2-carboxamide | |

Note:
Assay IC50 data are designated within the following ranges:
A: ≤10 nM
B: >10 nM to ≤100 nM
C: >100 nM to ≤1,000 Nm
D: >1 μM to ≤10 μM
E: >10 μM

1-42. (canceled)

43. A method of treating cancer in a subject, comprising administering to the subject a pharmaceutically effective amount of a compound having any of the structures of Formulas 1-5 or 91, or a pharmaceutically acceptable salt thereof. 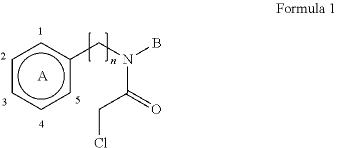
where n is 0 or 1; and
where B is selected from: 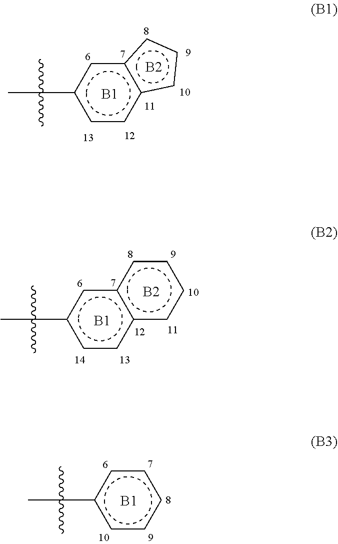
where each ring individually may be aliphatic or aromatic;
where each ring individually may substituted or unsubstituted;
where when n is 0 and B is B₁:
there are no ring substituents or ring heteroatoms; or
there are ring heteroatoms comprising [A:1,5 and/or (B1:6 or B1:13 or B1:6,13) and/or (B2:8,9 or B2:10 or B2:11,12) is a N]; and/or B2:8 is a S; and/or (B2:8 or B2:10 or B2:13 is an O); and/or
if substituted, substituents on rings A and/or B₁ and/or B₂ are as follows:
one or more F, Br, Cl, CN, Me, OMe at any position on the ring;
where when n is 0 and B is B₂:
there are no ring substituents or ring heteroatoms; or
there are ring heteroatoms comprising (B1:6 or B1:6,13 or B1:6,14) and/or B2:8 is an N; and/or
if substituted, substituents on rings A and/or B₁ and/or B₂ are as follows:
one or more F, Br, Cl, CN, Me at any position on the ring;
where when n is 1 and B is B₁:
there are no ring substituents or ring heteroatoms; or
there are ring heteroatoms comprising [(B1:6 or B1:13 or B1:6,13) and/or (B2:7,9 or B2:7,10 or B2:7,9,10 or B2:7,8,10 or B2:7,8,9,10 or B2:8 or B2:8,9 or B2:8,9,10 or B2:8, 11 or B2:8,10, 11 or B2:8,9, 10,11 or B2:9,11 or B2:10 or B2:10,11) is an N]; and/or (B2:8 or B2:10 or B2:13 is an O); and/or B2:8 is an S; and/or
if substituted, substituents on rings A and/or B₁ and/or B₂ are as follows:
one or more F, Br, Cl, CN, Me at any position on the ring;
where when n is 1 and B is B₂:
there are no ring substituents or ring heteroatoms; or
there are ring heteroatoms comprising [(B1:6 or B1:13 or B1:6,13 or B1:6,14) and/or (B2:8 or B2:9,11 or B2:11) is an N]; and/or
if substituted, substituents on rings A and/or B₁ and/or B₂ are as follows:
one or more F, Br, Cl, CN, Me at any position on the ring;
where when n is 1 and B is B₃:
there are no ring substituents or ring heteroatoms; or
there are ring heteroatoms comprising (B1:6; or B1:6,8 or B1:6,10 or B1:7 or B1:8 is a N); and/or B1:8 is an O; and/or
if substituted, substituents on rings A and/or B1 include:
one or more F, Br, Cl, CN, Me, OMe, trihalomethane, OH at any position on the ring. 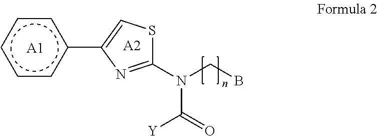
where n is 0 or 1; and
where B is selected from: 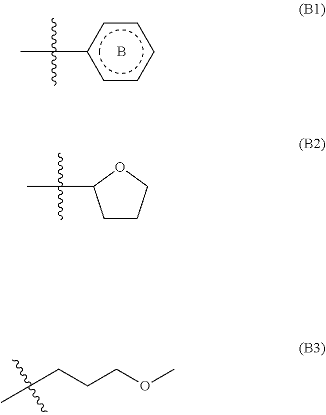
and where Y is selected from: 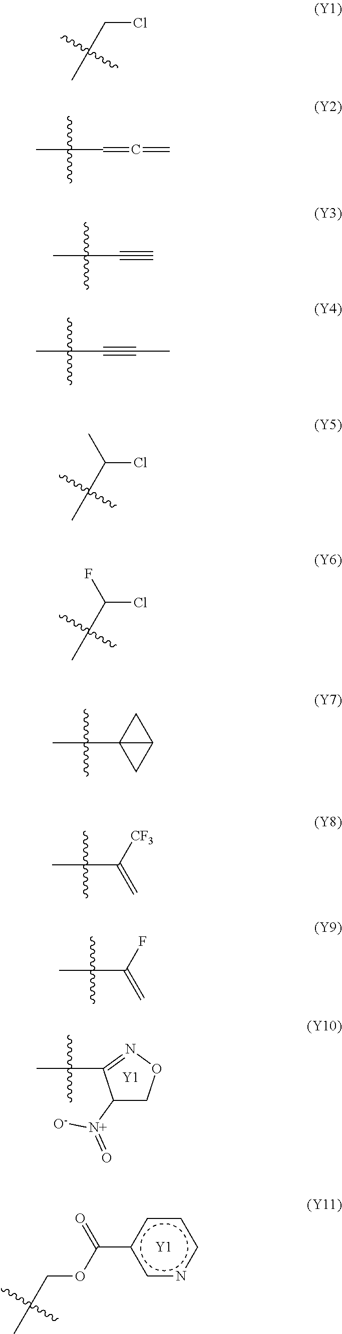
where each ring individually may be aliphatic or aromatic;
where each ring each ring individually may substituted or unsubstituted;
where when n is 0 or 1; and
where when B is B₁ or B₂; and
where when Y is any one of Y₁, Y₂, Y₃, Y₄, Y₅, Y₆, Y₇, Y₈, Y₉, Y₁₀, Y₁₁:
there are no additional ring substituents or ring heteroatoms; or
if substituted, substituents on A and/or B rings and/or ring Y1 are as follows:
one or more F, Br, Cl, CN, OMe, Me at any position on the ring;
where when n is 0 and Y is Y₁:
there are no additional ring substituents or ring heteroatoms; or
B is a 2-propenyl substituent; or B is a hexyl substituent; or B is a hydrogen; and/or
if substituted, substituents on ring A₁ are as follows:
one or more F, Br, Cl, CN, Me, OMe at any position on the ring;
where when n is 0, B is B₁ and Y is Y₁:
there are no additional ring substituents or ring heteroatoms; or
there are one or more isopropyl substituents on any position on the B ring; and/or
if substituted, substituents on rings A₁ and/or B₁ are as follows:
one or more F, Br, Cl, CN, Me, OMe at any position on the ring;
where when n is 0, B is B₃ and Y is Y₁:
there are no additional ring substituents or ring heteroatoms; or
if substituted, substituents on ring A₁ are as follows:
one or more F, Br, Cl, CN, Me, OMe at any position on the ring. 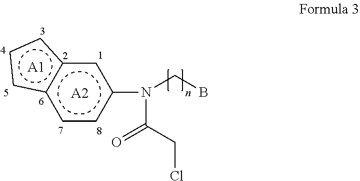
where n is 0 or 1; and
where B is selected from: 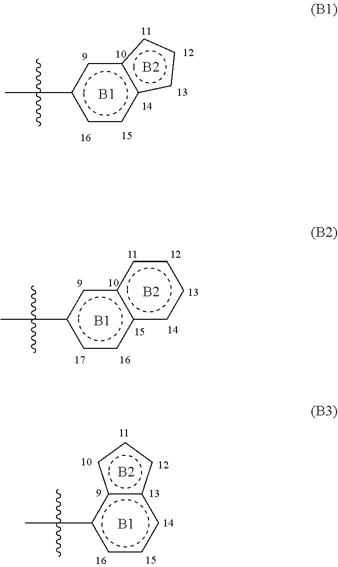
where each ring individually may be aliphatic or aromatic;
where each ring each ring individually may substituted or unsubstituted;
where when n is 0 and B is B₁:
there are no ring substituents or ring heteroatoms; or
there are ring heteroatoms comprising [(B1:9 or B1:16 or B1:9,16) and/or (B2:11,12 or B2:13) is an N]; and/or (B2:11 or B2:13 is an O); and/or B2:11 is an S;
if substituted, substituents on rings A1 and/or A2 and/or B1 and/or B2 are as follows:
one or more F, Br, Cl, CN, Me at any position on the ring;
where when n is 1 and B is B₁:
there are no ring substituents or ring heteroatoms; or
there are ring heteroatoms comprising [(A1:2,4 or A1:2,5 or A1:2,4,5 or A1:2,3,5 or A1:2,3,4,5 or A1:3,4 or A1:3,4,5 or A1:3,6 or A1:3,4,6 or A1:3,5,6 or A1:3,4,5,6 or A1:4,5 or A1:4,6 or A1:5 or A1:5,6) and/or (A2:1 or A2:1,8 or A2:8) is an N]; and/or A1:3 is an S; and/or (A1:3 or A1:5 is an O); and/or
if substituted, substituents on rings A₁ and/or A₂ and/or B₁ and/or B₂ are as follows:
one or more F, Br, Cl, CN, Me at any position on the ring;
where when n is 0 and B is B₂:
there are no ring substituents or ring heteroatoms; or
there are ring heteroatoms comprising [(B1:9 or B1:17 or B1:9,16 or B1:9,17) and/or B2:11 is an N]; and/or
if substituted, substituents on rings A₁ and/or A₂ and/or B₁ and/or B₂ are as follows:
one or more F, Br, Cl, CN, Me at any position on the ring;
where when n is 1 and B is B₂:
there are no ring substituents or ring heteroatoms; or
there are ring heteroatoms comprising [(A1:3 or A1:4 or A1:5 or A1:3,4 or A1:3,5 or A1:4,5 or A1:3,4,5) and/or (A2:2 or A2:6 or A2:8 or A2:6,8) and/or (B1:9 or B1:17 or B1:9,16 or B1:9,17) and/or (B2:11 or B2:12 or B2:14 or B2:12,14) is an N]; and/or A1:3 is an S; and/or
if substituted, substituents on rings A₁ and/or A₂ and/or B₁ and/or B₂ are as follows:
one or more F, Br, Cl, CN, Me at any position on the ring;
where when n is 1 and B is B₃:
there are no ring substituents or ring heteroatoms; or
there are ring heteroatoms comprising A1:3,4,6 and/or A2:8 is an N; and/or
if substituted, substituents on rings A₁ and/or A₂ and/or B₁ and/or B₂ are as follows:
one or more F, Br, Cl, CN, Me at any position on the ring. 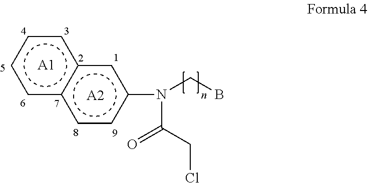
where n is 0 or 1; and
where B is selected from: 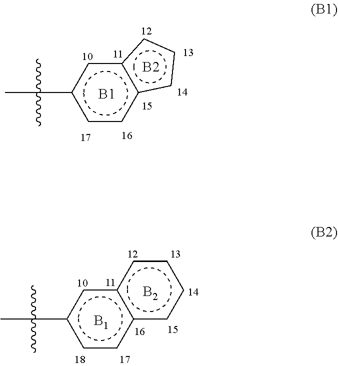
where each ring individually may be aliphatic or aromatic;
where each ring individually may substituted or unsubstituted;
where when n is 1 and B is B₁:
there are no ring substituents or ring heteroatoms; or
there are ring heteroatoms comprising [(A1:3 or A1:4 or A1:6) and/or (A2:1 or A2:1,8 or A2:1,9) is an N]; and/or
if substituted, substituents on rings A₁ and/or A₂ and/or B₁ and/or B₂ are as follows:
one or more F, Br, Cl, CN, Me at any position on the ring;
where when n is 1 and B is B₂:
there are no ring substituents or ring heteroatoms; or
there are ring heteroatoms comprising [(A1:4 or A1:6 or A1:4,6) and/or A2:1,8 and/or (B2:11 or B2:12 or B2:14 or B2:12,14) is an N]; and/or (A1:3 is an S); and/or
if substituted, substituents on rings A1 and/or A2 and/or B1 and/or B2 are as follows:
one or more F, Br, Cl, CN, Me at any position on the ring. 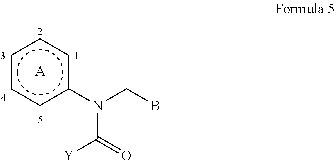
where B is selected from: 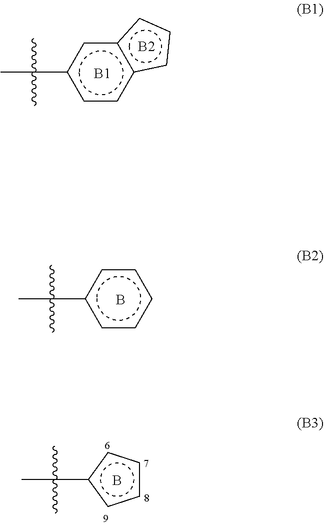
where each ring individually may be aliphatic or aromatic;
where each ring individually may substituted or unsubstituted;
and where Y is selected from: 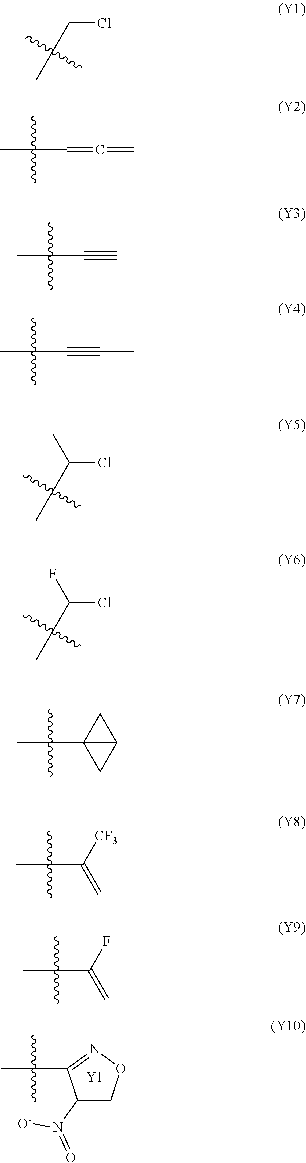
where when B is B₂; and
where when Y is any of Y₁, Y₂, Y₃, Y₄, Y₅, Y₆, Y₇, Y₈, Y₉, Y₁₀:
there are no additional ring substituents or ring heteroatoms; or
if substituted, substituents on rings A and/or B and/or Y1 are as follows:
one or more F, Br, Cl, CN, Me, OMe at any position on the ring;
where when B is B₁; and
where when Y is Y₁:
there are no additional ring substituents or ring heteroatoms; or
there are ring heteroatoms comprising A:1,5 is a N; and/or
if substituted, substituents on rings A and/or B are as follows:
one or more F, Br, Cl, CN, Me, OMe at any position on the ring;
where when B is B₃; and
where when Y is Y₁:
there are no additional ring substituents or ring heteroatoms; or
there are ring heteroatoms comprising (B:6,7; and/or B:9 is a N); and/or B:7 is an O; and/or B:6 is an S; and/or
if substituted, substituents on rings A and/or B are as follows:
one or more F, Br, Cl, CN, Me, OMe at any position on the ring.

44. The method of claim 43, wherein the method reduces the growth rate of a tumor in the subject, reduces the size of a tumor in the subject, eliminates a tumor in the subject, or delays progression of a cancer stage in the subject.

45. The method of claim 43, wherein the cancer comprises a mesenchymal cancer.

46. The method of claim 43, wherein the cancer is a sarcoma.

47. The method of claim 43, wherein the compound inhibits GPX4 enzyme.

48. The method of claim 43, wherein the compound induces ferroptosis in a cell.

49. The method of claim 48, wherein the cell is a cancer cell.

50. The method of claim 43, wherein the compound is selected from Compounds 1-643.

51. The method of claim 43, wherein the compound is represented by a structure in Table 1.

| Working | | | MS Data |
|---|---|---|---|
| Example | Structure | Name | (M + H) |
| A1 | 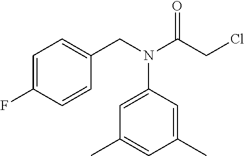 [SMILES: Cc1cc(C)cc(N(Cc2ccc(F)cc2)C(=O)CCl)c1] | 2-chloro-N-(3,5-dimethylphenyl)-N- [(4-fluorophenyl)methyl]acetamide | 306.1 |
| A2 | 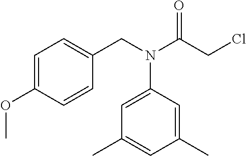 [SMILES: COc1ccc(CN(C(=O)CCl)c2cc(C)cc(C)c2)cc1] | 2-chloro-N-(3,5-dimethylphenyl)-N- [(4-methoxyphenyl)methyl]acetamide | 318.1 |
| A3 | 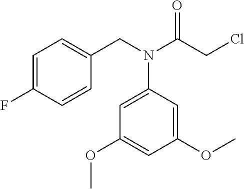 [SMILES: COc1cc(OC)cc(N(Cc2ccc(F)cc2)C(=O)CCl)c1] | 2-chloro-N-(3,5-dimethoxyphenyl)-N- [(4-fluorophenyl)methyl]acetamide | 338.0 |
| A4 | 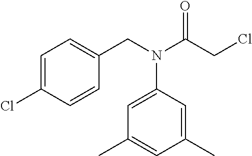 [SMILES: Cc1cc(C)cc(N(Cc2ccc(Cl)cc2)C(=O)CCl)c1] | 2-chloro-N-[(4-chlorophenyl)methyl]- N-(3,5-dimethylphenyl)acetamide | 332.0 |
| A5 | 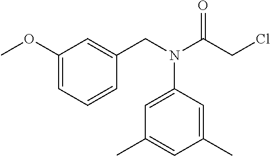 [SMILES: COc1cccc(CN(C(=O)CCl)c2cc(C)cc(C)c2)c1] | 2-chloro-N-(3,5-dimethylphenyl)-N- [(3-methoxyphenyl)methyl]acetamide | 318.0 |
| A6 | 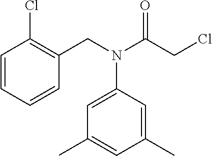 [SMILES: Cc1cc(C)cc(N(Cc2ccccc2Cl)C(=O)CCl)c1] | 2-chloro-N-[(2-chlorophenyl)methyl]- N-(3,5-dimethylphenyl)acetamide | 322.0 |
| A7 | 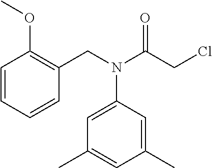 [SMILES: COc1ccccc1CN(C(=O)CCl)c1cc(C)cc(C)c1] | 2-chloro-N-(3,5-dimethylphenyl)-N- [(2-methoxyphenyl)methyl]acetamide | 318.1 |
| A8 | 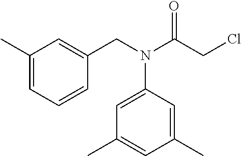 [SMILES: Cc1cccc(CN(C(=O)CCl)c2cc(C)cc(C)c2)c1] | 2-chloro-N-(3,5-dimethylphenyl)-N- (m-tolylmethyl)acetamide | 302.1 |
| A9 | 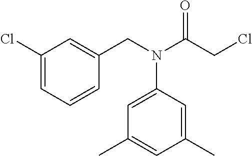 [SMILES: Cc1cc(C)cc(N(Cc2cccc(Cl)c2)C(=O)CCl)c1] | 2-chloro-N-[(3-chlorophenyl)methyl]- N-(3,5-dimethylphenyl)acetamide | 332.0 |
| A10 | 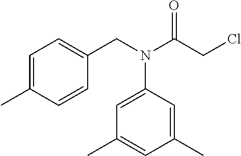 [SMILES: Cc1ccc(CN(C(=O)CCl)c2cc(C)cc(C)c2)cc1] | 2-chloro-N-(3,5-dimethylphenyl)-N- (p-tolylmethyl)acetamide | 302.1 |
| A11 | 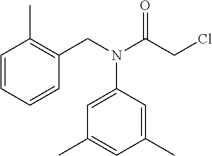 [SMILES: Cc1cc(C)cc(N(Cc2ccccc2C)C(=O)CCl)c1] | 2-chloro-N-(3,5-dimethylphenyl)-N- (o-tolylmethyl)acetamide | 302.1 |
| A12 | 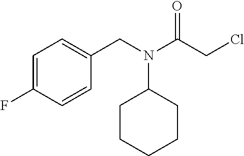 [SMILES: O=C(CCl)N(Cc1ccc(F)cc1)C1CCCCC1] | 2-chloro-N-cyclohexyl-N-[(4- fluorophenyl)methyl]acetamide | 284.1 |
| A13 | 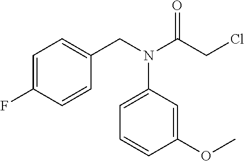 [SMILES: COc1cccc(N(Cc2ccc(F)cc2)C(=O)CCl)c1] | 2-chloro-N-[(4-fluorophenyl)methyl]- N-(3-methoxyphenyl)acetamide | 308.0 |
| A14 | 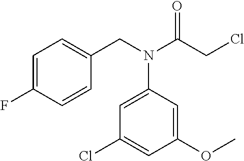 [SMILES: COc1cc(Cl)cc(N(Cc2ccc(F)cc2)C(=O)CCl)c1] | 2-chloro-N-(3-chloro-5-methoxy- phenyl)-N-[(4- fluorophenyl)methyl]acetamide | 342.0 |
| A15 | 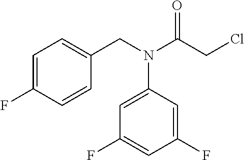 [SMILES: O=C(CCl)N(Cc1ccc(F)cc1)c1cc(F)cc(F)c1] | 2-chloro-N-(3,5-difluorophenyl)-N- [(4-fluorophenyl)methyl]acetamide | 314.0 |
| A16 | 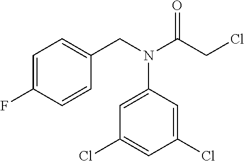 [SMILES: O=C(CCl)N(Cc1ccc(F)cc1)c1cc(Cl)cc(Cl)c1] | 2-chloro-N-(3,5-dichlorophenyl)-N- [(4-fluorophenyl)methyl]acetamide | 345.9 |
| A17 | 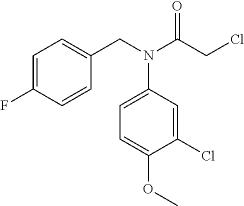 [SMILES: COc1ccc(N(Cc2ccc(F)cc2)C(=O)CCl)cc1Cl] | 2-chloro-N-(3-chloro-4-methoxy- phenyl)-N-[(4- fluorophenyl)methyl]acetamide | 342.0 |
| A18 | 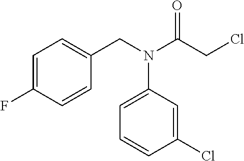 [SMILES: O=C(CCl)N(Cc1ccc(F)cc1)c1cccc(Cl)c1] | 2-chloro-N-(3-chlorophenyl)-N-[(4- fluorophenyl)methyl]acetamide | 312.0 |
| A19 | 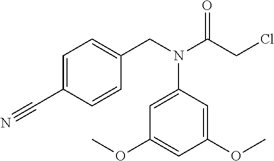 [SMILES: COc1cc(OC)cc(N(Cc2ccc(C#N)cc2)C(=O)CCl)c1] | 2-chloro-N-[(4-cyanophenyl)methyl]- N-(3,5-dimethoxyphenyl)acetamide | 345.1 |
| A20 | 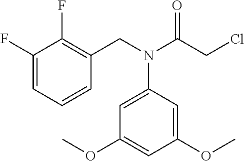 [SMILES: COc1cc(OC)cc(N(Cc2cccc(F)c2F)C(=O)CCl)c1] | 2-chloro-N-[(2,3- difluorophenyl)methyl]-N-(3,5- dimethoxyphenyl)acetamide | 356.0 |
| A21 | 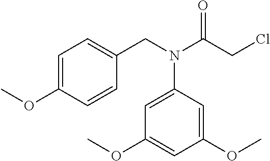 | 2-chloro-N-(3,5-dimethoxyphenyl)-N- [(4-methoxyphenyl)methyl]acetamide | 350.1 |
| A22 | 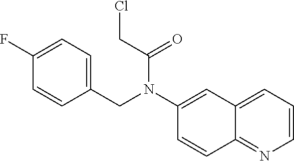 [SMILES: O=C(CCl)N(Cc1ccc(F)cc1)c1ccc2ncccc2c1] | 2-chloro-N-[(4-fluorophenyl)methyl]- N-(6-quinolyl)acetamide | 329.1 |
| A23 | 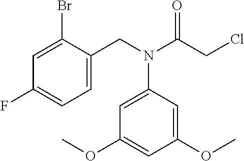 [SMILES: COc1cc(OC)cc(N(Cc2ccc(F)cc2Br)C(=O)CCl)c1] | N-[(2-bromo-4-fluoro- phenyl)methyl]-2-chloro-N-(3,5- dimethoxyphenyl)acetamide | 416.0 |
| A24 | 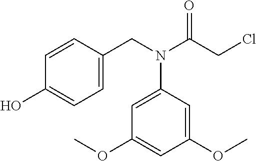 [SMILES: COc1cc(OC)cc(N(Cc2ccc(O)cc2)C(=O)CCl)c1] | 2-chloro-N-(3,5-dimethoxyphenyl)-N- [(4-hydroxyphenyl)methyl]acetamide | 336.1 |
| A25 | 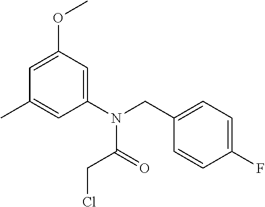 [SMILES: COc1cc(C)cc(N(Cc2ccc(F)cc2)C(=O)CCl)c1] | 2-chloro-N-[(4-fluorophenyl)methyl]- N-(3-methoxy-5-methyl- phenyl)acetamide | 322.1 |
| A26 | 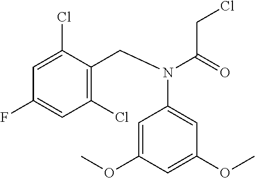 [SMILES: COc1cc(OC)cc(N(Cc2c(Cl)cc(F)cc2Cl)C(=O)CCl)c1] | 2-chloro-N-[(2,6-dichloro-4-fluoro- phenyl)methyl]-N-(3,5- dimethoxyphenyl)acetamide | 406.0 |
| A27 | 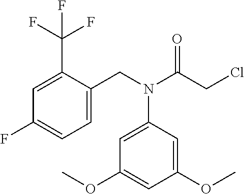 [SMILES: COc1cc(OC)cc(N(Cc2ccc(F)cc2C(F)(F)F)C(=O)CCl)c1] | 2-chloro-N-(3,5-dimethoxyphenyl)-N- [[4-fluoro-2-(trifluoro- methyl)phenyl]methyl]acetamide | 406.0 |
| A28 | 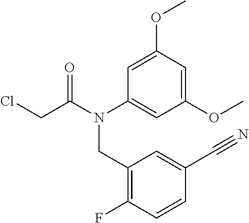 [SMILES: COc1cc(OC)cc(N(Cc2cc(C#N)ccc2F)C(=O)CCl)c1] | 2-chloro-N-[(5-cyano-2-fluoro- phenyl)methyl]-N-(3,5- dimethoxyphenyl)acetamide | 363.1 |
| A29 | 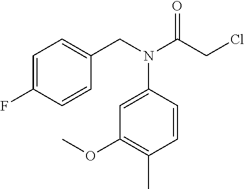 [SMILES: COc1cc(N(Cc2ccc(F)cc2)C(=O)CCl)ccc1C] | 2-chloro-N-[(4-fluorophenyl)methyl]- N-(3-methoxy-4-methyl- phenyl)acetamide | 322.1 |
| A30 | 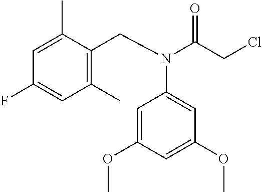 [SMILES: COc1cc(OC)cc(N(Cc2c(C)cc(F)cc2C)C(=O)CCl)c1] | 2-chloro-N-(3,5-dimethoxyphenyl)-N- [(4-fluoro-2,6-dimethyl- phenyl)methyl]acetamide | 366.1 |
| A31 | 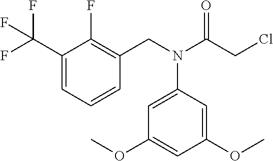 [SMILES: COc1cc(OC)cc(N(Cc2cccc(C(F)(F)F)c2F)C(=O)CCl)c1] | 2-chloro-N-(3,5-dimethoxyphenyl)-N- [[2-fluoro-3-(trifluoro- methyl)phenyl]methyl]acetamide | 406.1 |
| A32 | 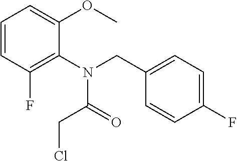 [SMILES: COc1cccc(F)c1N(Cc1ccc(F)cc1)C(=O)CCl] | 2-chloro-N-(2-fluoro-6-methoxy- phenyl)-N-[(4- fluorophenyl)methyl]acetamide | 326.1 |
| A33 | 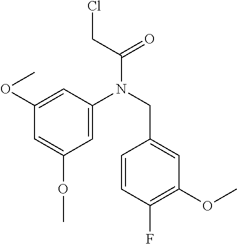 [SMILES: COc1cc(OC)cc(N(Cc2ccc(F)c(OC)c2)C(=O)CCl)c1] | 2-chloro-N-(3,5-dimethoxyphenyl)-N- [(4-fluoro-3-methoxy- phenyl)methyl]acetamide | 368.1 |
| A34 | 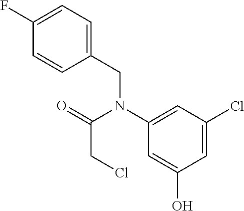 [SMILES: O=C(CCl)N(Cc1ccc(F)cc1)c1cc(O)cc(Cl)c1] | 2-chloro-N-(3-chloro-5-hydroxy- phenyl)-N-[(4- fluorophenyl)methyl]acetamide | 328.1 |
| A35 | 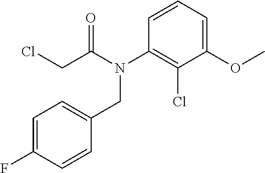 [SMILES: COc1cccc(N(Cc2ccc(F)cc2)C(=O)CCl)c1Cl] | 2-chloro-N-(2-chloro-3-methoxy- phenyl)-N-[(4- fluorophenyl)methyl]acetamide | 342.0 |
| A36 | 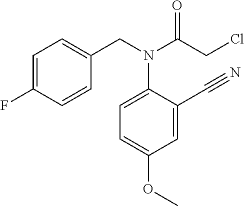 [SMILES: COc1ccc(N(Cc2ccc(F)cc2)C(=O)CCl)c(C#N)c1] | 2-chloro-N-(2-cyano-4-methoxy- phenyl)-N-[(4- fluorophenyl)methyl]acetamide | 333.1 |
| A37 | 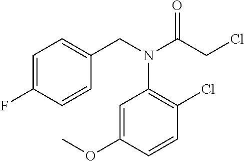 [SMILES: COc1ccc(Cl)c(N(Cc2ccc(F)cc2)C(=O)CCl)c1] | 2-chloro-N-(2-chloro-5-methoxy- phenyl)-N-[(4- fluorophenyl)methyl]acetamide | 342.0 |
| A38 | 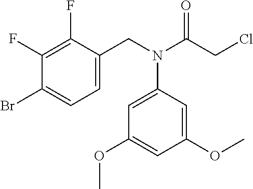 [SMILES: COc1cc(OC)cc(N(Cc2ccc(Br)c(F)c2F)C(=O)CCl)c1] | N-[(4-bromo-2,3-difluoro- phenyl)methyl]-2-chloro-N-(3,5- dimethoxyphenyl)acetamide | 434.0 |
| A39 | 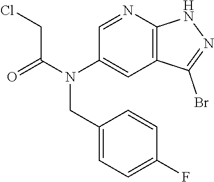 [SMILES: O=C(CCl)N(Cc1ccc(F)cc1)c1cnc2[nH]nc(Br)c2c1] | N-(3-bromo-1H-pyrazolo[3,4- b]pyridin-5-yl)-2-chloro-N-[(4- fluorophenyl)methyl]acetamide | 397.0 |
| A40 | 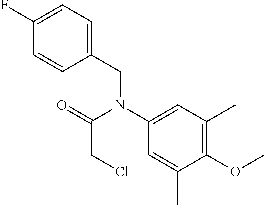 [SMILES: COc1c(C)cc(N(Cc2ccc(F)cc2)C(=O)CCl)cc1C] | 2-chloro-N-[(4-fluorophenyl)methyl]- N-(4-methoxy-3,5-dimethyl- phenyl)acetamide | 336.2 |
| A41 | 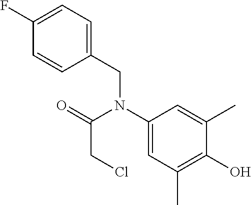 [SMILES: Cc1cc(N(Cc2ccc(F)cc2)C(=O)CCl)cc(C)c1O] | 2-chloro-N-[(4-fluorophenyl)methyl]- N-(4-hydroxy-3,5-dimethyl- phenyl)acetamide | 322.1 |
| A42 | 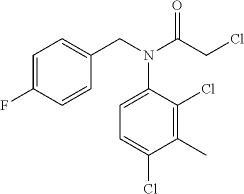 [SMILES: Cc1c(Cl)ccc(N(Cc2ccc(F)cc2)C(=O)CCl)c1Cl] | 2-chloro-N-(2,4-dichloro-3-methyl- phenyl)-N-[(4- fluorophenyl)methyl]acetamide | 360.0 |
| A43 | 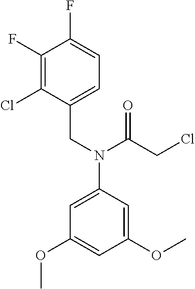 [SMILES: COc1cc(OC)cc(N(Cc2ccc(F)c(F)c2Cl)C(=O)CCl)c1] | 2-chloro-N-[(2-chloro-3,4-difluoro- phenyl)methyl]-N-(3,5- dimethoxyphenyl)acetamide | 390.1 |
| A44 | 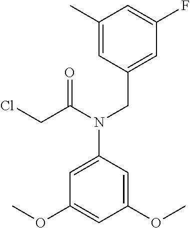 [SMILES: COc1cc(OC)cc(N(Cc2cc(C)cc(F)c2)C(=O)CCl)c1] | 2-chloro-N-(3,5-dimethoxyphenyl)-N- [(3-fluoro-5-methyl- phenyl)methyl]acetamide | 352.1 |
| A45 | 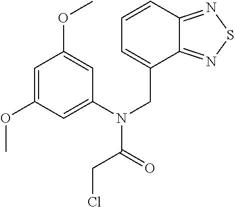 [SMILES: COc1cc(OC)cc(N(Cc2cccc3nsnc23)C(=O)CCl)c1] | N-(2,1,3-benzothiadiazol-4- ylmethyl)-2-chloro-N-(3,5- dimethoxyphenyl)acetamide | 378.1 |
| A46 | 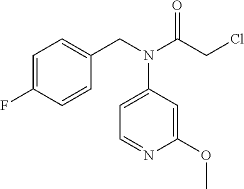 [SMILES: COc1cc(N(Cc2ccc(F)cc2)C(=O)CCl)ccn1] | 2-chloro-N-[(4-fluorophenyl)methyl]- N-(2-methoxy-4-pyridyl)acetamide | 309.1 |
| A47 | 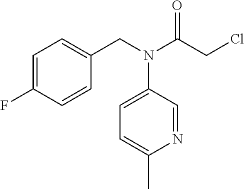 [SMILES: Cc1ccc(N(Cc2ccc(F)cc2)C(=O)CCl)cn1] | 2-chloro-N-[(4-fluorophenyl)methyl]- N-(6-methyl-3-pyridyl)acetamide | 293.1. |

52. The method of claim 43, wherein the compound is represented by a structure in Table 2.

| Working | | | MS Data |
|---|---|---|---|
| Example | Structure | Name | (M + H) |
| B1 | 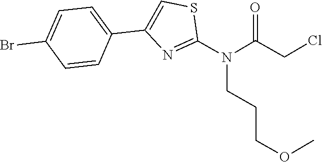 [SMILES: COCCCN(C(=O)CCl)c1nc(-c2ccc(Br)cc2)cs1] | N-[4-(4- bromophenyl)thiazol-2- yl]-2-chloro-N-(3- methoxypropyl)acetamide | 403.1 |
| B2 | 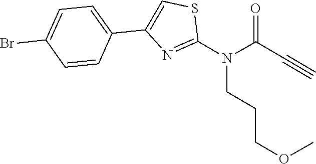 [SMILES: C#CC(=O)N(CCCOC)c1nc(-c2ccc(Br)cc2)cs1] | N-[4-(4- bromophenyl)thiazol-2- yl]-N-(3- methoxypropyl)prop-2- ynamide | 379.2 |
| B3 | 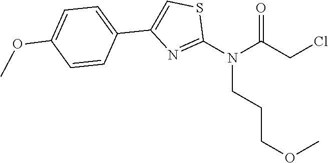 | 2-chloro-N-[4-(4- methoxyphenyl)thiazol- 2-yl]-N-(3- methoxypropyl)acetamide | 355.1 |
| B4 | 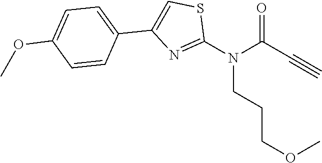 | N-[4-(4- methoxyphenyl)thiazol- 2-yl]-N-(3- methoxypropyl)prop-2- ynamide | 331.2 |
| B5 | 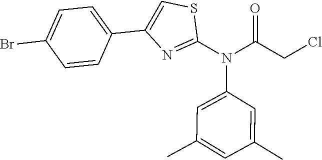 [SMILES: Cc1cc(C)cc(N(C(=O)CCl)c2nc(-c3ccc(Br)cc3)cs2)c1] | N-[4-(4- bromophenyl)thiazol-2- yl]-2-chloro-N-(3,5- dimethylphenyl)acetamide | 435.0 |
| B6 | 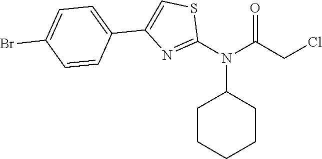 [SMILES: O=C(CCl)N(c1nc(-c2ccc(Br)cc2)cs1)C1CCCCC1] | N-[4-(4- bromophenyl)thiazol-2- yl]-2-chloro-N- cyclohexyl-acetamide | 413.0 |
| B7 | 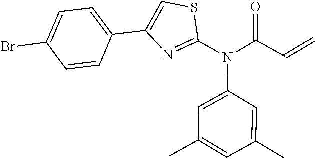 [SMILES: C=CC(=O)N(c1cc(C)cc(C)c1)c1nc(-c2ccc(Br)cc2)cs1] | N-[4-(4- bromophenyl)thiazol-2- yl]-N-(3,5- dimethylphenyl)prop-2- enamide | 413.0 |
| B8 | 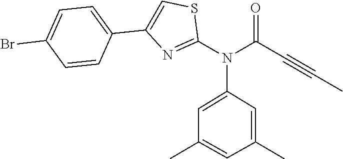 [SMILES: CC#CC(=O)N(c1cc(C)cc(C)c1)c1nc(-c2ccc(Br)cc2)cs1] | N-[4-(4- bromophenyl)thiazol-2- yl]-N-(3,5- dimethylphenyl)but-2- ynamide | 425.2 |
| B9 | 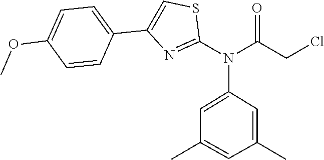 [SMILES: COc1ccc(-c2csc(N(C(=O)CCl)c3cc(C)cc(C)c3)n2)cc1] | 2-chloro-N-(3,5- dimethylphenyl)-N-[4-(4- methoxyphenyl)thiazol- 2-yl]acetamide | 387.2 |
| B10 | 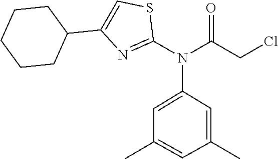 [SMILES: Cc1cc(C)cc(N(C(=O)CCl)c2nc(C3CCCCC3)cs2)c1] | 2-chloro-N-(4- cyclohexylthiazol-2-yl)- N-(3,5- dimethylphenyl)acetamide | 363.2 |
| B11 | 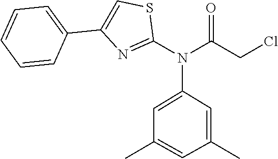 [SMILES: Cc1cc(C)cc(N(C(=O)CCl)c2nc(-c3ccccc3)cs2)c1] | 2-chloro-N-(3,5- dimethylphenyl)-N-(4- phenylthiazol-2- yl)acetamide | 357.1 |
| B12 | 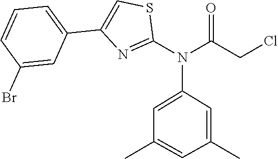 [SMILES: Cc1cc(C)cc(N(C(=O)CCl)c2nc(-c3cccc(Br)c3)cs2)c1] | N-[4-(3- bromophenyl)thiazol-2- yl]-2-chloro-N-(3,5- dimethylphenyl)acetamide | 435.1 |
| B13 | 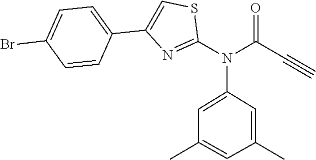 [SMILES: C#CC(=O)N(c1cc(C)cc(C)c1)c1nc(-c2ccc(Br)cc2)cs1] | N-[4-(4- bromophenyl)thiazol-2- yl]-N-(3,5- dimethylphenyl)prop-2- ynamide | 411.0 |
| B14 | 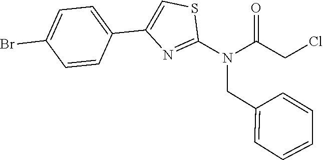 [SMILES: O=C(CCl)N(Cc1ccccc1)c1nc(-c2ccc(Br)cc2)cs1] | N-benzyl-N-[4-(4- bromophenyl)thiazol-2- yl]-2-chloro-acetamide | 421.1 |
| B15 | 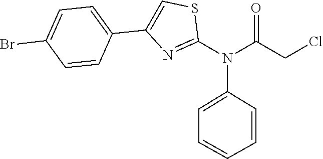 [SMILES: O=C(CCl)N(c1ccccc1)c1nc(-c2ccc(Br)cc2)cs1] | N-[4-(4- bromophenyl)thiazol-2- yl]-2-chloro-N-phenyl- acetamide | 407.1 |
| B16 | 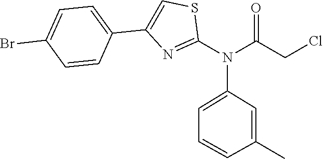 [SMILES: Cc1cccc(N(C(=O)CCl)c2nc(-c3ccc(Br)cc3)cs2)c1] | N-[4-(4- bromophenyl)thiazol-2- yl]-2-chloro-N-(m- tolyl)acetamide | 421.1 |
| B17 | 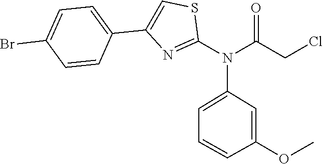 [SMILES: COc1cccc(N(C(=O)CCl)c2nc(-c3ccc(Br)cc3)cs2)c1] | N-[4-(4- bromophenyl)thiazol-2- yl]-2-chloro-N-(3- methoxyphenyl)acetamide | 437.1 |
| B18 | 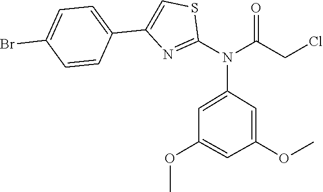 [SMILES: COc1cc(OC)cc(N(C(=O)CCl)c2nc(-c3ccc(Br)cc3)cs2)c1] | N-[4-(4- bromophenyl)thiazol-2- yl]-2-chloro-N-(3,5- dimethoxyphenyl)acetamide | 467.1 |
| B19 | 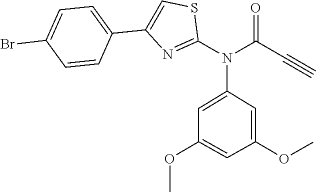 [SMILES: C#CC(=O)N(c1cc(OC)cc(OC)c1)c1nc(-c2ccc(Br)cc2)cs1] | N-[4-(4- bromophenyl)thiazol-2- yl]-N-(3,5- dimethoxyphenyl)prop-2- ynamide | 443.0 |
| B20 | 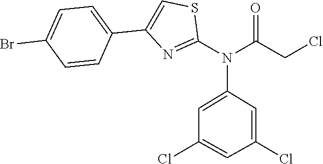 [SMILES: O=C(CCl)N(c1cc(Cl)cc(Cl)c1)c1nc(-c2ccc(Br)cc2)cs1] | N-[4-(4- bromophenyl)thiazol-2- yl]-2-chloro-N-(3,5- dichlorophenyl)acetamide | 476.7 |
| B21 | 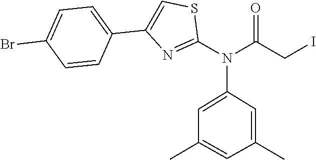 [SMILES: Cc1cc(C)cc(N(C(=O)CI)c2nc(-c3ccc(Br)cc3)cs2)c1] | N-[4-(4- bromophenyl)thiazol-2- yl]-N-(3,5- dimethylphenyl)-2-iodo- acetamide | 527.0 |
| B22 | 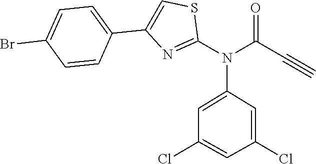 [SMILES: C#CC(=O)N(c1cc(Cl)cc(Cl)c1)c1nc(-c2ccc(Br)cc2)cs1] | N-[4-(4- bromophenyl)thiazol-2- yl]-N-(3,5- dichlorophenyl)prop-2- ynamide | 452.9 |
| B23 | 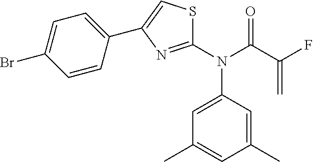 [SMILES: C=C(F)C(=O)N(c1cc(C)cc(C)c1)c1nc(-c2ccc(Br)cc2)cs1] | N-[4-(4- bromophenyl)thiazol-2- yl]-N-(3,5- dimethylphenyl)-2- fluoro-prop-2-enamide | 431.0 |
| B24 | 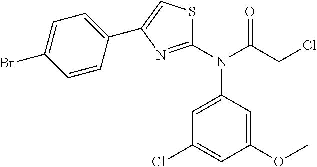 [SMILES: COc1cc(Cl)cc(N(C(=O)CCl)c2nc(-c3ccc(Br)cc3)cs2)c1] | N-[4-(4- bromophenyl)thiazol-2- yl]-2-chloro-N-(3-chloro- 5-methoxy- phenyl)acetamide | 472.9 |
| B25 | 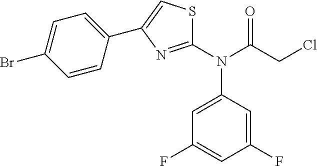 [SMILES: O=C(CCl)N(c1cc(F)cc(F)c1)c1nc(-c2ccc(Br)cc2)cs1] | N-[4-(4- bromophenyl)thiazol-2- yl]-2-chloro-N-(3,5- difluorophenyl)acetamide | 444.9 |
| B26 | 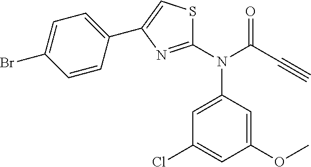 [SMILES: C#CC(=O)N(c1cc(Cl)cc(OC)c1)c1nc(-c2ccc(Br)cc2)cs1] | N-[4-(4- bromophenyl)thiazol-2- yl]-N-(3-chloro-5- methoxy-phenyl)prop-2- ynamide | 449.0 |
| B27 | 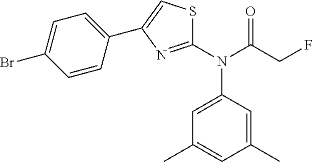 [SMILES: Cc1cc(C)cc(N(C(=O)CF)c2nc(-c3ccc(Br)cc3)cs2)c1] | N-[4-(4- bromophenyl)thiazol-2- yl]-N-(3,5- dimethylphenyl)-2- fluoro-acetamide | 419.0 |
| B28 | 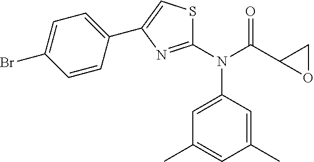 [SMILES: Cc1cc(C)cc(N(C(=O)C2CO2)c2nc(-c3ccc(Br)cc3)cs2)c1] | N-[4-(4- bromophenyl)thiazol-2- yl]-N-(3,5- dimethylphenyl)oxirane- 2-carboxamide | 429.0 |

53. The method of claim 43, wherein the compound is represented by a structure in Table 3.

| | |
|---|---|
| 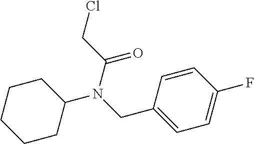 [SMILES: O=C(CCl)N(Cc1ccc(F)cc1)C1CCCCC1] | (Compound 1) |
| 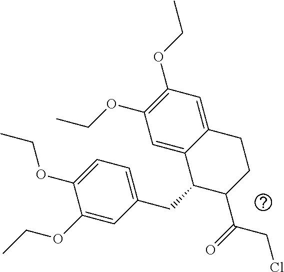 [SMILES: CCOc1ccc(C[C@@H]2c3cc(OCC)c(OCC)cc3CCC2C(=O)CCl)cc1OCC] | (Compound 2) |
| 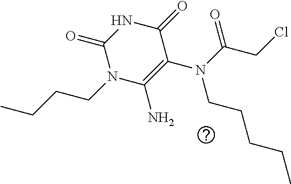 [SMILES: CCCCCN(C(=O)CCl)c1c(N)n(CCCC)c(=O)[nH]c1=O] | (Compound 3) |
| 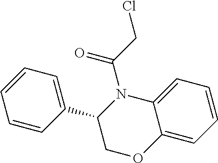 [SMILES: O=C(CCl)N1c2ccccc2OC[C@@H]1c1ccccc1] | (Compound 4) |
| 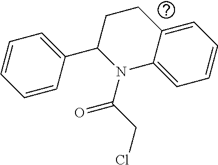 [SMILES: O=C(CCl)N1c2ccccc2CCC1c1ccccc1] | (Compound 5) |
| 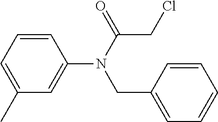 [SMILES: Cc1cccc(N(Cc2ccccc2)C(=O)CCl)c1] | (Compound 6) |
| 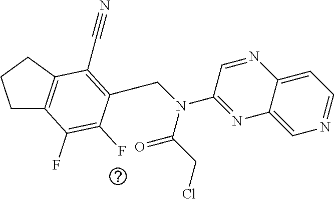 [SMILES: N#Cc1c(CN(C(=O)CCl)c2cnc3ccncc3n2)c(F)c(F)c2c1CCC2] | (Compound 7) |
| 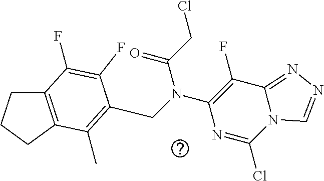 [SMILES: Cc1c(CN(C(=O)CCl)c2nc(Cl)n3cnnc3c2F)c(F)c(F)c2c1CCC2] | (Compound 8) |
| 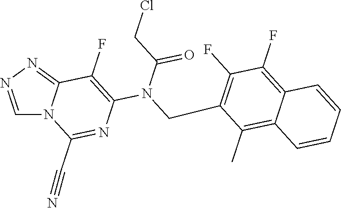 [SMILES: Cc1c(CN(C(=O)CCl)c2nc(C#N)n3cnnc3c2F)c(F)c(F)c2ccccc12] | (Compound 9) |
| 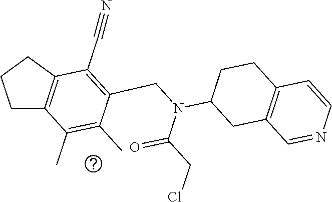 [SMILES: Cc1c(C)c2c(c(C#N)c1CN(C(=O)CCl)C1CCc3ccncc3C1)CCC2] | (Compound 10) |
| 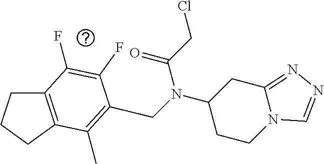 [SMILES: Cc1c(CN(C(=O)CCl)C2CCn3cnnc3C2)c(F)c(F)c2c1CCC2] | (Compound 11) |
| 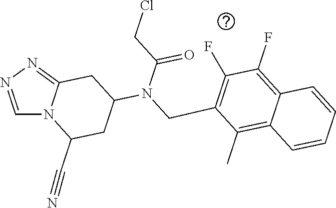 [SMILES: Cc1c(CN(C(=O)CCl)C2Cc3nncn3C(C#N)C2)c(F)c(F)c2ccccc12] | (Compound 12) |
| 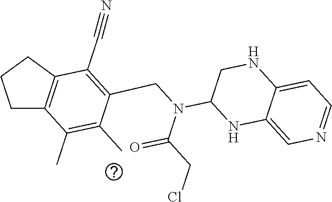 [SMILES: Cc1c(C)c2c(c(C#N)c1CN(C(=O)CCl)C1CNc3ccncc3N1)CCC2] | (Compound 13) |
| 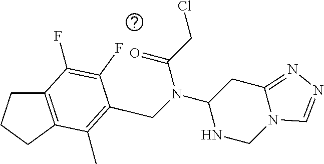 [SMILES: Cc1c(CN(C(=O)CCl)C2Cc3nncn3CN2)c(F)c(F)c2c1CCC2] | (Compound 14) |
| 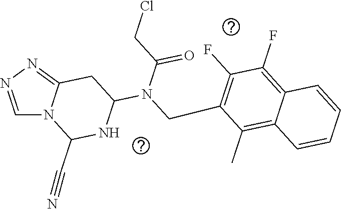 [SMILES: Cc1c(CN(C(=O)CCl)C2Cc3nncn3C(C#N)N2)c(F)c(F)c2ccccc12] | (Compound 15) |
| 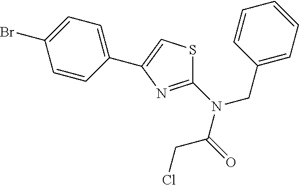 [SMILES: O=C(CCl)N(Cc1ccccc1)c1nc(-c2ccc(Br)cc2)cs1] | (Compound 16) |
| 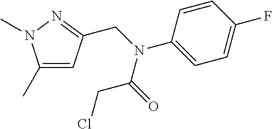 [SMILES: Cc1cc(CN(C(=O)CCl)c2ccc(F)cc2)nn1C] | (Compound 17) |
| 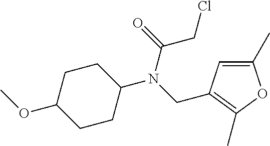 | (Compound 18) |
| 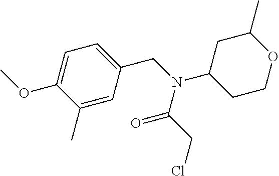 [SMILES: COc1ccc(CN(C(=O)CCl)C2CCOC(C)C2)cc1C] | (Compound 19) |
| 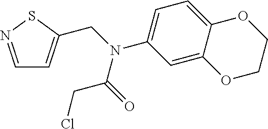 [SMILES: O=C(CCl)N(Cc1ccns1)c1ccc2c(c1)OCCO2] | (Compound 20) |
| 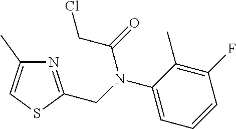 [SMILES: Cc1csc(CN(C(=O)CCl)c2cccc(F)c2C)n1] | (Compound 21) |
| 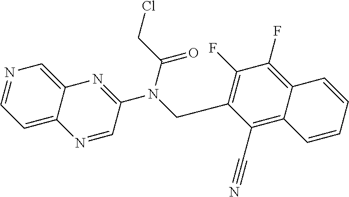 [SMILES: N#Cc1c(CN(C(=O)CCl)c2cnc3ccncc3n2)c(F)c(F)c2ccccc12] | (Compound 22) |
| 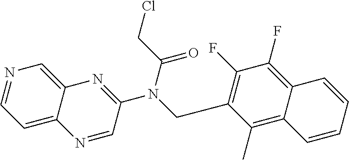 [SMILES: Cc1c(CN(C(=O)CCl)c2cnc3ccncc3n2)c(F)c(F)c2ccccc12] | (Compound 23) |
| 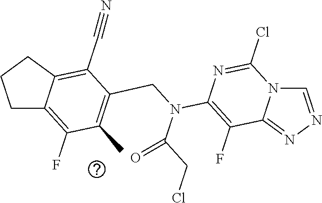 | (Compound 24) |
| 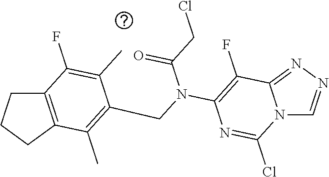 [SMILES: Cc1c(F)c2c(c(C)c1CN(C(=O)CCl)c1nc(Cl)n3cnnc3c1F)CCC2] | (Compound 25) |
| 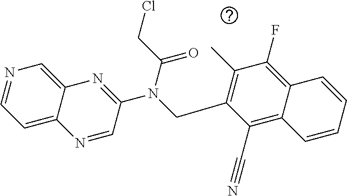 [SMILES: Cc1c(CN(C(=O)CCl)c2cnc3ccncc3n2)c(C#N)c2ccccc2c1F] | (Compound 27) |
| 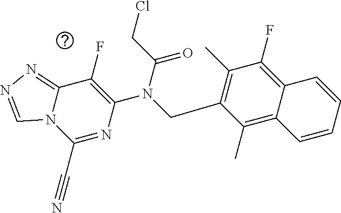 [SMILES: Cc1c(CN(C(=O)CCl)c2nc(C#N)n3cnnc3c2F)c(C)c2ccccc2c1F] | (Compound 28) |
| 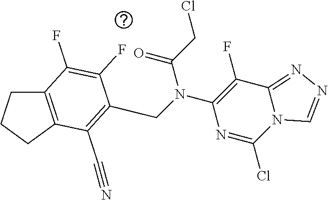 [SMILES: N#Cc1c(CN(C(=O)CCl)c2nc(Cl)n3cnnc3c2F)c(F)c(F)c2c1CCC2] | (Compound 29) |
| 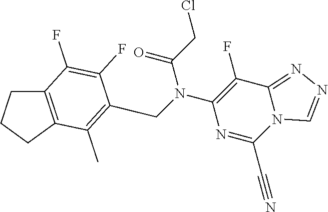 [SMILES: Cc1c(CN(C(=O)CCl)c2nc(C#N)n3cnnc3c2F)c(F)c(F)c2c1CCC2] | (Compound 30) |
| 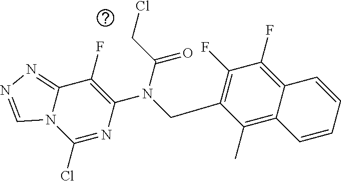 [SMILES: Cc1c(CN(C(=O)CCl)c2nc(Cl)n3cnnc3c2F)c(F)c(F)c2ccccc12] | (Compound 33) |
| 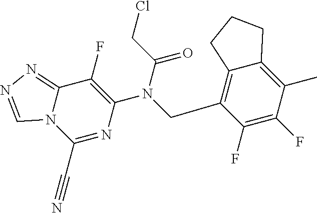 [SMILES: Cc1c(F)c(F)c(CN(C(=O)CCl)c2nc(C#N)n3cnnc3c2F)c2c1CCC2] | (Compound 43) |
| 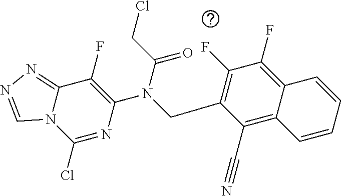 [SMILES: N#Cc1c(CN(C(=O)CCl)c2nc(Cl)n3cnnc3c2F)c(F)c(F)c2ccccc12] | (Compound 44) |
| 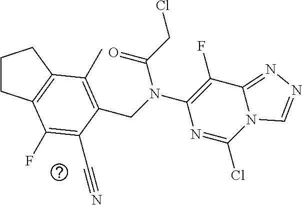 [SMILES: Cc1c(CN(C(=O)CCl)c2nc(Cl)n3cnnc3c2F)c(C#N)c(F)c2c1CCC2] | (Compound 47) |
| 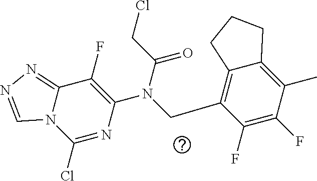 [SMILES: Cc1c(F)c(F)c(CN(C(=O)CCl)c2nc(Cl)n3cnnc3c2F)c2c1CCC2] | (Compound 51) |
| 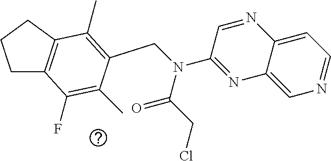 [SMILES: Cc1c(F)c2c(c(C)c1CN(C(=O)CCl)c1cnc3ccncc3n1)CCC2] | (Compound 68) |
| 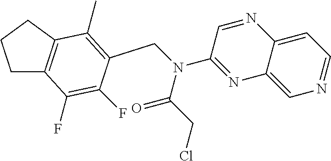 [SMILES: Cc1c(CN(C(=O)CCl)c2cnc3ccncc3n2)c(F)c(F)c2c1CCC2] | (Compound 70) |
| 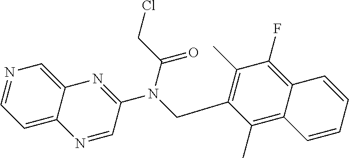 [SMILES: Cc1c(CN(C(=O)CCl)c2cnc3ccncc3n2)c(C)c2ccccc2c1F] | (Compound 73) |
| 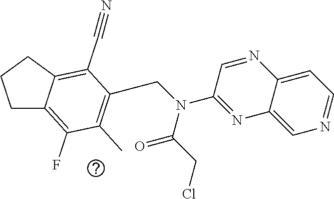 [SMILES: Cc1c(F)c2c(c(C#N)c1CN(C(=O)CCl)c1cnc3ccncc3n1)CCC2] | (Compound 75) |
| 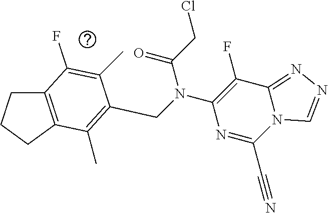 [SMILES: Cc1c(F)c2c(c(C)c1CN(C(=O)CCl)c1nc(C#N)n3cnnc3c1F)CCC2] | (Compound 79) |
| 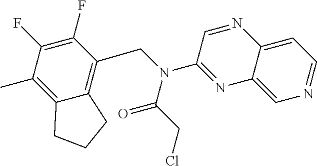 [SMILES: Cc1c(F)c(F)c(CN(C(=O)CCl)c2cnc3ccncc3n2)c2c1CCC2] | (Compound 84) |
| 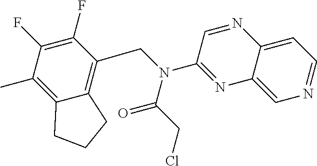 [SMILES: Cc1c(F)c(F)c(CN(C(=O)CCl)c2cnc3ccncc3n2)c2c1CCC2] | (Compound 85) |
| 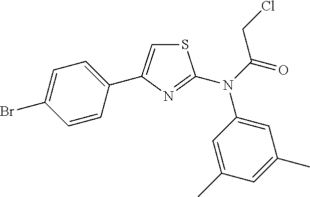 [SMILES: Cc1cc(C)cc(N(C(=O)CCl)c2nc(-c3ccc(Br)cc3)cs2)c1] | (Compound 90) |
| 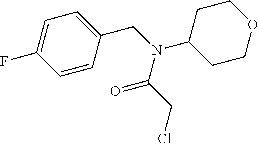 [SMILES: O=C(CCl)N(Cc1ccc(F)cc1)C1CCOCC1] | (Compound 92) |
| 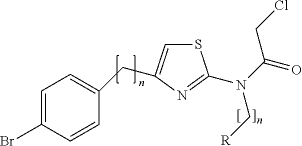 | (Formula 91) |
| 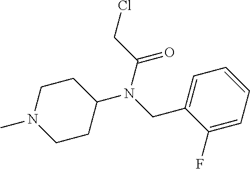 [SMILES: CN1CCC(N(Cc2ccccc2F)C(=O)CCl)CC1] | (Compound 94) |
| 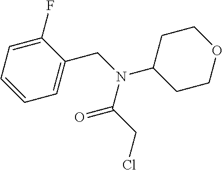 [SMILES: O=C(CCl)N(Cc1ccccc1F)C1CCOCC1] | (Compound 93) |
| 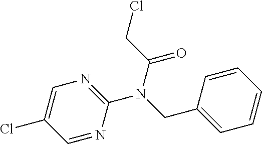 [SMILES: O=C(CCl)N(Cc1ccccc1)c1ncc(Cl)cn1] | (Compound 96) |
| 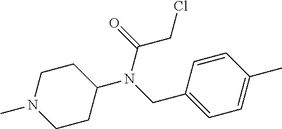 | (Compound 95) |
| 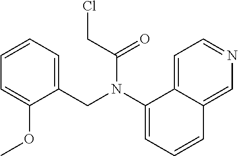 [SMILES: COc1ccccc1CN(C(=O)CCl)c1cccc2cnccc12] | (Compound 98) |
| 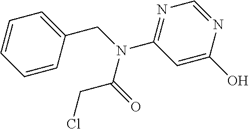 [SMILES: O=C(CCl)N(Cc1ccccc1)c1cc(O)ncn1] | (Compound 97) |
| 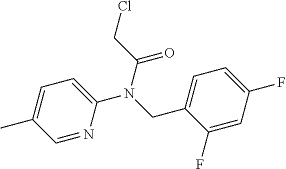 [SMILES: Cc1ccc(N(Cc2ccc(F)cc2F)C(=O)CCl)nc1] | (Compound 100) |
| 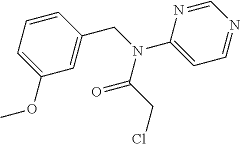 [SMILES: COc1cccc(CN(C(=O)CCl)c2ccncn2)c1] | (Compound 99) |
| 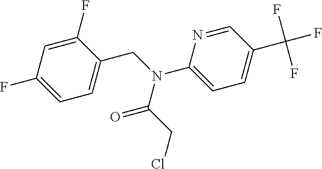 [SMILES: O=C(CCl)N(Cc1ccc(F)cc1F)c1ccc(C(F)(F)F)cn1] | (Compound 102) |
| 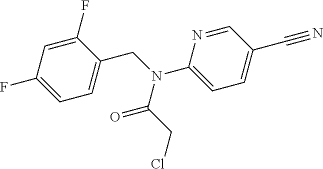 [SMILES: N#Cc1ccc(N(Cc2ccc(F)cc2F)C(=O)CCl)nc1] | (Compound 101) |
| 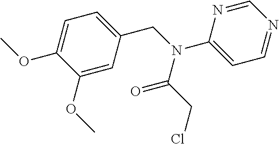 [SMILES: COc1ccc(CN(C(=O)CCl)c2ccncn2)cc1OC] | (Compound 104) |
| 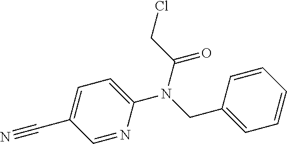 [SMILES: N#Cc1ccc(N(Cc2ccccc2)C(=O)CCl)nc1] | (Compound 103) |
| 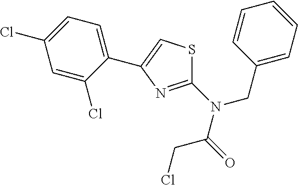 [SMILES: O=C(CCl)N(Cc1ccccc1)c1nc(-c2ccc(Cl)cc2Cl)cs1] | (Compound 106) |
| 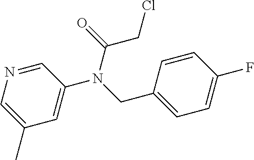 [SMILES: Cc1cncc(N(Cc2ccc(F)cc2)C(=O)CCl)c1] | (Compound 105) |
| 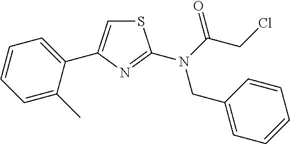 [SMILES: Cc1ccccc1-c1csc(N(Cc2ccccc2)C(=O)CCl)n1] | (Compound 108) |
| 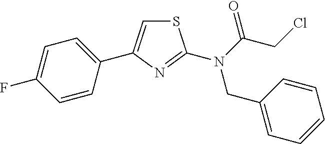 [SMILES: O=C(CCl)N(Cc1ccccc1)c1nc(-c2ccc(F)cc2)cs1] | (Compound 107) |
| 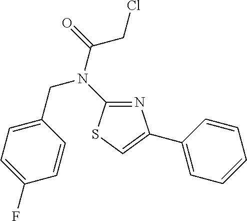 [SMILES: O=C(CCl)N(Cc1ccc(F)cc1)c1nc(-c2ccccc2)cs1] | (Compound 110) |
| 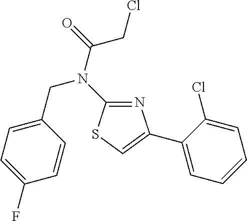 [SMILES: O=C(CCl)N(Cc1ccc(F)cc1)c1nc(-c2ccccc2Cl)cs1] | (Compound 109) |
| 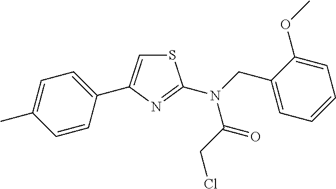 [SMILES: COc1ccccc1CN(C(=O)CCl)c1nc(-c2ccc(C)cc2)cs1] | (Compound 112) |
| 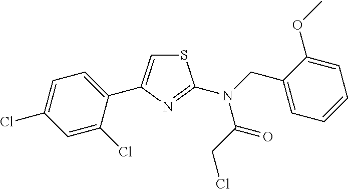 [SMILES: COc1ccccc1CN(C(=O)CCl)c1nc(-c2ccc(Cl)cc2Cl)cs1] | (Compound 111) |
| 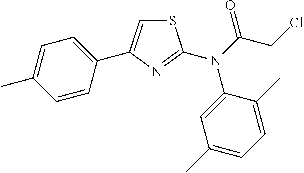 [SMILES: Cc1ccc(-c2csc(N(C(=O)CCl)c3cc(C)ccc3C)n2)cc1] | (Compound 114) |
| 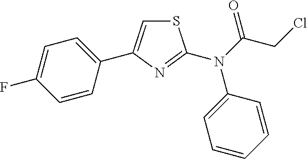 [SMILES: O=C(CCl)N(c1ccccc1)c1nc(-c2ccc(F)cc2)cs1] | (Compound 113) |
| 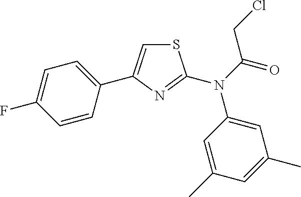 [SMILES: Cc1cc(C)cc(N(C(=O)CCl)c2nc(-c3ccc(F)cc3)cs2)c1] | (Compound 116) |
| 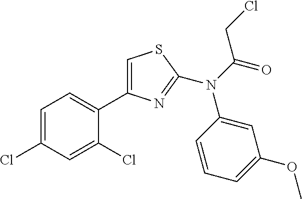 [SMILES: COc1cccc(N(C(=O)CCl)c2nc(-c3ccc(Cl)cc3Cl)cs2)c1] | (Compound 115) |
| 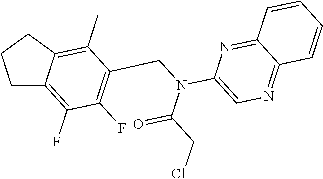 [SMILES: Cc1c(CN(C(=O)CCl)c2cnc3ccccc3n2)c(F)c(F)c2c1CCC2] | (Compound 118) |
| 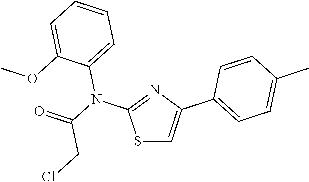 | (Compound 117) |
| 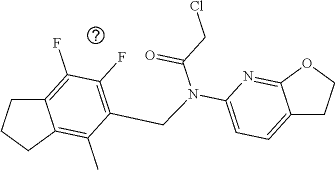 [SMILES: Cc1c(CN(C(=O)CCl)c2ccc3c(n2)OCC3)c(F)c(F)c2c1CCC2] | (Compound 120) |
| 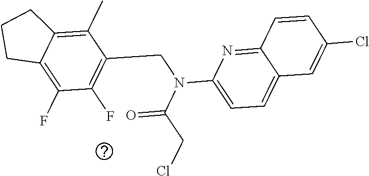 [SMILES: Cc1c(CN(C(=O)CCl)c2ccc3cc(Cl)ccc3n2)c(F)c(F)c2c1CCC2] | (Compound 119) |
| 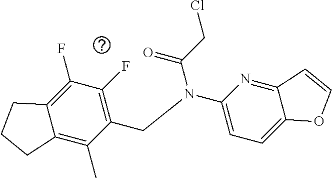 [SMILES: Cc1c(CN(C(=O)CCl)c2ccc3occc3n2)c(F)c(F)c2c1CCC2] | (Compound 122) |
| 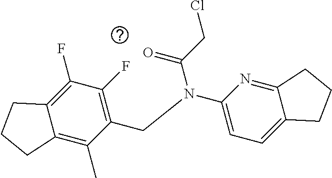 [SMILES: Cc1c(CN(C(=O)CCl)c2ccc3c(n2)CCC3)c(F)c(F)c2c1CCC2] | (Compound 121) |
| 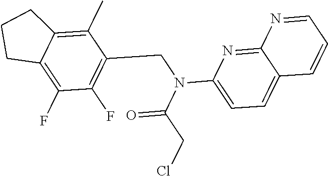 [SMILES: Cc1c(CN(C(=O)CCl)c2ccc3cccnc3n2)c(F)c(F)c2c1CCC2] | (Compound 124) |
| 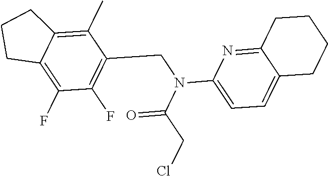 [SMILES: Cc1c(CN(C(=O)CCl)c2ccc3c(n2)CCCC3)c(F)c(F)c2c1CCC2] | (Compound 123) |
| 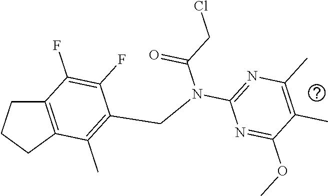 [SMILES: COc1nc(N(Cc2c(C)c3c(c(F)c2F)CCC3)C(=O)CCl)nc(C)c1C] | (Compound 126) |
| 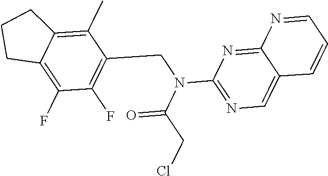 [SMILES: Cc1c(CN(C(=O)CCl)c2ncc3cccnc3n2)c(F)c(F)c2c1CCC2] | (Compound 125) |
| 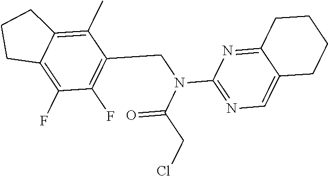 [SMILES: Cc1c(CN(C(=O)CCl)c2ncc3c(n2)CCCC3)c(F)c(F)c2c1CCC2] | (Compound 128) |
| 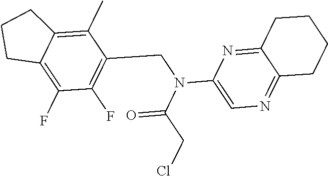 [SMILES: Cc1c(CN(C(=O)CCl)c2cnc3c(n2)CCCC3)c(F)c(F)c2c1CCC2] | (Compound 127) |
| 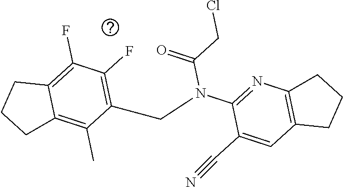 [SMILES: Cc1c(CN(C(=O)CCl)c2nc3c(cc2C#N)CCC3)c(F)c(F)c2c1CCC2] | (Compound 130) |
| 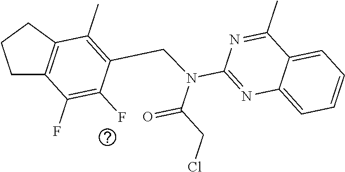 [SMILES: Cc1c(CN(C(=O)CCl)c2nc(C)c3ccccc3n2)c(F)c(F)c2c1CCC2] | (Compound 129) |
| 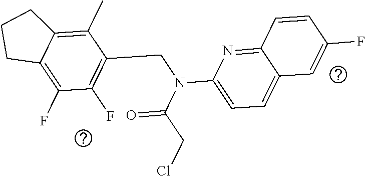 [SMILES: Cc1c(CN(C(=O)CCl)c2ccc3cc(F)ccc3n2)c(F)c(F)c2c1CCC2] | (Compound 132) |
| 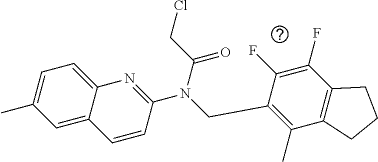 [SMILES: Cc1ccc2nc(N(Cc3c(C)c4c(c(F)c3F)CCC4)C(=O)CCl)ccc2c1] | (Compound 131) |
| 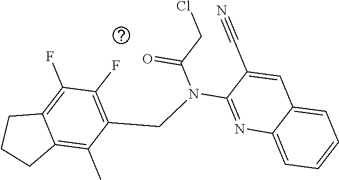 [SMILES: Cc1c(CN(C(=O)CCl)c2nc3ccccc3cc2C#N)c(F)c(F)c2c1CCC2] | (Compound 134) |
| 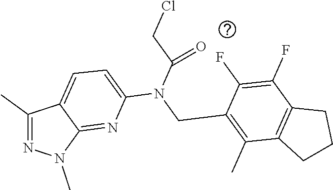 [SMILES: Cc1c(CN(C(=O)CCl)c2ccc3c(C)nn(C)c3n2)c(F)c(F)c2c1CCC2] | (Compound 133) |
| 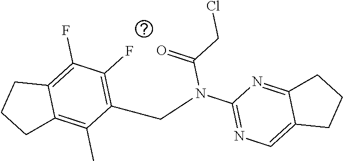 [SMILES: Cc1c(CN(C(=O)CCl)c2ncc3c(n2)CCC3)c(F)c(F)c2c1CCC2] | (Compound 136) |
| 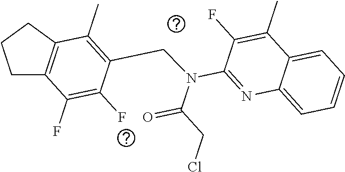 [SMILES: Cc1c(CN(C(=O)CCl)c2nc3ccccc3c(C)c2F)c(F)c(F)c2c1CCC2] | (Compound 135) |
| 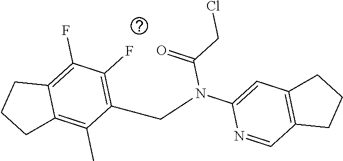 [SMILES: Cc1c(CN(C(=O)CCl)c2cc3c(cn2)CCC3)c(F)c(F)c2c1CCC2] | (Compound 138) |
| 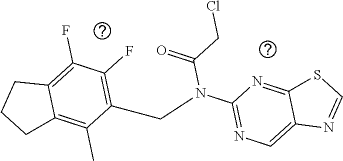 [SMILES: Cc1c(CN(C(=O)CCl)c2ncc3ncsc3n2)c(F)c(F)c2c1CCC2] | (Compound 137) |
| 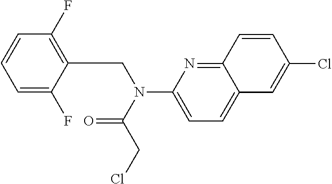 [SMILES: O=C(CCl)N(Cc1c(F)cccc1F)c1ccc2cc(Cl)ccc2n1] | (Compound 140) |
| 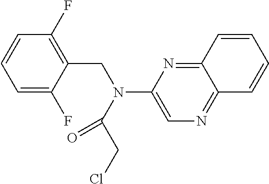 [SMILES: O=C(CCl)N(Cc1c(F)cccc1F)c1cnc2ccccc2n1] | (Compound 139) |
| 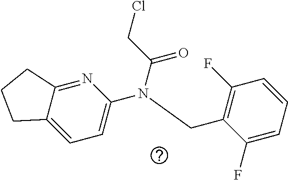 [SMILES: O=C(CCl)N(Cc1c(F)cccc1F)c1ccc2c(n1)CCC2] | (Compound 142) |
| 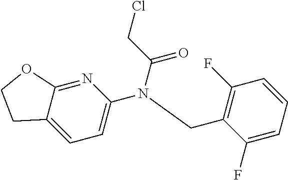 [SMILES: O=C(CCl)N(Cc1c(F)cccc1F)c1ccc2c(n1)OCC2] | (Compound 141) |
| 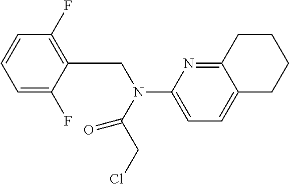 [SMILES: O=C(CCl)N(Cc1c(F)cccc1F)c1ccc2c(n1)CCCC2] | (Compound 144) |
| 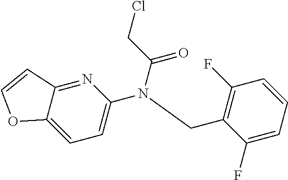 [SMILES: O=C(CCl)N(Cc1c(F)cccc1F)c1ccc2occc2n1] | (Compound 143) |
| 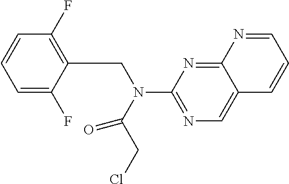 [SMILES: O=C(CCl)N(Cc1c(F)cccc1F)c1ncc2cccnc2n1] | (Compound 146) |
| 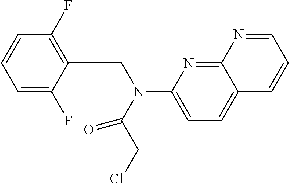 [SMILES: O=C(CCl)N(Cc1c(F)cccc1F)c1ccc2cccnc2n1] | (Compound 145) |
| 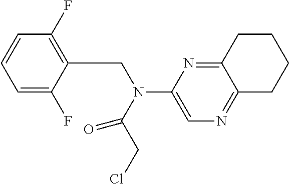 [SMILES: O=C(CCl)N(Cc1c(F)cccc1F)c1cnc2c(n1)CCCC2] | (Compound 148) |
| 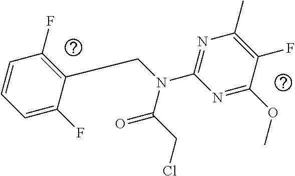 [SMILES: COc1nc(N(Cc2c(F)cccc2F)C(=O)CCl)nc(C)c1F] | (Compound 147) |
| 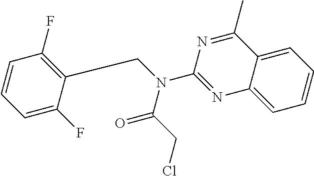 [SMILES: Cc1nc(N(Cc2c(F)cccc2F)C(=O)CCl)nc2ccccc12] | (Compound 150) |
| 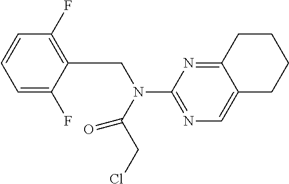 [SMILES: O=C(CCl)N(Cc1c(F)cccc1F)c1ncc2c(n1)CCCC2] | (Compound 149) |
| 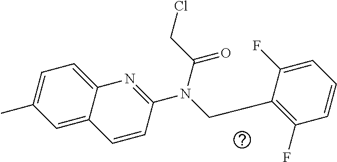 [SMILES: Cc1ccc2nc(N(Cc3c(F)cccc3F)C(=O)CCl)ccc2c1] | (Compound 152) |
| 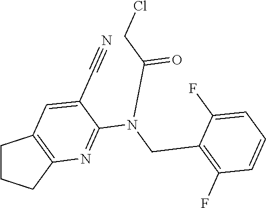 [SMILES: N#Cc1cc2c(nc1N(Cc1c(F)cccc1F)C(=O)CCl)CCC2] | (Compound 151) |
| 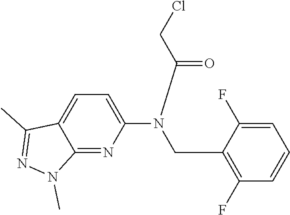 [SMILES: Cc1nn(C)c2nc(N(Cc3c(F)cccc3F)C(=O)CCl)ccc12] | (Compound 154) |
| 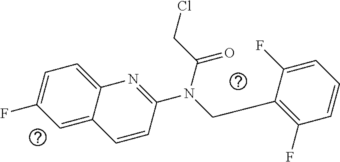 [SMILES: O=C(CCl)N(Cc1c(F)cccc1F)c1ccc2cc(F)ccc2n1] | (Compound 153) |
| 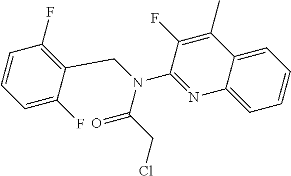 [SMILES: Cc1c(F)c(N(Cc2c(F)cccc2F)C(=O)CCl)nc2ccccc12] | (Compound 156) |
| 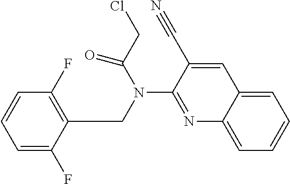 [SMILES: N#Cc1cc2ccccc2nc1N(Cc1c(F)cccc1F)C(=O)CCl] | (Compound 155) |
| 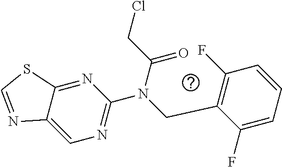 [SMILES: O=C(CCl)N(Cc1c(F)cccc1F)c1ncc2ncsc2n1] | (Compound 158) |
| 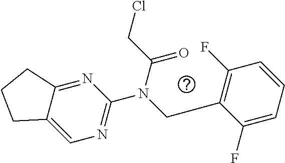 [SMILES: O=C(CCl)N(Cc1c(F)cccc1F)c1ncc2c(n1)CCC2] | (Compound 157) |
| 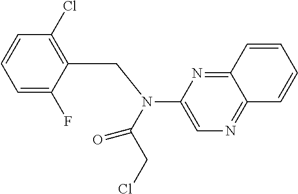 [SMILES: O=C(CCl)N(Cc1c(F)cccc1Cl)c1cnc2ccccc2n1] | (Compound 160) |
| 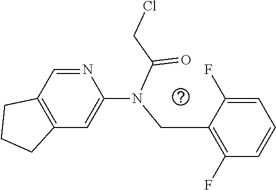 [SMILES: O=C(CCl)N(Cc1c(F)cccc1F)c1cc2c(cn1)CCC2] | (Compound 159) |
| 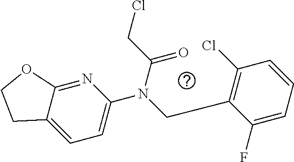 [SMILES: O=C(CCl)N(Cc1c(F)cccc1Cl)c1ccc2c(n1)OCC2] | (Compound 162) |
| 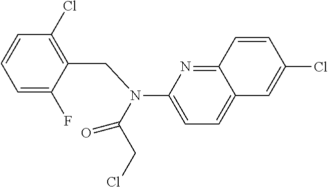 [SMILES: O=C(CCl)N(Cc1c(F)cccc1Cl)c1ccc2cc(Cl)ccc2n1] | (Compound 161) |
| 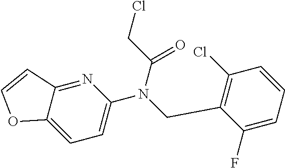 [SMILES: O=C(CCl)N(Cc1c(F)cccc1Cl)c1ccc2occc2n1] | (Compound 164) |
| 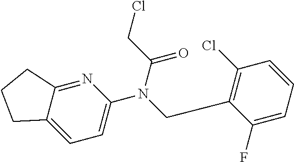 [SMILES: O=C(CCl)N(Cc1c(F)cccc1Cl)c1ccc2c(n1)CCC2] | (Compound 163) |
| 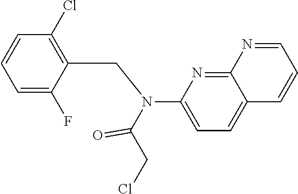 [SMILES: O=C(CCl)N(Cc1c(F)cccc1Cl)c1ccc2cccnc2n1] | (Compound 166) |
| 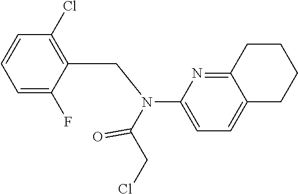 [SMILES: O=C(CCl)N(Cc1c(F)cccc1Cl)c1ccc2c(n1)CCCC2] | (Compound 165) |
| 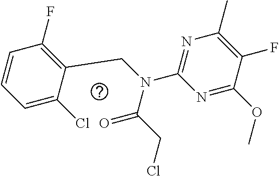 [SMILES: COc1nc(N(Cc2c(F)cccc2Cl)C(=O)CCl)nc(C)c1F] | (Compound 168) |
| 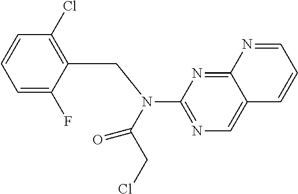 [SMILES: O=C(CCl)N(Cc1c(F)cccc1Cl)c1ncc2cccnc2n1] | (Compound 167) |
| 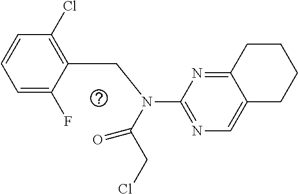 [SMILES: O=C(CCl)N(Cc1c(F)cccc1Cl)c1ncc2c(n1)CCCC2] | (Compound 170) |
| 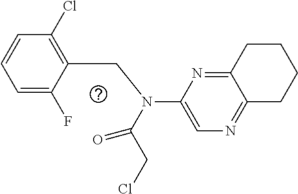 [SMILES: O=C(CCl)N(Cc1c(F)cccc1Cl)c1cnc2c(n1)CCCC2] | (Compound 169) |
| 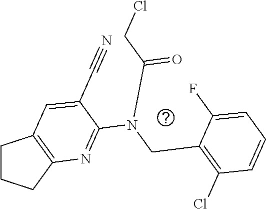 [SMILES: N#Cc1cc2c(nc1N(Cc1c(F)cccc1Cl)C(=O)CCl)CCC2] | (Compound 172) |
| 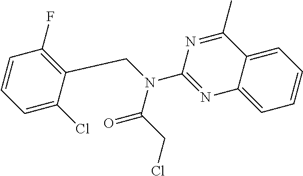 [SMILES: Cc1nc(N(Cc2c(F)cccc2Cl)C(=O)CCl)nc2ccccc12] | (Compound 171) |
| 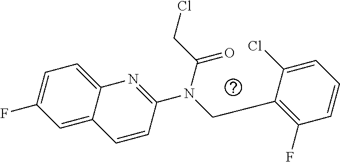 [SMILES: O=C(CCl)N(Cc1c(F)cccc1Cl)c1ccc2cc(F)ccc2n1] | (Compound 174) |
| 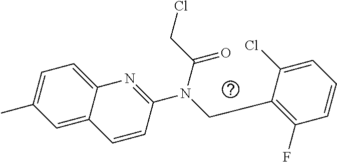 [SMILES: Cc1ccc2nc(N(Cc3c(F)cccc3Cl)C(=O)CCl)ccc2c1] | (Compound 173) |
| 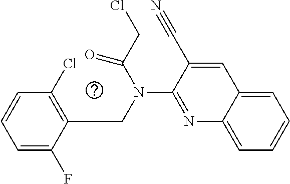 [SMILES: N#Cc1cc2ccccc2nc1N(Cc1c(F)cccc1Cl)C(=O)CCl] | (Compound 176) |
| 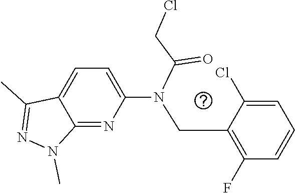 [SMILES: Cc1nn(C)c2nc(N(Cc3c(F)cccc3Cl)C(=O)CCl)ccc12] | (Compound 175) |
| 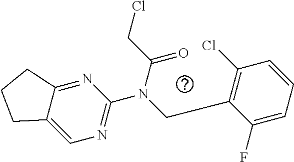 [SMILES: O=C(CCl)N(Cc1c(F)cccc1Cl)c1ncc2c(n1)CCC2] | (Compound 178) |
| 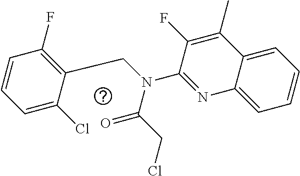 [SMILES: Cc1c(F)c(N(Cc2c(F)cccc2Cl)C(=O)CCl)nc2ccccc12] | (Compound 177) |
| 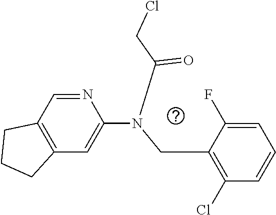 [SMILES: O=C(CCl)N(Cc1c(F)cccc1Cl)c1cc2c(cn1)CCC2] | (Compound 180) |
| 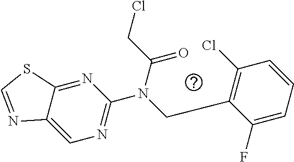 [SMILES: O=C(CCl)N(Cc1c(F)cccc1Cl)c1ncc2ncsc2n1] | (Compound 179) |
| 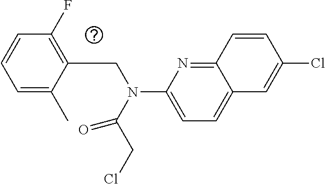 [SMILES: Cc1cccc(F)c1CN(C(=O)CCl)c1ccc2cc(Cl)ccc2n1] | (Compound 182) |
| 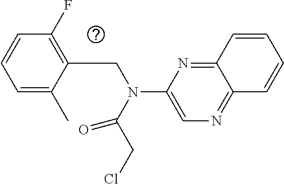 [SMILES: Cc1cccc(F)c1CN(C(=O)CCl)c1cnc2ccccc2n1] | (Compound 181) |
| 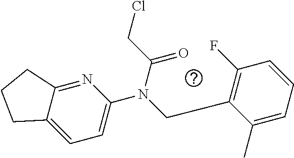 [SMILES: Cc1cccc(F)c1CN(C(=O)CCl)c1ccc2c(n1)CCC2] | (Compound 184) |
| 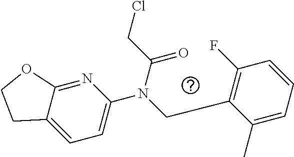 [SMILES: Cc1cccc(F)c1CN(C(=O)CCl)c1ccc2c(n1)OCC2] | (Compound 183) |
| 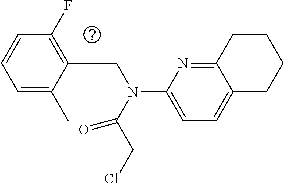 [SMILES: Cc1cccc(F)c1CN(C(=O)CCl)c1ccc2c(n1)CCCC2] | (Compound 186) |
| 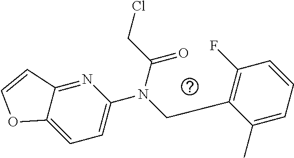 [SMILES: Cc1cccc(F)c1CN(C(=O)CCl)c1ccc2occc2n1] | (Compound 185) |
| 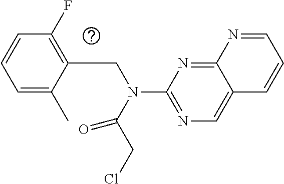 [SMILES: Cc1cccc(F)c1CN(C(=O)CCl)c1ncc2cccnc2n1] | (Compound 188) |
| 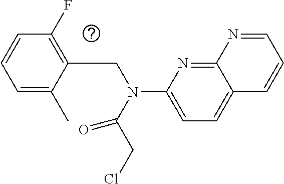 [SMILES: Cc1cccc(F)c1CN(C(=O)CCl)c1ccc2cccnc2n1] | (Compound 187) |
| 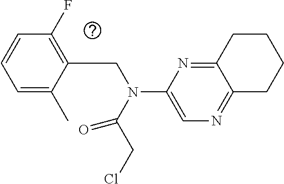 [SMILES: Cc1cccc(F)c1CN(C(=O)CCl)c1cnc2c(n1)CCCC2] | (Compound 190) |
| 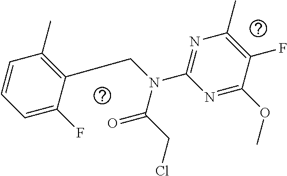 [SMILES: COc1nc(N(Cc2c(C)cccc2F)C(=O)CCl)nc(C)c1F] | (Compound 189) |
| 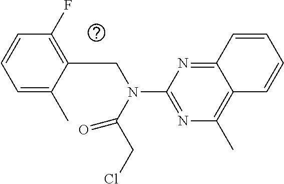 [SMILES: Cc1cccc(F)c1CN(C(=O)CCl)c1nc(C)c2ccccc2n1] | (Compound 192) |
| 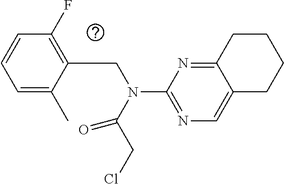 [SMILES: Cc1cccc(F)c1CN(C(=O)CCl)c1ncc2c(n1)CCCC2] | (Compound 191) |
| 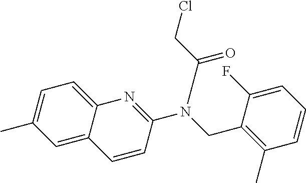 [SMILES: Cc1ccc2nc(N(Cc3c(C)cccc3F)C(=O)CCl)ccc2c1] | (Compound 194) |
| 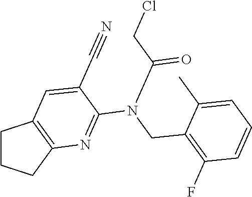 [SMILES: Cc1cccc(F)c1CN(C(=O)CCl)c1nc2c(cc1C#N)CCC2] | (Compound 193) |
| 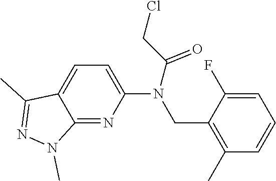 [SMILES: Cc1cccc(F)c1CN(C(=O)CCl)c1ccc2c(C)nn(C)c2n1] | (Compound 196) |
| 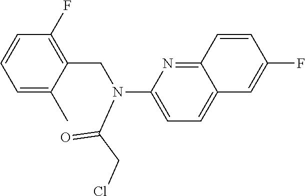 [SMILES: Cc1cccc(F)c1CN(C(=O)CCl)c1ccc2cc(F)ccc2n1] | (Compound 195) |
| 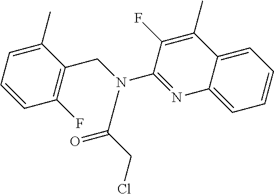 [SMILES: Cc1cccc(F)c1CN(C(=O)CCl)c1nc2ccccc2c(C)c1F] | (Compound 198) |
| 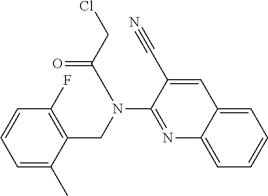 [SMILES: Cc1cccc(F)c1CN(C(=O)CCl)c1nc2ccccc2cc1C#N] | (Compound 197) |
| 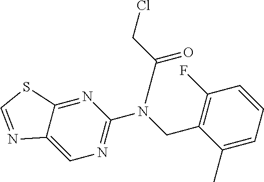 [SMILES: Cc1cccc(F)c1CN(C(=O)CCl)c1ncc2ncsc2n1] | (Compound 200) |
| 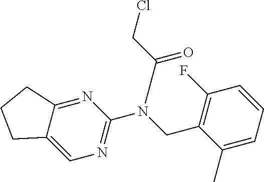 [SMILES: Cc1cccc(F)c1CN(C(=O)CCl)c1ncc2c(n1)CCC2] | (Compound 199) |
| 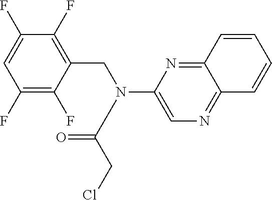 [SMILES: O=C(CCl)N(Cc1c(F)c(F)cc(F)c1F)c1cnc2ccccc2n1] | (Compound 202) |
| 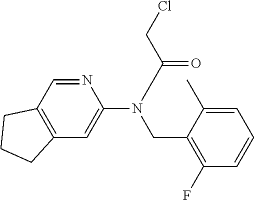 [SMILES: Cc1cccc(F)c1CN(C(=O)CCl)c1cc2c(cn1)CCC2] | (Compound 201) |
| 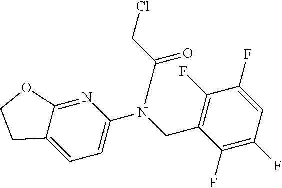 [SMILES: O=C(CCl)N(Cc1c(F)c(F)cc(F)c1F)c1ccc2c(n1)OCC2] | (Compound 204) |
| 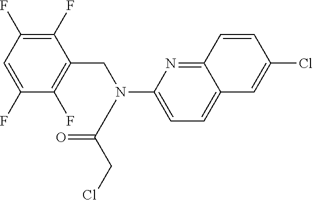 [SMILES: O=C(CCl)N(Cc1c(F)c(F)cc(F)c1F)c1ccc2cc(Cl)ccc2n1] | (Compound 203) |
| 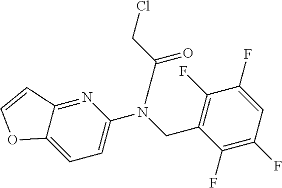 [SMILES: O=C(CCl)N(Cc1c(F)c(F)cc(F)c1F)c1ccc2occc2n1] | (Compound 206) |
| 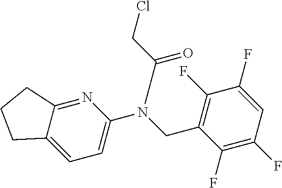 [SMILES: O=C(CCl)N(Cc1c(F)c(F)cc(F)c1F)c1ccc2c(n1)CCC2] | (Compound 205) |
| 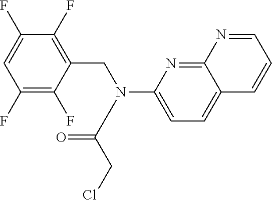 [SMILES: O=C(CCl)N(Cc1c(F)c(F)cc(F)c1F)c1ccc2cccnc2n1] | (Compound 208) |
| 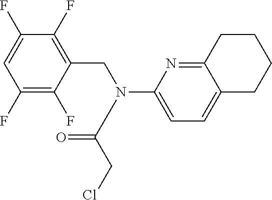 [SMILES: O=C(CCl)N(Cc1c(F)c(F)cc(F)c1F)c1ccc2c(n1)CCCC2] | (Compound 207) |
| 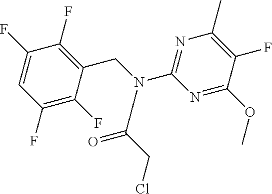 [SMILES: COc1nc(N(Cc2c(F)c(F)cc(F)c2F)C(=O)CCl)nc(C)c1F] | (Compound 210) |
| 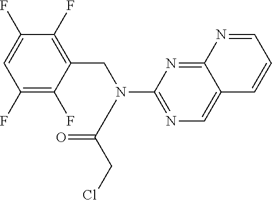 [SMILES: O=C(CCl)N(Cc1c(F)c(F)cc(F)c1F)c1ncc2cccnc2n1] | (Compound 209) |
| 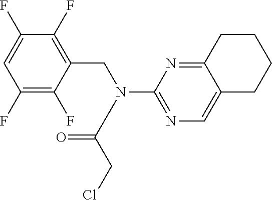 [SMILES: O=C(CCl)N(Cc1c(F)c(F)cc(F)c1F)c1ncc2c(n1)CCCC2] | (Compound 212) |
| 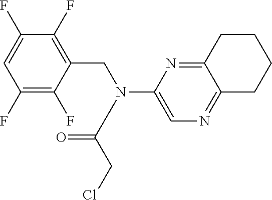 [SMILES: O=C(CCl)N(Cc1c(F)c(F)cc(F)c1F)c1cnc2c(n1)CCCC2] | (Compound 211) |
| 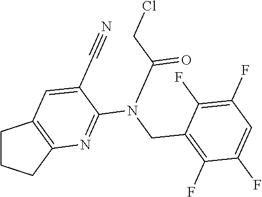 [SMILES: N#Cc1cc2c(nc1N(Cc1c(F)c(F)cc(F)c1F)C(=O)CCl)CCC2] | (Compound 214) |
| 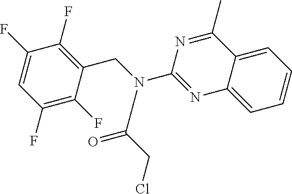 [SMILES: Cc1nc(N(Cc2c(F)c(F)cc(F)c2F)C(=O)CCl)nc2ccccc12] | (Compound 213) |
| 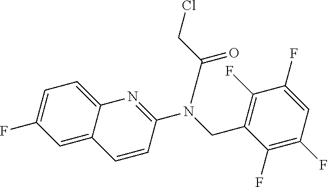 [SMILES: O=C(CCl)N(Cc1c(F)c(F)cc(F)c1F)c1ccc2cc(F)ccc2n1] | (Compound 216) |
| 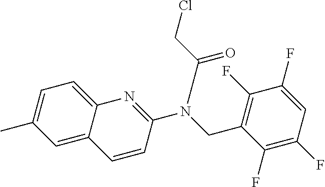 [SMILES: Cc1ccc2nc(N(Cc3c(F)c(F)cc(F)c3F)C(=O)CCl)ccc2c1] | (Compound 215) |
| 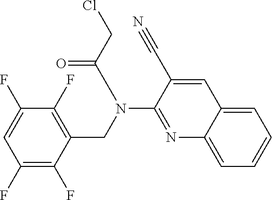 [SMILES: N#Cc1cc2ccccc2nc1N(Cc1c(F)c(F)cc(F)c1F)C(=O)CCl] | (Compound 218) |
| 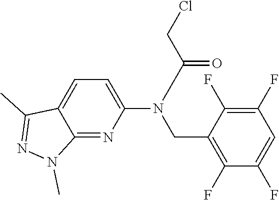 [SMILES: Cc1nn(C)c2nc(N(Cc3c(F)c(F)cc(F)c3F)C(=O)CCl)ccc12] | (Compound 217) |
| 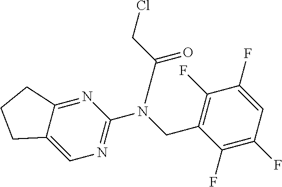 [SMILES: O=C(CCl)N(Cc1c(F)c(F)cc(F)c1F)c1ncc2c(n1)CCC2] | (Compound 220) |
| 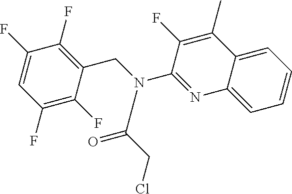 [SMILES: Cc1c(F)c(N(Cc2c(F)c(F)cc(F)c2F)C(=O)CCl)nc2ccccc12] | (Compound 219) |
| 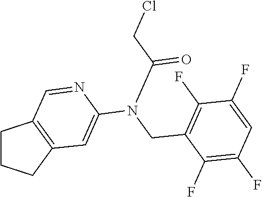 [SMILES: O=C(CCl)N(Cc1c(F)c(F)cc(F)c1F)c1cc2c(cn1)CCC2] | (Compound 222) |
| 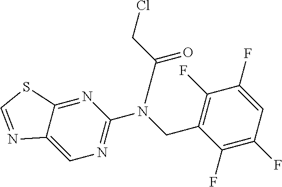 [SMILES: O=C(CCl)N(Cc1c(F)c(F)cc(F)c1F)c1ncc2ncsc2n1] | (Compound 221) |
| 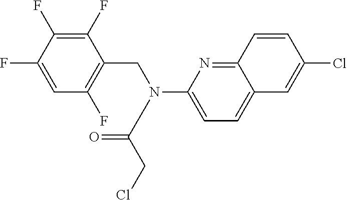 [SMILES: O=C(CCl)N(Cc1c(F)cc(F)c(F)c1F)c1ccc2cc(Cl)ccc2n1] | (Compound 224) |
| 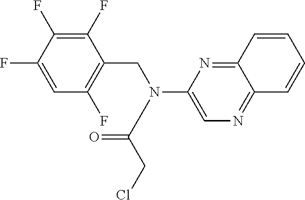 [SMILES: O=C(CCl)N(Cc1c(F)cc(F)c(F)c1F)c1cnc2ccccc2n1] | (Compound 223) |
| 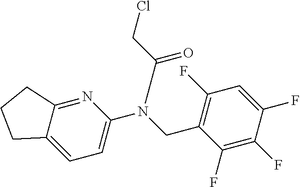 [SMILES: O=C(CCl)N(Cc1c(F)cc(F)c(F)c1F)c1ccc2c(n1)CCC2] | (Compound 226) |
| 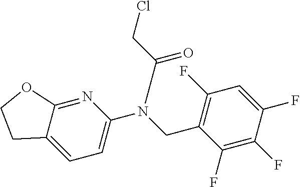 [SMILES: O=C(CCl)N(Cc1c(F)cc(F)c(F)c1F)c1ccc2c(n1)OCC2] | (Compound 225) |
| 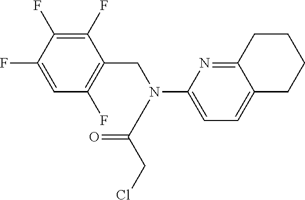 [SMILES: O=C(CCl)N(Cc1c(F)cc(F)c(F)c1F)c1ccc2c(n1)CCCC2] | (Compound 228) |
| 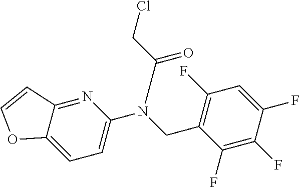 [SMILES: O=C(CCl)N(Cc1c(F)cc(F)c(F)c1F)c1ccc2occc2n1] | (Compound 227) |
| 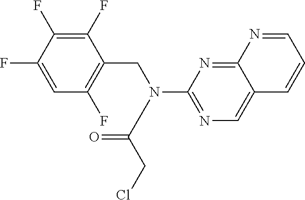 [SMILES: O=C(CCl)N(Cc1c(F)cc(F)c(F)c1F)c1ncc2cccnc2n1] | (Compound 230) |
| 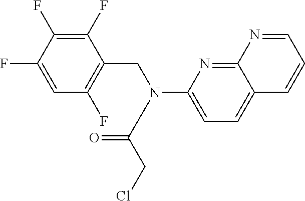 [SMILES: O=C(CCl)N(Cc1c(F)cc(F)c(F)c1F)c1ccc2cccnc2n1] | (Compound 229) |
| 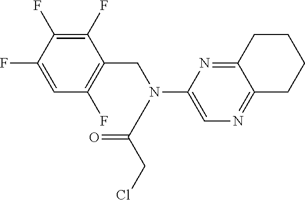 [SMILES: O=C(CCl)N(Cc1c(F)cc(F)c(F)c1F)c1cnc2c(n1)CCCC2] | (Compound 232) |
| 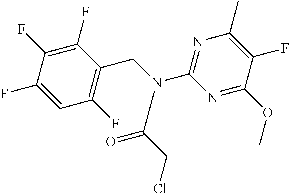 [SMILES: COc1nc(N(Cc2c(F)cc(F)c(F)c2F)C(=O)CCl)nc(C)c1F] | (Compound 231) |
| 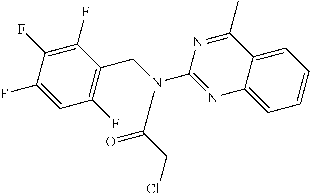 [SMILES: Cc1nc(N(Cc2c(F)cc(F)c(F)c2F)C(=O)CCl)nc2ccccc12] | (Compound 234) |
| 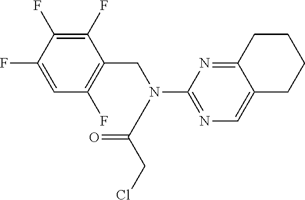 [SMILES: O=C(CCl)N(Cc1c(F)cc(F)c(F)c1F)c1ncc2c(n1)CCCC2] | (Compound 233) |
| 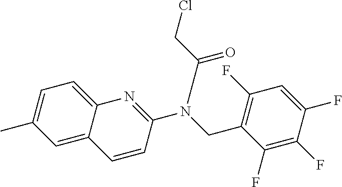 [SMILES: Cc1ccc2nc(N(Cc3c(F)cc(F)c(F)c3F)C(=O)CCl)ccc2c1] | (Compound 236) |
| 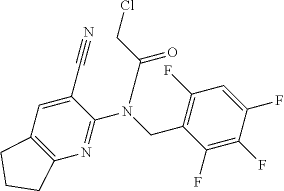 [SMILES: N#Cc1cc2c(nc1N(Cc1c(F)cc(F)c(F)c1F)C(=O)CCl)CCC2] | (Compound 235) |
| 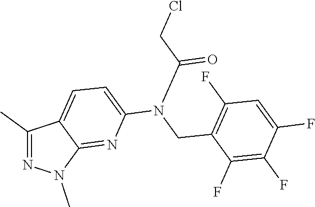 [SMILES: Cc1nn(C)c2nc(N(Cc3c(F)cc(F)c(F)c3F)C(=O)CCl)ccc12] | (Compound 238) |
| 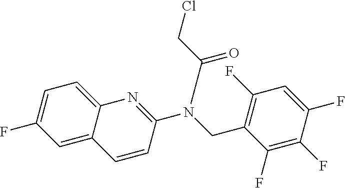 [SMILES: O=C(CCl)N(Cc1c(F)cc(F)c(F)c1F)c1ccc2cc(F)ccc2n1] | (Compound 237) |
| 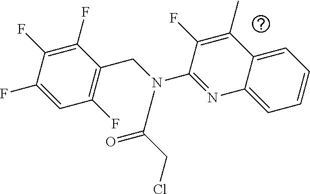 [SMILES: Cc1c(F)c(N(Cc2c(F)cc(F)c(F)c2F)C(=O)CCl)nc2ccccc12] | (Compound 240) |
| 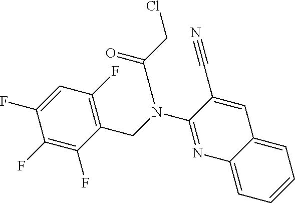 [SMILES: N#Cc1cc2ccccc2nc1N(Cc1c(F)cc(F)c(F)c1F)C(=O)CCl] | (Compound 239) |
| 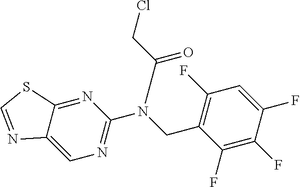 [SMILES: O=C(CCl)N(Cc1c(F)cc(F)c(F)c1F)c1ncc2ncsc2n1] | (Compound 242) |
| 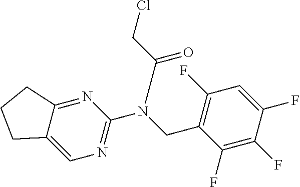 [SMILES: O=C(CCl)N(Cc1c(F)cc(F)c(F)c1F)c1ncc2c(n1)CCC2] | (Compound 241) |
| 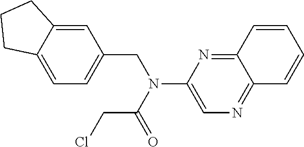 [SMILES: O=C(CCl)N(Cc1ccc2c(c1)CCC2)c1cnc2ccccc2n1] | (Compound 244) |
| 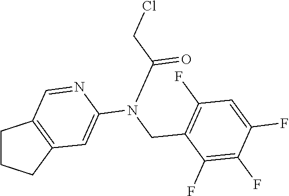 [SMILES: O=C(CCl)N(Cc1c(F)cc(F)c(F)c1F)c1cc2c(cn1)CCC2] | (Compound 243) |
| 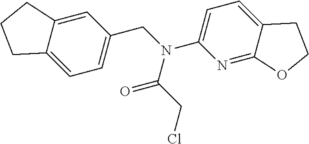 [SMILES: O=C(CCl)N(Cc1ccc2c(c1)CCC2)c1ccc2c(n1)OCC2] | (Compound 246) |
| 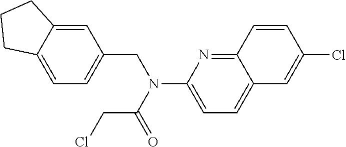 [SMILES: O=C(CCl)N(Cc1ccc2c(c1)CCC2)c1ccc2cc(Cl)ccc2n1] | (Compound 245) |
| 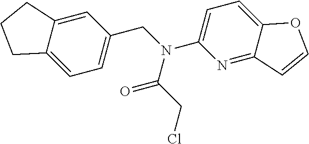 [SMILES: O=C(CCl)N(Cc1ccc2c(c1)CCC2)c1ccc2occc2n1] | (Compound 248) |
| 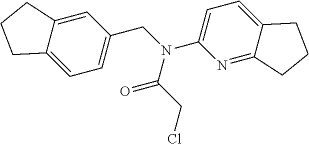 [SMILES: O=C(CCl)N(Cc1ccc2c(c1)CCC2)c1ccc2c(n1)CCC2] | (Compound 247) |
| 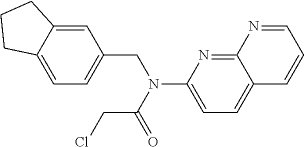 [SMILES: O=C(CCl)N(Cc1ccc2c(c1)CCC2)c1ccc2cccnc2n1] | (Compound 250) |
| 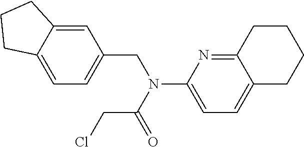 [SMILES: O=C(CCl)N(Cc1ccc2c(c1)CCC2)c1ccc2c(n1)CCCC2] | (Compound 249) |
| 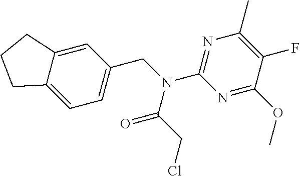 [SMILES: COc1nc(N(Cc2ccc3c(c2)CCC3)C(=O)CCl)nc(C)c1F] | (Compound 252) |
| 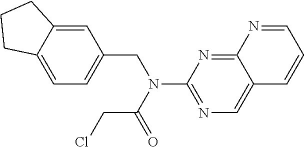 [SMILES: O=C(CCl)N(Cc1ccc2c(c1)CCC2)c1ncc2cccnc2n1] | (Compound 251) |
| 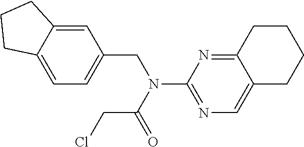 [SMILES: O=C(CCl)N(Cc1ccc2c(c1)CCC2)c1ncc2c(n1)CCCC2] | (Compound 254) |
| 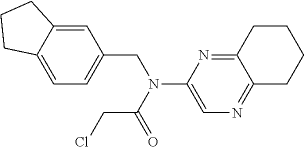 [SMILES: O=C(CCl)N(Cc1ccc2c(c1)CCC2)c1cnc2c(n1)CCCC2] | (Compound 253) |
| 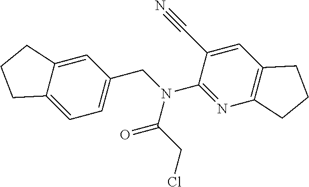 [SMILES: N#Cc1cc2c(nc1N(Cc1ccc3c(c1)CCC3)C(=O)CCl)CCC2] | (Compound 256) |
| 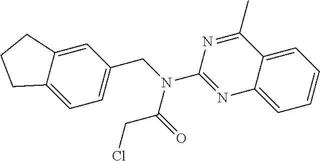 [SMILES: Cc1nc(N(Cc2ccc3c(c2)CCC3)C(=O)CCl)nc2ccccc12] | (Compound 255) |
| 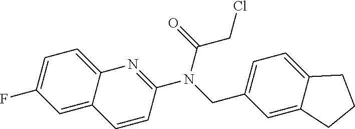 [SMILES: O=C(CCl)N(Cc1ccc2c(c1)CCC2)c1ccc2cc(F)ccc2n1] | (Compound 258) |
| 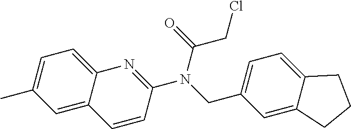 [SMILES: Cc1ccc2nc(N(Cc3ccc4c(c3)CCC4)C(=O)CCl)ccc2c1] | (Compound 257) |
| 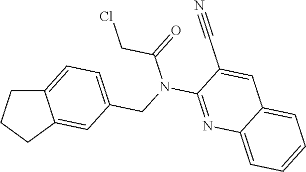 [SMILES: N#Cc1cc2ccccc2nc1N(Cc1ccc2c(c1)CCC2)C(=O)CCl] | (Compound 260) |
| 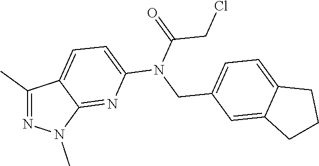 [SMILES: Cc1nn(C)c2nc(N(Cc3ccc4c(c3)CCC4)C(=O)CCl)ccc12] | (Compound 259) |
| 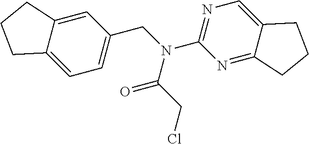 [SMILES: O=C(CCl)N(Cc1ccc2c(c1)CCC2)c1ncc2c(n1)CCC2] | (Compound 262) |
| 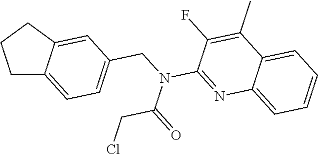 [SMILES: Cc1c(F)c(N(Cc2ccc3c(c2)CCC3)C(=O)CCl)nc2ccccc12] | (Compound 261) |
| 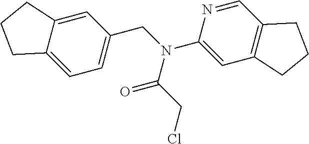 [SMILES: O=C(CCl)N(Cc1ccc2c(c1)CCC2)c1cc2c(cn1)CCC2] | (Compound 264) |
| 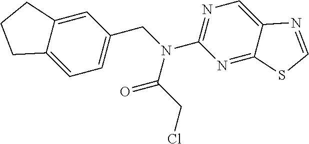 [SMILES: O=C(CCl)N(Cc1ccc2c(c1)CCC2)c1ncc2ncsc2n1] | (Compound 263) |
| 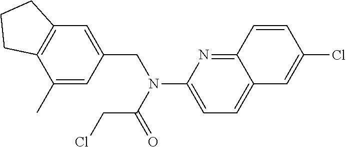 [SMILES: Cc1cc(CN(C(=O)CCl)c2ccc3cc(Cl)ccc3n2)cc2c1CCC2] | (Compound 266) |
| 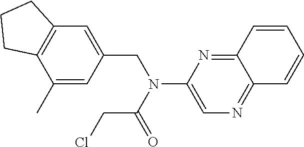 [SMILES: Cc1cc(CN(C(=O)CCl)c2cnc3ccccc3n2)cc2c1CCC2] | (Compound 265) |
| 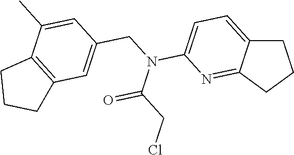 [SMILES: Cc1cc(CN(C(=O)CCl)c2ccc3c(n2)CCC3)cc2c1CCC2] | (Compound 268) |
| 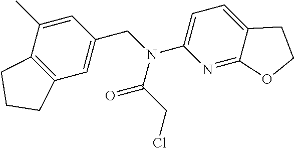 [SMILES: Cc1cc(CN(C(=O)CCl)c2ccc3c(n2)OCC3)cc2c1CCC2] | (Compound 267) |
| 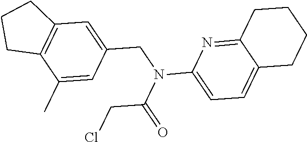 [SMILES: Cc1cc(CN(C(=O)CCl)c2ccc3c(n2)CCCC3)cc2c1CCC2] | (Compound 270) |
| 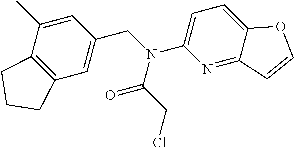 [SMILES: Cc1cc(CN(C(=O)CCl)c2ccc3occc3n2)cc2c1CCC2] | (Compound 269) |
| 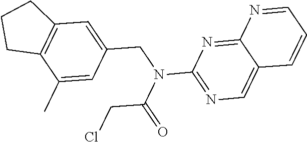 [SMILES: Cc1cc(CN(C(=O)CCl)c2ncc3cccnc3n2)cc2c1CCC2] | (Compound 272) |
| 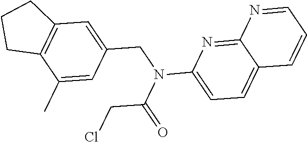 [SMILES: Cc1cc(CN(C(=O)CCl)c2ccc3cccnc3n2)cc2c1CCC2] | (Compound 271) |
| 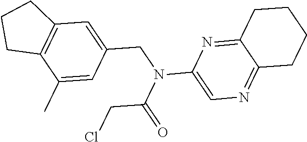 [SMILES: Cc1cc(CN(C(=O)CCl)c2cnc3c(n2)CCCC3)cc2c1CCC2] | (Compound 274) |
| 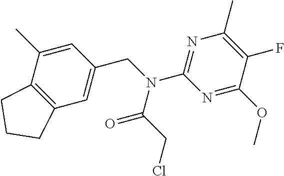 [SMILES: COc1nc(N(Cc2cc(C)c3c(c2)CCC3)C(=O)CCl)nc(C)c1F] | (Compound 273) |
| 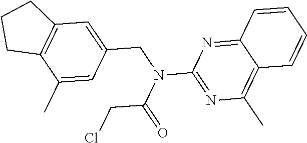 [SMILES: Cc1cc(CN(C(=O)CCl)c2nc(C)c3ccccc3n2)cc2c1CCC2] | (Compound 276) |
| 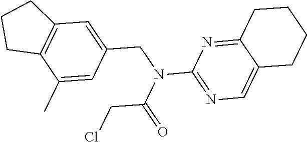 [SMILES: Cc1cc(CN(C(=O)CCl)c2ncc3c(n2)CCCC3)cc2c1CCC2] | (Compound 275) |
| 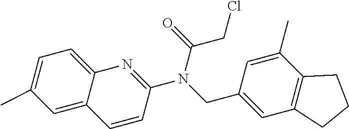 [SMILES: Cc1ccc2nc(N(Cc3cc(C)c4c(c3)CCC4)C(=O)CCl)ccc2c1] | (Compound 278) |
| 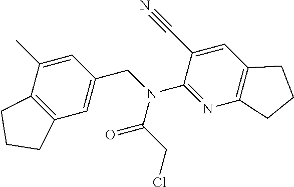 [SMILES: Cc1cc(CN(C(=O)CCl)c2nc3c(cc2C#N)CCC3)cc2c1CCC2] | (Compound 277) |
| 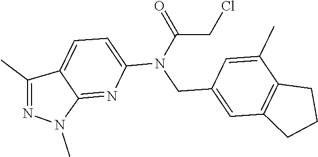 [SMILES: Cc1cc(CN(C(=O)CCl)c2ccc3c(C)nn(C)c3n2)cc2c1CCC2] | (Compound 280) |
| 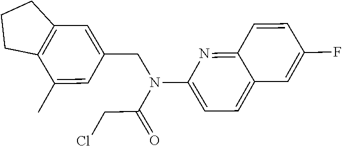 [SMILES: Cc1cc(CN(C(=O)CCl)c2ccc3cc(F)ccc3n2)cc2c1CCC2] | (Compound 279) |
| 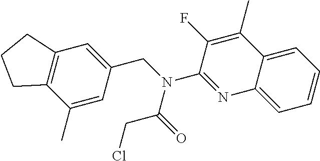 [SMILES: Cc1cc(CN(C(=O)CCl)c2nc3ccccc3c(C)c2F)cc2c1CCC2] | (Compound 282) |
| 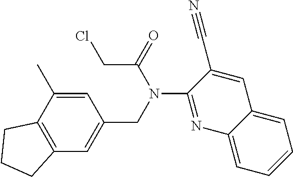 [SMILES: Cc1cc(CN(C(=O)CCl)c2nc3ccccc3cc2C#N)cc2c1CCC2] | (Compound 281) |
| 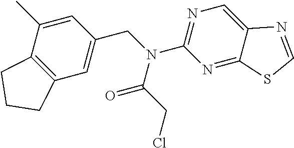 [SMILES: Cc1cc(CN(C(=O)CCl)c2ncc3ncsc3n2)cc2c1CCC2] | (Compound 284) |
| 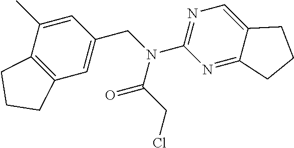 [SMILES: Cc1cc(CN(C(=O)CCl)c2ncc3c(n2)CCC3)cc2c1CCC2] | (Compound 283) |
| 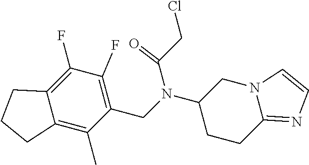 [SMILES: Cc1c(CN(C(=O)CCl)C2CCc3nccn3C2)c(F)c(F)c2c1CCC2] | (Compound 286) |
| 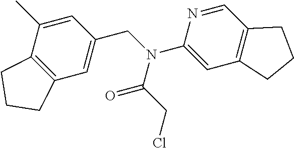 [SMILES: Cc1cc(CN(C(=O)CCl)c2cc3c(cn2)CCC3)cc2c1CCC2] | (Compound 285) |
| 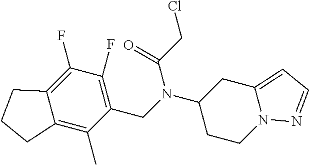 [SMILES: Cc1c(CN(C(=O)CCl)C2CCn3nccc3C2)c(F)c(F)c2c1CCC2] | (Compound 288) |
| 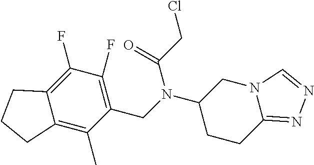 [SMILES: Cc1c(CN(C(=O)CCl)C2CCc3nncn3C2)c(F)c(F)c2c1CCC2] | (Compound 287) |
| 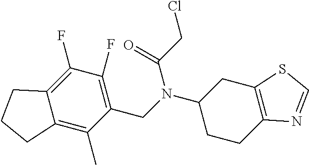 [SMILES: Cc1c(CN(C(=O)CCl)C2CCc3ncsc3C2)c(F)c(F)c2c1CCC2] | (Compound 290) |
| 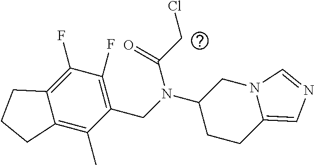 [SMILES: Cc1c(CN(C(=O)CCl)C2CCc3cncn3C2)c(F)c(F)c2c1CCC2] | (Compound 289) |
| 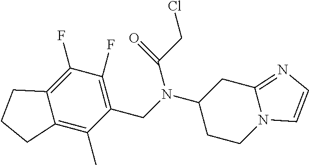 [SMILES: Cc1c(CN(C(=O)CCl)C2CCn3ccnc3C2)c(F)c(F)c2c1CCC2] | (Compound 292) |
| 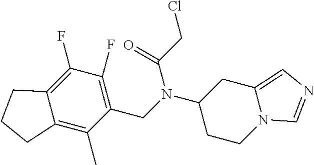 [SMILES: Cc1c(CN(C(=O)CCl)C2CCn3cncc3C2)c(F)c(F)c2c1CCC2] | (Compound 291) |
| 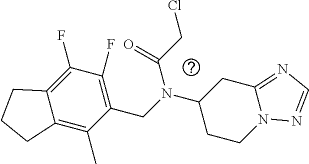 [SMILES: Cc1c(CN(C(=O)CCl)C2CCn3ncnc3C2)c(F)c(F)c2c1CCC2] | (Compound 294) |
| 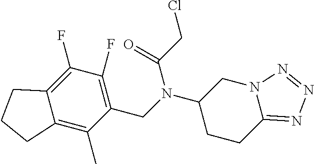 [SMILES: Cc1c(CN(C(=O)CCl)C2CCc3nnnn3C2)c(F)c(F)c2c1CCC2] | (Compound 293) |
| 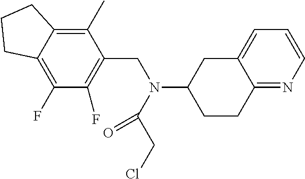 [SMILES: Cc1c(CN(C(=O)CCl)C2CCc3ncccc3C2)c(F)c(F)c2c1CCC2] | (Compound 296) |
| 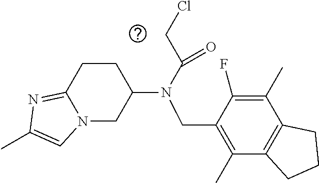 [SMILES: Cc1cn2c(n1)CCC(N(Cc1c(C)c3c(c(C)c1F)CCC3)C(=O)CCl)C2] | (Compound 295) |
| 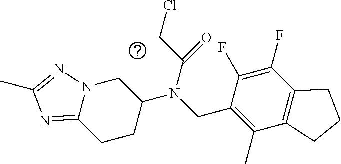 [SMILES: Cc1nc2n(n1)CC(N(Cc1c(C)c3c(c(F)c1F)CCC3)C(=O)CCl)CC2] | (Compound 298) |
| 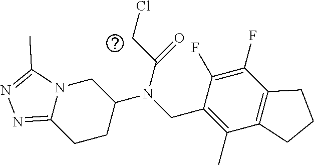 [SMILES: Cc1c(CN(C(=O)CCl)C2CCc3nnc(C)n3C2)c(F)c(F)c2c1CCC2] | (Compound 297) |
| 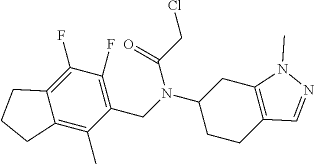 [SMILES: Cc1c(CN(C(=O)CCl)C2CCc3cnn(C)c3C2)c(F)c(F)c2c1CCC2] | (Compound 300) |
| 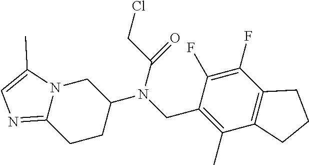 [SMILES: Cc1c(CN(C(=O)CCl)C2CCc3ncc(C)n3C2)c(F)c(F)c2c1CCC2] | (Compound 299) |
| 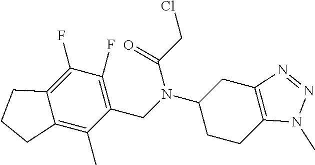 [SMILES: Cc1c(CN(C(=O)CCl)C2CCc3c(nnn3C)C2)c(F)c(F)c2c1CCC2] | (Compound 302) |
| 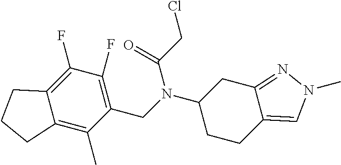 [SMILES: Cc1c(CN(C(=O)CCl)C2CCc3cn(C)nc3C2)c(F)c(F)c2c1CCC2] | (Compound 301) |
| 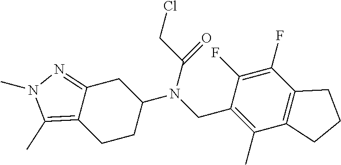 [SMILES: Cc1c(CN(C(=O)CCl)C2CCc3c(nn(C)c3C)C2)c(F)c(F)c2c1CCC2] | (Compound 304) |
| 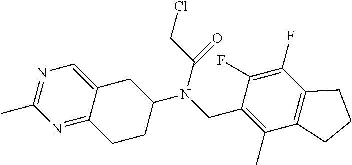 [SMILES: Cc1ncc2c(n1)CCC(N(Cc1c(C)c3c(c(F)c1F)CCC3)C(=O)CCl)C2] | (Compound 303) |
| 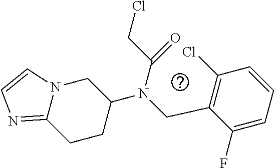 [SMILES: O=C(CCl)N(Cc1c(F)cccc1Cl)C1CCc2nccn2C1] | (Compound 306) |
| 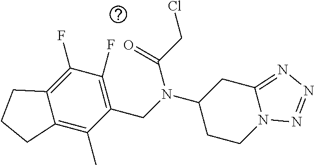 [SMILES: Cc1c(CN(C(=O)CCl)C2CCn3nnnc3C2)c(F)c(F)c2c1CCC2] | (Compound 305) |
| 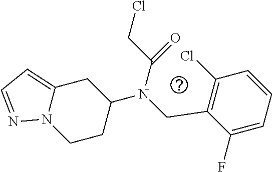 [SMILES: O=C(CCl)N(Cc1c(F)cccc1Cl)C1CCn2nccc2C1] | (Compound 308) |
| 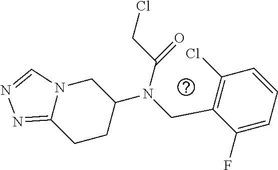 [SMILES: O=C(CCl)N(Cc1c(F)cccc1Cl)C1CCc2nncn2C1] | (Compound 307) |
| 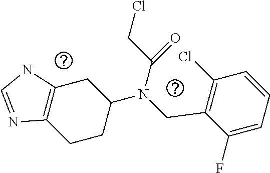 [SMILES: O=C(CCl)N(Cc1c(F)cccc1Cl)C1CCc2nc[nH]c2C1] | (Compound 310) |
| 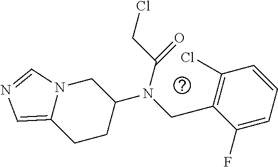 [SMILES: O=C(CCl)N(Cc1c(F)cccc1Cl)C1CCc2cncn2C1] | (Compound 309) |
| 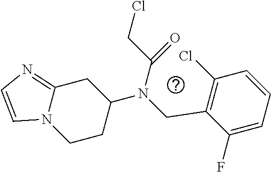 [SMILES: O=C(CCl)N(Cc1c(F)cccc1Cl)C1CCn2ccnc2C1] | (Compound 312) |
| 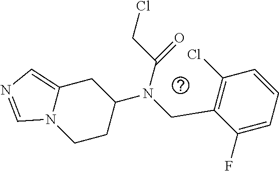 [SMILES: O=C(CCl)N(Cc1c(F)cccc1Cl)C1CCn2cncc2C1] | (Compound 311) |
| 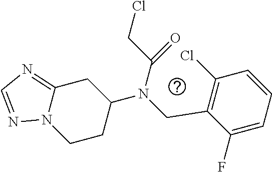 [SMILES: O=C(CCl)N(Cc1c(F)cccc1Cl)C1CCn2ncnc2C1] | (Compound 314) |
| 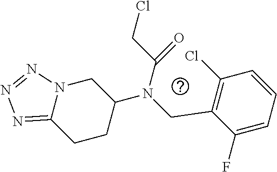 [SMILES: O=C(CCl)N(Cc1c(F)cccc1Cl)C1CCc2nnnn2C1] | (Compound 313) |
| 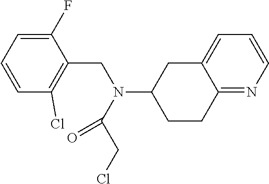 [SMILES: O=C(CCl)N(Cc1c(F)cccc1Cl)C1CCc2ncccc2C1] | (Compound 316) |
| 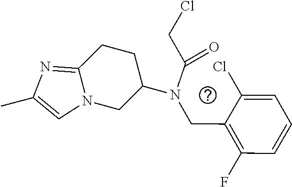 [SMILES: Cc1cn2c(n1)CCC(N(Cc1c(F)cccc1Cl)C(=O)CCl)C2] | (Compound 315) |
| 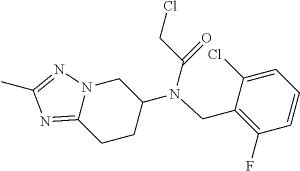 [SMILES: Cc1nc2n(n1)CC(N(Cc1c(F)cccc1Cl)C(=O)CCl)CC2] | (Compound 318) |
| 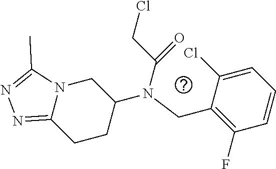 [SMILES: Cc1nnc2n1CC(N(Cc1c(F)cccc1Cl)C(=O)CCl)CC2] | (Compound 317) |
| 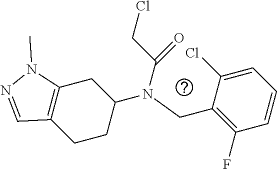 [SMILES: Cn1ncc2c1CC(N(Cc1c(F)cccc1Cl)C(=O)CCl)CC2] | (Compound 320) |
| 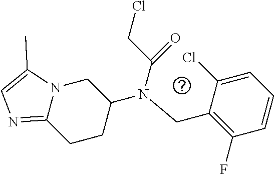 [SMILES: Cc1cnc2n1CC(N(Cc1c(F)cccc1Cl)C(=O)CCl)CC2] | (Compound 319) |
| 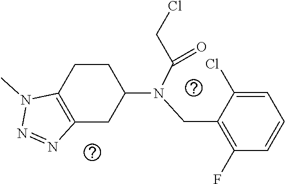 [SMILES: Cn1nnc2c1CCC(N(Cc1c(F)cccc1Cl)C(=O)CCl)C2] | (Compound 322) |
| 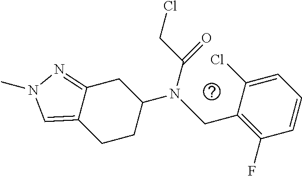 [SMILES: Cn1cc2c(n1)CC(N(Cc1c(F)cccc1Cl)C(=O)CCl)CC2] | (Compound 321) |
| 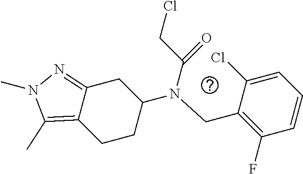 [SMILES: Cc1c2c(nn1C)CC(N(Cc1c(F)cccc1Cl)C(=O)CCl)CC2] | (Compound 324) |
| 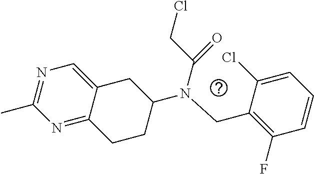 [SMILES: Cc1ncc2c(n1)CCC(N(Cc1c(F)cccc1Cl)C(=O)CCl)C2] | (Compound 323) |
| 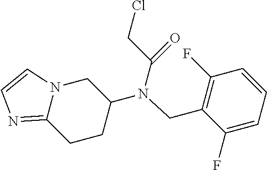 [SMILES: O=C(CCl)N(Cc1c(F)cccc1F)C1CCc2nccn2C1] | (Compound 326) |
| 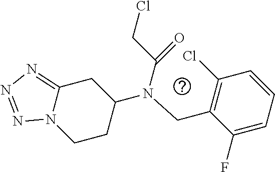 [SMILES: O=C(CCl)N(Cc1c(F)cccc1Cl)C1CCn2nnnc2C1] | (Compound 325) |
| 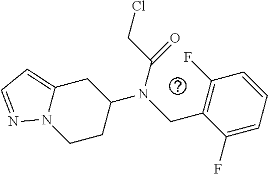 [SMILES: O=C(CCl)N(Cc1c(F)cccc1F)C1CCn2nccc2C1] | (Compound 328) |
| 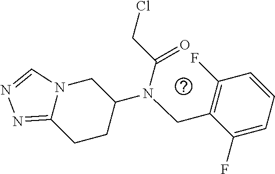 [SMILES: O=C(CCl)N(Cc1c(F)cccc1F)C1CCc2nncn2C1] | (Compound 327) |
| 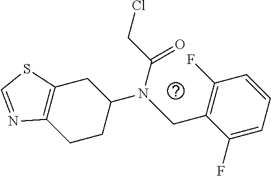 [SMILES: O=C(CCl)N(Cc1c(F)cccc1F)C1CCc2ncsc2C1] | (Compound 330) |
| 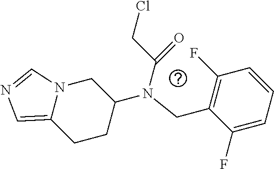 [SMILES: O=C(CCl)N(Cc1c(F)cccc1F)C1CCc2cncn2C1] | (Compound 329) |
| 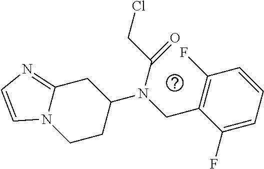 [SMILES: O=C(CCl)N(Cc1c(F)cccc1F)C1CCn2ccnc2C1] | (Compound 332) |
| 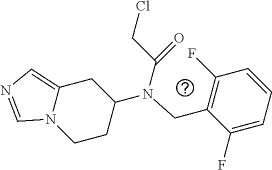 [SMILES: O=C(CCl)N(Cc1c(F)cccc1F)C1CCn2cncc2C1] | (Compound 331) |
| 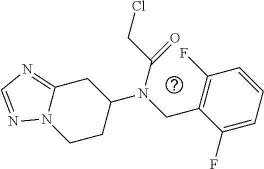 [SMILES: O=C(CCl)N(Cc1c(F)cccc1F)C1CCn2ncnc2C1] | (Compound 334) |
| 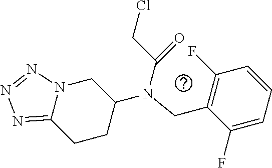 [SMILES: O=C(CCl)N(Cc1c(F)cccc1F)C1CCc2nnnn2C1] | (Compound 333) |
| 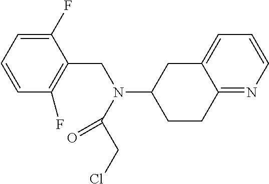 [SMILES: O=C(CCl)N(Cc1c(F)cccc1F)C1CCc2ncccc2C1] | (Compound 336) |
| 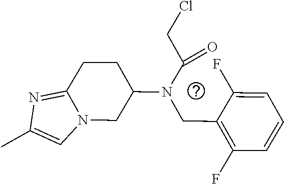 [SMILES: Cc1cn2c(n1)CCC(N(Cc1c(F)cccc1F)C(=O)CCl)C2] | (Compound 335) |
| 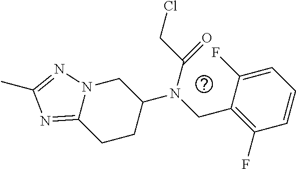 [SMILES: Cc1nc2n(n1)CC(N(Cc1c(F)cccc1F)C(=O)CCl)CC2] | (Compound 338) |
| 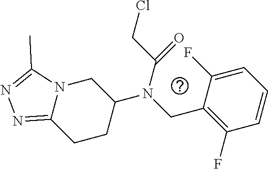 [SMILES: Cc1nnc2n1CC(N(Cc1c(F)cccc1F)C(=O)CCl)CC2] | (Compound 337) |
| 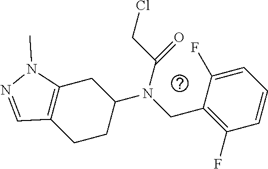 [SMILES: Cn1ncc2c1CC(N(Cc1c(F)cccc1F)C(=O)CCl)CC2] | (Compound 340) |
| 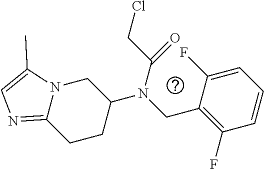 [SMILES: Cc1cnc2n1CC(N(Cc1c(F)cccc1F)C(=O)CCl)CC2] | (Compound 339) |
| 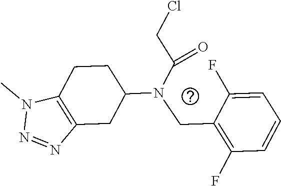 [SMILES: Cn1nnc2c1CCC(N(Cc1c(F)cccc1F)C(=O)CCl)C2] | (Compound 342) |
| 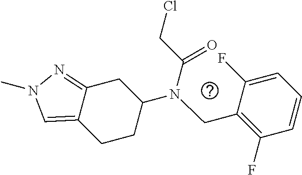 [SMILES: Cn1cc2c(n1)CC(N(Cc1c(F)cccc1F)C(=O)CCl)CC2] | (Compound 341) |
| 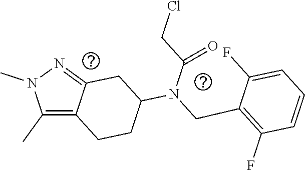 [SMILES: Cc1c2c(nn1C)CC(N(Cc1c(F)cccc1F)C(=O)CCl)CC2] | (Compound 344) |
| 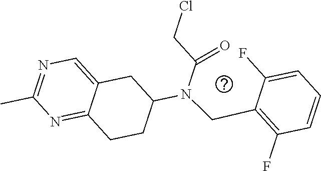 [SMILES: Cc1ncc2c(n1)CCC(N(Cc1c(F)cccc1F)C(=O)CCl)C2] | (Compound 343) |
| 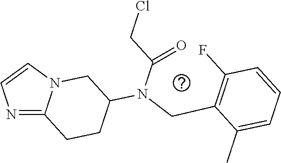 [SMILES: Cc1cccc(F)c1CN(C(=O)CCl)C1CCc2nccn2C1] | (Compound 346) |
| 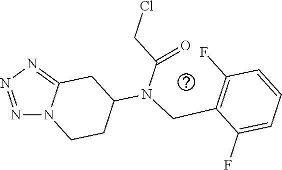 [SMILES: O=C(CCl)N(Cc1c(F)cccc1F)C1CCn2nnnc2C1] | (Compound 345) |
| 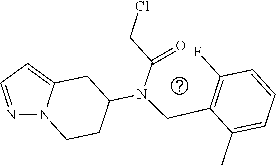 [SMILES: Cc1cccc(F)c1CN(C(=O)CCl)C1CCn2nccc2C1] | (Compound 348) |
| 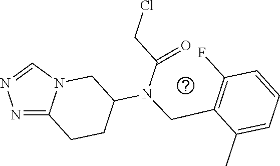 [SMILES: Cc1cccc(F)c1CN(C(=O)CCl)C1CCc2nncn2C1] | (Compound 347) |
| 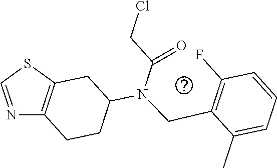 [SMILES: Cc1cccc(F)c1CN(C(=O)CCl)C1CCc2ncsc2C1] | (Compound 350) |
| 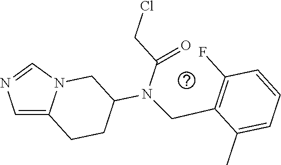 [SMILES: Cc1cccc(F)c1CN(C(=O)CCl)C1CCc2cncn2C1] | (Compound 349) |
| 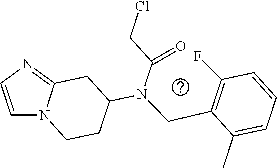 [SMILES: Cc1cccc(F)c1CN(C(=O)CCl)C1CCn2ccnc2C1] | (Compound 352) |
| 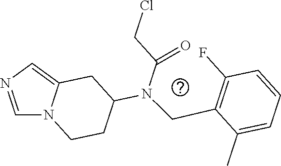 [SMILES: Cc1cccc(F)c1CN(C(=O)CCl)C1CCn2cncc2C1] | (Compound 351) |
| 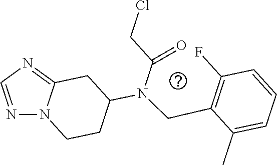 [SMILES: Cc1cccc(F)c1CN(C(=O)CCl)C1CCn2ncnc2C1] | (Compound 354) |
| 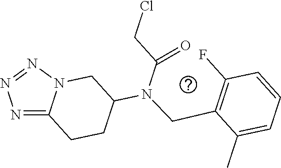 [SMILES: Cc1cccc(F)c1CN(C(=O)CCl)C1CCc2nnnn2C1] | (Compound 353) |
| 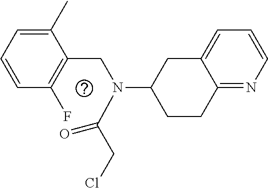 [SMILES: Cc1cccc(F)c1CN(C(=O)CCl)C1CCc2ncccc2C1] | (Compound 356) |
| 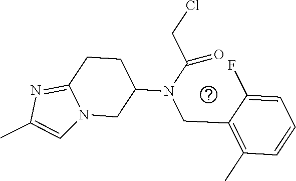 [SMILES: Cc1cn2c(n1)CCC(N(Cc1c(C)cccc1F)C(=O)CCl)C2] | (Compound 355) |
| 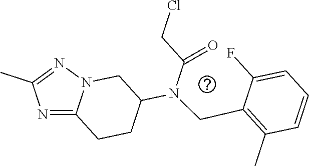 [SMILES: Cc1nc2n(n1)CC(N(Cc1c(C)cccc1F)C(=O)CCl)CC2] | (Compound 358) |
| 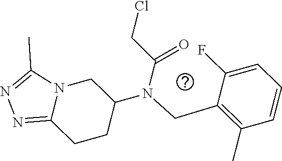 [SMILES: Cc1cccc(F)c1CN(C(=O)CCl)C1CCc2nnc(C)n2C1] | (Compound 357) |
| 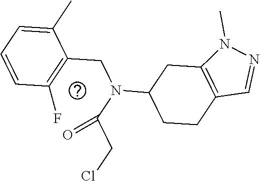 [SMILES: Cc1cccc(F)c1CN(C(=O)CCl)C1CCc2cnn(C)c2C1] | (Compound 360) |
| 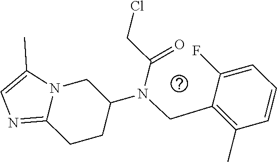 [SMILES: Cc1cccc(F)c1CN(C(=O)CCl)C1CCc2ncc(C)n2C1] | (Compound 359) |
| 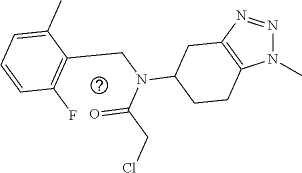 [SMILES: Cc1cccc(F)c1CN(C(=O)CCl)C1CCc2c(nnn2C)C1] | (Compound 362) |
| 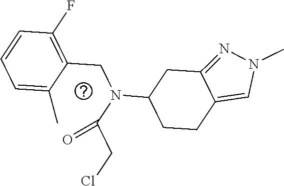 [SMILES: Cc1cccc(F)c1CN(C(=O)CCl)C1CCc2cn(C)nc2C1] | (Compound 361) |
| 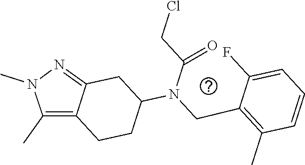 [SMILES: Cc1cccc(F)c1CN(C(=O)CCl)C1CCc2c(nn(C)c2C)C1] | (Compound 364) |
| 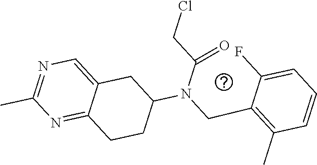 [SMILES: Cc1ncc2c(n1)CCC(N(Cc1c(C)cccc1F)C(=O)CCl)C2] | (Compound 363) |
| 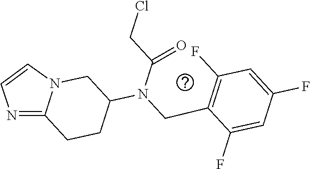 [SMILES: O=C(CCl)N(Cc1c(F)cc(F)cc1F)C1CCc2nccn2C1] | (Compound 366) |
| 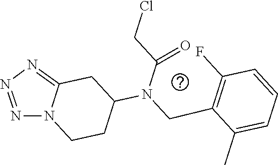 [SMILES: Cc1cccc(F)c1CN(C(=O)CCl)C1CCn2nnnc2C1] | (Compound 365) |
| 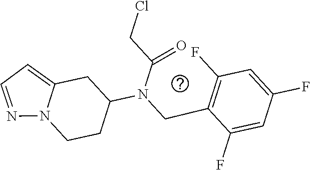 [SMILES: O=C(CCl)N(Cc1c(F)cc(F)cc1F)C1CCn2nccc2C1] | (Compound 368) |
| 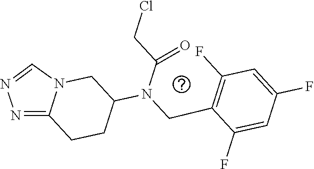 [SMILES: O=C(CCl)N(Cc1c(F)cc(F)cc1F)C1CCc2nncn2C1] | (Compound 367) |
| 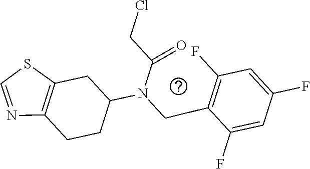 [SMILES: O=C(CCl)N(Cc1c(F)cc(F)cc1F)C1CCc2ncsc2C1] | (Compound 370) |
| 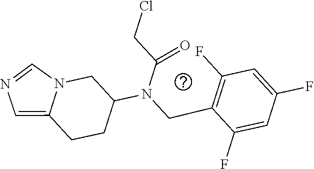 [SMILES: O=C(CCl)N(Cc1c(F)cc(F)cc1F)C1CCc2cncn2C1] | (Compound 369) |
| 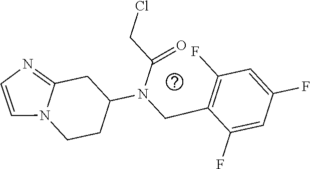 [SMILES: O=C(CCl)N(Cc1c(F)cc(F)cc1F)C1CCn2ccnc2C1] | (Compound 372) |
| 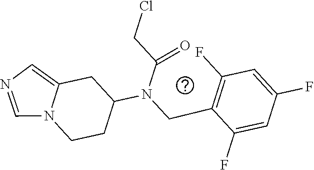 [SMILES: O=C(CCl)N(Cc1c(F)cc(F)cc1F)C1CCn2cncc2C1] | (Compound 371) |
| 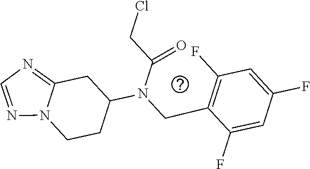 [SMILES: O=C(CCl)N(Cc1c(F)cc(F)cc1F)C1CCn2ncnc2C1] | (Compound 374) |
| 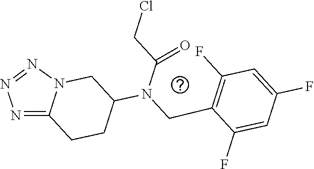 [SMILES: O=C(CCl)N(Cc1c(F)cc(F)cc1F)C1CCc2nnnn2C1] | (Compound 373) |
| 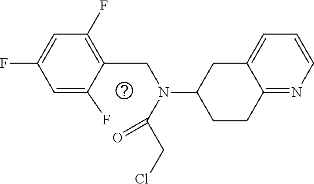 [SMILES: O=C(CCl)N(Cc1c(F)cc(F)cc1F)C1CCc2ncccc2C1] | (Compound 376) |
| 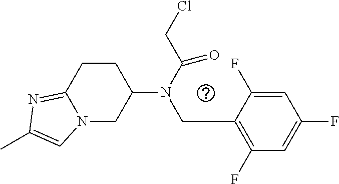 [SMILES: Cc1cn2c(n1)CCC(N(Cc1c(F)cc(F)cc1F)C(=O)CCl)C2] | (Compound 375) |
| 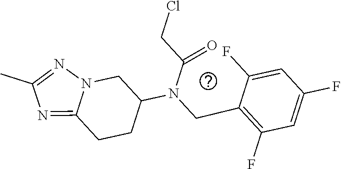 [SMILES: Cc1nc2n(n1)CC(N(Cc1c(F)cc(F)cc1F)C(=O)CCl)CC2] | (Compound 378) |
| 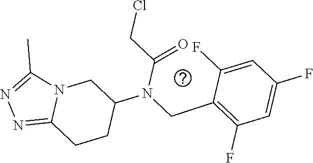 [SMILES: Cc1nnc2n1CC(N(Cc1c(F)cc(F)cc1F)C(=O)CCl)CC2] | (Compound 377) |
| 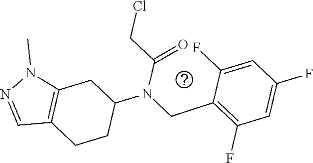 [SMILES: Cn1ncc2c1CC(N(Cc1c(F)cc(F)cc1F)C(=O)CCl)CC2] | (Compound 380) |
| 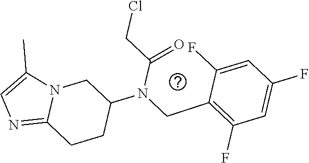 [SMILES: Cc1cnc2n1CC(N(Cc1c(F)cc(F)cc1F)C(=O)CCl)CC2] | (Compound 379) |
| 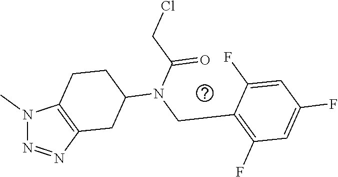 [SMILES: Cn1nnc2c1CCC(N(Cc1c(F)cc(F)cc1F)C(=O)CCl)C2] | (Compound 382) |
| 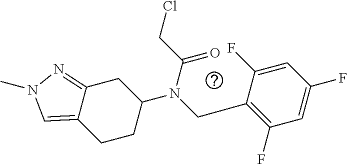 [SMILES: Cn1cc2c(n1)CC(N(Cc1c(F)cc(F)cc1F)C(=O)CCl)CC2] | (Compound 381) |
| 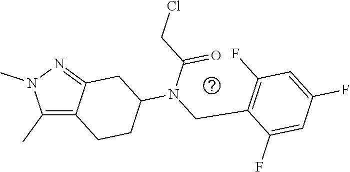 [SMILES: Cc1c2c(nn1C)CC(N(Cc1c(F)cc(F)cc1F)C(=O)CCl)CC2] | (Compound 384) |
| 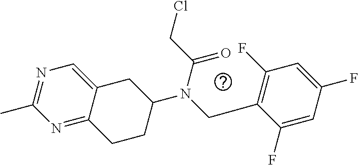 [SMILES: Cc1ncc2c(n1)CCC(N(Cc1c(F)cc(F)cc1F)C(=O)CCl)C2] | (Compound 383) |
| 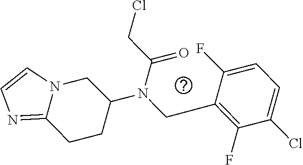 [SMILES: O=C(CCl)N(Cc1c(F)ccc(Cl)c1F)C1CCc2nccn2C1] | (Compound 386) |
| 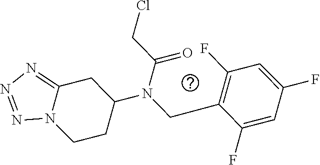 [SMILES: O=C(CCl)N(Cc1c(F)cc(F)cc1F)C1CCn2nnnc2C1] | (Compound 385) |
| 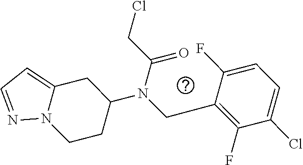 [SMILES: O=C(CCl)N(Cc1c(F)ccc(Cl)c1F)C1CCn2nccc2C1] | (Compound 388) |
| 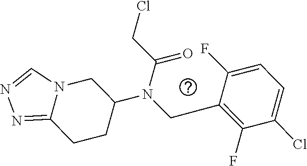 [SMILES: O=C(CCl)N(Cc1c(F)ccc(Cl)c1F)C1CCc2nncn2C1] | (Compound 387) |
| 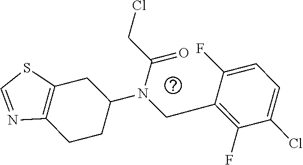 [SMILES: O=C(CCl)N(Cc1c(F)ccc(Cl)c1F)C1CCc2ncsc2C1] | (Compound 390) |
| 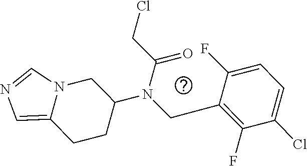 [SMILES: O=C(CCl)N(Cc1c(F)ccc(Cl)c1F)C1CCc2cncn2C1] | (Compound 389) |
| 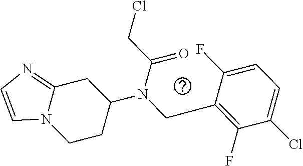 [SMILES: O=C(CCl)N(Cc1c(F)ccc(Cl)c1F)C1CCn2ccnc2C1] | (Compound 392) |
| 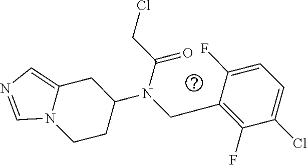 [SMILES: O=C(CCl)N(Cc1c(F)ccc(Cl)c1F)C1CCn2cncc2C1] | (Compound 391) |
| 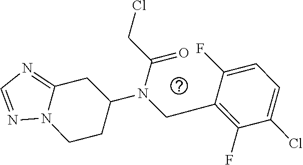 [SMILES: O=C(CCl)N(Cc1c(F)ccc(Cl)c1F)C1CCn2ncnc2C1] | (Compound 394) |
| 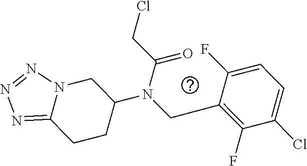 [SMILES: O=C(CCl)N(Cc1c(F)ccc(Cl)c1F)C1CCc2nnnn2C1] | (Compound 393) |
| 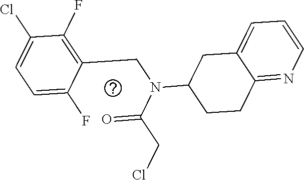 [SMILES: O=C(CCl)N(Cc1c(F)ccc(Cl)c1F)C1CCc2ncccc2C1] | (Compound 396) |
| 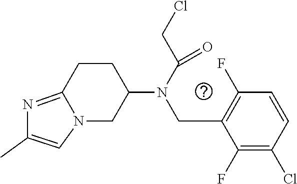 [SMILES: Cc1cn2c(n1)CCC(N(Cc1c(F)ccc(Cl)c1F)C(=O)CCl)C2] | (Compound 395) |
| 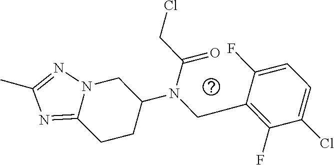 [SMILES: Cc1nc2n(n1)CC(N(Cc1c(F)ccc(Cl)c1F)C(=O)CCl)CC2] | (Compound 398) |
| 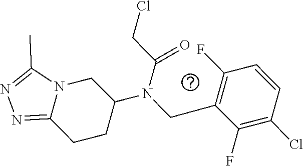 [SMILES: Cc1nnc2n1CC(N(Cc1c(F)ccc(Cl)c1F)C(=O)CCl)CC2] | (Compound 397) |
| 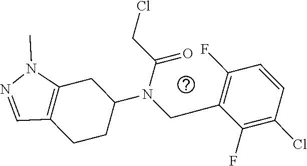 [SMILES: Cn1ncc2c1CC(N(Cc1c(F)ccc(Cl)c1F)C(=O)CCl)CC2] | (Compound 400) |
| 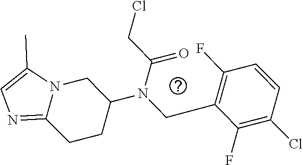 [SMILES: Cc1cnc2n1CC(N(Cc1c(F)ccc(Cl)c1F)C(=O)CCl)CC2] | (Compound 399) |
| 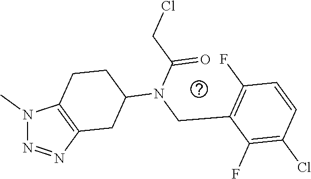 [SMILES: Cn1nnc2c1CCC(N(Cc1c(F)ccc(Cl)c1F)C(=O)CCl)C2] | (Compound 402) |
| 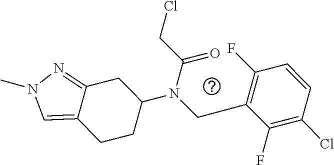 [SMILES: Cn1cc2c(n1)CC(N(Cc1c(F)ccc(Cl)c1F)C(=O)CCl)CC2] | (Compound 401) |
| 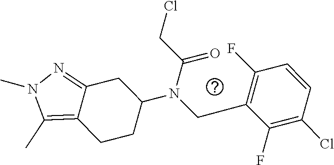 [SMILES: Cc1c2c(nn1C)CC(N(Cc1c(F)ccc(Cl)c1F)C(=O)CCl)CC2] | (Compound 404) |
| 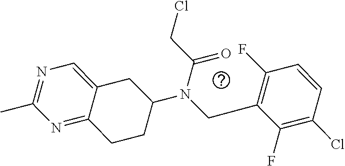 [SMILES: Cc1ncc2c(n1)CCC(N(Cc1c(F)ccc(Cl)c1F)C(=O)CCl)C2] | (Compound 403) |
| 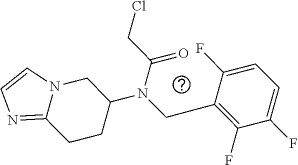 [SMILES: O=C(CCl)N(Cc1c(F)ccc(F)c1F)C1CCc2nccn2C1] | (Compound 406) |
| 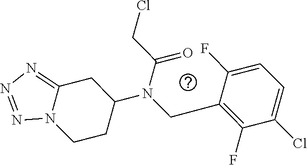 [SMILES: O=C(CCl)N(Cc1c(F)ccc(Cl)c1F)C1CCn2nnnc2C1] | (Compound 405) |
| 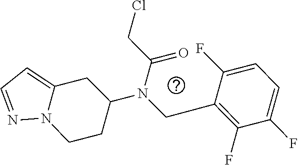 [SMILES: O=C(CCl)N(Cc1c(F)ccc(F)c1F)C1CCn2nccc2C1] | (Compound 408) |
| 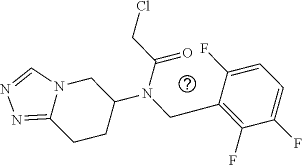 [SMILES: O=C(CCl)N(Cc1c(F)ccc(F)c1F)C1CCc2nncn2C1] | (Compound 407) |
| 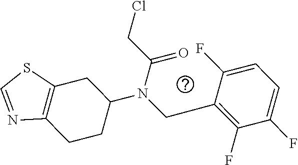 [SMILES: O=C(CCl)N(Cc1c(F)ccc(F)c1F)C1CCc2ncsc2C1] | (Compound 410) |
| 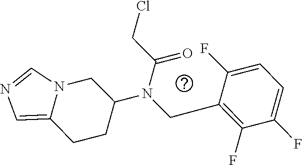 [SMILES: O=C(CCl)N(Cc1c(F)ccc(F)c1F)C1CCc2cncn2C1] | (Compound 409) |
| 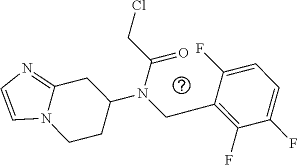 [SMILES: O=C(CCl)N(Cc1c(F)ccc(F)c1F)C1CCn2ccnc2C1] | (Compound 412) |
| 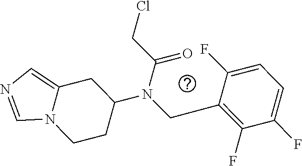 [SMILES: O=C(CCl)N(Cc1c(F)ccc(F)c1F)C1CCn2cncc2C1] | (Compound 411) |
| 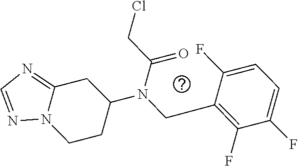 [SMILES: O=C(CCl)N(Cc1c(F)ccc(F)c1F)C1CCn2ncnc2C1] | (Compound 414) |
| 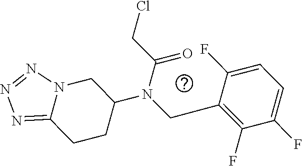 [SMILES: O=C(CCl)N(Cc1c(F)ccc(F)c1F)C1CCc2nnnn2C1] | (Compound 413) |
| 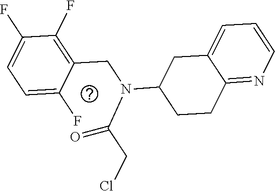 [SMILES: O=C(CCl)N(Cc1c(F)ccc(F)c1F)C1CCc2ncccc2C1] | (Compound 416) |
| 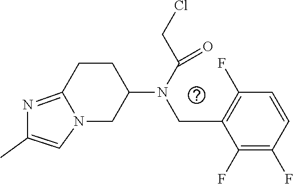 [SMILES: Cc1cn2c(n1)CCC(N(Cc1c(F)ccc(F)c1F)C(=O)CCl)C2] | (Compound 415) |
| 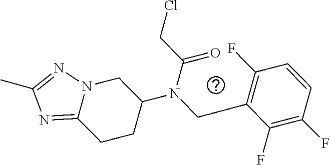 [SMILES: Cc1nc2n(n1)CC(N(Cc1c(F)ccc(F)c1F)C(=O)CCl)CC2] | (Compound 418) |
| 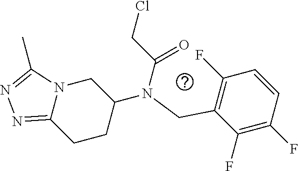 [SMILES: Cc1nnc2n1CC(N(Cc1c(F)ccc(F)c1F)C(=O)CCl)CC2] | (Compound 417) |
| 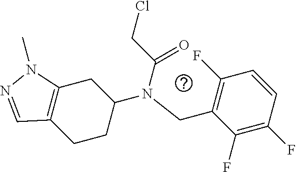 [SMILES: Cn1ncc2c1CC(N(Cc1c(F)ccc(F)c1F)C(=O)CCl)CC2] | (Compound 420) |
| 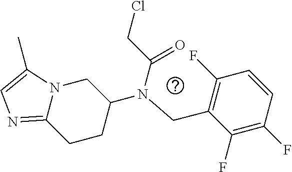 [SMILES: Cc1cnc2n1CC(N(Cc1c(F)ccc(F)c1F)C(=O)CCl)CC2] | (Compound 419) |
| 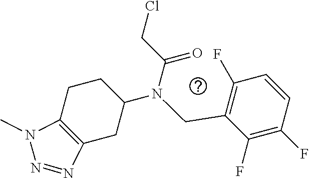 [SMILES: Cn1nnc2c1CCC(N(Cc1c(F)ccc(F)c1F)C(=O)CCl)C2] | (Compound 422) |
| 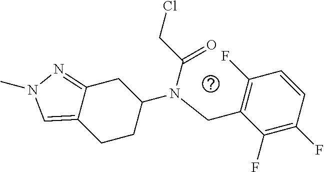 [SMILES: Cn1cc2c(n1)CC(N(Cc1c(F)ccc(F)c1F)C(=O)CCl)CC2] | (Compound 421) |
| 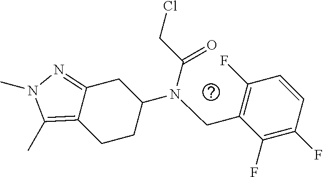 [SMILES: Cc1c2c(nn1C)CC(N(Cc1c(F)ccc(F)c1F)C(=O)CCl)CC2] | (Compound 424) |
| 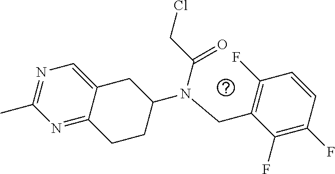 [SMILES: Cc1ncc2c(n1)CCC(N(Cc1c(F)ccc(F)c1F)C(=O)CCl)C2] | (Compound 423) |
| 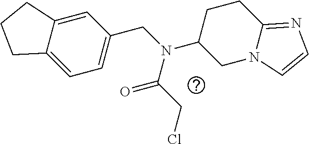 [SMILES: O=C(CCl)N(Cc1ccc2c(c1)CCC2)C1CCc2nccn2C1] | (Compound 426) |
| 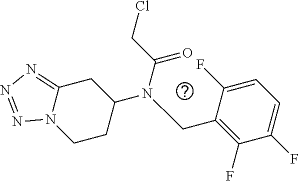 [SMILES: O=C(CCl)N(Cc1c(F)ccc(F)c1F)C1CCn2nnnc2C1] | (Compound 425) |
| 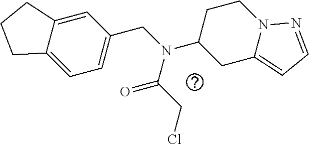 [SMILES: O=C(CCl)N(Cc1ccc2c(c1)CCC2)C1CCn2nccc2C1] | (Compound 428) |
| 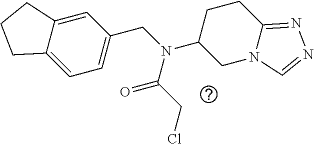 [SMILES: O=C(CCl)N(Cc1ccc2c(c1)CCC2)C1CCc2nncn2C1] | (Compound 427) |
| 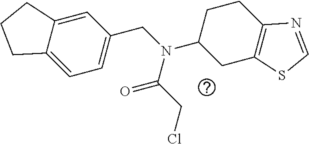 [SMILES: O=C(CCl)N(Cc1ccc2c(c1)CCC2)C1CCc2ncsc2C1] | (Compound 430) |
| 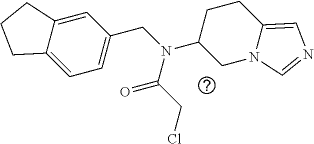 [SMILES: O=C(CCl)N(Cc1ccc2c(c1)CCC2)C1CCc2cncn2C1] | (Compound 429) |
| 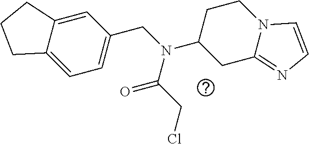 [SMILES: O=C(CCl)N(Cc1ccc2c(c1)CCC2)C1CCn2ccnc2C1] | (Compound 432) |
| 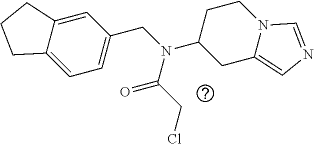 [SMILES: O=C(CCl)N(Cc1ccc2c(c1)CCC2)C1CCn2cncc2C1] | (Compound 431) |
| 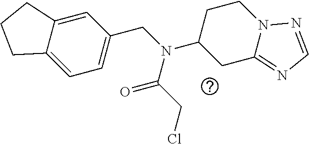 [SMILES: O=C(CCl)N(Cc1ccc2c(c1)CCC2)C1CCn2ncnc2C1] | (Compound 434) |
| 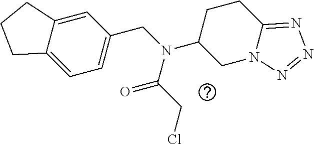 [SMILES: O=C(CCl)N(Cc1ccc2c(c1)CCC2)C1CCc2nnnn2C1] | (Compound 433) |
| 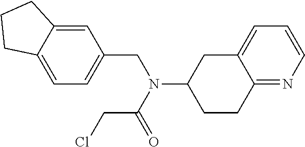 [SMILES: O=C(CCl)N(Cc1ccc2c(c1)CCC2)C1CCc2ncccc2C1] | (Compound 436) |
| 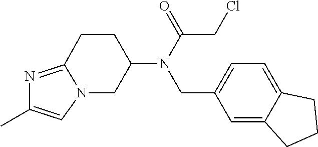 [SMILES: Cc1cn2c(n1)CCC(N(Cc1ccc3c(c1)CCC3)C(=O)CCl)C2] | (Compound 435) |
| 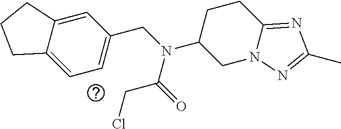 [SMILES: Cc1nc2n(n1)CC(N(Cc1ccc3c(c1)CCC3)C(=O)CCl)CC2] | (Compound 438) |
| 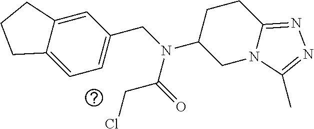 [SMILES: Cc1nnc2n1CC(N(Cc1ccc3c(c1)CCC3)C(=O)CCl)CC2] | (Compound 437) |
| 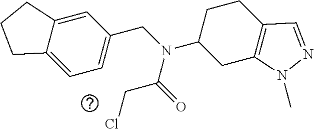 [SMILES: Cn1ncc2c1CC(N(Cc1ccc3c(c1)CCC3)C(=O)CCl)CC2] | (Compound 440) |
| 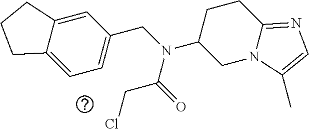 [SMILES: Cc1cnc2n1CC(N(Cc1ccc3c(c1)CCC3)C(=O)CCl)CC2] | (Compound 439) |
| 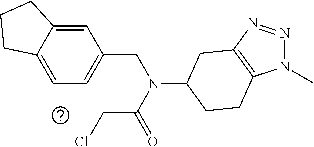 [SMILES: Cn1nnc2c1CCC(N(Cc1ccc3c(c1)CCC3)C(=O)CCl)C2] | (Compound 442) |
| 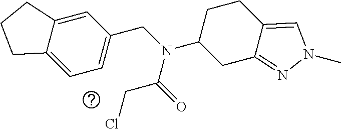 [SMILES: Cn1cc2c(n1)CC(N(Cc1ccc3c(c1)CCC3)C(=O)CCl)CC2] | (Compound 441) |
| 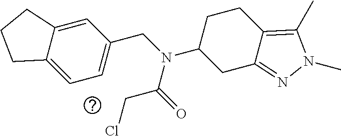 [SMILES: Cc1c2c(nn1C)CC(N(Cc1ccc3c(c1)CCC3)C(=O)CCl)CC2] | (Compound 444) |
| 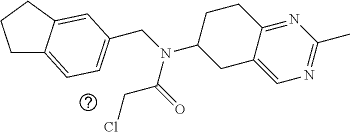 [SMILES: Cc1ncc2c(n1)CCC(N(Cc1ccc3c(c1)CCC3)C(=O)CCl)C2] | (Compound 443) |
| 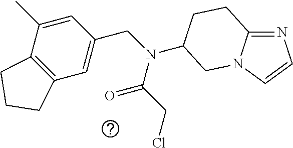 [SMILES: Cc1cc(CN(C(=O)CCl)C2CCc3nccn3C2)cc2c1CCC2] | (Compound 446) |
| 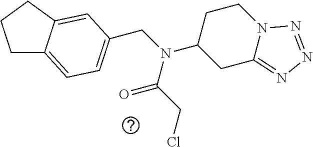 [SMILES: O=C(CCl)N(Cc1ccc2c(c1)CCC2)C1CCn2nnnc2C1] | (Compound 445) |
| 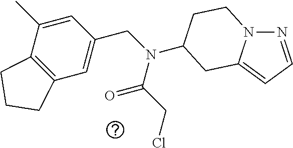 [SMILES: Cc1cc(CN(C(=O)CCl)C2CCn3nccc3C2)cc2c1CCC2] | (Compound 448) |
| 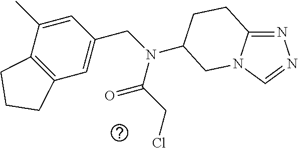 [SMILES: Cc1cc(CN(C(=O)CCl)C2CCc3nncn3C2)cc2c1CCC2] | (Compound 447) |
| 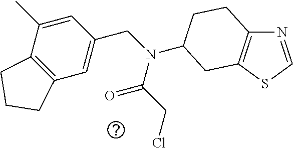 [SMILES: Cc1cc(CN(C(=O)CCl)C2CCc3ncsc3C2)cc2c1CCC2] | (Compound 450) |
| 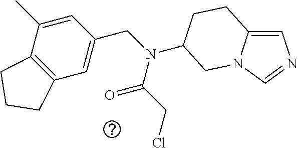 [SMILES: Cc1cc(CN(C(=O)CCl)C2CCc3cncn3C2)cc2c1CCC2] | (Compound 449) |
| 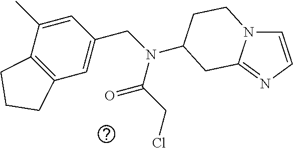 [SMILES: Cc1cc(CN(C(=O)CCl)C2CCn3ccnc3C2)cc2c1CCC2] | (Compound 452) |
| 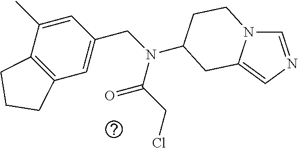 [SMILES: Cc1cc(CN(C(=O)CCl)C2CCn3cncc3C2)cc2c1CCC2] | (Compound 451) |
| 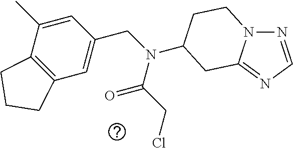 [SMILES: Cc1cc(CN(C(=O)CCl)C2CCn3ncnc3C2)cc2c1CCC2] | (Compound 454) |
| 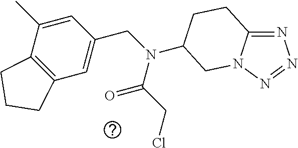 [SMILES: Cc1cc(CN(C(=O)CCl)C2CCc3nnnn3C2)cc2c1CCC2] | (Compound 453) |
| 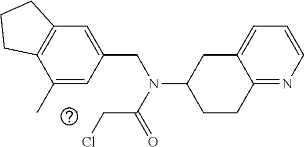 [SMILES: Cc1cc(CN(C(=O)CCl)C2CCc3ncccc3C2)cc2c1CCC2] | (Compound 456) |
| 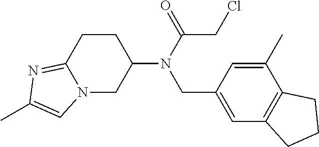 [SMILES: Cc1cn2c(n1)CCC(N(Cc1cc(C)c3c(c1)CCC3)C(=O)CCl)C2] | (Compound 455) |
| 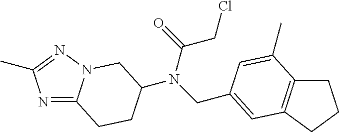 [SMILES: Cc1nc2n(n1)CC(N(Cc1cc(C)c3c(c1)CCC3)C(=O)CCl)CC2] | (Compound 458) |
| 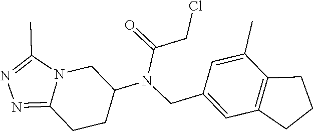 [SMILES: Cc1cc(CN(C(=O)CCl)C2CCc3nnc(C)n3C2)cc2c1CCC2] | (Compound 457) |
| 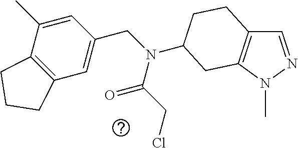 [SMILES: Cc1cc(CN(C(=O)CCl)C2CCc3cnn(C)c3C2)cc2c1CCC2] | (Compound 460) |
| 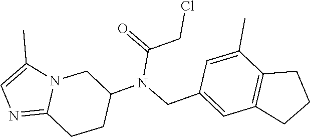 [SMILES: Cc1cc(CN(C(=O)CCl)C2CCc3ncc(C)n3C2)cc2c1CCC2] | (Compound 459) |
| 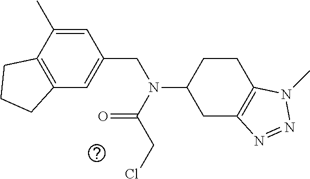 [SMILES: Cc1cc(CN(C(=O)CCl)C2CCc3c(nnn3C)C2)cc2c1CCC2] | (Compound 462) |
| 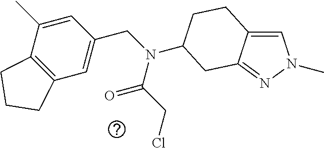 [SMILES: Cc1cc(CN(C(=O)CCl)C2CCc3cn(C)nc3C2)cc2c1CCC2] | (Compound 461) |
| 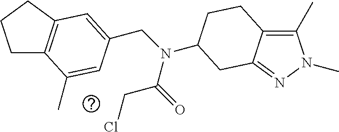 [SMILES: Cc1cc(CN(C(=O)CCl)C2CCc3c(nn(C)c3C)C2)cc2c1CCC2] | (Compound 464) |
| 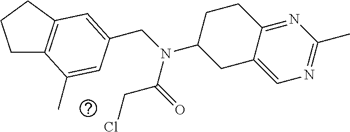 [SMILES: Cc1ncc2c(n1)CCC(N(Cc1cc(C)c3c(c1)CCC3)C(=O)CCl)C2] | (Compound 463) |
| 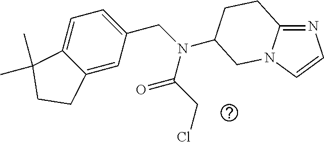 [SMILES: CC1(C)CCc2cc(CN(C(=O)CCl)C3CCc4nccn4C3)ccc21] | (Compound 466) |
| 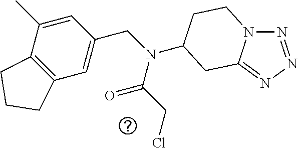 [SMILES: Cc1cc(CN(C(=O)CCl)C2CCn3nnnc3C2)cc2c1CCC2] | (Compound 465) |
| 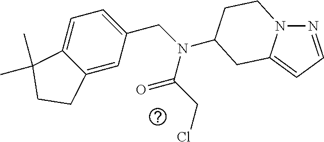 [SMILES: CC1(C)CCc2cc(CN(C(=O)CCl)C3CCn4nccc4C3)ccc21] | (Compound 468) |
| 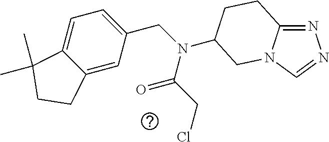 [SMILES: CC1(C)CCc2cc(CN(C(=O)CCl)C3CCc4nncn4C3)ccc21] | (Compound 467) |
| 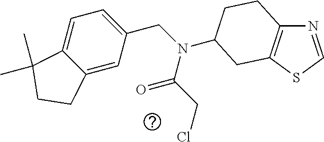 [SMILES: CC1(C)CCc2cc(CN(C(=O)CCl)C3CCc4ncsc4C3)ccc21] | (Compound 470) |
| 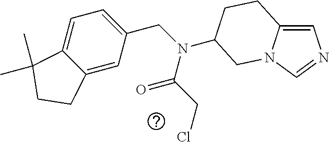 [SMILES: CC1(C)CCc2cc(CN(C(=O)CCl)C3CCc4cncn4C3)ccc21] | (Compound 469) |
| 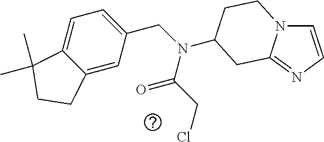 [SMILES: CC1(C)CCc2cc(CN(C(=O)CCl)C3CCn4ccnc4C3)ccc21] | (Compound 472) |
| 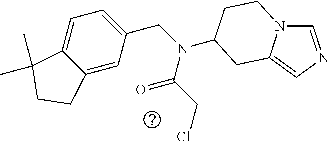 [SMILES: CC1(C)CCc2cc(CN(C(=O)CCl)C3CCn4cncc4C3)ccc21] | (Compound 471) |
| 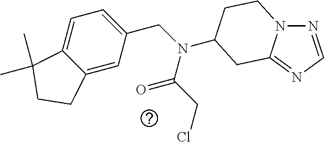 [SMILES: CC1(C)CCc2cc(CN(C(=O)CCl)C3CCn4ncnc4C3)ccc21] | (Compound 474) |
| 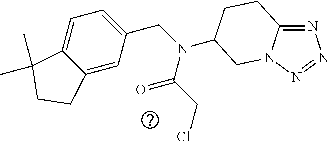 [SMILES: CC1(C)CCc2cc(CN(C(=O)CCl)C3CCc4nnnn4C3)ccc21] | (Compound 473) |
| 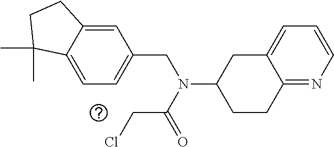 [SMILES: CC1(C)CCc2cc(CN(C(=O)CCl)C3CCc4ncccc4C3)ccc21] | (Compound 476) |
| 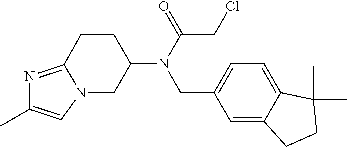 | (Compound 475) |
| 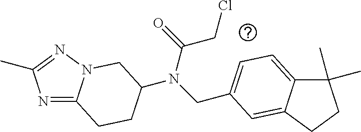 | (Compound 478) |
| 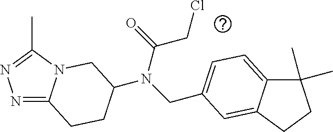 [SMILES: Cc1nnc2n1CC(N(Cc1ccc3c(c1)CCC3(C)C)C(=O)CCl)CC2] | (Compound 477) |
| 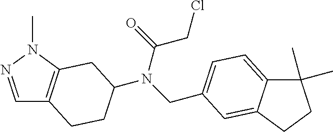 [SMILES: Cn1ncc2c1CC(N(Cc1ccc3c(c1)CCC3(C)C)C(=O)CCl)CC2] | (Compound 480) |
| 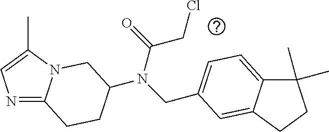 [SMILES: Cc1cnc2n1CC(N(Cc1ccc3c(c1)CCC3(C)C)C(=O)CCl)CC2] | (Compound 479) |
| 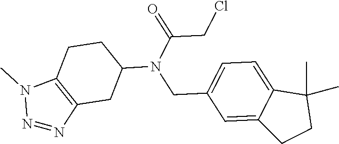 | (Compound 482) |
| 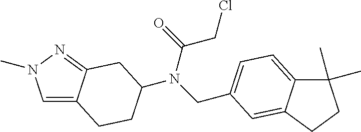 | (Compound 481) |
| 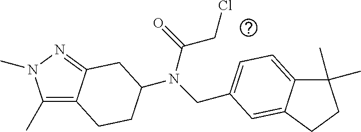 | (Compound 484) |
| 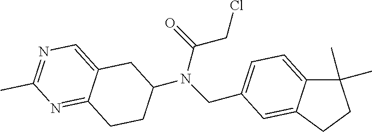 | (Compound 483) |
| 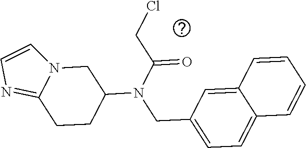 [SMILES: O=C(CCl)N(Cc1ccc2ccccc2c1)C1CCc2nccn2C1] | (Compound 486) |
| 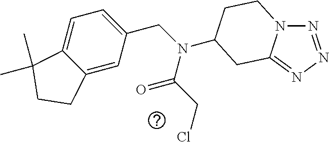 [SMILES: CC1(C)CCc2cc(CN(C(=O)CCl)C3CCn4nnnc4C3)ccc21] | (Compound 485) |
| 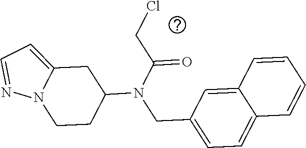 [SMILES: O=C(CCl)N(Cc1ccc2ccccc2c1)C1CCn2nccc2C1] | (Compound 488) |
| 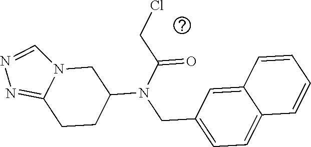 [SMILES: O=C(CCl)N(Cc1ccc2ccccc2c1)C1CCc2nncn2C1] | (Compound 487) |
| 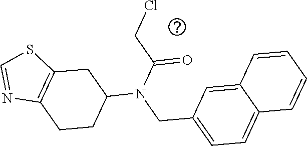 [SMILES: O=C(CCl)N(Cc1ccc2ccccc2c1)C1CCc2ncsc2C1] | (Compound 490) |
| 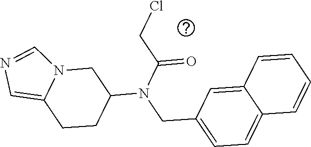 [SMILES: O=C(CCl)N(Cc1ccc2ccccc2c1)C1CCc2cncn2C1] | (Compound 489) |
| 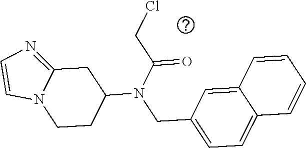 [SMILES: O=C(CCl)N(Cc1ccc2ccccc2c1)C1CCn2ccnc2C1] | (Compound 492) |
| 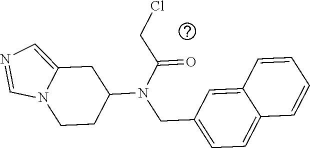 [SMILES: O=C(CCl)N(Cc1ccc2ccccc2c1)C1CCn2cncc2C1] | (Compound 491) |
| 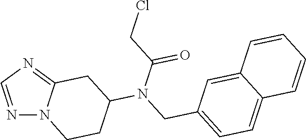 [SMILES: O=C(CCl)N(Cc1ccc2ccccc2c1)C1CCn2ncnc2C1] | (Compound 494) |
| 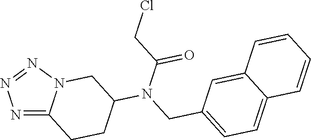 [SMILES: O=C(CCl)N(Cc1ccc2ccccc2c1)C1CCc2nnnn2C1] | (Compound 493) |
| 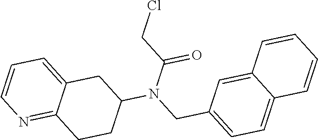 [SMILES: O=C(CCl)N(Cc1ccc2ccccc2c1)C1CCc2ncccc2C1] | (Compound 496) |
| 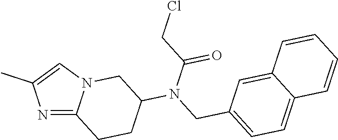 [SMILES: Cc1cn2c(n1)CCC(N(Cc1ccc3ccccc3c1)C(=O)CCl)C2] | (Compound 495) |
| 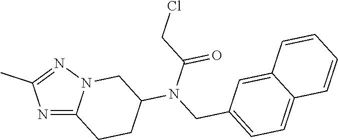 [SMILES: Cc1nc2n(n1)CC(N(Cc1ccc3ccccc3c1)C(=O)CCl)CC2] | (Compound 498) |
| 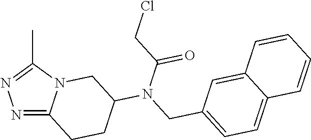 [SMILES: Cc1nnc2n1CC(N(Cc1ccc3ccccc3c1)C(=O)CCl)CC2] | (Compound 497) |
| 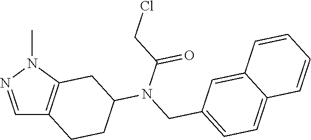 [SMILES: Cn1ncc2c1CC(N(Cc1ccc3ccccc3c1)C(=O)CCl)CC2] | (Compound 500) |
| 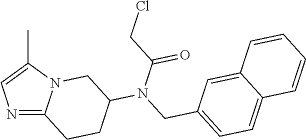 [SMILES: Cc1cnc2n1CC(N(Cc1ccc3ccccc3c1)C(=O)CCl)CC2] | (Compound 499) |
| 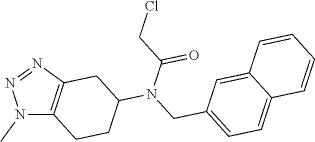 [SMILES: Cn1nnc2c1CCC(N(Cc1ccc3ccccc3c1)C(=O)CCl)C2] | (Compound 502) |
| 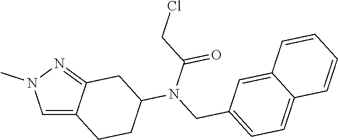 [SMILES: Cn1cc2c(n1)CC(N(Cc1ccc3ccccc3c1)C(=O)CCl)CC2] | (Compound 501) |
| 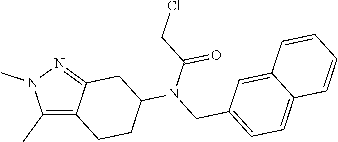 [SMILES: Cc1c2c(nn1C)CC(N(Cc1ccc3ccccc3c1)C(=O)CCl)CC2] | (Compound 504) |
| 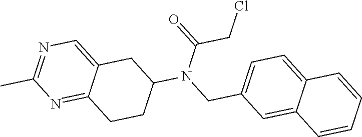 [SMILES: Cc1ncc2c(n1)CCC(N(Cc1ccc3ccccc3c1)C(=O)CCl)C2] | (Compound 503) |
| 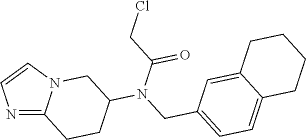 [SMILES: O=C(CCl)N(Cc1ccc2c(c1)CCCC2)C1CCc2nccn2C1] | (Compound 506) |
| 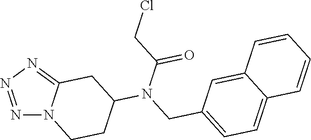 [SMILES: O=C(CCl)N(Cc1ccc2ccccc2c1)C1CCn2nnnc2C1] | (Compound 505) |
| 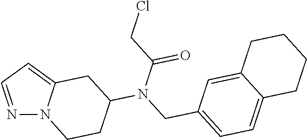 [SMILES: O=C(CCl)N(Cc1ccc2c(c1)CCCC2)C1CCn2nccc2C1] | (Compound 508) |
| 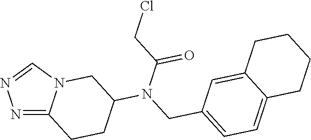 [SMILES: O=C(CCl)N(Cc1ccc2c(c1)CCCC2)C1CCc2nncn2C1] | (Compound 507) |
| 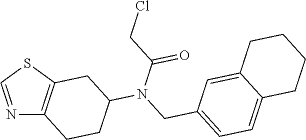 [SMILES: O=C(CCl)N(Cc1ccc2c(c1)CCCC2)C1CCc2ncsc2C1] | (Compound 510) |
| 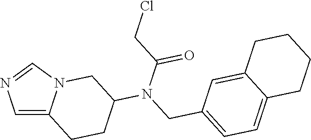 [SMILES: O=C(CCl)N(Cc1ccc2c(c1)CCCC2)C1CCc2cncn2C1] | (Compound 509) |
| 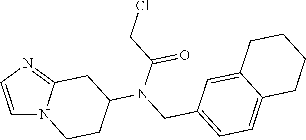 [SMILES: O=C(CCl)N(Cc1ccc2c(c1)CCCC2)C1CCn2ccnc2C1] | (Compound 512) |
| 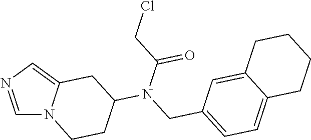 [SMILES: O=C(CCl)N(Cc1ccc2c(c1)CCCC2)C1CCn2cncc2C1] | (Compound 511) |
| 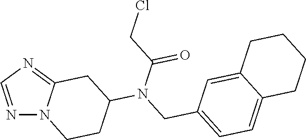 [SMILES: O=C(CCl)N(Cc1ccc2c(c1)CCCC2)C1CCn2ncnc2C1] | (Compound 514) |
| 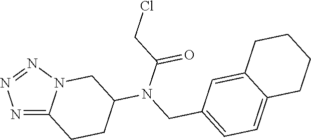 [SMILES: O=C(CCl)N(Cc1ccc2c(c1)CCCC2)C1CCc2nnnn2C1] | (Compound 513) |
| 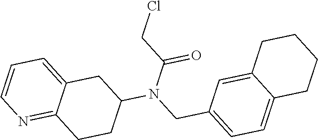 [SMILES: O=C(CCl)N(Cc1ccc2c(c1)CCCC2)C1CCc2ncccc2C1] | (Compound 516) |
| 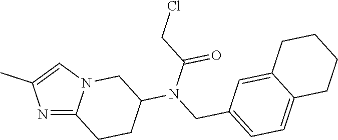 [SMILES: Cc1cn2c(n1)CCC(N(Cc1ccc3c(c1)CCCC3)C(=O)CCl)C2] | (Compound 515) |
| 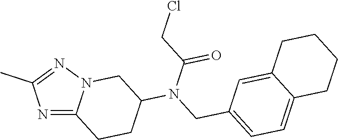 [SMILES: Cc1nc2n(n1)CC(N(Cc1ccc3c(c1)CCCC3)C(=O)CCl)CC2] | (Compound 518) |
| 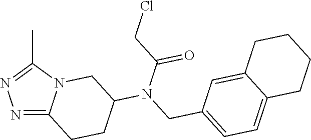 [SMILES: Cc1nnc2n1CC(N(Cc1ccc3c(c1)CCCC3)C(=O)CCl)CC2] | (Compound 517) |
| 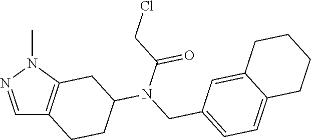 [SMILES: Cn1ncc2c1CC(N(Cc1ccc3c(c1)CCCC3)C(=O)CCl)CC2] | (Compound 520) |
| 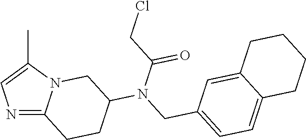 [SMILES: Cc1cnc2n1CC(N(Cc1ccc3c(c1)CCCC3)C(=O)CCl)CC2] | (Compound 519) |
| 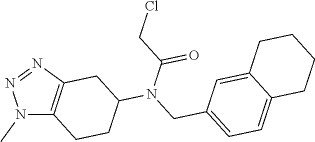 [SMILES: Cn1nnc2c1CCC(N(Cc1ccc3c(c1)CCCC3)C(=O)CCl)C2] | (Compound 522) |
| 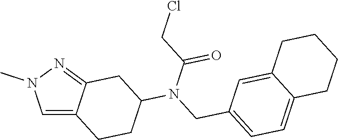 [SMILES: Cn1cc2c(n1)CC(N(Cc1ccc3c(c1)CCCC3)C(=O)CCl)CC2] | (Compound 521) |
| 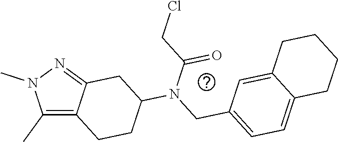 [SMILES: Cc1c2c(nn1C)CC(N(Cc1ccc3c(c1)CCCC3)C(=O)CCl)CC2] | (Compound 524) |
| 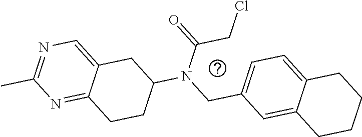 [SMILES: Cc1ncc2c(n1)CCC(N(Cc1ccc3c(c1)CCCC3)C(=O)CCl)C2] | (Compound 523) |
| 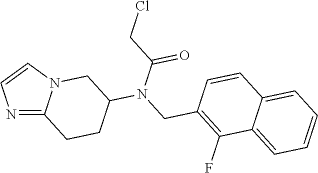 [SMILES: O=C(CCl)N(Cc1ccc2ccccc2c1F)C1CCc2nccn2C1] | (Compound 526) |
| 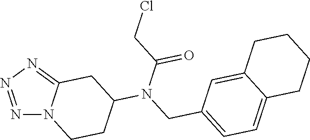 [SMILES: O=C(CCl)N(Cc1ccc2c(c1)CCCC2)C1CCn2nnnc2C1] | (Compound 525) |
| 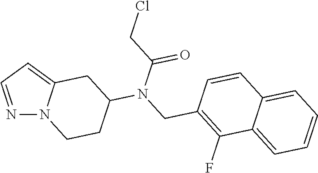 [SMILES: O=C(CCl)N(Cc1ccc2ccccc2c1F)C1CCn2nccc2C1] | (Compound 528) |
| 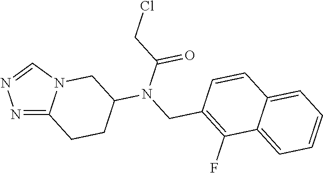 [SMILES: O=C(CCl)N(Cc1ccc2ccccc2c1F)C1CCc2nncn2C1] | (Compound 527) |
| 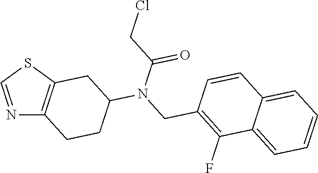 [SMILES: O=C(CCl)N(Cc1ccc2ccccc2c1F)C1CCc2ncsc2C1] | (Compound 530) |
| 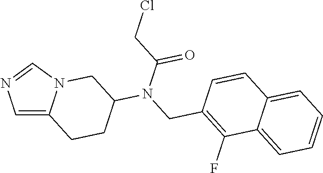 [SMILES: O=C(CCl)N(Cc1ccc2ccccc2c1F)C1CCc2cncn2C1] | (Compound 529) |
| 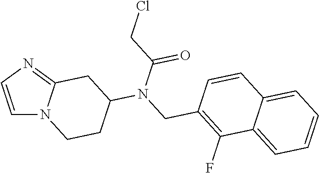 [SMILES: O=C(CCl)N(Cc1ccc2ccccc2c1F)C1CCn2ccnc2C1] | (Compound 532) |
| 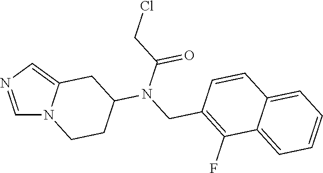 [SMILES: O=C(CCl)N(Cc1ccc2ccccc2c1F)C1CCn2cncc2C1] | (Compound 531) |
| 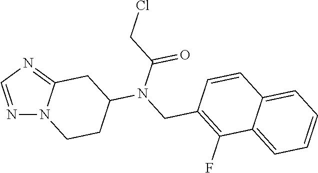 [SMILES: O=C(CCl)N(Cc1ccc2ccccc2c1F)C1CCn2ncnc2C1] | (Compound 534) |
| 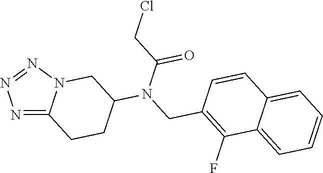 [SMILES: O=C(CCl)N(Cc1ccc2ccccc2c1F)C1CCc2nnnn2C1] | (Compound 533) |
| 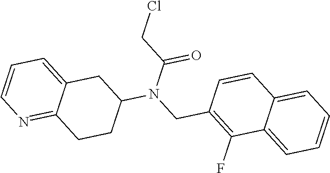 [SMILES: O=C(CCl)N(Cc1ccc2ccccc2c1F)C1CCc2ncccc2C1] | (Compound 536) |
| 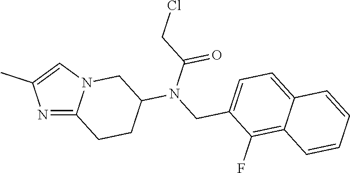 [SMILES: Cc1cn2c(n1)CCC(N(Cc1ccc3ccccc3c1F)C(=O)CCl)C2] | (Compound 535) |
| 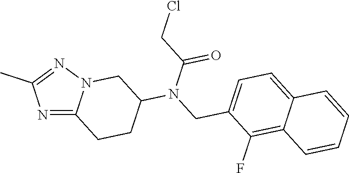 [SMILES: Cc1nc2n(n1)CC(N(Cc1ccc3ccccc3c1F)C(=O)CCl)CC2] | (Compound 538) |
| 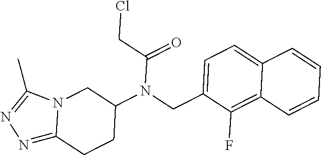 [SMILES: Cc1nnc2n1CC(N(Cc1ccc3ccccc3c1F)C(=O)CCl)CC2] | (Compound 537) |
| 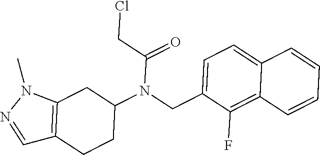 [SMILES: Cn1ncc2c1CC(N(Cc1ccc3ccccc3c1F)C(=O)CCl)CC2] | (Compound 540) |
| 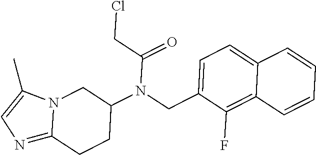 [SMILES: Cc1cnc2n1CC(N(Cc1ccc3ccccc3c1F)C(=O)CCl)CC2] | (Compound 539) |
| 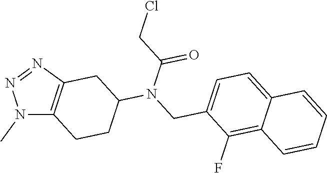 [SMILES: Cn1nnc2c1CCC(N(Cc1ccc3ccccc3c1F)C(=O)CCl)C2] | (Compound 542) |
| 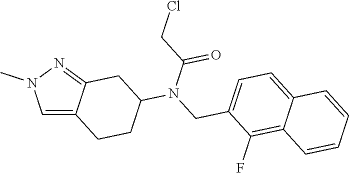 [SMILES: Cn1cc2c(n1)CC(N(Cc1ccc3ccccc3c1F)C(=O)CCl)CC2] | (Compound 541) |
| 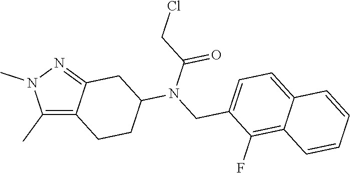 [SMILES: Cc1c2c(nn1C)CC(N(Cc1ccc3ccccc3c1F)C(=O)CCl)CC2] | (Compound 544) |
| 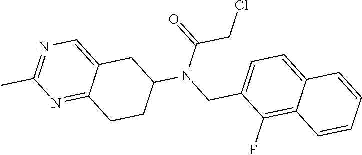 [SMILES: Cc1ncc2c(n1)CCC(N(Cc1ccc3ccccc3c1F)C(=O)CCl)C2] | (Compound 543) |
| 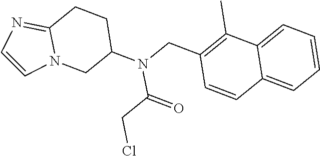 [SMILES: Cc1c(CN(C(=O)CCl)C2CCc3nccn3C2)ccc2ccccc12] | (Compound 546) |
| 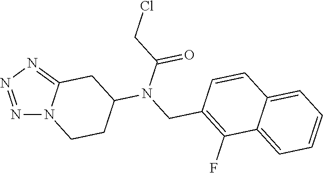 [SMILES: O=C(CCl)N(Cc1ccc2ccccc2c1F)C1CCn2nnnc2C1] | (Compound 545) |
| 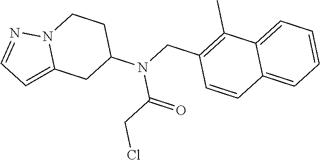 [SMILES: Cc1c(CN(C(=O)CCl)C2CCn3nccc3C2)ccc2ccccc12] | (Compound 548) |
| 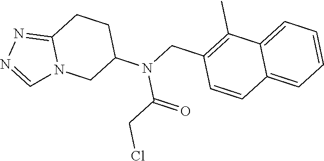 [SMILES: Cc1c(CN(C(=O)CCl)C2CCc3nncn3C2)ccc2ccccc12] | (Compound 547) |
| 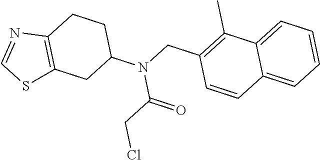 [SMILES: Cc1c(CN(C(=O)CCl)C2CCc3ncsc3C2)ccc2ccccc12] | (Compound 550) |
| 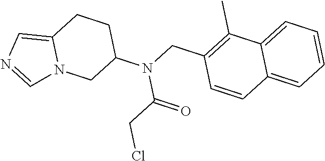 [SMILES: Cc1c(CN(C(=O)CCl)C2CCc3cncn3C2)ccc2ccccc12] | (Compound 549) |
| 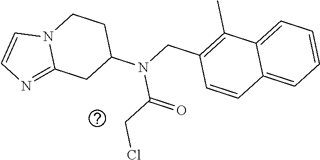 [SMILES: Cc1c(CN(C(=O)CCl)C2CCn3ccnc3C2)ccc2ccccc12] | (Compound 552) |
| 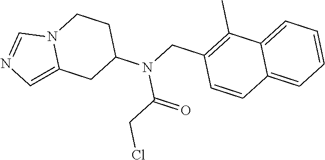 [SMILES: Cc1c(CN(C(=O)CCl)C2CCn3cncc3C2)ccc2ccccc12] | (Compound 551) |
| 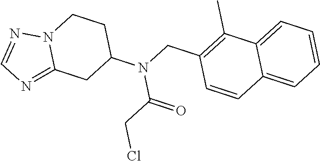 [SMILES: Cc1c(CN(C(=O)CCl)C2CCn3ncnc3C2)ccc2ccccc12] | (Compound 554) |
| 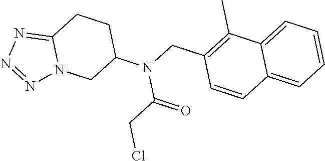 [SMILES: Cc1c(CN(C(=O)CCl)C2CCc3nnnn3C2)ccc2ccccc12] | (Compound 553) |
| 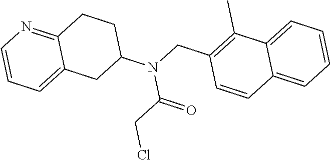 [SMILES: Cc1c(CN(C(=O)CCl)C2CCc3ncccc3C2)ccc2ccccc12] | (Forumla 556) |
| 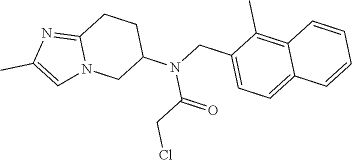 [SMILES: Cc1cn2c(n1)CCC(N(Cc1ccc3ccccc3c1C)C(=O)CCl)C2] | (Compound 555) |
| 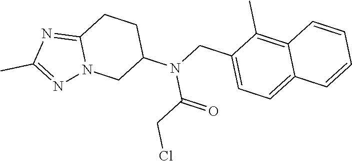 [SMILES: Cc1nc2n(n1)CC(N(Cc1ccc3ccccc3c1C)C(=O)CCl)CC2] | (Compound 558) |
| 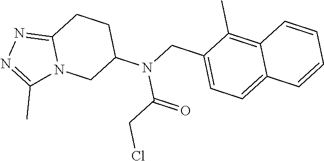 [SMILES: Cc1c(CN(C(=O)CCl)C2CCc3nnc(C)n3C2)ccc2ccccc12] | (Compound 557) |
| 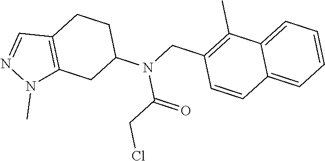 [SMILES: Cc1c(CN(C(=O)CCl)C2CCc3cnn(C)c3C2)ccc2ccccc12] | (Compound 560) |
| 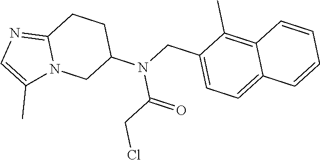 [SMILES: Cc1c(CN(C(=O)CCl)C2CCc3ncc(C)n3C2)ccc2ccccc12] | (Compound 559) |
| 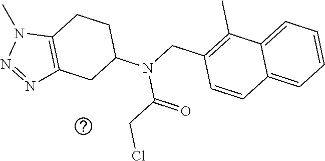 [SMILES: Cc1c(CN(C(=O)CCl)C2CCc3c(nnn3C)C2)ccc2ccccc12] | (Compound 562) |
| 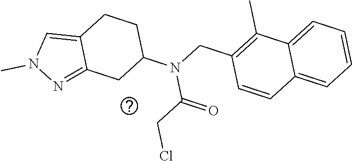 [SMILES: Cc1c(CN(C(=O)CCl)C2CCc3cn(C)nc3C2)ccc2ccccc12] | (Compound 561) |
| 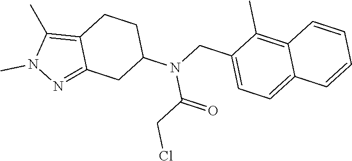 [SMILES: Cc1c(CN(C(=O)CCl)C2CCc3c(nn(C)c3C)C2)ccc2ccccc12] | (Compound 564) |
| 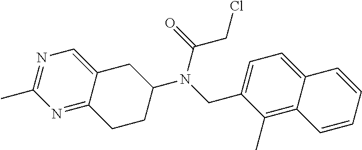 [SMILES: Cc1ncc2c(n1)CCC(N(Cc1ccc3ccccc3c1C)C(=O)CCl)C2] | (Compound 563) |
| 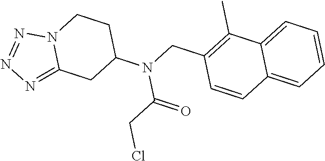 [SMILES: Cc1c(CN(C(=O)CCl)C2CCn3nnnc3C2)ccc2ccccc12] | (Compound 565) |
| 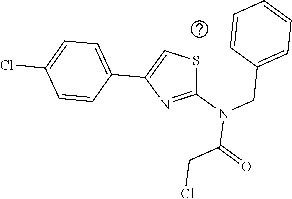 [SMILES: O=C(CCl)N(Cc1ccccc1)c1nc(-c2ccc(Cl)cc2)cs1] | (Compound 566) |
| 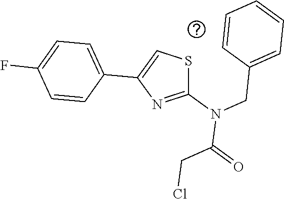 [SMILES: O=C(CCl)N(Cc1ccccc1)c1nc(-c2ccc(F)cc2)cs1] | (Compound 567) |
| 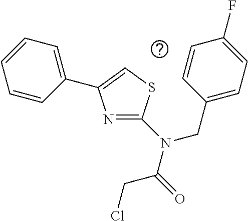 [SMILES: O=C(CCl)N(Cc1ccc(F)cc1)c1nc(-c2ccccc2)cs1] | (Compound 568) |
| 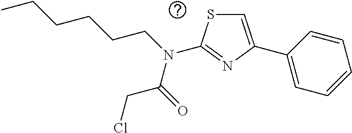 [SMILES: CCCCCCN(C(=O)CCl)c1nc(-c2ccccc2)cs1] | (Compound 569) |
| 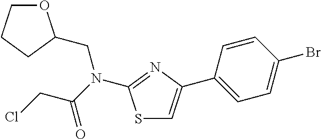 [SMILES: O=C(CCl)N(CC1CCCO1)c1nc(-c2ccc(Br)cc2)cs1] | (Compound 570) |
| 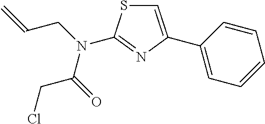 [SMILES: C=CCN(C(=O)CCl)c1nc(-c2ccccc2)cs1] | (Compound 571) |
| 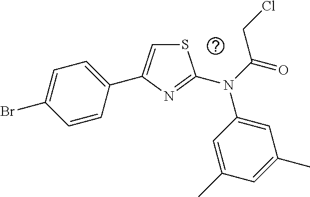 [SMILES: Cc1cc(C)cc(N(C(=O)CCl)c2nc(-c3ccc(Br)cc3)cs2)c1] | (Compound 572) |
| 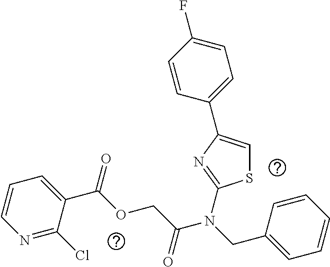 [SMILES: O=C(OCC(=O)N(Cc1ccccc1)c1nc(-c2ccc(F)cc2)cs1)c1cccnc1Cl] | (Compound 573) |
| 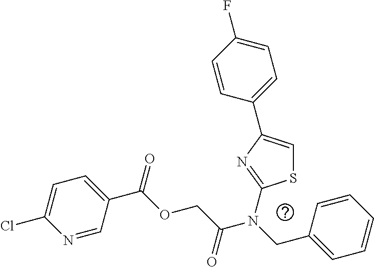 [SMILES: O=C(OCC(=O)N(Cc1ccccc1)c1nc(-c2ccc(F)cc2)cs1)c1ccc(Cl)nc1] | (Compound 574) |
| 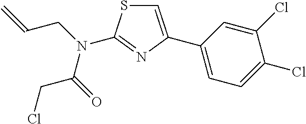 [SMILES: C=CCN(C(=O)CCl)c1nc(-c2ccc(Cl)c(Cl)c2)cs1] | (Compound 575) |
| 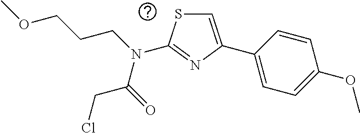 | (Compound 576) |
| 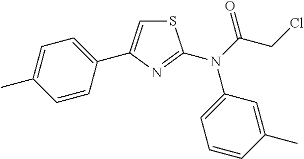 | (Compound 577) |
| 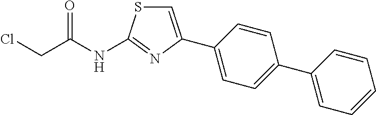 [SMILES: O=C(CCl)Nc1nc(-c2ccc(-c3ccccc3)cc2)cs1] | (Compound 578) |
| 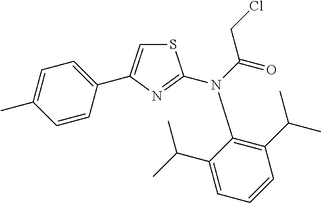 [SMILES: Cc1ccc(-c2csc(N(C(=O)CCl)c3c(C(C)C)cccc3C(C)C)n2)cc1] | (Compound 579) |
| 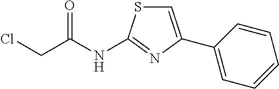 [SMILES: O=C(CCl)Nc1nc(-c2ccccc2)cs1] | (Compound 580) |
| 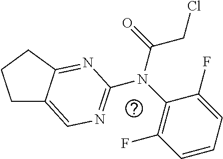 [SMILES: O=C(CCl)N(c1ncc2c(n1)CCC2)c1c(F)cccc1F] | (Compound 581) |
| 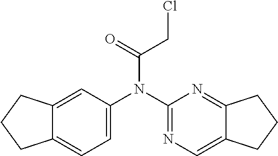 [SMILES: O=C(CCl)N(c1ccc2c(c1)CCC2)c1ncc2c(n1)CCC2] | (Compound 582) |
| 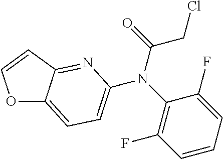 [SMILES: O=C(CCl)N(c1ccc2occc2n1)c1c(F)cccc1F] | (Compound 583) |
| 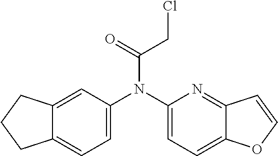 [SMILES: O=C(CCl)N(c1ccc2c(c1)CCC2)c1ccc2occc2n1] | (Compound 584) |
| 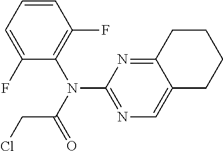 [SMILES: O=C(CCl)N(c1ncc2c(n1)CCCC2)c1c(F)cccc1F] | (Compound 585) |
| 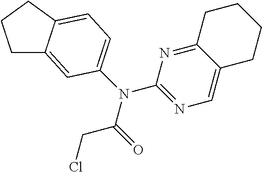 [SMILES: O=C(CCl)N(c1ccc2c(c1)CCC2)c1ncc2c(n1)CCCC2] | (Compound 586) |
| 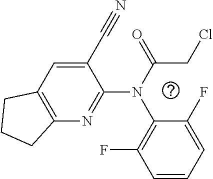 [SMILES: N#Cc1cc2c(nc1N(C(=O)CCl)c1c(F)cccc1F)CCC2] | (Compound 587) |
| 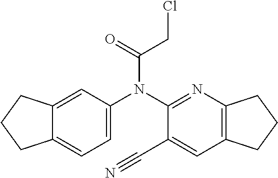 [SMILES: N#Cc1cc2c(nc1N(C(=O)CCl)c1ccc3c(c1)CCC3)CCC2] | (Compound 588) |
| 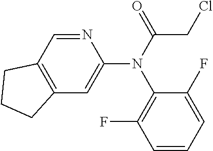 [SMILES: O=C(CCl)N(c1cc2c(cn1)CCC2)c1c(F)cccc1F] | (Compound 589) |
| 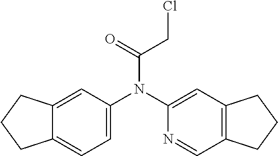 [SMILES: O=C(CCl)N(c1ccc2c(c1)CCC2)c1cc2c(cn1)CCC2] | (Compound 590) |
| 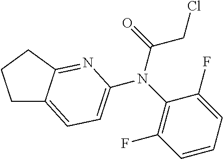 [SMILES: O=C(CCl)N(c1ccc2c(n1)CCC2)c1c(F)cccc1F] | (Compound 591) |
| 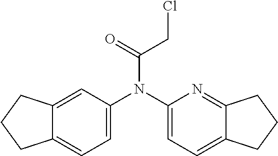 [SMILES: O=C(CCl)N(c1ccc2c(c1)CCC2)c1ccc2c(n1)CCC2] | (Compound 592) |
| 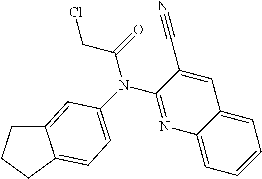 [SMILES: N#Cc1cc2ccccc2nc1N(C(=O)CCl)c1ccc2c(c1)CCC2] | (Compound 593) |
| 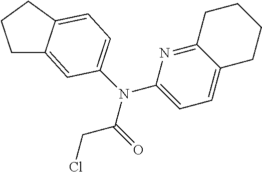 [SMILES: O=C(CCl)N(c1ccc2c(c1)CCC2)c1ccc2c(n1)CCCC2] | (Compound 594) |
| 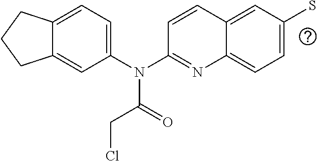 [SMILES: O=C(CCl)N(c1ccc2c(c1)CCC2)c1ccc2cc(S)ccc2n1] | (Compound 595) |
| 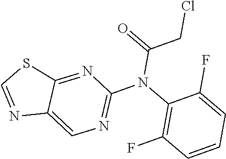 [SMILES: O=C(CCl)N(c1ncc2ncsc2n1)c1c(F)cccc1F] | (Compound 596) |
| 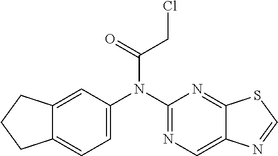 [SMILES: O=C(CCl)N(c1ccc2c(c1)CCC2)c1ncc2ncsc2n1] | (Compound 597) |
| 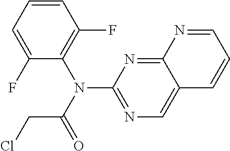 [SMILES: O=C(CCl)N(c1ncc2cccnc2n1)c1c(F)cccc1F] | (Compound 598) |
| 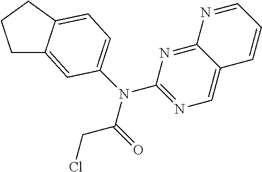 [SMILES: O=C(CCl)N(c1ccc2c(c1)CCC2)c1ncc2cccnc2n1] | (Compound 599) |
| 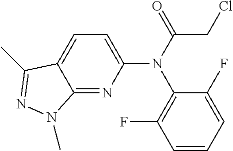 [SMILES: Cc1nn(C)c2nc(N(C(=O)CCl)c3c(F)cccc3F)ccc12] | (Compound 600) |
| 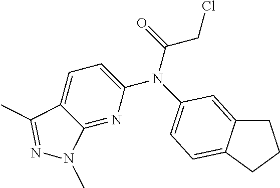 [SMILES: Cc1nn(C)c2nc(N(C(=O)CCl)c3ccc4c(c3)CCC4)ccc12] | (Compound 601) |
| 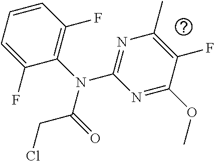 [SMILES: COc1nc(N(C(=O)CCl)c2c(F)cccc2F)nc(C)c1F] | (Compound 602) |
| 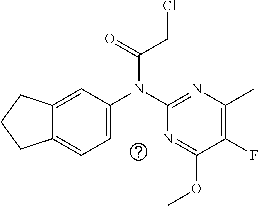 [SMILES: COc1nc(N(C(=O)CCl)c2ccc3c(c2)CCC3)nc(C)c1F] | (Compound 603) |
| 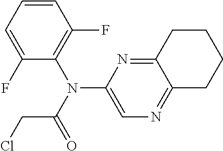 [SMILES: O=C(CCl)N(c1cnc2c(n1)CCCC2)c1c(F)cccc1F] | (Compound 604) |
| 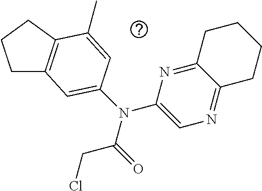 [SMILES: Cc1cc(N(C(=O)CCl)c2cnc3c(n2)CCCC3)cc2c1CCC2] | (Compound 605) |
| 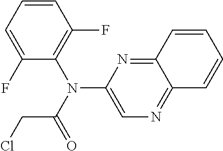 [SMILES: O=C(CCl)N(c1cnc2ccccc2n1)c1c(F)cccc1F] | (Compound 606) |
| 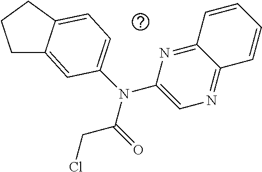 [SMILES: O=C(CCl)N(c1ccc2c(c1)CCC2)c1cnc2ccccc2n1] | (Compound 607) |
| 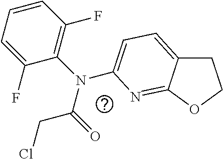 [SMILES: O=C(CCl)N(c1ccc2c(n1)OCC2)c1c(F)cccc1F] | (Compound 608) |
| 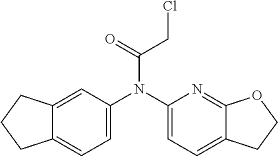 [SMILES: O=C(CCl)N(c1ccc2c(c1)CCC2)c1ccc2c(n1)OCC2] | (Compound 609) |
| 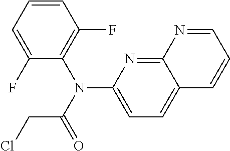 [SMILES: O=C(CCl)N(c1ccc2cccnc2n1)c1c(F)cccc1F] | (Compound 610) |
| 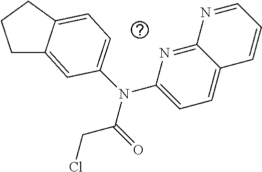 [SMILES: O=C(CCl)N(c1ccc2c(c1)CCC2)c1ccc2cccnc2n1] | (Compound 611) |
| 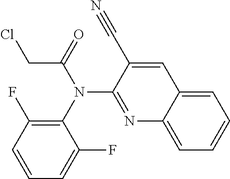 [SMILES: N#Cc1cc2ccccc2nc1N(C(=O)CCl)c1c(F)cccc1F] | (Compound 612) |
| 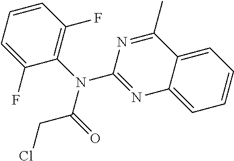 [SMILES: Cc1nc(N(C(=O)CCl)c2c(F)cccc2F)nc2ccccc12] | (Compound 613) |
| 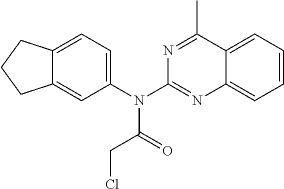 [SMILES: Cc1nc(N(C(=O)CCl)c2ccc3c(c2)CCC3)nc2ccccc12] | (Compound 614) |
| 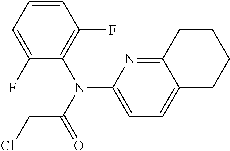 [SMILES: O=C(CCl)N(c1ccc2c(n1)CCCC2)c1c(F)cccc1F] | (Compound 615) |
| 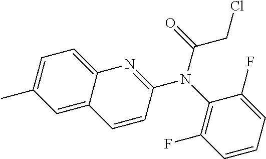 [SMILES: Cc1ccc2nc(N(C(=O)CCl)c3c(F)cccc3F)ccc2c1] | (Compound 616) |
| 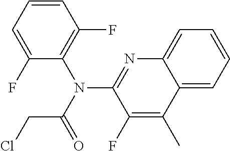 [SMILES: Cc1c(F)c(N(C(=O)CCl)c2c(F)cccc2F)nc2ccccc12] | (Compound 617) |
| 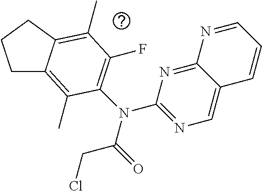 [SMILES: Cc1c(F)c(N(C(=O)CCl)c2ncc3cccnc3n2)c(C)c2c1CCC2] | (Compound 618) |
| 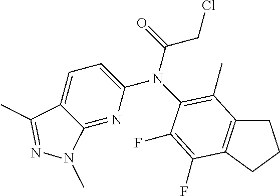 [SMILES: Cc1c2c(c(F)c(F)c1N(C(=O)CCl)c1ccc3c(C)nn(C)c3n1)CCC2] | (Compound 619) |
| 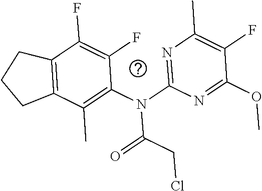 [SMILES: COc1nc(N(C(=O)CCl)c2c(C)c3c(c(F)c2F)CCC3)nc(C)c1F] | (Compound 620) |
| 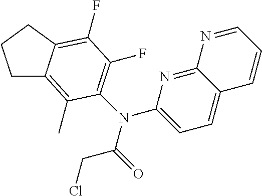 [SMILES: Cc1c2c(c(F)c(F)c1N(C(=O)CCl)c1ccc3cccnc3n1)CCC2] | (Compound 621) |
| 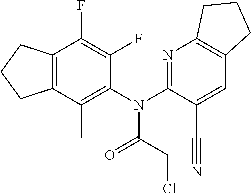 [SMILES: Cc1c2c(c(F)c(F)c1N(C(=O)CCl)c1nc3c(cc1C#N)CCC3)CCC2] | (Compound 622) |
| 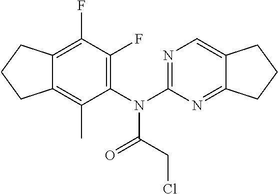 [SMILES: Cc1c2c(c(F)c(F)c1N(C(=O)CCl)c1ncc3c(n1)CCC3)CCC2] | (Compound 623) |
| 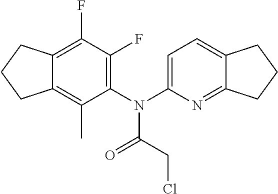 [SMILES: Cc1c2c(c(F)c(F)c1N(C(=O)CCl)c1ccc3c(n1)CCC3)CCC2] | (Compound 624) |
| 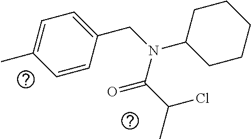 [SMILES: Cc1ccc(CN(C(=O)C(C)Cl)C2CCCCC2)cc1] | (Compound 625) |
| 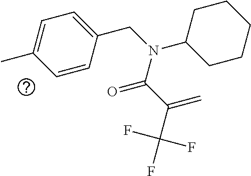 [SMILES: C=C(C(=O)N(Cc1ccc(C)cc1)C1CCCCC1)C(F)(F)F] | (Compound 626) |
| 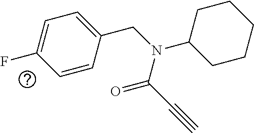 [SMILES: C#CC(=O)N(Cc1ccc(F)cc1)C1CCCCC1] | (Compound 627) |
| 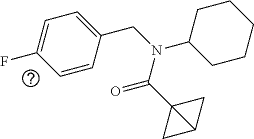 [SMILES: O=C(N(Cc1ccc(F)cc1)C1CCCCC1)C12CC1C2] | (Compound 628) |
| 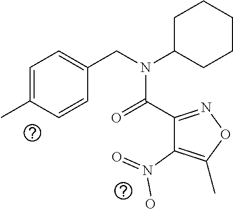 | (Compound 629) |
| 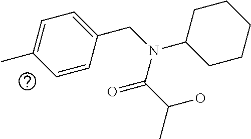 [SMILES: Cc1ccc(CN(C(=O)C(C)O)C2CCCCC2)cc1] | (Compound 630) |
| 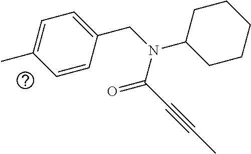 [SMILES: CC#CC(=O)N(Cc1ccc(C)cc1)C1CCCCC1] | (Compound 631) |
| 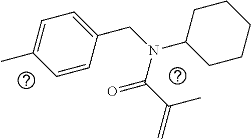 [SMILES: C=C(C)C(=O)N(Cc1ccc(C)cc1)C1CCCCC1] | (Compound 632) |
| 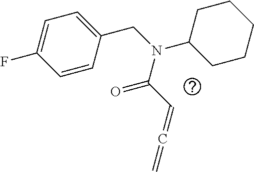 [SMILES: C=C=CC(=O)N(Cc1ccc(F)cc1)C1CCCCC1] | (Compound 633) |
| 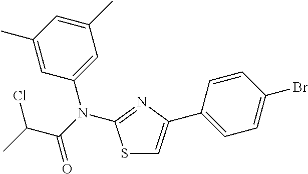 [SMILES: Cc1cc(C)cc(N(C(=O)C(C)Cl)c2nc(-c3ccc(Br)cc3)cs2)c1] | (Compound 634) |
| 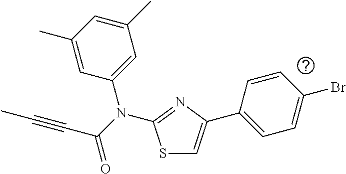 [SMILES: CC#CC(=O)N(c1cc(C)cc(C)c1)c1nc(-c2ccc(Br)cc2)cs1] | (Compound 635) |
| 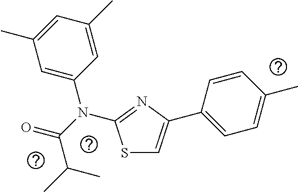 | (Compound 636) |
| 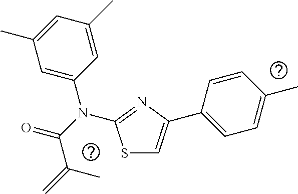 | (Compound 637) |
| 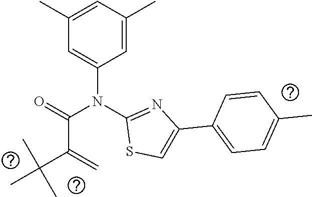 [SMILES: C=C(C(=O)N(c1cc(C)cc(C)c1)c1nc(-c2ccc(C)cc2)cs1)C(C)(C)C] | (Compound 638) |
| 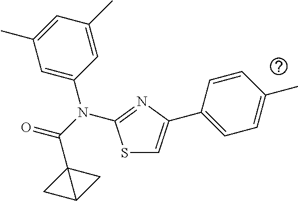 | (Compound 639) |
| 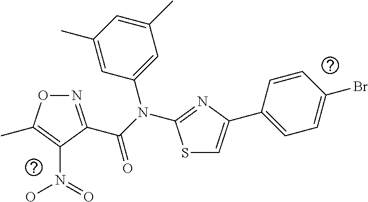 | (Compound 640) |
| 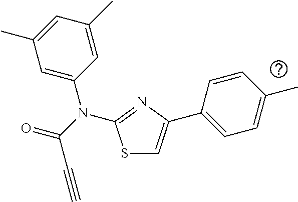 | (Compound 641) |
| 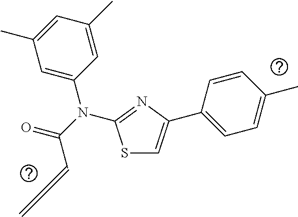 | (Compound 642) |

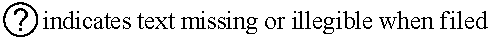

54. The method of claim 44, wherein the compound is selected from the group consisting of:

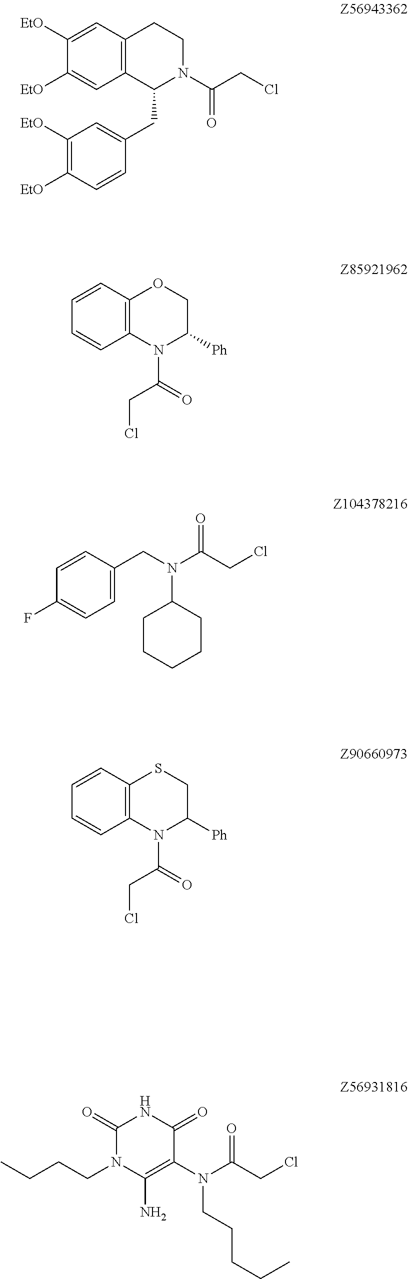 [SMILES: CCOc1ccc(C[C@@H]2c3cc(OCC)c(OCC)cc3CCN2C(=O)CCl)cc1OCC ; O=C(CCl)N1c2ccccc2OC[C@@H]1c1ccccc1 ; O=C(CCl)N(Cc1ccc(F)cc1)C1CCCCC1 ; O=C(CCl)N1c2ccccc2SCC1c1ccccc1 ; CCCCCN(C(=O)CCl)c1c(N)n(CCCC)c(=O)[nH]c1=O]

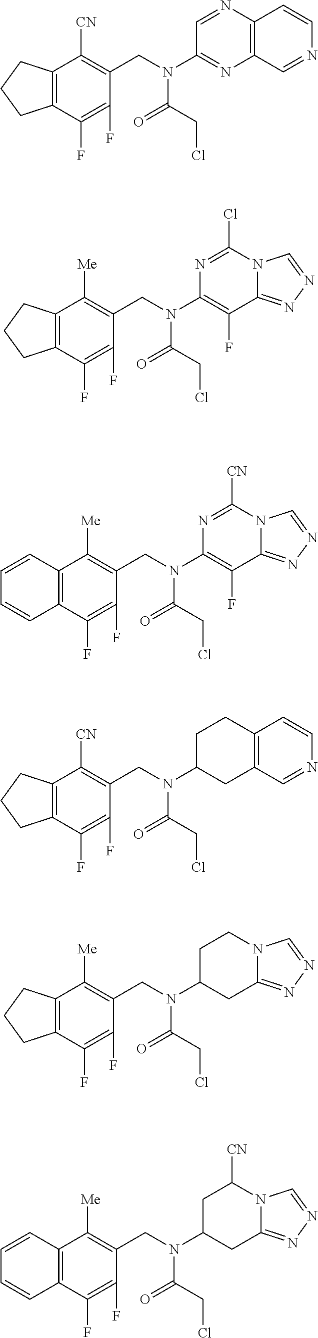 [SMILES: N#Cc1c(CN(C(=O)CCl)c2cnc3ccncc3n2)c(F)c(F)c2c1CCC2 ; Cc1c(CN(C(=O)CCl)c2nc(Cl)n3cnnc3c2F)c(F)c(F)c2c1CCC2 ; Cc1c(CN(C(=O)CCl)c2nc(C#N)n3cnnc3c2F)c(F)c(F)c2ccccc12 ; N#Cc1c(CN(C(=O)CCl)C2CCc3ccncc3C2)c(F)c(F)c2c1CCC2 ; Cc1c(CN(C(=O)CCl)C2CCn3cnnc3C2)c(F)c(F)c2c1CCC2 ; Cc1c(CN(C(=O)CCl)C2Cc3nncn3C(C#N)C2)c(F)c(F)c2ccccc12]

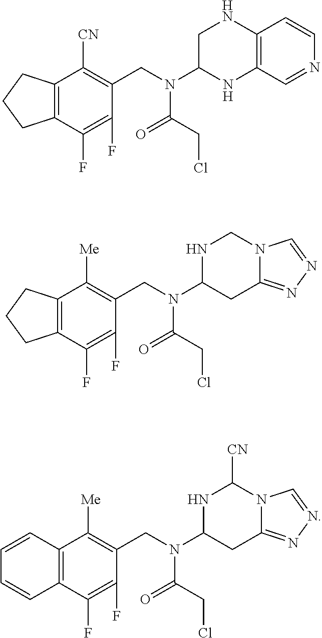 [SMILES: N#Cc1c(CN(C(=O)CCl)C2CNc3ccncc3N2)c(F)c(F)c2c1CCC2 ; Cc1c(CN(C(=O)CCl)C2Cc3nncn3CN2)c(F)c(F)c2c1CCC2 ; Cc1c(CN(C(=O)CCl)C2Cc3nncn3C(C#N)N2)c(F)c(F)c2ccccc12]

55. A method for inhibiting GPX4 enzyme, the method comprising contacting the GPX4 enzyme with a compound selected from the group:

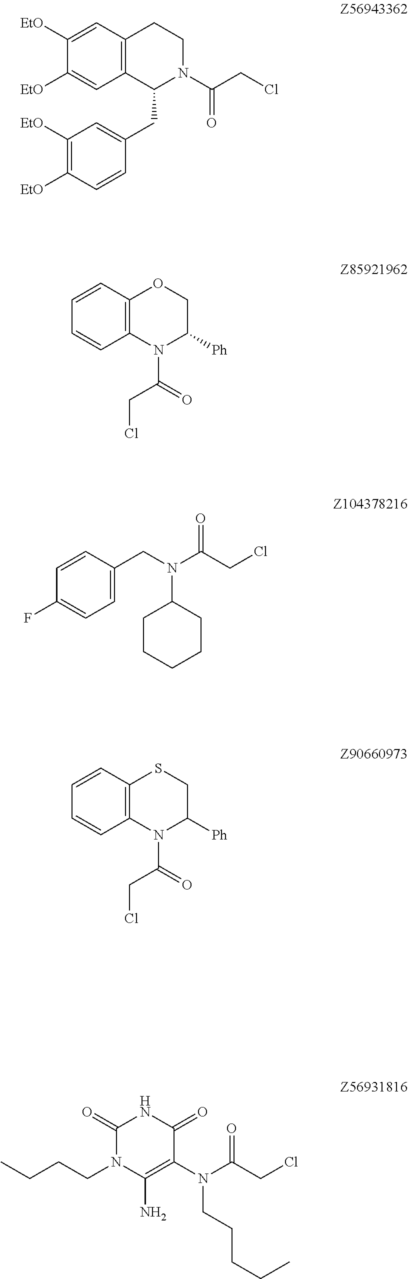 [SMILES: CCOc1ccc(C[C@@H]2c3cc(OCC)c(OCC)cc3CCN2C(=O)CCl)cc1OCC ; O=C(CCl)N1c2ccccc2OC[C@@H]1c1ccccc1 ; O=C(CCl)N(Cc1ccc(F)cc1)C1CCCCC1 ; O=C(CCl)N1c2ccccc2SCC1c1ccccc1 ; CCCCCN(C(=O)CCl)c1c(N)n(CCCC)c(=O)[nH]c1=O]

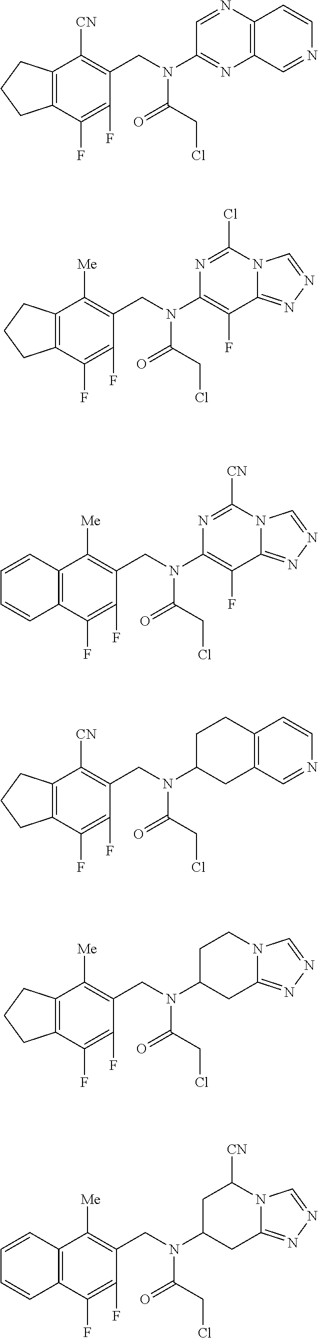 [SMILES: N#Cc1c(CN(C(=O)CCl)c2cnc3ccncc3n2)c(F)c(F)c2c1CCC2 ; Cc1c(CN(C(=O)CCl)c2nc(Cl)n3cnnc3c2F)c(F)c(F)c2c1CCC2 ; Cc1c(CN(C(=O)CCl)c2nc(C#N)n3cnnc3c2F)c(F)c(F)c2ccccc12 ; N#Cc1c(CN(C(=O)CCl)C2CCc3ccncc3C2)c(F)c(F)c2c1CCC2 ; Cc1c(CN(C(=O)CCl)C2CCn3cnnc3C2)c(F)c(F)c2c1CCC2 ; Cc1c(CN(C(=O)CCl)C2Cc3nncn3C(C#N)C2)c(F)c(F)c2ccccc12]

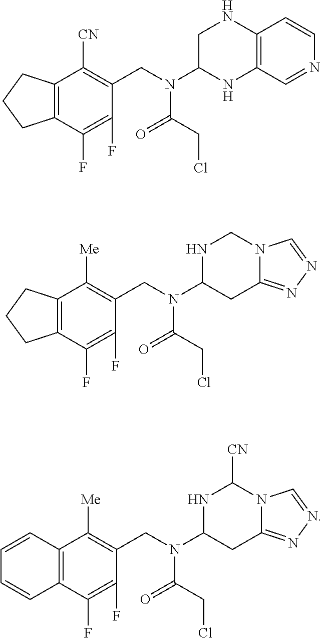 [SMILES: N#Cc1c(CN(C(=O)CCl)C2CNc3ccncc3N2)c(F)c(F)c2c1CCC2 ; Cc1c(CN(C(=O)CCl)C2Cc3nncn3CN2)c(F)c(F)c2c1CCC2 ; Cc1c(CN(C(=O)CCl)C2Cc3nncn3C(C#N)N2)c(F)c(F)c2ccccc12]

56. A compound, or a pharmaceutically acceptable salt thereof, having a structure selected from the group:

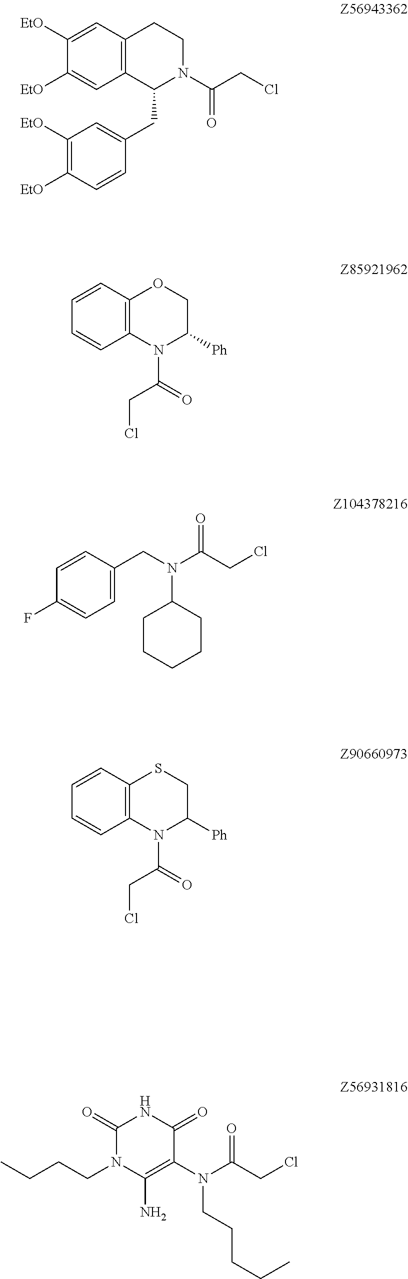 [SMILES: CCOc1ccc(C[C@@H]2c3cc(OCC)c(OCC)cc3CCN2C(=O)CCl)cc1OCC ; O=C(CCl)N1c2ccccc2OC[C@@H]1c1ccccc1 ; O=C(CCl)N(Cc1ccc(F)cc1)C1CCCCC1 ; O=C(CCl)N1c2ccccc2SCC1c1ccccc1 ; CCCCCN(C(=O)CCl)c1c(N)n(CCCC)c(=O)[nH]c1=O]

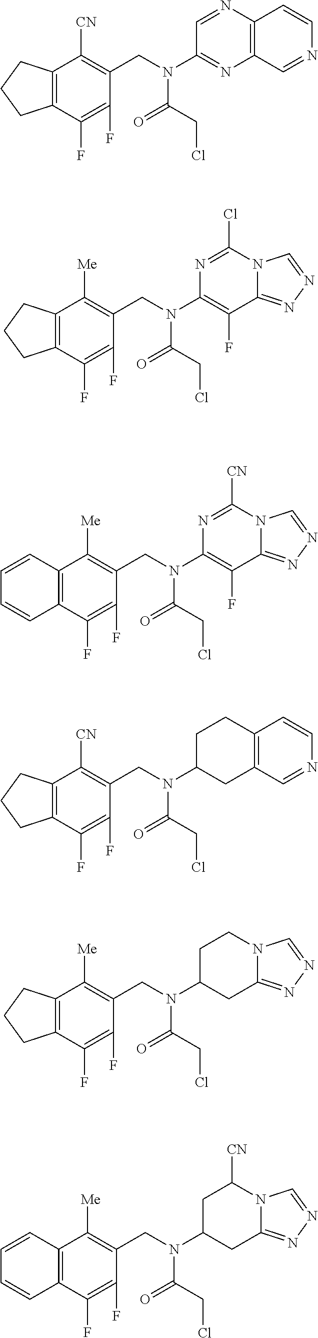 [SMILES: N#Cc1c(CN(C(=O)CCl)c2cnc3ccncc3n2)c(F)c(F)c2c1CCC2 ; Cc1c(CN(C(=O)CCl)c2nc(Cl)n3cnnc3c2F)c(F)c(F)c2c1CCC2 ; Cc1c(CN(C(=O)CCl)c2nc(C#N)n3cnnc3c2F)c(F)c(F)c2ccccc12 ; N#Cc1c(CN(C(=O)CCl)C2CCc3ccncc3C2)c(F)c(F)c2c1CCC2 ; Cc1c(CN(C(=O)CCl)C2CCn3cnnc3C2)c(F)c(F)c2c1CCC2 ; Cc1c(CN(C(=O)CCl)C2Cc3nncn3C(C#N)C2)c(F)c(F)c2ccccc12]

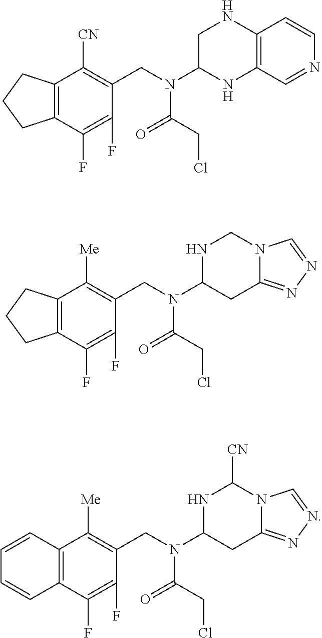 [SMILES: N#Cc1c(CN(C(=O)CCl)C2CNc3ccncc3N2)c(F)c(F)c2c1CCC2 ; Cc1c(CN(C(=O)CCl)C2Cc3nncn3CN2)c(F)c(F)c2c1CCC2 ; Cc1c(CN(C(=O)CCl)C2Cc3nncn3C(C#N)N2)c(F)c(F)c2ccccc12]

57. A pharmaceutical composition comprising a compound, or a pharmaceutically acceptable salt thereof of claim 56.
